(19) United States
(12) Patent Application Publication
(10) Pub. No.: US 2005/0032873 A1
(43) Pub. Date: Feb. 10, 2005

(54) 3-Amino chroman and 2-amino tetralin derivatives

(75) Inventors: Nicole Theriault Hatzenbuhler, Bridgewater, NJ (US); Deborah Ann Evrard, Hamilton Square, NJ (US); Richard Eric Mewshaw, King of Prussia, PA (US); Dahui Zhou, East Brunswick, NJ (US); Uresh Shantilal Shah, Cranbury, NJ (US); Jennifer Ann Inghrim, Hellertown, PA (US); Steven Edward Lenicek, Plainsboro, NJ (US); Reinhardt Bernhard Baudy, Buckingham, PA (US); John Butera, Clarksburg, NJ (US); Annmarie L. Sabb, Pennington, NJ (US); Amedeo Arturo Failli, Princeton Junction, NJ (US); Pudukkaraipudur Sivaramakrishnan Ramamoorthy, Plainsboro, NJ (US)

Correspondence Address: WOODCOCK WASHBURN LLP, ONE LIBERTY PLACE - 46TH FLOOR, PHILADELPHIA, PA, 19103, US

(73) Assignee: Wyeth, Madison, NJ (US)

(21) Appl. No.: 10/898,866

(22) Filed: Jul. 26, 2004

Related U.S. Application Data
(60) Provisional application No. 60/491,137, filed on Jul. 30, 2003.
(60) Provisional application No. 60/491,794, filed on Aug. 1, 2003.

(51) Int. Cl. A61K 31/405; A61K 31/381; A61K 31/353; C07D 49/02; C07D 45/02

(52) U.S. Cl. 514/414; 514/443; 514/456; 514/469; 548/454; 549/403; 549/49

(57) ABSTRACT

3-Amino chroman and 2-amino tetralin derivatives and compositions containing such compounds are disclosed. Methods of using the 3-amino chroman and 2-amino tetralin compounds and compositions containing such compounds in the treatment of serotonin disorders, such as depression and anxiety, are also disclosed.

### CROSS REFERENCE TO RELATED APPLICATIONS

The present application claims benefit of Provisional Application Ser. No. 60/491,137 filed Jul. 30, 2003 and Provisional Application Ser. No. 60/491,794 filed Aug. 1, 2003, the complete disclosures of which are hereby incorporated by reference in their entirety.

### FIELD OF THE INVENTION

The present invention relates to novel 3-amino chroman and 2-amino tetralin derivatives, and in particular, to their activity as both serotonin reuptake inhibitors and as 5-HT_{1A} receptor agonists or antagonists, and to their related use for, inter alia, the treatment/and or prevention of depression and other conditions related to or affected by the reuptake of serotonin and the 5-HT_{1A} receptor.

### BACKGROUND OF THE INVENTION

Major depressive disorder affects an estimated 340 million people worldwide. Depression is the most frequently diagnosed psychiatric disorder and, according to the World Health Organization, is the fourth greatest public health problem. If left untreated, the effects of depression can be devastating, robbing people of the energy or motivation to perform everyday activities and, in some cases, leading to suicide. Symptoms of the disorder include feelings of sadness or emptiness, lack of interest or pleasure in nearly all activities, and feelings of worthlessness or inappropriate guilt. In addition to the personal costs of depression, the disorder also has been estimated to result in more than $40 billion in annual costs in the United States alone, due to premature death, lost productivity, and absenteeism.

Selective serotonin reuptake inhibitors (SSRIs) have had significant success in treating depression and related illnesses and have become among the most prescribed drugs since the 1980s. Some of the most widely known SSRIs are fluoxetine, sertraline, paroxetine, fluvoxamine and citalopram. Although they have a favorable side effect profile compared to tricyclic antidepressants (TCAs), they have their own particular set of side effects due to the non-selective stimulation of serotonergic sites. They typically have a slow onset of action, often taking several weeks to produce their full therapeutic effect. Furthermore, they have generally been found to be effective in less than two-thirds of patients.

SSRIs are believed to work by blocking the neuronal reuptake of serotonin, increasing the concentration of serotonin in the synaptic space, and thus increasing the activation of postsynaptic serotonin receptors. Although a single dose of a SSRI can inhibit the neuronal serotonin transporter, and thus would be expected to increase synaptic serotonin, clinical improvement has generally been observed only after long-term treatment. It has been suggested that the delay in onset of antidepressant action of the SSRIs is the result of an increase in serotonin levels in the vicinity of the serotonergic cell bodies. This excess serotonin is believed to activate somatodendritic autoreceptors, i.e., 5-HT_{1A} receptors, reduce cell firing activity and, in turn, decrease serotonin release in major forebrain areas. This negative feedback limits the increment of synaptic serotonin that can be induced by antidepressants acutely. Over time, the somatodendritic autoreceptors become desensitized, allowing the full effect of the SSRIs to be expressed in the forebrain. This time period has been found to correspond to the latency for the onset of antidepressant activity [Perez, V., et al., The Lancet, 1997, 349: 1594-1597].

In contrast to the SSRIs, a 5-HT_{1A} agonist or partial agonist acts directly on postsynaptic serotonin receptors to increase serotonergic neurotransmission during the latency period for the SSRI effect. Accordingly, the 5-HT_{1A} partial agonists, buspirone and gepirone [Feiger, A., Psychopharmacol. Bull., 1996, 32(4): 659-665; Wilcox, C., Psychopharmacol. Bull., 1996, 32(93): 335-342], and the 5-HT_{1A} agonist, flesinoxan [Grof, P., International Clinical Psychopharmacology, 1993, 8(3): 167-172], have shown efficacy in clinical trials for the treatment of depression. Furthermore, such agents are believed to stimulate the somatodendritic autoreceptors, thus hastening their desensitization and decreasing the SSRI latency period. An agent with a dual mechanism of antidepressant action would be expected to have greater efficacy and thus reduce the number of patients refractory to treatment. Indeed, buspirone augmentation to standard SSRI therapy has been shown to produce marked clinical improvement in patients initially unresponsive to standard antidepressant therapy [Dimitriou, E., J. Clinical Psychopharmacol., 1998, 18(6): 465-469].

There is still an unfilled need for a single agent with a dual mechanism of antidepressant action, i.e., one that not only inhibits or blocks serotonin reuptake (to increase levels of serotonin in the synapse) but also antagonizes the 5-HT_{1A} receptors (to reduce the latency period). The present invention is directed to these, as well as other important ends.

### SUMMARY OF THE INVENTION

This invention relates to 3-amino chroman and 2-amino tetralin derivatives, and in particular, to methods of their use in the treatment and/or prevention of serotonin-related disorders, such as depression (including, but not limited to major depressive disorder, childhood depression and dysthymia), anxiety, panic disorder, post-traumatic stress disorder, premenstrual dysphoric disorder (also known as premenstrual syndrome), attention deficit disorder (with or without hyperactivity), obsessive-compulsive disorder, social anxiety disorder, generalized anxiety disorder, obesity, eating disorders such as anorexia nervosa and bulimia nervosa, vasomotor flushing, cocaine and alcohol addiction, sexual dysfunction, cognitive deficits resulting from neurodegenerative disorders like Alzheimer's disease, and related illnesses. Preferred compounds have the ability to bind 5-HT_{1A} receptors, act as agonists, partial agonists or antagonists at the 5-HT_{1A} receptors, and act as serotonin reuptake inhibitors.

In one aspect, the present invention provides 3-amino chroman and 2-amino tetralin derivatives having formula I: 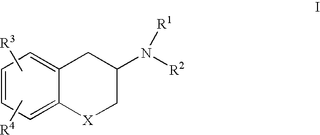
or a prodrug, stereoisomer or pharmaceutically-acceptable salt thereof;
wherein:
X is O or CH₂;
R¹ is hydrogen, alkyl, cycloalkyl or oxetane;
R² is —(CH₂)ₐ—R⁵, M, 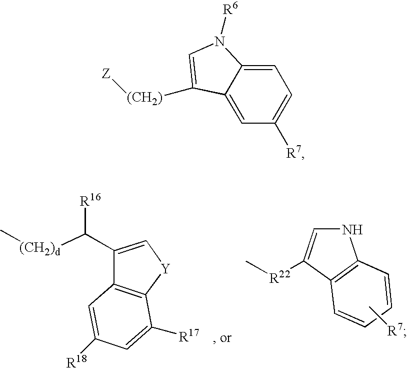
where a is an integer of 2 to 4 and R⁵ is A, B, C, D, K, L, or U;
a is an integer of 2 and R⁵ is E, G or J;
a is an integer of 3 or 4 and R⁵ is P;
A is 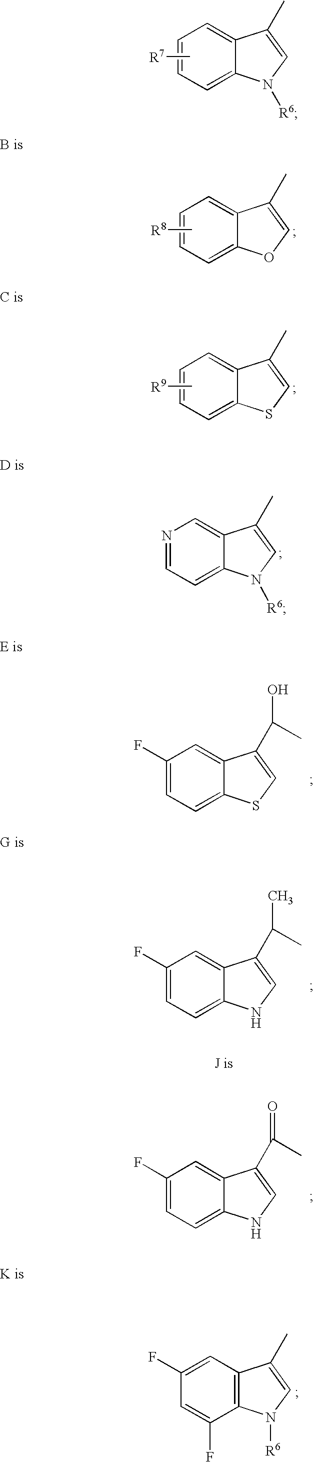 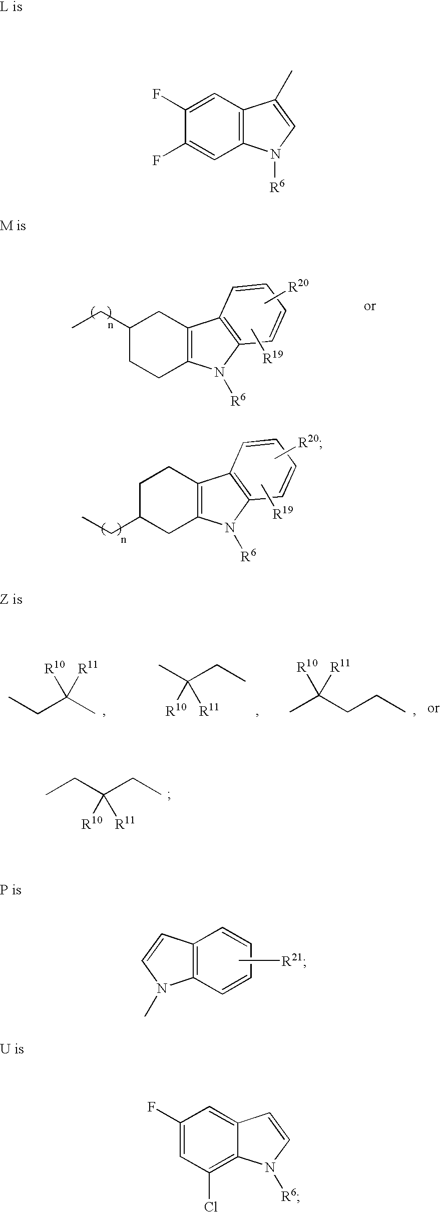
R is —OCH₃, —COR¹², —SO₂NR¹³R¹⁴, or heterocycle;
R⁴ is hydrogen or halo;
R⁶ is hydrogen or alkyl;
R⁷ is hydrogen, fluoro, chloro, cyano or alkoxy at either the 4-, 5-, 6-, or 7-position;
R⁸ is hydrogen, halo, C₁-C₃ alkoxy or C₁-C₃ alkyl;
R⁹ is hydrogen, halo, C₁-C₃ alkoxy or C₁-C₃ alkyl;
R¹⁰ is hydrogen and R¹¹ is methyl; or R¹⁰ and R¹¹ are methyl;
R¹² is C₁-C₄ alkyl, alkoxy or NR¹³R¹⁴;
R¹³ and R₁₄ are independently hydrogen, alkyl, cycloalkyl, methylcyclopropyl, phenyl, or benzyl;
R¹⁹ and R²⁰ are independently hydrogen, fluoro, chloro, cyano, or C₁-C₆ alkyl at either the 5-, 6-, 7-, or 8-position;
R²¹ is hydrogen or fluoro at either the 4-, 5-, 6- or 7-position;
R²² is a 3- to 7-membered ring;
n is an integer of 1 or 2;
Y is O, S, or NH;
   wherein, when Y is O, then
   R¹⁶ is hydrogen;
   R¹⁷ is hydrogen or OCH₃;
   R¹⁸ is hydrogen; and
   d is an-integer of 2 or 3;
   when Y is S, then
   R¹⁶ is hydrogen or hydroxyl;
   R¹⁷ is hydrogen;
   R¹⁸ is hydrogen or fluoro; and
   d is an integer of 2;
   when Y is NH, then
   R¹⁶ is keto or methyl;
   R¹⁷ is hydrogen;
   R¹⁸ is fluoro; and
   d is an integer of 2.

In some preferred embodiments, the present invention provides compounds of formula Ia: 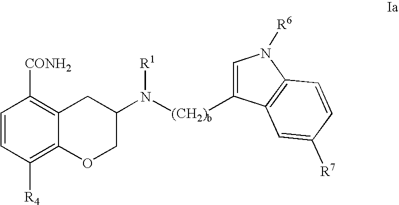
or a prodrug, stereoisomer or pharmaceutically acceptable-salt thereof;
wherein:
R¹ is ethyl, propyl, cyclobutyl, or methylcyclopropyl;
R⁴ is chloro or fluoro;
R⁶ is hydrogen or methyl;
R⁷ is hydrogen, fluoro or cyano; and
b is an integer of 3 or 4.

In other preferred embodiments, the present invention is directed to compounds of formula Ib or Ic: 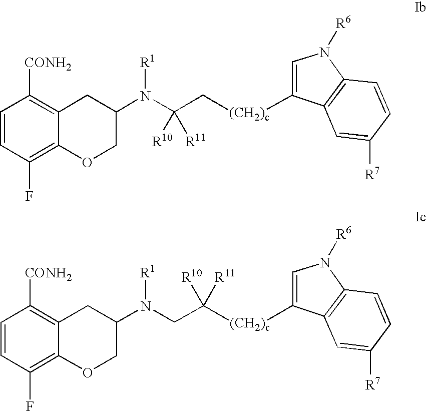
or a prodrug, stereoisomer or pharmaceutically-acceptable salt thereof;
wherein:
R¹ is ethyl, propyl, cyclobutyl, or methylcyclopropyl;
R⁶ is hydrogen or methyl;
R⁷ is hydrogen, fluoro or cyano;
R¹⁰ is hydrogen;
R¹¹ is methyl; and
c is an integer of 1 or 2.

In other preferred embodiments, the present invention provides compounds of formula Id: 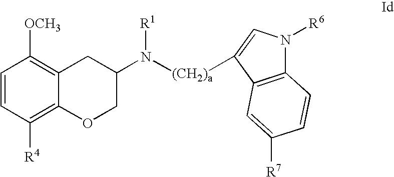
or a prodrug, stereoisomer or pharmaceutically-acceptable salt thereof;
wherein:
R¹ is methyl, ethyl, propyl, isopropyl, 2-methylpropyl, cyclopropyl, cyclobutyl, cyclopentyl, methylcyclopropyl, or methylcyclobutyl;
R⁴ is hydrogen or fluoro;
R⁶ is hydrogen or methyl;
R⁷ is hydrogen, fluoro or cyano; and
a is an integer of 2 to 4.

In other preferred embodiments, the present invention is directed to compounds of formula Ie: 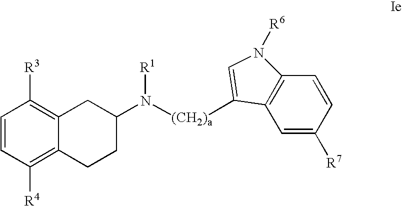
or a prodrug, stereoisomer or pharmaceutically-acceptable salt thereof;
wherein:
R¹ is ethyl, propyl, cyclobutyl, or methylcyclopropyl;
R⁶ is hydrogen or methyl;
R⁷ is hydrogen or fluoro;
R³ is —OCH₃ or —COR¹²;
R¹² is C₁-C₄ alkyl, alkoxy, or NR¹³R¹⁴;
R¹³ and R¹⁴ are independently hydrogen or alkyl;
R⁴ is hydrogen or fluoro; and
a is an integer of 2 to 4.

In other preferred embodiments of the invention is provided compounds of formula If: 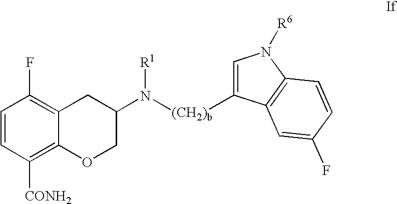
or a prodrug, stereoisomer or pharmaceutically-acceptable salt thereof;
wherein:
R¹ is propyl, cyclobutyl or methylcyclopropyl;
R⁶ is hydrogen or methyl; and
b is an integer of 3 or 4.

In other preferred embodiments of the invention is provided compounds of formula Ig: 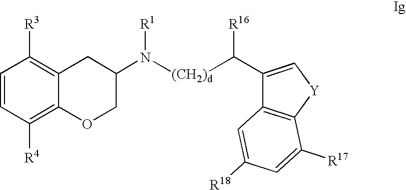
a prodrug, stereoisomer or pharmaceutically-acceptable salt thereof;
wherein:
R¹ is hydrogen, ethyl, propyl, cyclobutyl or methylcyclopropyl;
R³ is —OCH₃ or —CONH₂;
R⁴ is hydrogen or fluoro;
Y is O, S or NH;
   wherein, when Y is O, then
   R¹⁶ is hydrogen;
   R¹⁷ is hydrogen or OCH₃;
   R¹⁸ is hydrogen; and
   d is an integer of 1, 2 or 3;
   when Y is S., then
   R¹⁶ is hydrogen or hydroxyl;
   R¹⁷ is hydrogen;
   R¹⁸ is hydrogen or fluoro; and
   d is an integer of 2;
   when Y is NH, then
   R¹⁶ is keto or methyl;
   R¹⁷ is hydrogen;
   R¹⁸ is fluoro; and
   d is an integer of 2.

In other preferred embodiments, the present invention provides compounds of formula Ih or Ij: 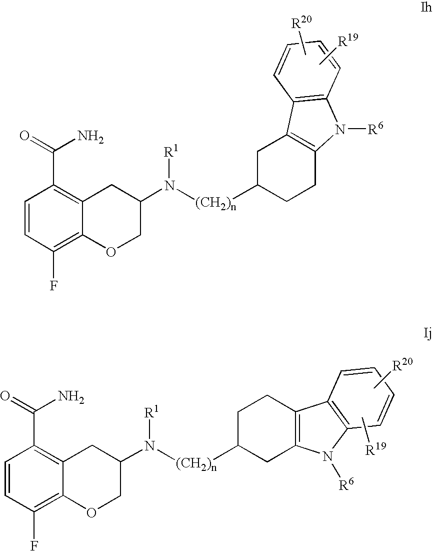
or a prodrug, stereoisomer or pharmaceutically-acceptable salt thereof;
wherein:
R¹ is hydrogen, propyl, methylcyclopropyl and cyclobutyl;
R⁶ is hydrogen or methyl;
R¹⁹ and R²⁰ are independently hydrogen, fluoro or cyano at either the 5-, 6-, 7- or 8-position; and
n is an integer of 1 or 2.

In other preferred embodiments, the present invention provides compounds of formula Ik: 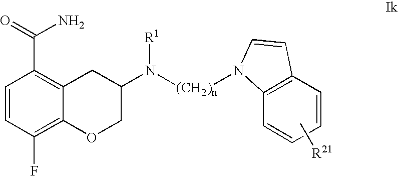
or a prodrug, stereoisomer or pharmaceutically-acceptable salt thereof;
wherein:
R¹ is hydrogen, ethyl, propyl, cyclobutyl, or methylcyclopropyl;
R²¹ is hydrogen or fluoro at either the 4-, 5-, 6- or 7-position; and
b is an integer of 3 or 4.

In other preferred embodiments, the present invention provides compounds of formula Im: 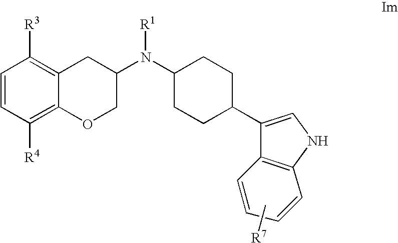
or a prodrug, stereoisomer or pharmaceutically-acceptable salt thereof;
wherein:
R¹ is hydrogen, ethyl, propyl, methylcyclopropyl or cyclobutyl;
R³ is —OCH₃ or CONH₂;
R⁴ is hydrogen or fluoro; and
R⁷ is hydrogen or fluoro at either the 4-, 5-, 6-, or 7-position.

In another aspect, the present invention is directed to compositions comprising a compound of formula I, Ia, Ib, Ic, Id, Ie, If, Ig, Ih, Ij, Ik, or Im and one or more pharmaceutically acceptable carriers.

The present invention also provides methods of treating and/or preventing a serotonin-related disorder in a patient suspected of suffering from a serotonin-related disorder, comprising the step of administering to the patient a therapeutically effective amount of a compound of formula I.

The present invention is also directed to a method of agonizing 5-HT_{1A} receptors in a patient in need thereof, comprising the step of administering to the patient a therapeutically effective amount of a compound of formula I.

In another aspect, the present invention also provides for a method of antagonizing 5-HT_{1A} receptors in a patient in need thereof, comprising the step of administering to the patient a therapeutically effective amount of a compound of formula I.

In yet another aspect, the present invention is also directed to methods of inhibiting the reuptake of serotonin in a patient in need thereof, comprising the step of administering to the patient a therapeutically effective amount of a compound of formula I.

As 5-HT_{1A} agonists, partial agonists, or antagonists, the novel compounds of this invention are useful for the treatment and/or prevention of several diseases and disorders, including depression (including, but not limited to major depressive disorder, childhood depression and dysthymia), anxiety, panic disorder, post-traumatic stress disorder, premenstrual dysphoric disorder (also known as premenstrual syndrome), attention deficit disorder (with or without hyperactivity), obsessive compulsive disorder, social anxiety disorder, generalized anxiety disorder, obesity, eating disorders such as anorexia nervosa and bulimia nervosa, vasomotor flushing, cocaine and alcohol addiction, sexual dysfunction, cognitive deficits resulting from neurodegenerative disorders like Alzheimer's disease, and related illnesses.

### DETAILED DESCRIPTION OF ILLUSTRATIVE EMBODIMENTS

The term “alkyl”, as used herein, whether used alone or as part of another group, refers to a substituted or unsubstituted aliphatic hydrocarbon chain, and includes, but is not limited to, straight and branched chains containing 1 to 6 carbon atoms, unless explicitly stated otherwise. For example, methyl, ethyl, n-propyl, isopropyl, and 2-methylpropyl are encompassed by the term “alkyl”. Specifically included within the definition of “alkyl” are those aliphatic hydrocarbon chains that are optionally substituted.

The carbon number, as used in the definitions herein, refers to carbon backbone and carbon branching, but does not include carbon atoms of the substituents, such as alkoxy substitutions and the like.

The term “cycloalkyl”, as used herein, whether used alone or as part of another group, refers to a substituted or unsubstituted alicyclic hydrocarbon group having 3 to 6 carbon atoms. Specifically included within the definition of “cycloalkyl” are those aliphatic hydrocarbon chains that are optionally substituted, and include, but are not limited to methylcyclopropyl, methylcyclobutyl and cyclobutyl.

The term “alkoxy”, as used herein, whether used alone or as part of another group, refers to the group Rₐ—O—, where Rₐ is an alkyl group containing 1 to 4 carbon atoms, as defined above, unless explicitly stated otherwise.

The term “heterocycle”, as used herein, refers to a substituted or unsubstituted monocylic aromatic heterocyclic ring system where the heteroaryl moiety is imidazole, 1,2,4-triazole, tetrazole, 1,2,4-oxadiazole, or 1,3,4-oxadiazole.

The term “halo”, as used herein, refers to chloro, fluoro or bromo.

The term “pharmaceutically acceptable salt”, as used herein, refers to salts derived from organic and inorganic acids such as, for example, acetic, lactic, citric, cinnamic, tartaric, succinic, fumaric, maleic, malonic, mandelic, malic, oxalic, propionic, hydrochloric, hydrobromic, phosphoric, nitric, sulfuric, glycolic, pyruvic, methanesulfonic, ethanesulfonic, toluenesulfonic, salicylic, benzoic, and similarly known acceptable acids.

The term “patient”, as used herein, refers to a mammal, preferably a human.

The terms “administer”, “administering” or “administration”, as used herein, refer to either directly administering a compound or composition to a patient, or administering a prodrug derivative or analog of the compound to the patient, which will form an equivalent amount of the active compound or substance within the patient's body.

The term “carrier”, as used herein, shall encompass carriers, excipients, and diluents.

The term “prodrug”, as used herein means a compound which is convertible in vivo by metabolic means (e.g. by hydrolysis) to a compound of formula I, Ia, Ib, Ic, Id, Ie, If Ig, Ih, Ij, Ik, or Im.

This invention relates to both the R and S stereoisomers of the 3-amino-chroman or 2-amino-tetralin derivatives, as well as to mixtures of the R and S stereoisomers. Throughout this application, the name of the product of this invention, where the absolute configuration of the 3-amino-chromans or 2-amino tetralins is not indicated, is intended to embrace the individual R and S enantiomers as well as mixtures of the two.

This invention also relates to both the R and S stereoisomers at the carbon alpha or beta from the basic nitrogen. Throughout this application, the name of the product of this invention, where the absolute configuration at the above two positions is not indicated, is intended to embrace the individual R and S enantiomers.

Where a stereoisomer is preferred, it may in some embodiments be provided substantially free of the corresponding enantiomer. Thus, an enantiomer substantially free of the corresponding enantiomer refers to a compound that is isolated or separated via separation techniques or prepared free of the corresponding enantiomer. “Substantially free”, as used herein, means that the compound is made up of a significantly greater proportion of one stereoisomer, preferably less than about 50%, more preferably less than about 75%, and even more preferably less than about 90%. The preferred stereoisomer was isolated from raceimic mixtures by high performance liquid chromatography (HPLC) using a chiral column.

Preferred among the above noted R¹ groups in formula I are hydrogen, alkyl, cycloalkyl, and methylcyclopropyl. Particularly preferred are hydrogen, ethyl, propyl, methylcyclopropyl, and cyclobutyl.

Preferred among the above noted R² groups in formula I are —(CH₂)ₐ—R⁵, and

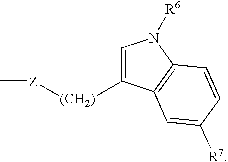

Preferred among the above noted R³ groups in formula I are —OCH₃, and —COR¹². Particularly preferred are —COR¹².

Preferred among the above noted R⁴ groups in formula I are hydrogen, fluoro, and chloro. Particularly preferred are fluoro and chloro.

Preferred among the above noted R⁵ groups in formula I are A, B, K, and P. Particularly preferred are A and K.

Preferred among the above noted R⁶ groups in formula I are hydrogen and alkyl. Particularly preferred are hydrogen and methyl.

Preferred among the above noted R⁷ groups in formula I are hydrogen, fluoro and cyano at either the 5-, 6-, or 7-position. Particularly preferred are hydrogen, cyano, fluoro at the 5-position.

Preferred among the above noted R⁸ groups in formula I are hydrogen and C₁-C₃ alkoxy. Particularly preferred are hydrogen and methoxy.

Preferred among the above noted R⁹ groups in formula I are hydrogen and fluoro. Preferred among the above noted R groups in formula I are alkoxy, and NR³R¹⁴. Particularly preferred are methoxy, NH₂, and NHMe.

Preferred among the above noted R¹³ groups in formula I is hydrogen.

Preferred among the above noted R¹⁴ groups in formula I are hydrogen and methyl.

Preferred among the above noted Z groups in formula I are

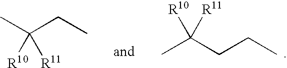

Preferred among the above noted X groups in formula I are O and methylene.

Preferred among the above noted R¹⁶ groups in formula I, are hydrogen when Y is O or S, and methyl when Y is NH.

Preferred among the above noted R¹⁷ groups in formula I are hydrogen when Y is O, S, or NH and methoxy when Y is O.

Preferred among the above noted R¹⁹ and R²⁰ groups in formula I is fluoro.

Preferred among the above noted R²¹ groups in formula I is fluoro.

Preferred among the above noted R²² groups in formula I are 4-, 5- and 6-membered rings.

The following compounds are particularly preferred:
8-fluoro-3-{[3-(5-fluoro-1H-indol-3-yl)propyl]amino}chromane-5-carboxamide;
(+)-8-fluoro-3-{[3-(5-fluoro-1H-indol-3-yl)propyl]amino}chromane-5-carboxamide;
(−)-8-fluoro-3-{[3-(5-fluoro-1H-indol-3-yl)propyl]amino}chromane-5-carboxamide;
8-fluoro-3-{[3-(5-fluoro-1H-indol-3-yl)propyl](propyl)amino}chromane-5-carboxamide;
(−)-8-fluoro-3-{[3-(5-fluoro-1H-indol-3-yl)propyl](propyl)amino}-3,4-dihydro-2H-chromene-5-carboxamide;
(+)-8-fluoro-3-{[3-(5-fluoro-1H-indol-3-yl)propyl](propyl)amino}-3,4-dihydro-2H-chromene-5-carboxamide;
(−)-3-{ethyl[3-(5-fluoro-1H-indol-3-yl)propyl]amino}-8-fluoro-3,4-dihydro-2H-chromene-5-carboxamide;
(+)-3-{ethyl[3-(5-fluoro-1H-indol-3-yl)propyl]amino}-8-fluoro-3,4-dihydro-2H-chromene-5-carboxamide;
3-{cyclobutyl[3-(5-fluoro-1H-indol-3-yl)propyl]amino}-8-fluorochromane-5-carboxamide;
(+)-3-{cyclobutyl[3-(5-fluoro-1H-indol-3-yl)propyl]amino}-8-fluorochromane-5-carboxamide;
(−)3-{cyclobutyl[3-(5-fluoro-1H-indol-3-yl)propyl]amino}-8-fluorochromane-5-carboxamide;
3-{cyclopropylmethyl[3-(5-fluoro-1H-indol-3-yl)propyl]amino}-8-fluorochromane-5-carboxamide;
(−)-3-{cyclolropylmethyl[3-(5-fluoro-1H-indol-3-yl)propyl]amino}-8-fluorochromane-5-carboxamide;
(+)-3-{cyclopropylmethyl[3-(5-fluoro-1H-indol-3-yl)propyl]amino}-8-fluorochromane-5-carboxamide;
3-{(1-cyclopropylethyl)[3-(5-fluoro-1H-indol-3-yl)propyl]aminol}-8-fluorochromane-5-carboxamide;
8-chloro-3-{cyclobutyl[3-(5-fluoro-1H-indol-3-yl)propyl]amino}chromane-5-carboxamide;
3-{cyclobutyl[3-(5-fluoro-1H-indol-3-yl)-3-oxopropyl]amino}-8-fluorochromane-5-carboxamide;
(−)-3-{cyclobutyl[3-(5,7-difluoro-1H-indol-3-yl)propyl]amino}-8-fluorochromane-5-carboxamide;
(+)-3-{cyclobutyl[3-(5,7-difluoro-1H-indol-3-yl)propyl]amino}-8-fluorochromane-5-carboxamide;
methyl-8-fluoro-3-{[3-(5-fluoro-1H-indol-3-yl)propyl]amino}chromane-5-carboxylate;
methyl-3-{cyclobutyl[3-(5-fluoro-1H-indol-3-yl)propyl]amino}-8-fluorochromane-5-carboxylate;
8-fluoro-3-{[3-(5-fluoro-1H-indol-3-yl)-1-methylpropyl](propyl)amino}-3,4-dihydro-2H-chromene-5-carboxamide;
(3R)-8-fluoro-3-[[(1S)-3-(5-fluoro-1H-indol-3-yl)-1-methylpropyl](propyl)amino]chromane-5-carboxamide;
(3R)-8-fluoro-3-[[(1R)-3-(5-fluoro-1H-indol-3-yl)-1-methylpropyl](propyl)amino]chromane-5-carboxamide;
(3S)-8-fluoro-3-[[(1R)-3-(5-fluoro-1H-indol-3-yl)-1-methylpropyl](propyl)amino]chromane-5-carboxamide;
(3S)-8-fluoro-3-[[(1S)-3-(5-fluoro-1H-indol-3-yl)-1-methylpropyl](propyl)amino]chromane-5-carboxamide;
3-{[3-(5-cyano-1H-indol-3-yl)-1-methylpropyl](propyl)amino}-8-fluoro-3,4-dihydro-2H-chromene-5-carboxamide;
(3R)-3-[[(1R)-3-(5-cyano-1H-indol-3-yl)-1-methylpropyl](propyl)amino]-8-fluorochromane-5-carboxamide;
(3S)-3-[[(1S)-3-(5-cyano-1H-indol-3-yl)-1-methylpropyl](propyl)amino]-8-fluorochromane-5-carboxamide;
(3R)-3-[[(1S)-3-(5-cyano-1H-indol-3-yl)-1-methylpropyl](propyl)amino]-8-fluorochromane-5-carboxamide;
(3S)-3-[[(1R)-3-(5-cyano-1H-indol-3-yl)-1 methylpropyl](propyl)amino]-8-fluorochromane-5-carboxamide;
8-fluoro-3-{[4-(5-fluoro-1H-indol-3-yl)-1-methylbutyl](propyl)amino}-chromane-5-carboxamide;
(+)-8-fluoro-3-{[4-(5-fluoro-1H-indol-3-yl)-1-methylbutyl](propyl)amino}-chromane-5-carboxamide;
(−)-8-fluoro-3-{[4-(5-fluoro-1H-indol-3-yl)-1-methylbutyl](propyl)amino}-chromane-5-carboxamide;
(−)-3-{(cyclopropylmethyl)[3-(5-fluoro-1H-indol-3-yl)-1-methylpropyl]amino}-8-fluorochromane-5-carboxamide;
(+)-3-{(cyclopropylmethyl)[3-(5-fluoro-1H-indol-3-yl)-1-methylpropyl]amino}-8-fluorochromane-5-carboxamide;
(−)-3-(ethyl[3-(5-fluoro-1H-indol-3-yl)-1-methylpropyl]amino}-8-fluorochromane-5-carboxamide;
(+)-3-{ethyl[3-(5-fluoro-1H-indol-3-yl)-1-methylpropyl]amino}-8-fluorochromane-5-carboxamide;
8-fluoro-3-{[3-(5-fluoro-1-benzothien-3-yl)-3-hydroxypropyl](propyl)amino}-chromane-5-carboxamide;
N-[3-(1-benzothien-3-yl)propyl]-N-ethyl-5-methoxychroman-3-amine;
N-[3-(5-fluoro-1-benzothien-3-yl)propyl]-5-methoxy-N-propylchroman-3-amine;
3-{[3-(1-benzofuran-3-yl)propyl](propyl)amino}-8-fluorochromane-5-carboxamide;
N-[3-(1-benzofuran-3-yl)propyl]-N-ethyl-5-methoxychroman-3-amine;
N-[4-(1-benzofuran-3-yl)butyl]-N-ethyl-N-(5-methoxy-0,3,4-dihydro-2H-chromen-3-yl)amine;
[3-(5-fluoro-1H-indol-3-yl)propyl]-N-(5-methoxy-chroman-3-yl)propylamine;
[3-(5-fluoro-1H-indol-3-yl)propyl]((3R)-5-methoxychroman-3-yl)propylamine;
[3-(5-fluoro-1H-indol-3-yl)propyl]((3S)-5-methoxychroman-3-yl)propylamine;
[3-(5-fluoro-1H-indol-3-yl)propyl]-(8-fluoro-5-methoxychroman-3-yl)propylamine;
(3S)-8-fluoro-N-[3-(5-fluoro-1H-indol-3-yl)propyl]-5-methoxy-N-propylchroman-3-amine;
(3R)-8-fluoro-N-[3-(5-fluoro-1H-indol-3-yl)propyl]-5-methoxy-N-propylchroman-3-amine;
N-[2-(5-fluoro-1H-indol-3-yl)ethyl]-5-methoxy-N-propylchroman-3-amine;
N-[4-(5-fluoro-1H-indol-3-yl)butyl]-5-methoxy-N-propylchroman-3-amine;
N-ethyl-N-[3-(5-fluoro-1H-indol-3-yl)propyl]-5-methoxy chroman-3-amine;
N-ethyl-N-[4-(5-fluoro-1H-indol-3-yl)butyl]-5-methoxychroman-3-amine;
N-[3-(5-fluoro-1H-indol-3-yl)prop yl]-5-methoxy-N-methylchroman-3-amine;
N-cyclobutyl-N-[3-(5-fluoro-1H-indol-3-yl)propyl]-5-methoxychroman-3 amine;
(3R)-N-cyclobutyl-N-[3-(5-fluoro-1H-indol-3-yl)propyl]-5-methoxy-3,4-dihydro-2H-chromen-3-amine;
N-cyclobutyl-N-[4-(5-fluoro- H-indol-3-yl)butyl]-N-(5-methoxy-3,4-dihydro-2H-chromen-3-yl)amine;
N-(cyclopropylmethyl)-N-[3-(5-fluoro-1H-indol-3-yl)propyl]-N-(5-methoxy-3,4-dihydro-2H-chromen-3-yl)amine;
N-(cyclopropylmethyl)-N-[3-(5-fluoro-1-methyl- 1H-indol-3-yl)propyl]-N-(5-methoxy-3,4-dihydro-2H-chromen-3-yl)amine;
N-(cyclopentyl)-N-[3-(5-fluoro-1H-indol-3-yl)propyl]-N-(5-methoxy-3,4-dihydro-2H-chromen-3-yl)amine;
N-[3-(5-fluoro-1H-indol-3-yl)propyl]-N-isopropyl-N-(5-methoxy-3,4-dihydro-2H-chromen-3-yl)amine;
N-cyclopropyl-N-[3-(5-fluoro-1H-indol-3-yl)propyl]-N-(5-methoxy-3,4-dihydro-2H-chromen-3-yl)amine;
N-(cyclobutylmethyl)-N-[3-(5-fluoro-1H-indol-3-yl)propyl]-N-(5-methoxy-3,4-dihydro-2H-chromen-3-yl)amine;
N-(cyclopropylmethyl)-N-[3-(1H-indol-3-yl)propyl]-N-(5-methoxy-3,4-dihydro-2H-chromen-3-yl)amine;
N-cyclobutyl-N-[3-(1H-indol-3-yl)propyl]-N-(5-methoxy-3,4-dihydro-2H-chromen-3-yl)amine;
3-{3-[(cyclopropylmethyl)(5-methoxy-3,4-dihydro-2H-chromen-3-yl)amino]propyl}-1H-indole-5-carbonitrile;
3-{3-[cyclobutyl(5-methoxy-3,4-dihydro-2H-chromen-3-yl)amino]propyl}-1H-indole-5-carbonitrile;
N-[3-(5-fluoro-1H-indol-3yl)propyl]-N-(8-methoxy-1,2,3,4-tetrahydronaphthalen-2-yl)-N-propylamine;
(−)-N-[3-(5-fluoro-1H-indol-3yl)propyl]-8-methoxy-N-propyl-1,2,3,4-tetrahydronaphthalen-2-amine;
(+)-N-[3-(5-fluoro-1H-indol-3yl)propyl]-8-methoxy-N-propyl-1,2,3,4-tetrahydronaphthalen-2-amine;
N-[2-(5-fluoro-1H-indol-3-yl)ethyl]-N-(8-methoxy-1,2,3,4-tetrahydronaphthalen-2-yl)-N-propylamine;
N-ethyl-N-[2-(5-fluoro-1H-indol-3-yl)ethyl]-N-(8-methoxy-1,2,3,4-tetrahydronaphthalen-2-yl)amine;
N-[3-(1H-indol-3-yl)propyl]-8-methoxy-N-propyl-1,2,3,4-tetrahydronaphthalen-2-amine;
N-[3-(5-fluoro-1H-indol-3-yl)propyl]-N-(5-fluoro-8-methoxy-1,2,3,4-tetrahydronaphthalen-2-yl-N-propylamine;
(+)-5-fluoro-N-[3-(5-fluoro-1H-indol-3-yl)propyl]-8-methoxy-N-propyl-1,2,3,4-tetrahydro-2-naphthalenlamine;
(−)-5-fluoro-N-[3-(5-fluoro-1H-indol-3-yl)propyl]-8-methoxy-N-propyl-1,2,3,4-tetrahydro-2-naphthalenamine;
8-fluoro-3-{[3-(6-fluoro-1H-indol-3-yl)propyl]amino}chromane-5-carboxamide;
3-{(cyclopropylmethyl)[3-(6-fluoro- H-indol-3-yl)propyl]amino}-8-fluorochromane-5-carboxamide;
3-{cyclobutyl[3-(6-fluoro-1H-indol-3-yl)propyl]amino}-8-fluorochromane-5-carboxamide;
Methyl 8-fluoro-3-{[3-(5-fluoro-1H-indol-3-yl)propyl]amino}chromane-5-carboxylate;
Methyl 3-{cyclobutyl[3-(5-fluoro-1H-indol-3-yl)propyl]amino}-8-fluorochromane-5-carboxylate;
3-{cyclobutyl[3-(5-fluoro-1H-indol-3-yl)propyl]amino}-8-fluorochromane-5-carboxylic acid;
Methyl 8-fluoro-3-{[4-(5-fluoro-1H-indol-3-yl)butyl]amino}chromane-5-carboxylate;
Methyl (3S)-8-fluoro-3-{[4-(5-fluoro-1H-indol-3-yl)butyl]amino}chromane-5-carboxylate;
Methyl (3R)-8-fluoro-3-{[4-(5-fluoro-1H-indol-3-yl)butyl]amino}chromane-5-carboxylate;
Methyl 3-{cyclobutyl[4-(5-fluoro-1H-indol-3-yl)butyl]amino}-8-fluorochromane-5-carboxylate;
Methyl (3S)-3-{cyclobutyl[4-(5-fluoro-1H-indol-3-yl)butyl]amino}-8-fluorochromane-5-carboxylate;
Methyl (3R)-3-{cyclobutyl[4-(5-fluoro-1H-indol-3-yl)butyl]amino}-8-fluorochromane-5-carboxylate;
3-{cyclobutyl[3-(5-fluoro-1H-indol-3-yl)propyl]amino}-8-fluoro-N-methylchromane-5-carboxamide;
3-{cyclobutyl[3-(5-fluoro-1H-indol-3-yl)propyl]amino}-N-ethyl-8-fluorochromane-5-carboxamide;
3-{cyclobutyl[3-(5-fluoro-1H-indol-3-yl)propyl]amino}-8-fluoro-N-propylchromane-5-carboxamide;
N-butyl-3-{cyclobutyl[3-(5-fluoro-1H-indol-3-yl)propyl]amino}-8-fluorochromane-5-carboxamide;
3-{cyclobutyl[3-(5-fluoro-1H-indol-3-yl)propyl]amino}-8-fluoro-N-isopropylchromane-5-carboxamide;
3-{cyclobutyl[3-(5-fluoro-1H-indol-3-yl)propyl]amino}-N-cyclopropyl-8-fluorochromane-5-carboxamide;
N-cyclobutyl-3-{cyclobutyl[3-(5-fluoro-1H-indol-3-yl)propyl]amino}-8-fluorochromane-5-carboxamide;
3-{cyclobutyl[3-(5-fluoro-1H-indol-3-yl)propyl]amino}-N-cyclopentyl-8-fluorochromane-5-carboxamide;
3-{cyclobutyl[3-(5-fluoro-1H-indol-3-yl)propyl]amino}-N-cyclohexyl-8-fluorochromane-5-carboxamide;
3-{cyclobutyl[3-(5-fluoro-1H-indol-3-yl)propyl]amino}-N-(cyclopropylmethyl)-8-fluorochromane-5-carboxamide;
N-benzyl-3-{cyclobutyl[3-(5-fluoro-1H-indol-3-yl)propyl]amino}-8-fluorochromane-5-carboxamide;
3-{cyclobutyl[3-(5-fluoro-1H-indol-3-yl)propyl]amino}-8-fluoro-N-phenylchromane-5-carboxamide
8-fluoro-3-[[3-(5-fluoro-1H-indol-3-yl)propyl](pentyl)amino]chromane-5-carboxamide;
3-{butyl[3-(5-fluoro-1H-indol-3-yl)propyl]amino}-8-fluorochromane-5-carboxamide;
3-{cyclobutyl[3-(5-fluoro-1H-indol-3-yl)propyl]amino}-8-fluoro-N,N-dimethylchrormane-5-carboxamide;
3-{benzyl[3-(5-fluoro-1H-indol-3-yl)propyl]amino}-8-fluorochromane-5-carboxamide;
8-fluoro-3- ([2-(5-fluoro-1H-indol-3-yl)ethyl]amino}chromane-5-carboxamide;
3-{ethyl[3-(5-fluoro-1H-indol-3-yl)propyl]amino}-8-fluorochromane-5-carboxamide;
8-fluoro-3-[[2-(5-fluoro-1H-indol-3-yl)ethyl](propyl)amino]chromane-5-carboxamide;
3-{(cyclopropylmethyl)[2-(5-fluoro-1H-indol-3-yl)ethyl]amino}-8-fluorochromane-5-carboxamide;
8-fluoro-3-{[4-(5-fluoro-1H-indol-3-yl)butyl]amino}chromane-5-carboxamide;
3-{ethyl[4-(5-fluoro-1H-indol-3-yl)butyl]amino}-8-fluorochromane-5-carboxamide;
8-fluoro-3-[[4-(5-fluoro-1H-indol-3-yl)butyl](propyl)amino]chromane-5-carboxamide;
3-{(cyclopropylmethyl)[4-(5-fluoro-1H-indol-3-yl)butyl]amino}-8-fluorochromane-5-carboxamide;
3-{cyclobutyl[4-(5-fluoro-1H-indol-3-yl)butyl]amino}-8-fluorochromane-5-carboxamide;
(3R)-3-{cyclobutyl[4-(5-fluoro-1H-indol-3-yl)butyl]amino}-8-fluorochromane-5-carboxamide;
(3S)-3-{cyclobutyl[4-(5-fluoro-1H-indol-3-yl)butyl]amino}-8-fluorochromane-5-carboxamide;
8-fluoro-3-{[2-(5-fluoro-1H-indol-3-yl)ethyl]amino}-N-methylchromane-5-carboxamide;
3-{ethyl[2-(5-fluoro-1H-indol-3-yl)ethyl]amino}-8-fluoro-N-methylchromane-5-carboxamide;
3-{(cyclopropylmethyl)[2-(5-fluoro-1H-indol-3-yl)ethyl]amino}-8-fluoro-N-methylchromane-5-carboxamide;
3-{cyclobutyl[2-(5-fluoro-1H-indol-3-yl)ethyl]amino}-8-fluoro-N-methylchromane-5-carboxamide;
8-fluoro-3-{[3-(5-fluoro-1H-indol-3-yl)propyl]amino}-N-methylchromane-5-carboxamide;
3-{ethyl[3-(5-fluoro-1H-indol-3-yl)propyl]amino}-8-fluoro-N-methylchromane-5-carboxamide;
8-fluoro-3-[[3-(5-fluoro-1H-indol-3-yl)propyl](propyl)amino]-N-methylchromane-5-carboxamide;
3-{(cyclopropylmethyl)[3-(5-fluoro-1H-indol-3-yl)propyl]amino}-8-fluoro-N-methylchromane-5-carboxamide;
8-fluoro-3-{[4-(5-fluoro-1H-indol-3-yl)butyl]amino}-N-methylchromane-5-carboxamide;
8-fluoro-3-[[4-(5-fluoro-1H-indol-3-yl)butyl](propyl)amino]-N-methylchromane-5-carboxamide;
3-{(cyclopropylmethyl)[4-(5-fluoro-1H-indol-3-yl)butyl]amino}-8-fluoro-N-methylchromane-5-carboxamide;
3-{cyclobutyl[4-(5-fluoro-1H-indol-3-yl)butyl]amino}-8-fluoro-N-methylchromane-5-carboxamide;
3-{[3-(5-cyano-1H-indol-3-yl)propyl]amino}-8-fluorochromane-5-carboxamide;
3-[[3-(5-cyano-1H-indol-3-yl)propyl](cyclobutyl)amino]-8-fluorochromane-5-carboxamide;
(3S)-3-[[3-(5-cyano- 1H-indol-3-yl)propyl](cyclobutyl)amino]-8-fluorochromane-5-carboxamide;
(3R)-3-[[3-(5-cyano-1H-indol-3-yl)propyl](cyclobutyl)amino]-8-fluorochromane-5-carboxamide;
3-[[3-(5-cyano-1H-indol-3-yl)propyl](cyclopropylmethyl)amino]-8-fluorochromane-5-carboxamide;
(3S)-3-[[3-(5-cyano-1H-indol-3-yl)propyl](cyclopropylmethyl)amino]-8-fluorochromane-5-carboxamide
(3R)-3-[[3-(5-cyano-1H-indol-3-yl)propyl](cyclopropylmethyl)amino]-8-fluorochromane-5-carboxamide;
8-fluoro-3-{[3-(7-methoxy-1H-indol-3-yl)propyl]amino}chromane-5-carboxamide;
8-fluoro-3-[[3-(7-methoxy- H-indol-3-yl)propyl](propyl)amino]chromane-5-carboxamide;
3-{ethyl[3-(7-methoxy-1H-indol-3-yl)propyl]amino}-8-fluorochromane-5-carboxamide;
   3-{cyclobutyl[3-(7-methoxy-1H-indol-3-yl)propyl]amino}-8-fluorochromane-5-carboxamide;
3-{(cyclopropylmethyl)[3-(7-methoxy- H indol-3-yl)propyl]amino)}-8-fluorochromane-5-carboxamide;
8-fluoro-3-{[3-(5-methoxy-1H-indol-3-yl)propyl]amino}chromane-5-carboxamide;
3-{ethyl[3-(5-methoxy-1H-indol-3-yl)propyl]amino}-8-fluorochromane-5-carboxamide;
8-fluoro-3-[[3-(5-methoxy-1H-indol-3-yl)propyl](propyl)amino]chromane-5-carboxamide;
3-{cyclobutyl[3-(5-methoxy-1H-indol-3-yl)propyl]amino}-8-fluorochromane-5-carboxamide;
3-{(cyclopropylmethyl)[3-(5-methoxy- 1H-indol-3-yl)propyl]amino}-8-fluorochromane-5-carboxamide;
3-{[3-(7-chloro-1H-indol-3-yl)propyl]amino}-8-fluorochromane-5-carboxamide
3-[[3-(7-chloro-1H-indol-3-yl)propyl](ethyl)amino]-8-fluorochromane-5-carboxamide
3-[[3-(7-chloro-1H-indol-3-yl)propyl](cyclobutyl)amino]-8-fluorochromane-5-carboxamide
3-[[3-(7-chloro-1H-indol-3-yl)propyl](cyclopropylmethyl)amino]-8-fluorochromane-5-carboxamide
3-[[3-(7-chloro-1H-indol-3-yl)propyl](propyl)amino]-8-fluorochromane-5-carboxamide;
3-{[3-(5-chloro-1H-indol-3-yl)propyl]amino}-8-fluorochromane-5-carboxamide;
3-[[3-(5-chloro-1H-indol-3-yl)propyl](ethyl)amino]-8-fluorochromane-5-carboxamide;
3-[[3-(5-chloro-1H-indol-3-yl)propyl](propyl)amino]-8-fluorochromane-5-carboxamide;
3-[[3-(5-chloro-1H-indol-3-yl)propyl](cyclobutyl)amino]-8-fluorochromane-5-carboxamide;
3-[[3-(5-chloro- 1H-indol-3-yl)propyl](cyclopropylmethyl)amino]-8-fluorochromane-5-carboxamide;
5-fluoro-3-{[3-(5-fluoro-1H-indol-3-yl)propyl]amino}chromane-8-carboxamide;
5-fluoro-3-[[3-(5-fluoro-1H-indol-3-yl)propyl](propyl)amino]chromane-8-carboxamide;
3-{(cyclopropylmethyl)[3-(5-fluoro-1H-indol-3-yl)propyl]amino}-5-fluorochromane-8-carboxamide;
3-{cyclobutyl[3-(5-fluoro-1H-indol-3-yl)propyl]amino}-5-fluorochromane-8-carboxamide;
5-fluoro-3-{[4-(5-fluoro-1H-indol-3-yl)butyl]amino}chromane-8-carboxamide;
5-fluoro-3-[[4-(5-fluoro-1H-indol-3-yl)butyl](propyl)amino]chromane-8-carboxamide;
3-{(cyclopropylmethyl)[4-(5-fluoro-1H-indol-3-yl)butyl]amino}-5-fluorochromane-8-carboxamide;
3-{cyclobutyl[4-(5-fluoro-1H-indol-3-yl)butyl]amino}-5-fluorochromane-8-carboxamide;
3-{ethyl[4-(5-fluoro-1H-indol-3-yl)butyl]amino}-5-fluorochromane-8-carboxamide;
3-{[3-(5,7-difluoro-1H-indol-3-yl)propyl]amino}-5-fluorochromane-8-carboxamide;
3-[[3-(5,7-difluoro-1H-indol-3-yl)propyl](propyl)amino]-5-fluorochromane-8-carboxamide;
3-{(cyclopropylmethyl)[3-(5,7-difluoro-1H-indol-3-yl)propyl]amino}-5-fluorochromane-8-carboxamide;
3-{cyclobutyl[3-(5,7-difluoro-1H-indol-3-yl)propyl]amino}-5-fluorochromane-8-carboxamide;
3-[[3-(5,7-difluoro-1H-indol-3-yl)propyl](ethyl)amino]-5-fluorochromane-8-carboxamide;
3-{[3-(5-fluoro-1H-indol-3-yl)propyl]amino}chromane-5-carboxamide;
(3S)-3-{[3-(5-fluoro-1H-indol-3-yl)propyl]amino}chromane-5-carboxamide;
(3R)-3-{[3-(5-fluoro-1H-indol-3-yl)propyl]amino}chromane-5-carboxamide;
3-{cyclobutyl[3-(5-fluoro-1H-indol-3-yl)propyl]amino}chromane-5-carboxamide;
(−)-3-{cyclobutyl[3-(5-fluoro-1H-indol-3-yl)propyl]amino}chromane-5-carboxamide;
(+)-3-{cyclobutyl[3-(5-fluoro-1H-indol-3-yl)propyl]amino}chromane-5-carboxamide;
Methyl 3-{[3-(5,7-difluoro-1H-indol-3-yl)propyl]amino}chromane-5-carboxylate;
Methyl 3-{cyclobutyl[3-(5,7-difluoro-1H-indol-3-yl)propyl]amino}chromane-5-carboxylate;
3-{cyclobutyl[3-(5,7-difluoro-1H-indol-3-yl)propyl]amino}chromane-5-carboxylic acid;
3-{cyclobutyl[3-(5,7-difluoro-1H-indol-3-yl)propyl]amino}-N-methylchromane-5-carboxamide;
3-{cyclobutyl[3-(5,7-difluoro-1H-indol-3-yl)propyl]amino}-N-ethylchromane-5-carboxamide;
3-{cyclobutyl[3-(5,7-difluoro-1H-indol-3-yl)propyl]amino}-N-propylchromane-5-carboxamide;
3-{cyclobutyl[3-(5,7-difluoro-1H-indol-3-yl)propyl]amino}-N-isopropylchromane-5-carboxamide;
3-{cyclobutyl[3-(5,7-difluoro-1H-indol-3-yl)propyl]amino}-N-cyclopropylchromane-5-carboxamide;
N-cyclobutyl-3-{cyclobutyl[3-(5,7-difluoro-1H-indol-3-yl)propyl]amino}chromane-5-carboxamide;
3-{cyclobutyl[3-(5,7-difluoro-1H-indol-3-yl)propyl]amino}-N-(cyclopropylmethyl)chromane-5-carboxamide;
(3S)-3-{cyclobutyl[4-(5-fluoro-1H-indol-3-yl)butyl]amino}-8-fluoro-N-methylchromane-5-carboxamide;
(3R)-3-{cyclobutyl[4-(5-fluoro-1H-indol-3-yl)butyl]amino}-8-fluoro-N-methylchromane-5-carboxamide;
(3R)-3-{[3-(5,7-difluoro-1H-indol-3-yl)propyl]amino}-8-fluorochromane-5-carboxamide;
(3R)-3-{(cyclopropylmethyl)[3-(5,7-difluoro-1H-indol-3-yl)propyl]amino}-8-fluorochromane-5-carboxamide;
3-{(cyclopropylmethyl)[3-(5-fluoro-1H-indol-3-yl)propyl]amino}chromane-5-carboxamide;
3-{ethyl[3-(5-fluoro-1H-indol-3-yl)propyl]amino}chromane-5-carboxamide;
3-[[3-(5-fluoro-1H-indol-3-yl)propyl](propyl)amino]chromane-5-carboxamide;
3-[[3-(5-fluoro-1H-indol-3-yl)propyl](isobutyl)amino]chromane-5-carboxamide;
8-fluoro-3-{[(3R)-3-(5-fluoro-1H-indol-3-yl)butyl]amino}chromane-5-carboxamide;
(3R)-3-{cyclobutyl[(3R)-3-(5-fluoro-1H-indol-3-yl)butyl]amino}-8-fluorochromane-5-carboxamide;
(3S)-3-{cyclobutyl[(3R)-3-(5-fluoro-1H-indol-3-yl)butyl]amino}-8-fluorochromane-5-carboxamide;
8-fluoro-3-{[(3S)-3-(5-fluoro-1H-indol-3-yl)butyl]amino)}chromane-5-carboxamide;
(3R)-3-{cyclobutyl[(3S)-3-(5-fluoro-1H-indol-3-yl)butyl]amino}-8-fluorochromane-5-carboxamide;
(3S)-3-{cyclobutyl[(3S)-3-(5-fluoro-1H-indol-3-yl)butyl]amino}-8-fluorochromane-5-carboxamide;
3-[3-(5,7-Difluoro-1H-indol-3-yl)-1-methyl-propylamino]-8-fluoro-chroman-5-carboxylic acid amide;
(3R)-3-{(cyclopropylmethyl)[(1R)-3-(5,7-difluoro-1H-indol-3-yl)-1-methylpropyl]amino}-8-fluorochromane-5-carboxamide;
(3R)-3-{(cyclopropylmethyl)[(1S)-3-(5,7-difluoro-1H-indol-3-yl)-1-methylpropyl]amino}-8-fluorochromane-5-carboxamide;
(3S)-3-{(cyclopropylmethyl)[(15)-3-(5,7-difluoro-1H-indol-3-yl)-1-methylpropyl]amino}-8-fluorochromane-5-carboxamide;
(3S)-3-{(cyclopropylmethyl)[(1R)-3-(5,7-difluoro-1H-indol-3-yl)-1-methylpropyl]amino}-8-fluorochromane-5-carboxamide;
(3R)-8-fluoro-3-{[3-(5-fluoro-1H-indol-3-yl)-2-methylpropyl]amino}chromane-5-carboxamide;
(3R)-3-{(cyclopropylmethyl)[(2S)-3-(5-fluoro-1H-indol-3-yl)-2-methylpropyl]amino}-8-fluorochromane-5-carboxamide;
(3R)-3-{(cyclopropylmethyl)[(2R)-3-(5-fluoro-1H-indol-3-yl)-2-methylpropyl]amino}-8-fluorochromane-5-carboxamide;
8-fluoro-3-{[2-(7-methoxy-1-benzofuran-3-yl)ethyl]amino}chromane-5-carboxamide;
3-{ethyl[2-(7-methoxy-1-benzofuran-3-yl)ethyl]amino}-8-fluorochromane-5-carboxamide;
8-fluoro-3-[[2-(7-methoxy-1-benzofuran-3-yl)ethyl](propyl)amino]chromane-5-carboxamide;
3-{cyclobutyl[2-(7-methoxy-1-benzofuran-3-yl)ethyl]amino}-8-fluorochromane-5-carboxamide;
3-{(cyclopropylmethyl)[2-(7-methoxy-1-benzofuran-3-yl)ethyl]amino}-8-fluorochromane-5-carboxamide;
8-fluoro-3-{[3-(7-methoxy-1-benzofuran-3-yl)propyl]amino}chromane-5-carboxamide;
3-{ethyl[3-(7-methoxy-1-benzofuran-3-yl)propyl]amino}-8-fluorochromane-5-carboxamide;
8-fluoro-3-[[3-(7-methoxy-1-benzofuran-3-yl)propyl](propyl)amino]chromane-5-carboxamide;
3-{cyclobutyl[3-(7-methoxy-1-benzofuran-3-yj)propyl]amino}-8-fluorochromane-5-carboxamide;
3-{(cyclopropylmethyl)[3-(7-methoxy-1-benzofuran-3-yl)propyl]amino}-8-fluorochromane-5-carboxamide;
3-{butyl[3-(7-methoxy-1-benzofuran-3-yl)propyl]amino}-8-fluorochromane-5-carboxamide;
8-fluoro-3-{[4-(7-methoxy-1-benzofuran-3-yl)butyl]amino) chromane-5-carboxamide;
3-{ethyl[4-(7-methoxy-1-benzofuran-3-yl)butyl]amino}-8-fluorochromane-5-carboxamide;
8-fluoro-3-[[4-(7-methoxy-1-benzofuran-3-yl)butyl](propyl)amino]chromane-5-carboxamide;
3-{(cyclopropylmethyl) [4-(7-methoxy-1-benzofuran-3-yl)butyl]amino}-8-fluorochromane-5-carboxamide;
3-{cyclobutyl[4-(7-methoxy-1-benzofuran-3-yl)butyl]amino}-8-fluorochromane-5-carboxamide;
(3R)-8-fluoro-3-[[4-(7-methoxy-1-benzofuran-3-yl)butyl](propyl)amino]chromane-5-carboxamide;
8-fluoro-3-{[(6-fluoro-2,3,4,9-tetrahydro- 1H-carbazol-3-yl)methyl]amino) chromane-5-carboxamide;
(3R)-3-(cyclobutyl{[(3S)-6-fluoro-2,3,4,9-tetrahydro-1H-carbazol-3-yl]methyl}amino)-8-fluorochromane-5-carboxamide;
(3R)-3-(cyclobutyl{[(3R)-6-fluoro-2,3,4,9-tetrahydro-1H-carbazol-3-yl]methyl}amino)-8-fluorochromane-5-carboxamide;
(3S)-3-(cyclobutyl{[(3S)-6-fluoro-2,3,4,9-tetrahydro-1H-carbazol-3-yl]methyl}amino)-8-fluorochromane-5-carboxamide;
(3S)-3-(cyclobutyl{[(3R)-6-fluoro-2,3,4,9-tetrahydro- 1H-carbazol-3-yl]methyl}amino)-8-fluorochromane-5-carboxamide;
(−)-(3R)-8-fluoro-3-({[6-fluoro-2,3,4,9-tetrahydro-1H-carbazol-3-yl]methyl}amino)chromane-5-carboxamide;
(+)-(3R)-8-fluoro-3-({[6-fluoro-2,3,4,9-tetrahydro-1H-carbazol-3-yl]methyl}amino)chromane-5-carboxamide;
(−)-(3R)-3-(ethyl{[6-fluoro-2,3,4,9-tetrahydro- 1H-carbazol-3-yl]methyl}amino)-8-fluorochromane-5-carboxamide;
(+)-(3R)-3-(ethyl{[6-fluoro-2,3,4,9-tetrahydro- 1H-carbazol-3-yl]methyl}amino)-8-fluorochromane-5-carboxamide;
(−)-(3R)-8-fluoro-3-[{[6-fluoro-2,3,4,9-tetrahydro- 1H-carbazol-3-yl]methyl}(propyl)amino]chromane-5-carboxamide;
(+)-(3R)-8-fluoro-3-[{[6-fluoro-2,3,4,9-tetrahydro- 1H-carbazol-3-yl]methyl}(propyl)amino]chromane-5-carboxamide;
(−)-(3R)-3-((cyclopropylmethyl){[6-fluoro-2,3,4,9-tetrahydro- 1H-carbazol-3-yl]methyl}amino)-8-fluorochromane-5-carboxamide;
(+)-(3R)-3-((cyclopropylmethyl){[6-fluoro-2,3,4,9-tetrahydro- 1H-carbazol-3-yl]methyl}amino)-8-fluorochromane-5-carboxamide;
(+)-(3R)-8-fluoro-3-({[6-fluoro-2,3,4,9-tetrahydro-1H-carbazol-2-yl]methyl}amino)chromane-5-carboxamide;
(−)-(3R)-8-fluoro-3-({[6-fluoro-2,3,4,9-tetrahydro-1H-carbazol-2-yl]methyl}amino)chromane-5-carboxamide;
3-[(1,4-cis)-4-(5-Methoxy-chroman-3-ylamino)-cyclohexyl]-1H-indole-5-carbonitrile;
3-[(1,4-trans)-4-(5-Methoxy-chroman-3-ylamino)-cyclohexyl]-1H-indole-5-carbonitrile;
cis-N-[4-(5-fluoro-1H-indol-3-yl)cyclohexyl]-5-methoxychroman-3-amine;
trans-N-[4-(5-fluoro-1H-indol-3-yl)cyclohexyl]-5-methoxychroman-3-amine;
cis-N-[4-(5-fluoro-1H-indol-3-yl)cyclohexyl]-5-methoxy-N-propylchroman-3-amine;
trans-N-[4-(5-fluoro-1H-indol-3-yl)cyclohexyl]-5-methoxy-N-propylchroman-3-amine;
8-Fluoro-3-{[3-(1H-indol-1-yl)propyl]amino}chromane-5-carboxamide;
8-Fluoro-3-[4-(indol-1-yl)-butylamino]-chroman-5-carboxylic acid amide;
8-Fluoro-3-[4-(5-fluoro-indol-1-yl)-butylamino]-chroman-5-carboxylic acid amide;
8-Fluoro-3-[4-(6-fluoro-indol-1-yl)-butylamino]-chroman-5-carboxylic acid amide;
8-Fluoro-3-{[4-(7-fluoro-1H-indol-1-yl)butyl]amino}chromane-5-carboxamide;
3-{Ethyl[4-(7-fluoro-1H-indol-1-yl)butylamino}-8-fluorochromane-5-carboxamide;
8-Fluoro-3-[[4-(7-fluoro-1H-indol-1-yl)butyl](propyl)amino]chromane-5-carboxamide;
3-{(Cyclopropylmethyl)[4-(7-fluoro-1H-indol-1-yl)butyl]amino}-8-fluorochromane-5-carboxamide;
3-{Cyclobutyl[4-(7-fluoro-1H-indol-1-yl)butyl]amino}-8-fluorochromane-5-carboxamide;
3-{Ethyl[4-(6-fluoro-1H-indol-1-yl)butyl]amino}-8-fluorochromane-5-carboxamide;
8-Fluoro-3-[[4-(6-fluoro-1H-indol-1-yl)butyl](propyl)amino]chromane-5-carboxamide;
3-{(Cyclopropylmethyl)[4-(6-fluoro-1H-indol-1-yl)butyl]amino}-8-fluorochromane-5-carboxamide;
3-{Cyclobutyl[4-(6-fluoro-1H-indol-1-yl)butyl]amino}-8-fluorochromane-5-carboxamide;
3-{Ethyl[4-(5-fluoro-1H-indol-1-yl)butyl]amino}-8-fluorochromane-5-carboxamide;
8-Fluoro-3-[[4-(5-fluoro-1H-indol-1-yl)butyl](propyl)amino]chromane-5-carboxamide;
3-{(Cyclopropylmethyl)[4-(5-fluoro-1H-indol-1-yl)butyl]amino}-8-fluorochromane-5-carboxamide;
3-{Cyclobutyl[4-(5-fluoro-1H-indol-1-yl)butyl]amino}-8-fluorochromane-5-carboxamide;
3-{Ethyl[4-(4-fluoro-1H-indol-1-yl)butyl]amino}-8-fluorochromane-5-carboxamide;
8-Fluoro-3-[[4-(4-fluoro-1H-indol-1-yl)butyl](propyl)amino]chromane-5-carboxamide;
3-{(Cyclopropylmethyl)[4-(4-fluoro-1H-indol-1-yl)butyl]amino}-8-fluorochromane-5-carboxamide;
3-{Cyclobutyl[4-(4-fluoro-1H-indol-1-yl)butyl]amino}-8-fluorochromane-5-carboxamide, and pharmaceutical salts thereof.

The compound of general formula I and compounds of structures Ia, Ib, Ic, Id, Ie, If, Ig, Ih, Ij, Ik, or Im may be prepared by conventional synthetic techniques. In the following synthetic techniques, suitable aprotic polar solvents include, but are not limited to, dimethyl sulfoxide, dimethylformamide, tetrahydrofuran, acetone and ethanol. Suitable acid binding agents include, but are not limited to, organic tertiary bases, such as, for example, triethylamine, triethanolamine, 1,8-diazabicyclo[5.4.0]undec-7-ene (DBU), and diisopropylethylamine (DIPEA); and alkaline metal carbonates, such as, for example, potassium carbonate and sodium carbonates. Suitable reducing agents include, but are not limited to, sodium cyanoborohydride and sodium triacetoxyborohydride.

Compounds of structures Ia, Id, Ie and If may be prepared by conventional methods as illustrated in Scheme I below. The appropriate bromoalkyl indole 3 is combined with either a 3-amino chroman derivative 1 or a 2-amino tetralin 2 in an aprotic, polar solvent, in the presence of acid binding agents and heated to a temperature of 60-100° C. for several hours to generate the desired products Ia, Id, Ie and If (where R¹ is hydrogen). This is then followed by reductive amination using sodium cyanoborohydride or sodium triacetoxyborohydride and the desired alkyl aldehyde or cycloalkyl ketone to introduce the appropriate alkyl chain R¹ on the basic nitrogen if desired.

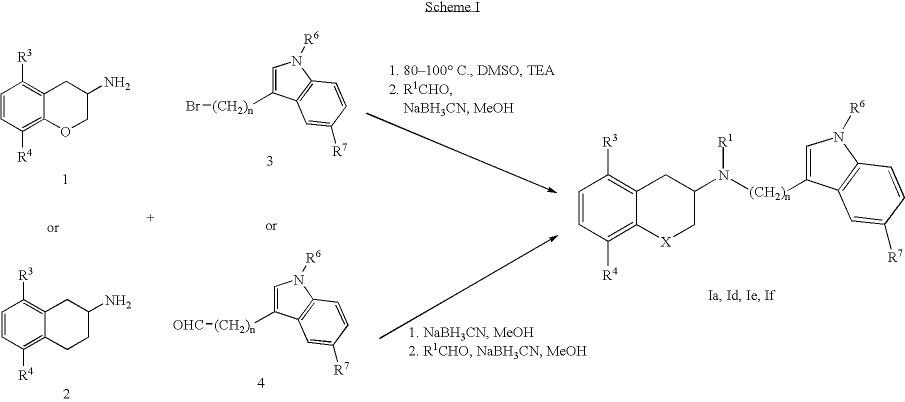

Alternatively, an aldehyde alkyl indole 4 can be used as starting material and combined with either a 3-amino chroman derivative 1 or a 2-amino tetralin 2 in the presence of a reducing agent to generate the desired products Ia, Id, Ie and If (where R¹ is hydrogen). This is then followed by a second reductive amination using sodium cyanoborohydride or sodium triacetoxyborohydride and the desired alkyl aldehyde or cycloalkyl ketone to introduce the appropriate alkyl chain R¹ on the basic nitrogen if desired. The compounds of the invention may be resolved into their enantiomers by conventional methods. Alternatively, compounds 1 and 2 may be resolved into their two enantiomers either by chiral resolution or chiral HPLC to generate pure enantiomers.

Alternatively, as illustrated in Scheme II, a 3-aminoalkyl indole 5 can be used as starting material and combined with either a chroman 3-carbonyl derivative 6 or a tetralin 2-carbonyl derivative 7 in the presence of a reducing agent to generate the desired products Ia, Id, Ie and If (where R¹ is hydrogen). This is then followed by a second reductive amination using sodium cyanoborohydride or sodium triacetoxyborohydride and the desired alkyl aldehyde or cycloalkyl ketone to introduce the appropriate alkyl chain R¹ oh the basic nitrogen if desired.

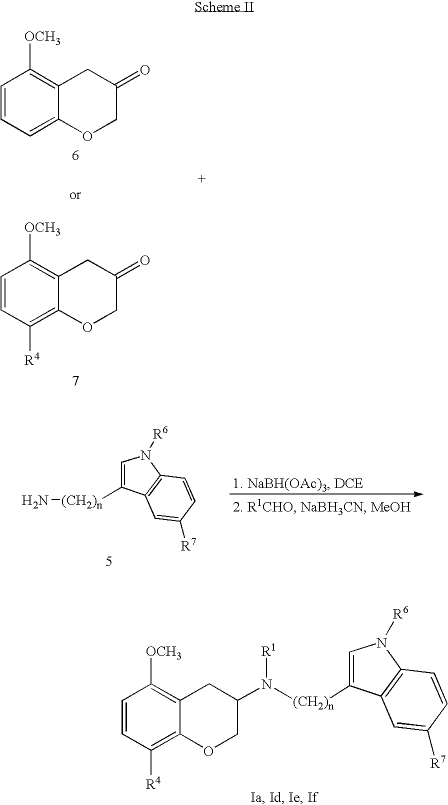

Compounds of structure Ib can be prepared by conventional methods, as illustrated in Scheme III. The appropriate ketone alkyl indole 8 is combined with a 3-aminochroman derivative 1c in the presence of a reducing agent such as sodium cyanoborohydride or sodium triacetoxyborohydride to generate the desired product Ib (where R¹ is hydrogen). This is then followed by a second reductive amination using sodium cyanoborohydride and the desired alkyl aldehyde or cycloalkyl ketone to introduce the appropriate alkyl chain R¹ on the basic nitrogen if desired. The compounds of the invention may then be resolved into their enantiomers and diastereomers by chiral HPLC.

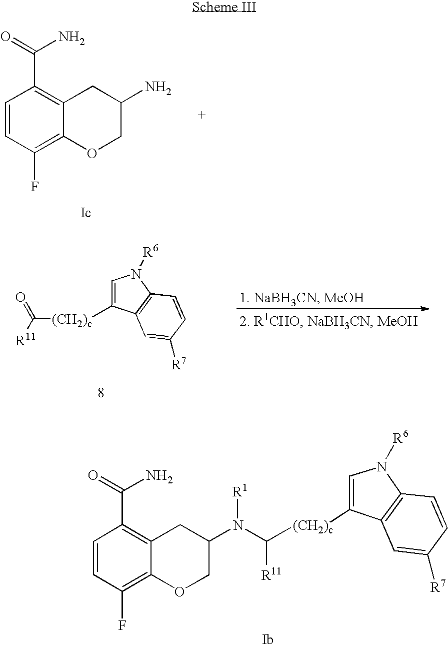

Compounds of structure Ic can be prepared by conventional methods, as illustrated in Scheme IV. The appropriate branched bromoalkyl indole 9 is combined with a 3-amino chroman derivative 1c in an aprotic polar solvent, in the presence of triethylamine and heated to a temperature of 60-100° C. for several hours to generate the desired product Ic (where R¹ is hydrogen). This is then followed by a reductive amination using sodium cyanoborohydride and the desired alkyl aldehyde or cycloalkyl ketone to introduce the appropriate alkyl chain R¹ on the basic nitrogen if desired.

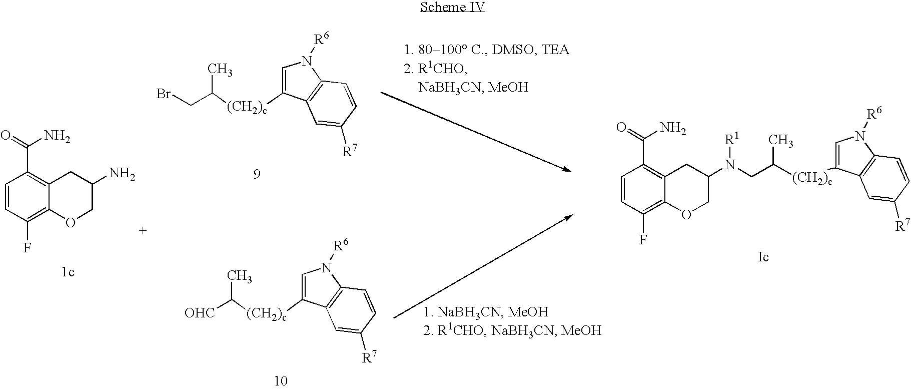

Alternatively, a branched aldehyde alkyl indole 10 can be used as starting material and combined with a 3-amino chroman derivative 1c in the presence of a reducing agent such as sodium cyanoborohydride or sodium triacetoxyborohydride to generate the desired product Ic (where R¹ is hydrogen). This is then followed by a second reductive amination using sodium cyanoborohydride and the desired alkyl aldehyde or cycloalkyl ketone to introduce the appropriate alkyl chain R¹ on the basic nitrogen if desired. The compounds of the invention may be resolved into their enantiomers and diastereomers by chiral HPLC.

Compounds of structure Ig (where Y is oxygen or sulfur) are prepared by conventional methods as illustrated in Scheme V. Benzothiophene intermediate 11 is combined with a 3-amino chroman derivative 1 in a solvent such as THF or DMF in the presence of potassium carbonate at room temperature for several days to generate the desired product Ig (where R¹ is hydrogen and R¹⁶ is a keto group). The ketone is then reduced to hydroxyl in the presence of sodium borohydride in methanol (R¹⁶ is hydroxyl). This is then followed by a reductive amination, using sodium cyanoborohydride and the desired alkyl aldehyde or cycloalkyl ketone to introduce the appropriate alkyl chain R¹ on the basic nitrogen if desired. The hydroxyl is then reduced to a methylene in the presence of triethylsilane and trifluoroacetic acid in a solvent such as dichloromethane (R¹⁶ is hydrogen). For the benzofuran series of compounds, the 3-bromoalkylbenzofuran intermediate 12 is combined with a 3-amino chroman derivative 1 in a solvent such as dimethylsulfoxide in the presence of triethylamine and heated to a temperature of 60-100° C. for several hours to generate product Ig (where R¹ and R¹⁶ are each hydrogen). This is then followed by a reductive amination using sodium cyanoborohydride and the desired alkyl aldehyde or cycloalkyl ketone to introduce the appropriate alkyl chain R¹ on the basic nitrogen if desired.

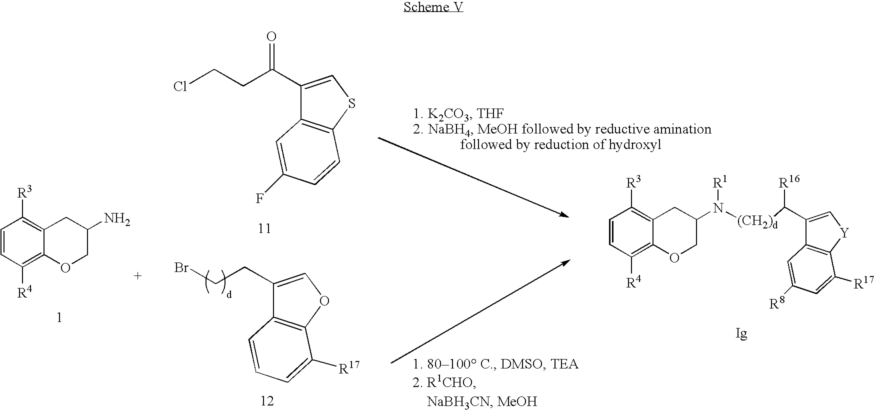

Alternatively, as illustrated in Scheme VI, the appropriate 3-aminoalkyl benzothiophene 13 or 3-aminoalkyl benzofuran 14 is combined with a chroman 3-carbonyl derivative 6 in the presence of a reducing agent such as sodium cyanoborohydride or sodium triacetoxyborohydride to generate the desired product Ig (where R¹ and R¹⁶ are each hydrogen). This is then followed by a second reductive amination using sodium cyanoboro hydride and the desired alkyl aldehyde or cycloalkyl ketone to introduce the appropriate alkyl chain R¹ on the basic nitrogen if desired. The compounds of the invention may be resolved into their enantiomers by chiral HPLC.

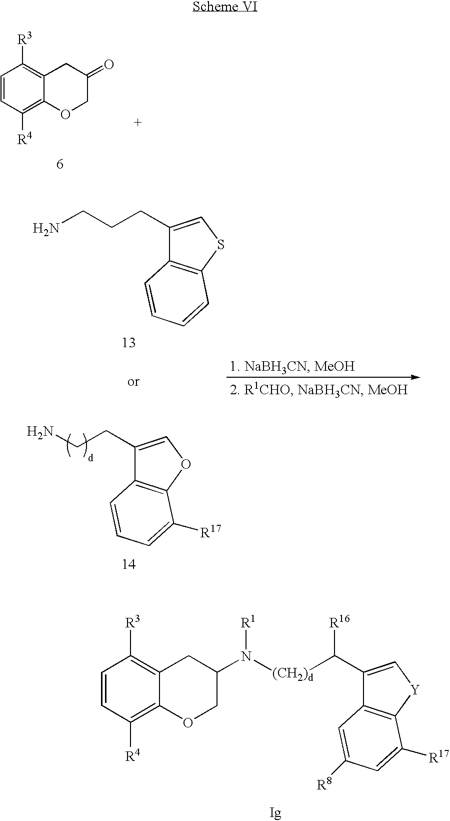

Compounds of structure Ig (where Y is NH) are prepared by conventional methods as illustrated in Scheme VII. The N-benzenesulfonyl protected indole intermediate 15 is combined with a 3-amino chroman derivative 1 in a solvent such as DMF in the presence of potassium carbonate at room temperature to generate Ig (where R¹ is hydrogen, R¹⁶ is a keto group and Y is N-benzenesulfonyl). Treatment with potassium carbonate in methanol under reflux followed by reductive amination using sodium cyanoborohydride and the desired aldehyde or cycloalkyl ketone to introduce the appropriate alkyl chain R¹ on the basic nitrogen generates the desired product Ig (where R¹⁶ is a keto group and Y is NH). The compounds of the invention may be resolved into their enantiomers and diastereomers by chiral HPLC.

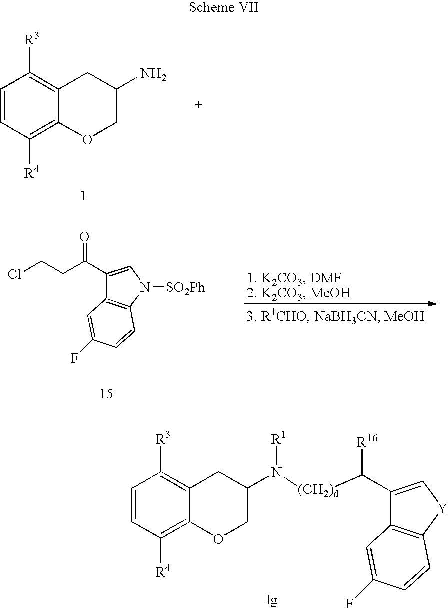

Compounds of structure Ig (where Y is NH and R¹⁶ is methyl) are prepared by conventional methods as illustrated in Scheme VIII. The appropriate branched aldehyde alkyl indole 16 is combined with a 3-amino chroman derivative 1c in the presence of a reducing agent such as sodium cyanoborohydride or sodium triacetoxyborohydride to generate the desired product Ig (where R¹ is hydrogen). This is then followed-by a second reductive amination using sodium cyanoborohydride and the desired alkyl aldehyde or cycloalkyl ketone to introduce the appropriate alkyl chain R¹ on the basic nitrogen if desired. The compounds of this invention may be resolved into their enantiomers and diastereomers by chiral HPLC.

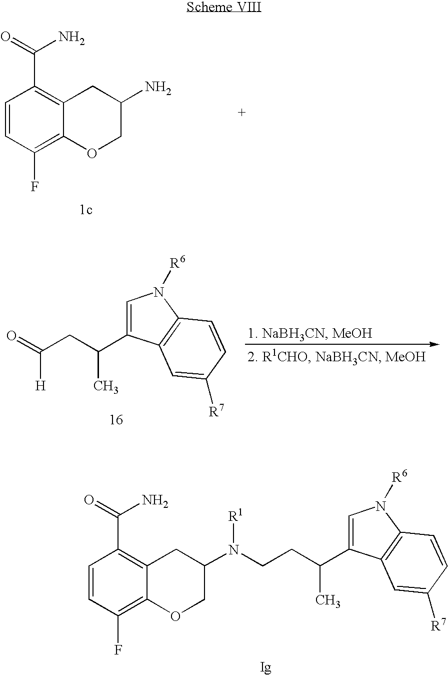

The bromoalkyl indoles 3 and amino alkyl indoles 5, required to prepare the compounds of the invention, are known compounds and were prepared as described in U.S. Pat. No. 6,121,307, which is incorporated herein by reference. The aldehyde alkyl indole 4 is a known compound and was prepared by the procedure illustrated in Scheme IX.

3-(5-fluoro-1H-indol-3-yl)propan-1-ol 17a or 3-(6-fluoro-1H-indol-3-yl)propan-1-ol 17b, generated from a Fisher-Indole synthesis, were subjected to oxidation conditions to afford the desired 3-(5-fluoro-1H-indol-3-yl)propanal 4a or 3-(6-fluoro-1H-indol-3-yl)propanal 4b.

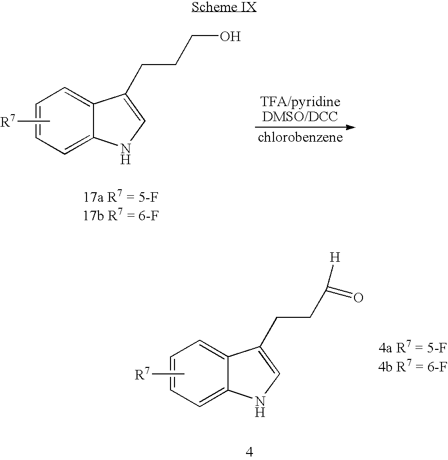

The 3-amino-5-methoxychroman derivative la and the 3-amino-8-fluoro-5-methoxychroman derivative lb are known compounds, and were prepared as illustrated in Scheme X according to a procedure in U.S. Pat. No. 5,616,610, which is incorporated herein by reference.

The commercially available 2-hydroxy-6-methoxybenzaldehyde 18 is first converted to 5-methoxy-3-nitro-2H-chromene 19 by reaction with 2-nitroethanol in isoamylacetate in the presence of di-n-butylammonium chloride under reflux. The double bond in derivative 19 is reduced with sodium borohydride to generate 5-methoxy-3-nitrochromane 20, which is then converted to (5-methoxy-3,4-dihydro-2H-chromen-3-yl)amine 1a under phase transfer hydrogenation conditions using hydrazine hydrate and Raney-Nickel. Derivative 1a was reacted with benzyl bromide generating N,N-dibenzyl-N-(5-methoxy-3,4-dihydro-2H-chromen-3-yl) amine 21, which is then brominated using NBS yielding N,N-dibenzyl-N-(8-bromo-5-methoxy-3,4-dihydro-2H-chromen-3-yl)amine 22. The bromine is then displaced by a fluorine using n-butyl lithium and N-fluorobenzenesulfonimide to generate N,N-dibenzyl-N-(8-fluoro-5-methoxy-3,4-dihydro-2H-chromen-3-yl)amine 23, which is then deprotected to yield the desired (8-fluoro-5-methoxy-3,4-dihydro-2H-chromen-3-yl)amine 1b.

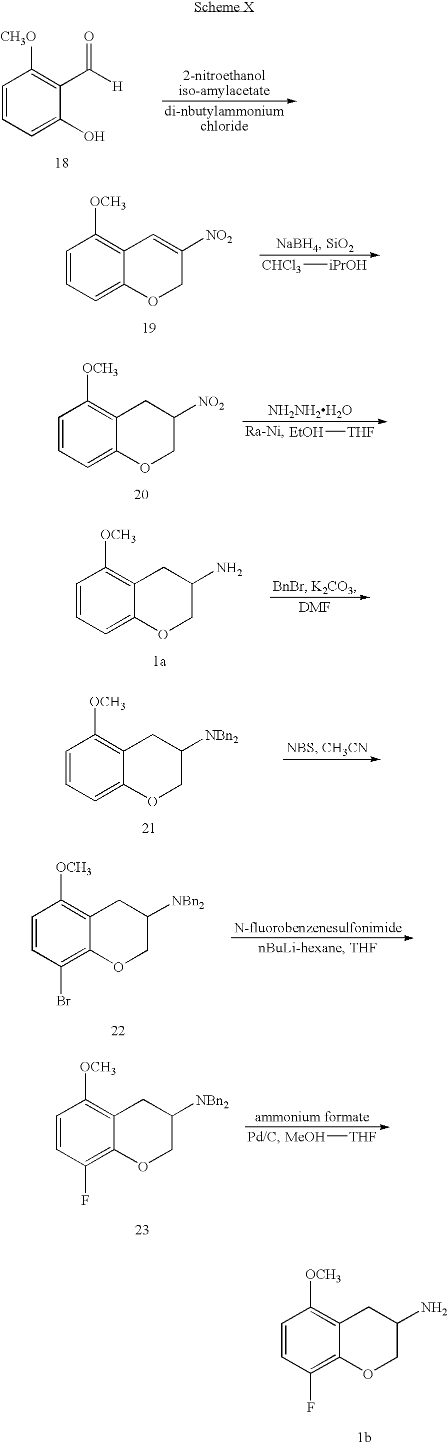

The 3-amino-8-fluorochromane-5-carboxamide 1c is a known compound, and was prepared by the procedure illustrated in Scheme XI with modifications of the reaction conditions of the original synthesis elaborated in U.S. Pat. No. 6,197,978, which is incorporated herein by reference. Similarly, 3-amino-8-chlorochromane-5-carboxamide 1d was prepared using 2-chloro-5-(trifluoromethyl)phenol, 24d, as starting material while 3-aminochromane-5-carboxamide 1e was prepared using 3-hydroxybenzoic acid as starting material. The similar procedure was also used for the preparation of 3-amino-5-fluorochromane-8-carboxamide 1f except that 4-fluoro-2-hydroxybenzoic acid was used as starting material and the synthesis is elaborated in Scheme XXVIII below.

The commercially available 4-fluoro-3-hydroxybenzoic acid 24c is first converted to the methyl ester, and the resulting methyl 4-fluoro-3-hydroxybenzoate 25c reacted with propargyl bromide to generate methyl 4-fluoro-3-(prop-2-ynyloxy)benzoate 26c. Compound 26c is then cyclized in the presence of N,N-diethylaniline at 180-220° C. generating methyl 8-fluoro-2H-chromene-5-carboxylate 27c. The methyl ester was then cleaved under basic conditions producing 8-fluoro-2H-chromene-5-carboxylic acid 28c, and the resulting acid converted to the carboxamide via a carbonyldiimidazole derivative, which was then displaced with ammonia to generate 8-fluoro-2H-chromene-5-carboxamide 29c. The nitration of the double bond was carried out using a phase transfer reagent, 18-crown-6, in the presence of potassium nitrite and iodide. Sonication was used in this reaction to increase the solubility of the nitrite ion and speed up the reaction. 8-fluoro-3-nitro-2H-chromene-5-carboxamide 30c was then isolated, and the double bond reduced with sodium borohydride to generate 8-fluoro-3-nitrochromane-5-carboxamide 31c. Finally, phase transfer hydrogenation using hydrazine hydrate and Raney-Nickel generated the desired 3-amino-8-fluorochromane-5-carboxamide 1c.

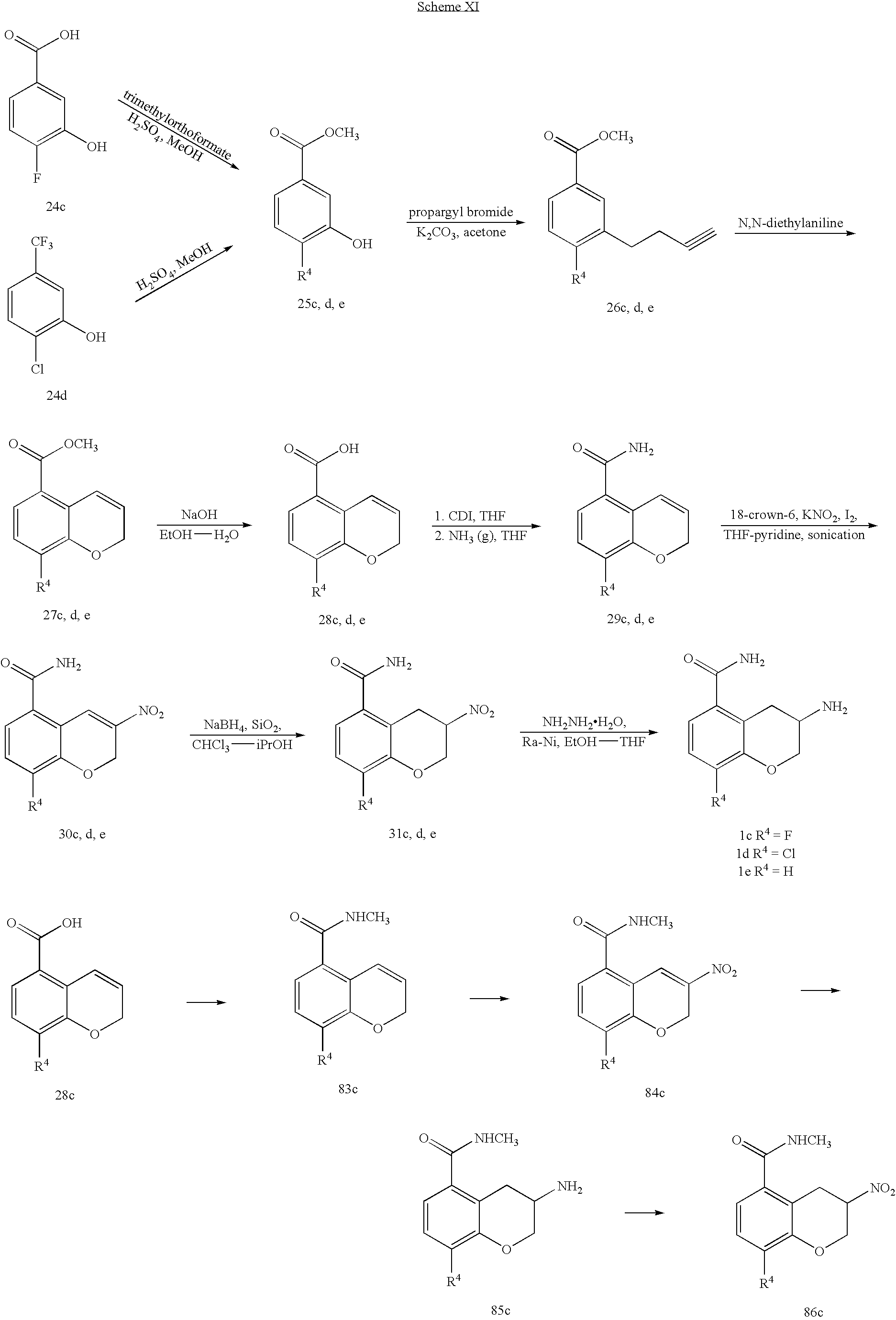

Compound 6 is also a known compound and was prepared by generally following the procedure elaborated in U.S. Pat. No. 5,306,830, incorporated herein by reference, as illustrated in Scheme XII. The commercially available 2-hydroxy-6-methoxybenzaldehyde 18 is first converted to 5-methoxy-2H-chromene-3-carbonitrile 32 by O-cyanoethylation and aldol cyclization in the presence of Dabco in acrylonitrile. Hydrolysis of the cyano group in 32 under basic conditions afforded 5-methoxy-2H-chromene-3-carboxylic acid 33, which was then subjected to a Curtius rearrangement followed by acid-catalyzed hydrolysis of the resulting vinyl isocyanate generating the desired 5-methoxy-2H-chromen-3(4H)-one 6.

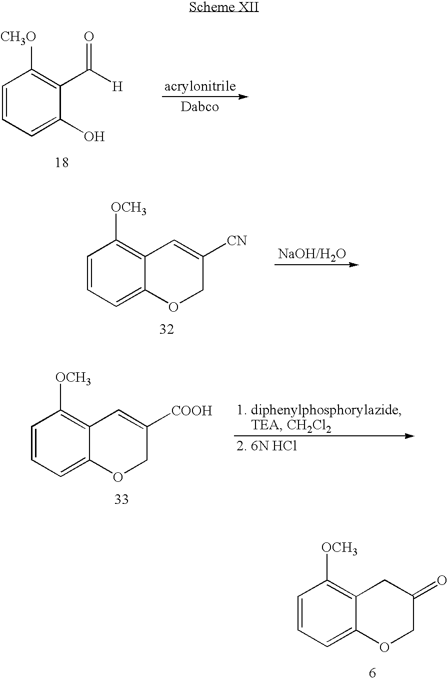

Compound 7a is also a known compound and was prepared by the procedure illustrated in Scheme XIII. The commercially available 1,7-dihydroxynaphthalene 34 was methylated with iodomethane in the presence of potassium carbonate generating 1,7-dimethoxynaphthalene 35. Derivative 35 was reduced to give the desired 8-methoxy-3,4-dihydronaphthalen-2(1H)-one 7a upon acid hydrolysis.

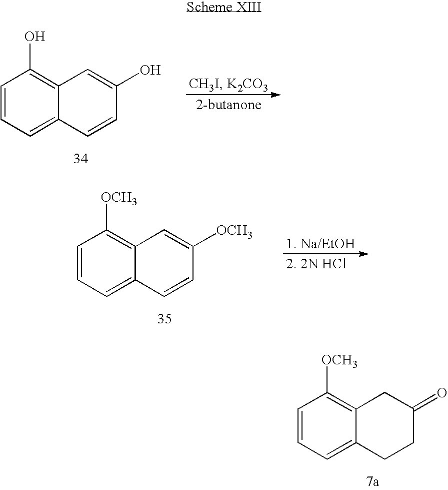

Compound 7b is also a known compound and was prepared by the procedure illustrated in Scheme XIV. Esterification of the commercially available 4-flurophenol 36 with 3-chloropropionyl chloride generated 4-fluorophenyl-3-chloro propanoate 37, which was subjected to a Fries rearrangement in the presence of aluminum trichloride affording 4-fluoro-7-hydroxyindan-1-one 38. Methylation of derivative 38 generated 4-fluoro-7-methoxyindan-1-one 39, which was converted to 4-fluoro-7-methoxy-1-methyleneindane 40 through a Wittig reaction. Ring expansion using thallium (III) nitrate followed by acid hydrolysis of the resulting dimethyl ketal afforded the desired 5-fluoro-8-methoxy-3,4-dihydronaphthalen-2(1H)-one 7b.

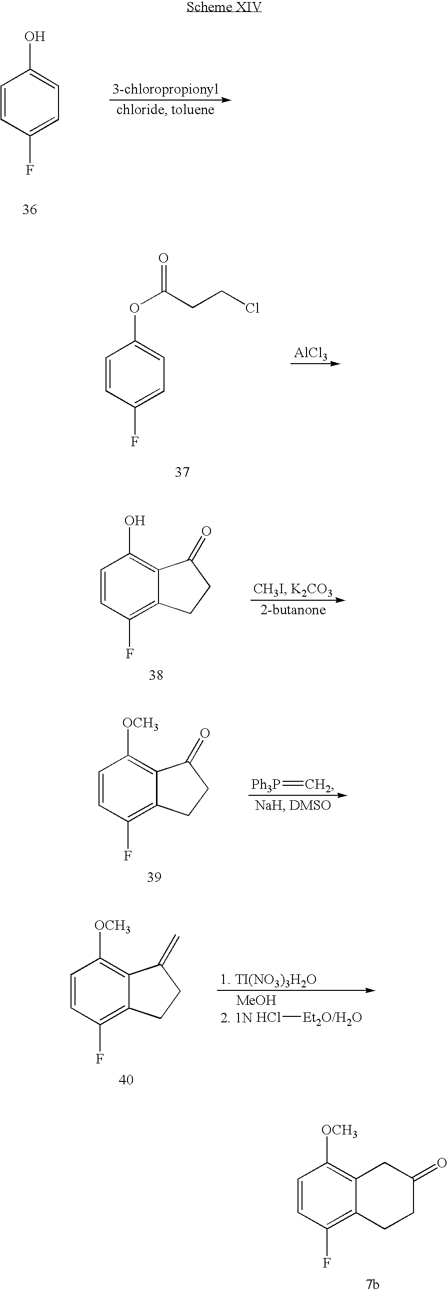

Compound 2a is a known compound, described in U.S. Pat. No. 5,376,687, incorporated herein by reference, and was prepared by the procedure illustrated in Scheme XV. Compound 2b is a new entity and was prepared from 2a by the procedure illustrated in Scheme XV. Derivative 7b was subjected to reductive amination conditions generating N-benzyl-N-(5-fluoro-8-methoxy-1,2,3,4-tetrahydro-naphthalen-2yl)amine 41, which was then converted to the desired (5-fluoro-8-methoxy-1,2,3,4-tetrahydronaphathalen-2-yl)amine 2a upon cleavage of the benzyl protecting group. Cleavage of the methoxy group under BBr₃ conditions generated 42 which was then converted to the triflate derivative 7-(benzylamino)-4-fluoro-5,6,7,8-tetrahydronaphthalene-1-yl trifluoromethanesulfonate 43. Displacement of the triflate with carbon monoxide using palladium acetate and 1,3-bis-(diphenylphosphine) propane generated methyl-7-(benzylamino)-4-fluoro-5,6,7,8-tetrahydronaphthalene-1-carboxylate 44. Cleavage of the methyl ester under basic conditions afforded 7-(benzylamino)-4-fluoro-5,6,7,8-tetrahydronaphthalene-1-carboxylic acid 45. The acid was converted to the carboxamide via a carbonyldiimidazole derivative, which was then displaced with ammonia to generate 7-(benzylamino)-4-fluoro-5,6,7,8-tetrahydronaphthalene-1-carboxamide 46. Finally the amine was deprotected under hydrogenation conditions to generate the desired 7-amino-4-fluoro-5,6,7,8-tetrahydronaphthalene-1-carboxamide 2b.

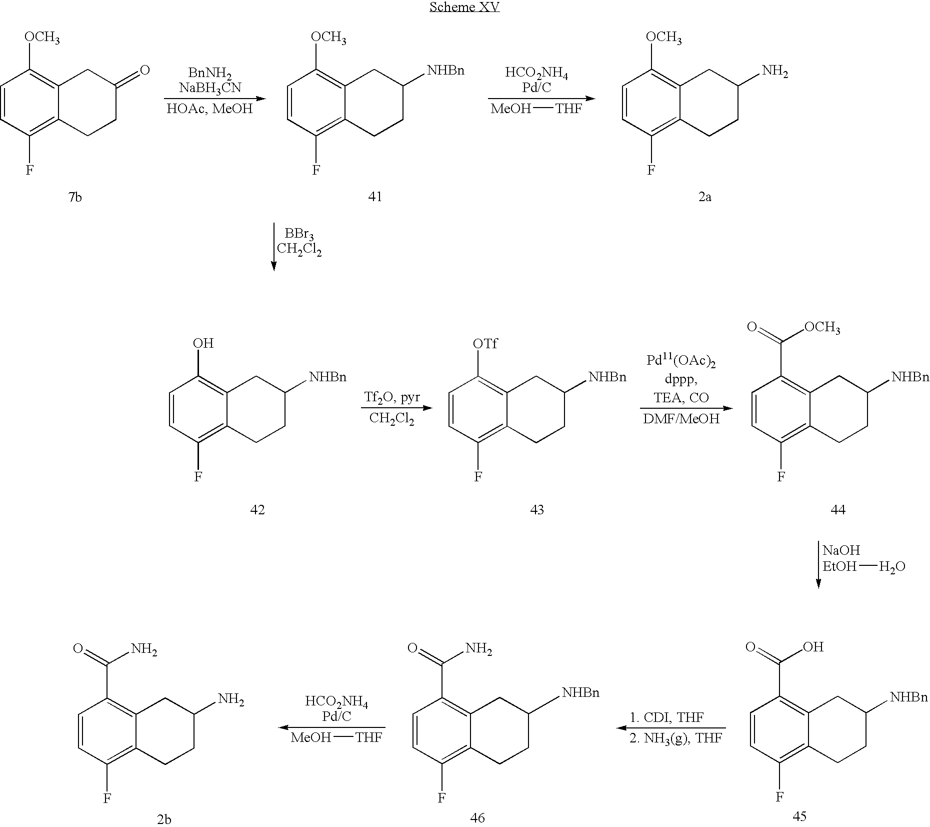

Compounds 9 and 10 are new entities and were prepared by the procedure illustrated in Scheme XVI. 5-Fluorogramine was reacted with diethyl methylmalonate in the presence of tributylphosphine to generate diethyl[(5-fluoro-1H-indol-3-yl)methyl](methyl)malonate 47, which was then converted to the di-acid in the presence of base affording compound 48, [(5-fluoro-1H-indol-3-yl)methyl](methyl)malonic acid. Derivative 48 was decarboxylated in bromobenzene under reflux followed by reduction with lithium aluminum -hydride to generate 3 (5-fluoro-1H-indol-3-yl)-2-methylpropan-1-ol 49. Derivative 49 can either be converted to desired compound 9,3-(3-bromo-2-methylpropyl)-5-fluoro-1H-indole under standard bromination conditions, or to the desired aldehyde 10, 3-(5-fluoro-1H-indol-3-yl)-2-methylpropanal using modified Swern conditions as described earlier in this patent.

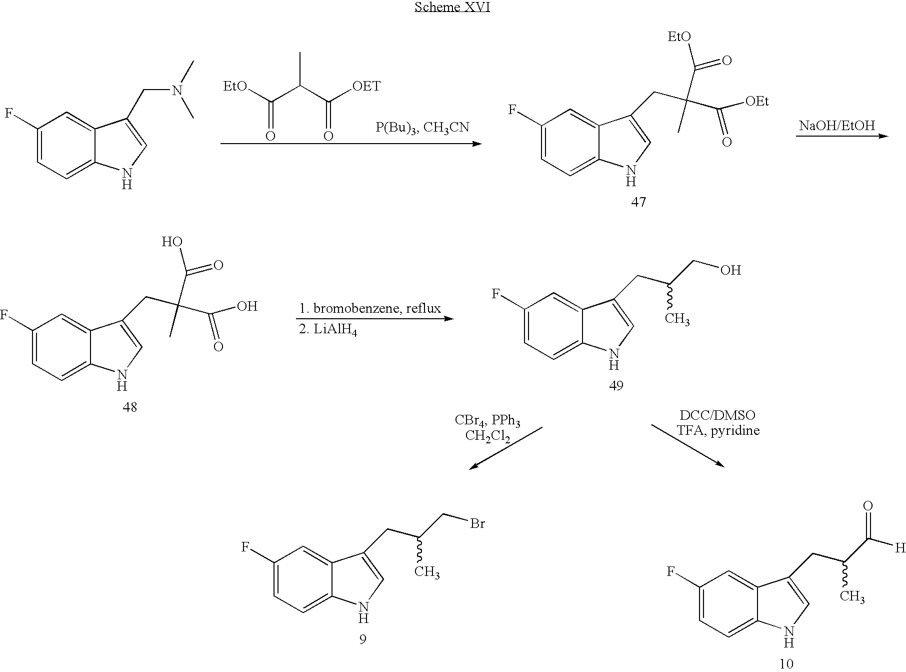

The 3-chloro-1-(5-fluoro-1-benzothien-3-yl)propan-1-one 11 is a known compound, and was prepared by the procedure illustrated in Scheme XVII. Commercially available 4-fluorobenzenethiol 54 was converted to 1-[(2,2-diethoxyethyl)thio]-4-fluorobenzene 55 by reaction with bromoacetaldehyde diethyl acetal in the presence of potassium carbonate. Cyclization using polyphosphoric acid (PPA) generated 5-fluoro-1-benzothiophene 56, which was then subjected to Friedel-Crafts acylation with 3-chloropropionyl chloride affording the desired 3-chloro-1-(5-fluoro-1-benzothien-3-yl)propan-1-one 11.

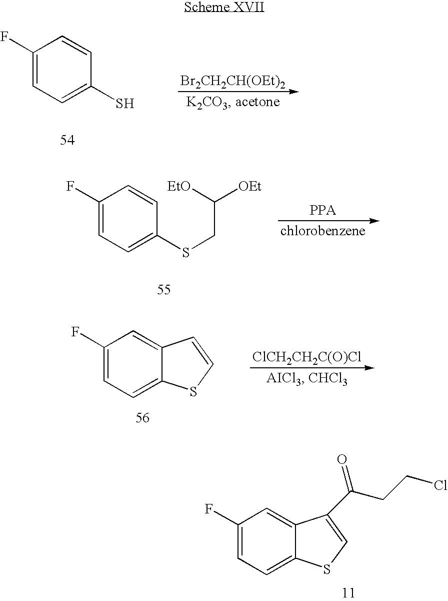

The [3-(1-benzothien-3-yl)propyl]amine 13 was prepared by the procedure illustrated in Scheme XVIII. The commercially available 1-benzothien-3-ylacetic acid 57 was reduced to the alcohol in the presence of lithium aluminum, hydride generating 2-(1-benzothien-3-yl)ethanol 58. Tosylation of the hydroxyl group under standard conditions afforded 2-(1-benzothien-3-yl)ethyl-4-methylbenzene sulfonate 59, which was then converted to the cyano derivative, 3-(1-benzothien-3-yl)propanenitrile 60. Final reduction under hydrogenation conditions generated the desired [3-(1-benzothien-3-yl)propyl]amine 13.

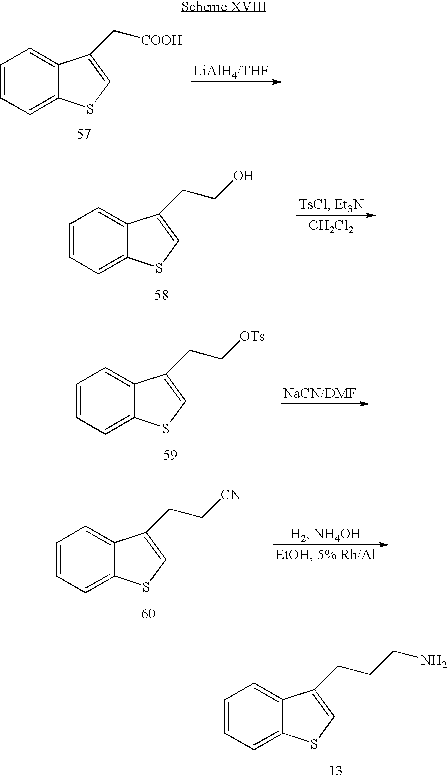

The [3-(1-benzofuran-3-yl)propyl]amine 14 (R¹⁷=H) was prepared by the procedure illustrated in Scheme XIX. The commercially available 1-benzofuran-3-(2H)-one 61 was subjected to a Wittig reaction with methyl (triphenyphosphoranylidene) acetate to generate methyl-1-benzofuran-3-ylacetate 62. Cleavage of the methyl ester under basic conditions afforded 1-benzofuran-3-ylacetic acid 63. Reduction under lithium aluminum hydride conditions generated 2-(1-benzofuran-3-yl)ethanol 64a, which was then converted to the bromide under standard conditions affording 3-(2-bromoethyl)-1-benzofuran 12a. Conversion to 3-(1-benzofuran-3-yl)propanenitrile 65a, and reduction under hydrogenation conditions generated the desired [3-(1-benzofuran-3-yl)propyl]amine 14 (R¹⁷ is hydrogen). The same conditions were used for the synthesis of [3-(7-methoxy-1-benzofuran-3-yl)propyl]amine 14 (R¹⁷ is methoxy).

The 3-(3-bromopropyl)-1-benzofuran 12b (R¹⁷ is hydrogen) was prepared from derivative 65a by hydrolysis of the nitrile to the carboxylic acid under basic conditions generating 3-(1-benzofuran-3-yl) propanoic acid 63a. This was followed by reduction to the alcohol 64b followed by conversion to the desired bromide derivative 12b under standard conditions described above.

The 3-(4-bromobutyl)-1-benzofuran 12c (R¹⁷ is hydrogen) was prepared as described for compound 12b using the same sequence of reactions as illustrated in Scheme XIX.

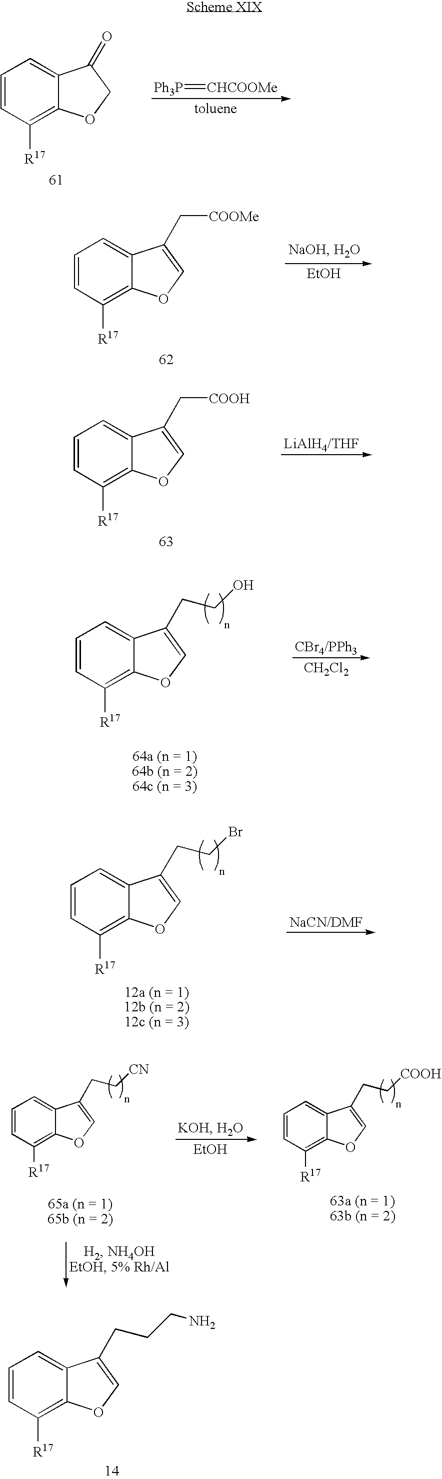

The 3-chloro-1-[5-fluoro-1-(phenylsulfonyl)- 1H-indol-3-yl]propan-1-one 15 was prepared by the procedure illustrated in Scheme XX. The commercially available 5-fluoro-1H-indole 66 was N-protected with benzenesulfonyl chloride in the presence of n-butyl lithium to generate 5-fluoro-1-(phenylsulfonyl)-1H-indole 67. Friedel-Crafts acylation with 3-chloropropionyl chloride afforded the desired product 15. Compound 15 was subjected to alkylation conditions with 3-amino-8-fluorochromane-5-carboxamide 1c to generate desired product 50. Upon reduction of the keto derivative using sodium borohydride compound 51 was isolated and it was then subjected to reductive amination to generate the desired intermediate 52.

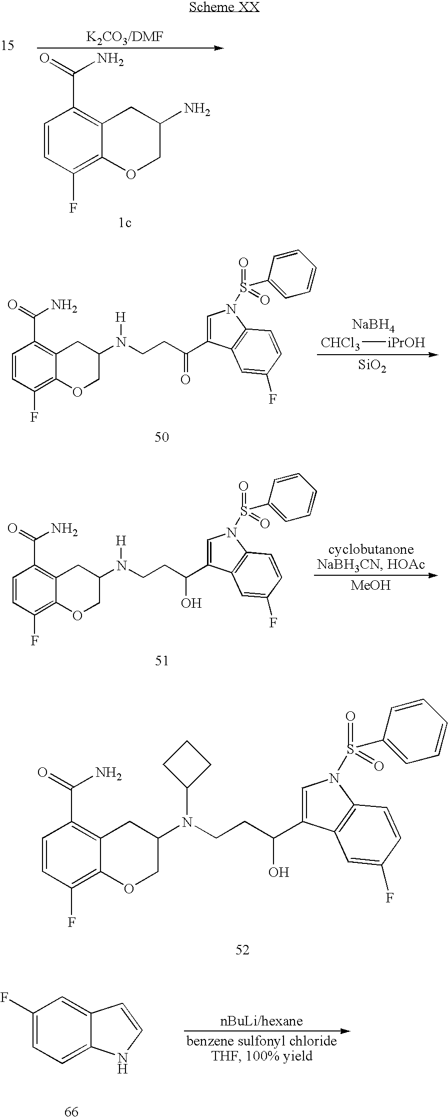

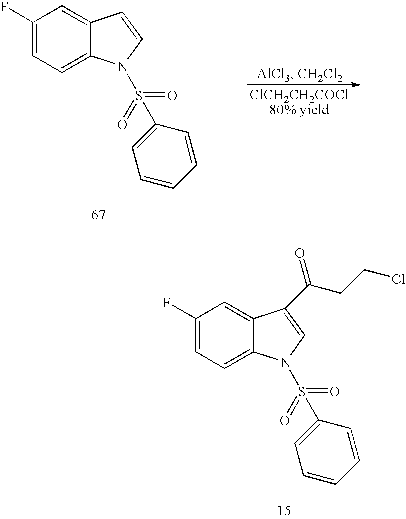

The 3-(5-fluoro-1H-indol-3-yl)butanal 16 was prepared by the procedure illustrated in Scheme XXI. The commercially available 5-fluoro-1H-indole 66 was combined with Meldrum's acid and acetaldehyde in acetonitrile to generate the condensation product 5-[1-(5-fluoro-1H-indol-3-yl)ethyl]-2,2-dimethyl-1,3-dioxane-4,6-dione 68. Upon heating 68 in ethanol-pyridine in the presence of Cu powder, ethanolysis took place with concomitant decarboxylation to generate ethyl 3-(5-fluoro-1H-indol-3yl) butanoate 69. Reduction of the ester in the presence of lithium aluminum hydride generated 3-(5-fluoro-1H-indol-3-yl)butan-1-ol 70 which was then oxidized to the desired product 16.

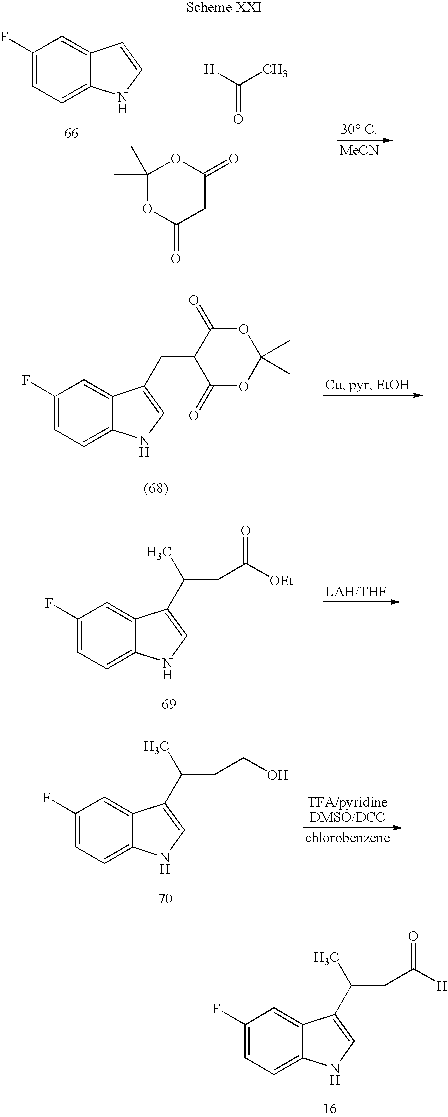

The aldehyde alkyl indole 73 is a novel compound and was prepared by the procedure illustrated in Scheme XXII. The commercially available 2,4-difluorophenylhydrazine HCl 71 was converted to 3-(5,7-difluoro-1H-indol-3-yl)-propan-1-ol 72 by reaction with 3,4-dihydropyran and dioxane in water under reflux. Derivative 72 was then subjected to oxidation conditions to afford the desired 3-(5,7-difluoro-1H-indol-3-yl)-propionaldehyde 73.

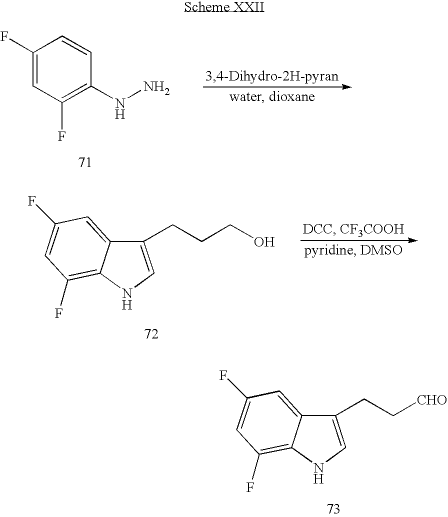

Compounds 80a and 80b are novel compounds and were prepared following the procedure illustrated in Scheme XXIII. Commercially available (4-fluoro-phenyl)-hydrazine hydrochloride 74 and 4-oxo-cyclohexanecarboxylic acid ethyl ester 75a or 3-oxo-cyclohexanecarboxylic acid ethyl ester 75b were combined in the presence of ethanol under reflux, to yield intermediate 76a, 6-fluoro-2,3,4,9-tetrahydro-1H-carbazole-3-carboxylic acid, or 76b 2-ethoxymethyl-6-fluoro-2,3,4,9-tetrahydro-1H-carbazole, respectively, as described in WO 01/07409, incorporated by reference herein. Intermediate 76a or 76b was then dissolved in THF in a nitrogen atmosphere and 1M LAH was added. The reaction mixture was warmed to reflux, to yield intermediate 77a, (6-fluoro-2,3,4,9-tetrahydro-1H-carbazol-3-yl)-methanol or 77b (6-fluoro-2,3,4,9-tetrahydro-1H-carbazol-2-yl)-methanol. Intermediate 77a or 77b was then treated with DMSO/TFA/pyridine/benzene/DCC to yield 6-fluoro-2,3,4,4a,9,9a-hexahydro-1H-carbazole-3-carbaldehyde, intermediate 78a or 6-fluoro-2,3,4,4a,9,9a-hexahydro-1H-carbazole-2-carbaldehyde, 78b respectively, which was then reacted with compound 79, to yield product 80a or 80b.

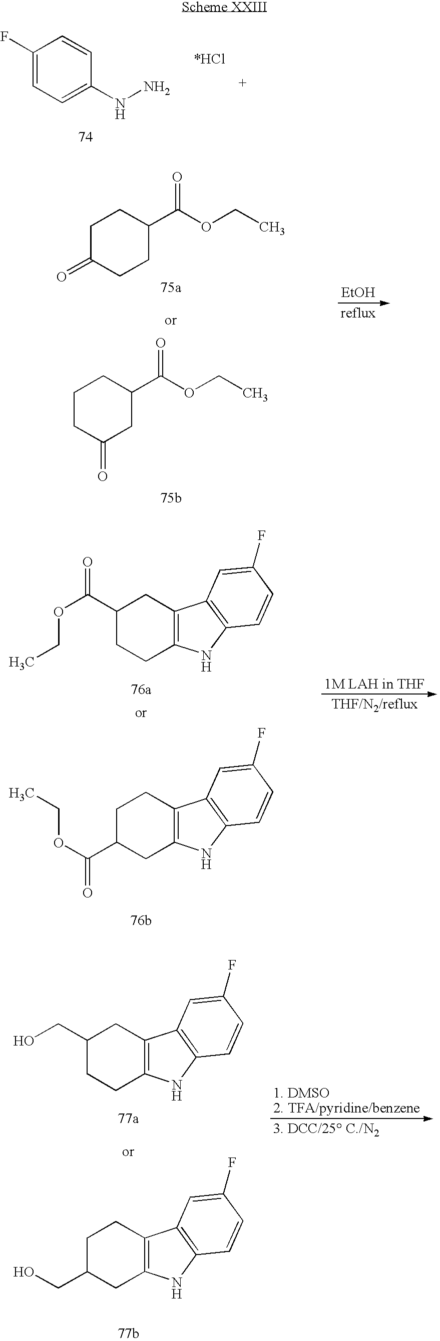

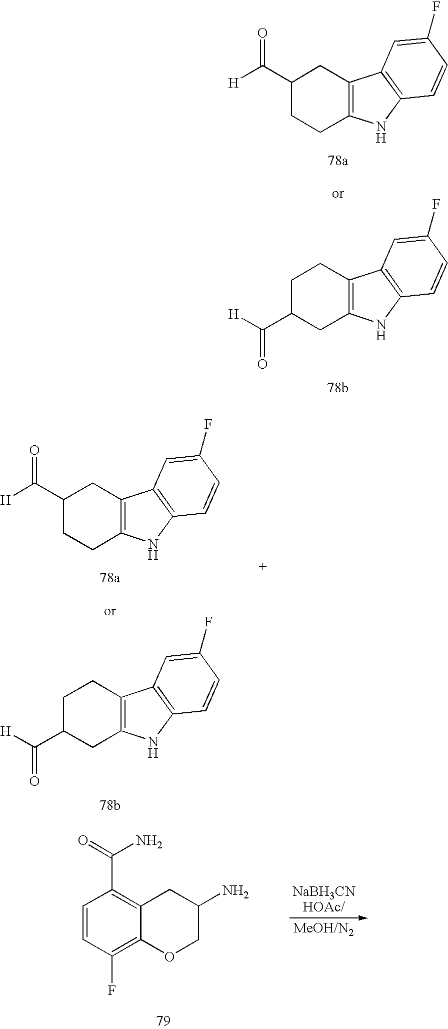

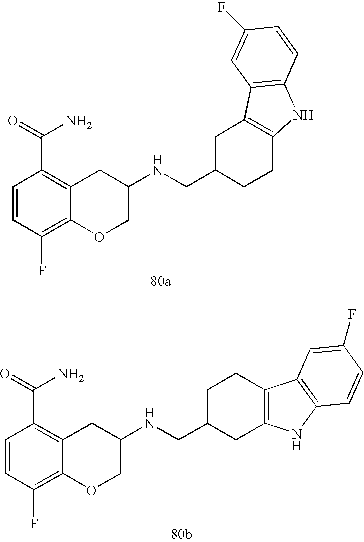

Compounds of structure Im were prepared by conventional methods as illustrated in Scheme XXIV. The appropriate cycloalkylindole 81 was combined with a 3-amino chroman derivative 1a in the presence of a reducing agent such as sodium cyanoborohydride or sodium triacetoxyborohydride to generate the desired product Im (where R¹ is hydrogen). This was then followed by a second reductive amination using sodium cyanoborohydride and the desired alkyl aldehyde to introduce the appropriate alkyl chain R¹ on the basic nitrogen if desired. The compounds of this invention may be resolved into their enantiomers and diastereomers by chiral HPLC.

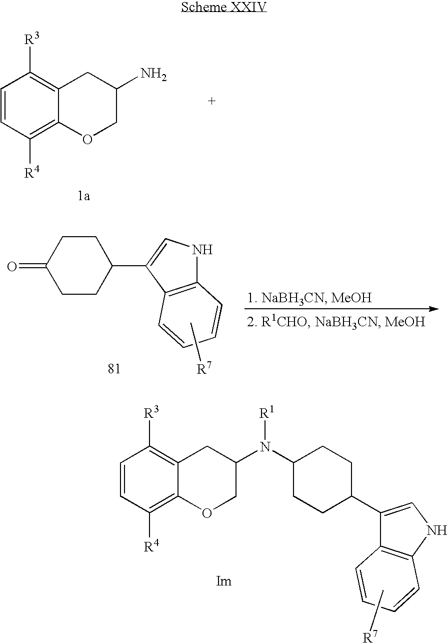

Compounds of formula Ik can be prepared by the methods illustrated in Schemes XXV and XXVI below. N-alkylation of the appropriate indole 115 is achieved with 1,4-dibromo-butane or TsO—(CH₂)₃—OTs in the presence of a strong base, such as NaH, and an aprotic polar solvent such as DMF. The resulting intermediate 116 or 118 is combined with a 3-amino chroman derivative 1c in in the presence of acid binding agents and heated to a temperature of about 85° C. for several hours to generate the intermediate 117 or 119. Reductive amination of intermediate 117 or 119 produces the desired product Ik.

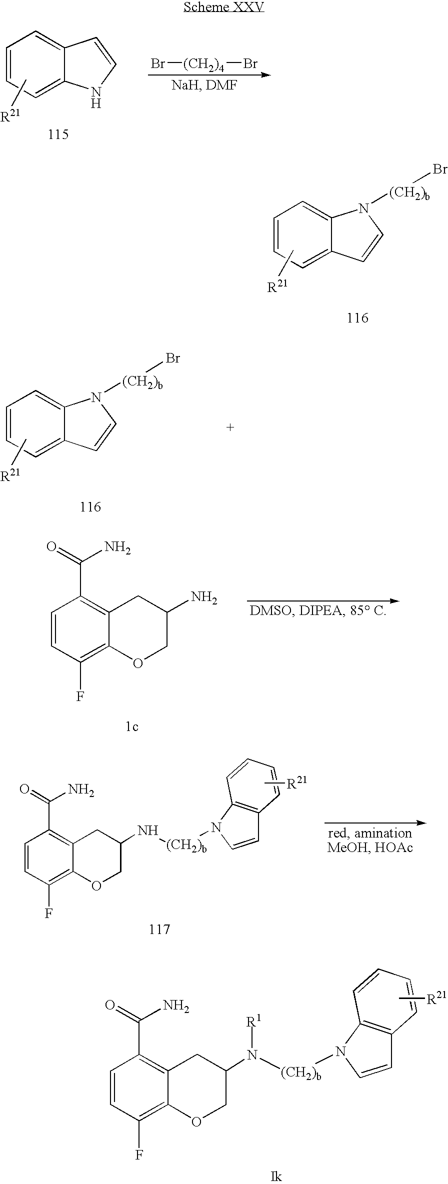

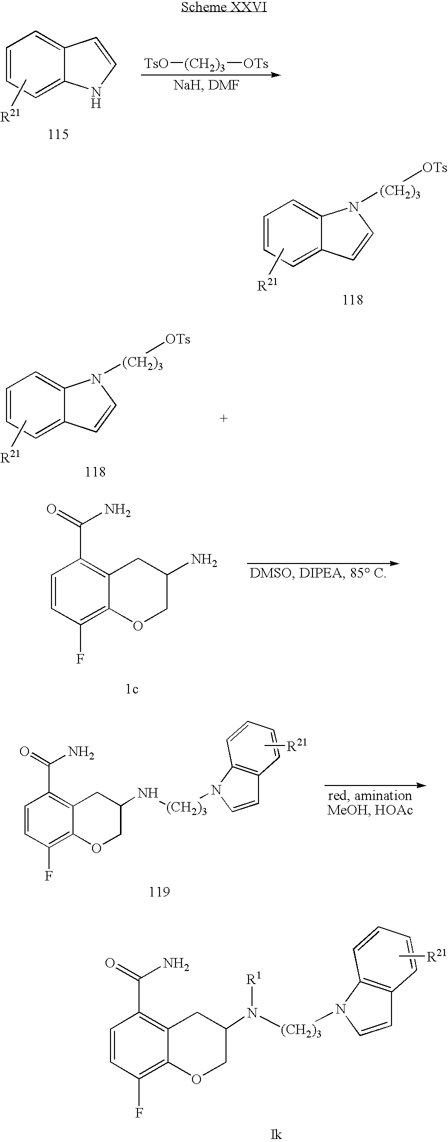

The methylketone alkyl indoles 8a (5-F) are also known compounds and were prepared as described in U.S. Pat. No. 3,671,544 for the three-carbon chain analog, and U.S. Pat. Nos. 4,235,903 and 4,319,029 for the four-carbon chain analog, each of which is incorporated herein by reference. The methylketone alkyl indole 8b (5,7-diF) is, a new compound and was prepared as shown in Scheme XXVII.

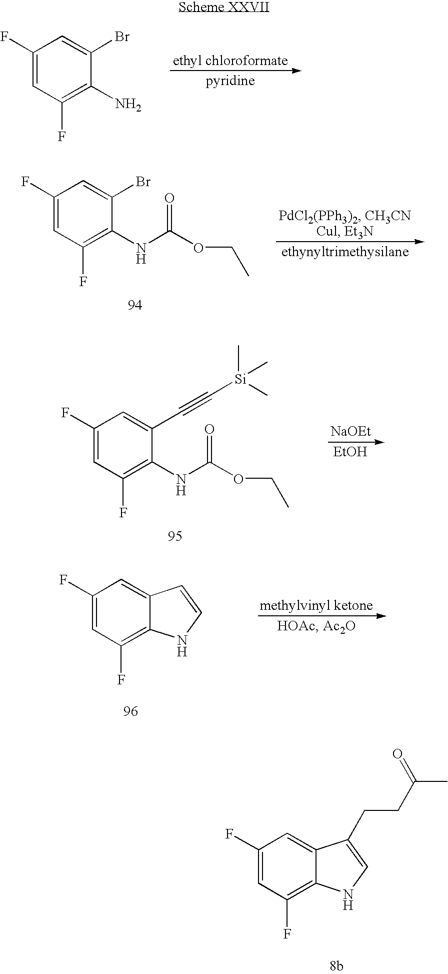

Commercially available 2-bromo-4,6,-difluoroaniline was protected with ethyl chloroformate to generate ethyl (2-bromo-4,6-difluorophenyl) carbamate 94. Displacement of the bromine with ethynyltrimethylsilane in the presence of palladium catalyst, copper iodide and triethylamine produced ethyl{2,4-difluoro-6-[(trimethylsilyl)ethynyl]phenyl}carbamate 95, which was then cyclized in the presence of sodium ethoxide to 5,7-difluoro-1H-indole 96. Finally reaction with methylvinyl ketone in the presence of acetic acid and acetic anhydride generated the desired 4-(5,7-difluoro-1H-indol-3-yl)butan-2-one 8b. As indicated in Scheme XI above, 3-amino-5-fluorochromane-8-carboxamide If was prepared via the procedure outlined in Scheme XI, except that 4-fluoro-2-hydroxybenzoic acid was used as starting material. This synthesis is further elaborated in Scheme XXVIII.

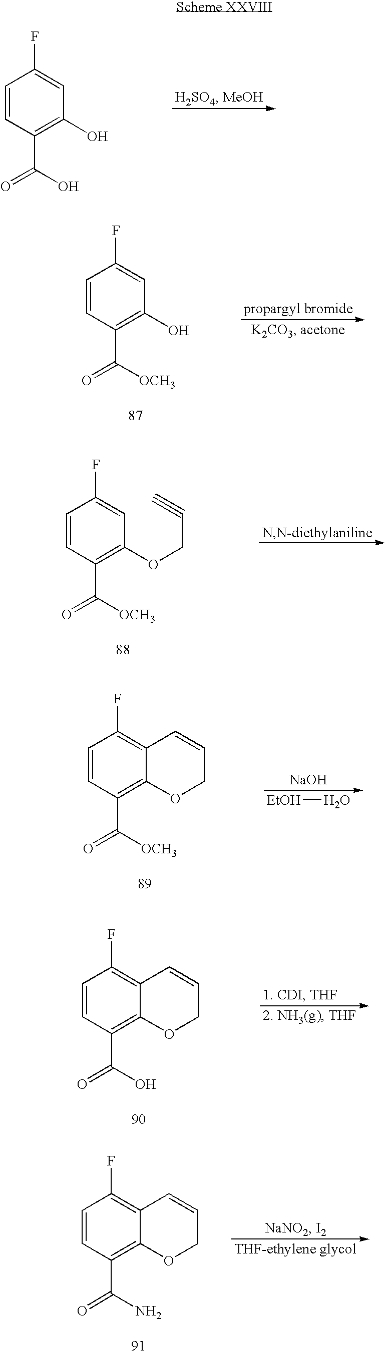

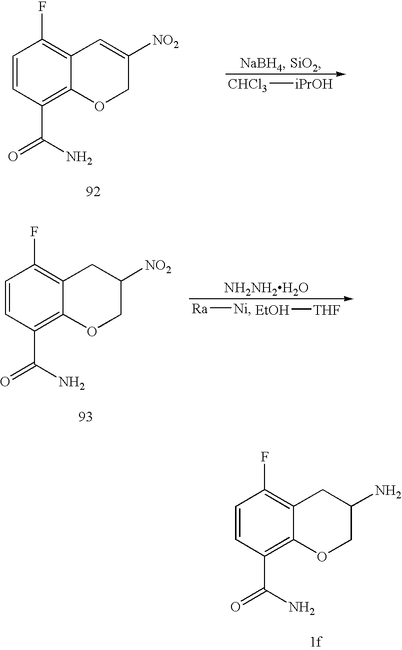

Compounds 106a and 106b are new entities and were prepared chirally pure as described in Scheme XXIX. 3-(5-fluoro-1H-indol-3-yl)propan-1-ol was subjected to standard bromination conditions to generate 3-(3-bromopropyl)-5-fluoro-1H-indole 97, which was then treated with sodium cyanide in N,N-dimethylformamide to afford 5-fluoro-3-(3-isocyanopropyl)-1H-indole 98. The cyano was converted to the carboxylic acid in the presence of potassium hydroxide in water-ethanol generating 4-(5-fluoro-1H-indol-3-yl)butanoic acid 99. Conversion of the acid to the methyl ester under standard conditions, followed by Boc protection of the indole nitrogen and hydrolysis of the methyl ester under lithium hydroxide conditions afforded 4-[1-(tert-butoxycarbonyl)-5-fluoro-1H-indol-3-yl]butanoic acid 102. Compound 102 was then converted to the mixed anhydride with pivaloyl chloride and reacted with the desired chiral oxazolidinone (R- or S-) to generate derivatives 103a or 103b. Methylation in the presence of sodium bis (trimethylsilyl) amide and iodomethane afforded compounds 104a or 104b. Reduction of the oxazolidinone group followed by cleavage of the Boc group and conversion of the alcohol to the aldehyde using the modified Swern conditions generated the desired intermediates 106a or 106b.

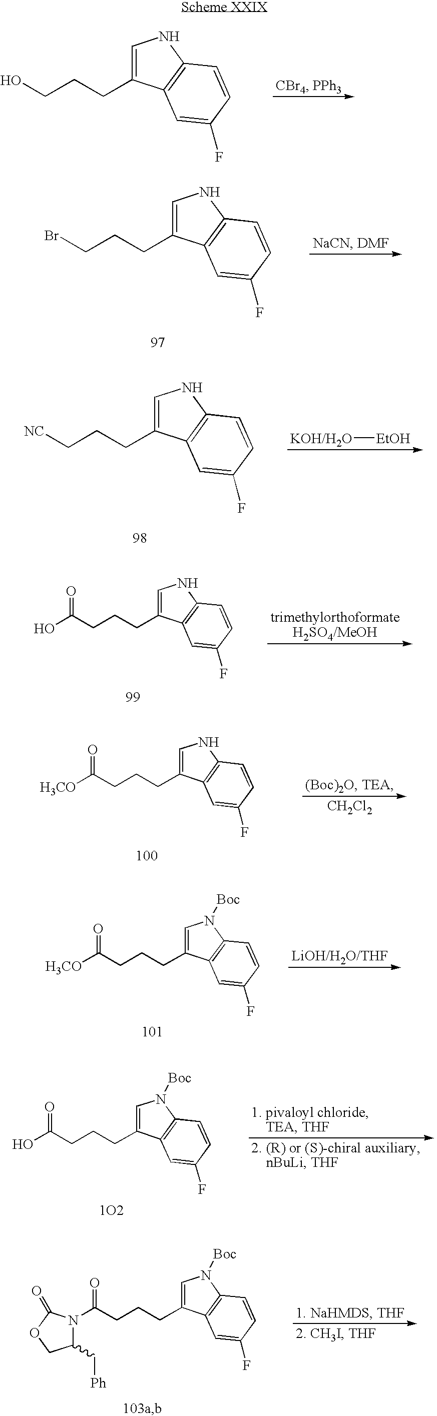

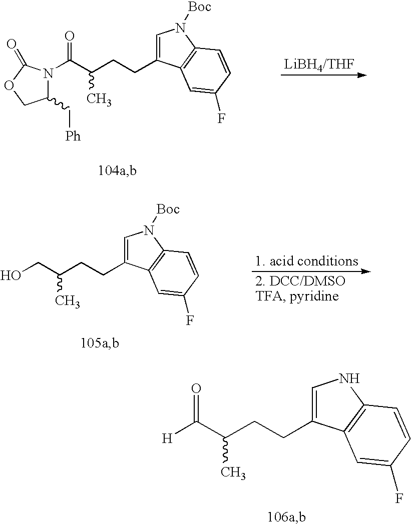

Compound 107 was prepared according to literature procedures as illustrated below in Scheme XXX. Treatment of compound 107 with the previously described 1c under standard reductive amination conditions yielded compound 108 as a mixture of two diastereomers. Compound 108 was then protected as a trifluroacetamide by treating with trifluoroacetic anhydride and DMAP under standard conditions to give compound 109. Hydrogenolysis of 109 with palladium hydroxide and cyclohexene in ethanol at reflux yielded the primary alcohol 110. The primary alcohol is converted to the bromide by treatment with CBr₄ and triphenylphosphine to give 111. 5-Fluoro indole is treated with EtMgBr in order to deprotonate and then alkylated with bromide 111 to yield 112. Subsequent removal of the trifluoroacetamide group using K₂CO₃ in methanol yields the secondary amine 113. Compound 113 is then subjected to standard reductive amination conditions with cyclopropanecarboxaldehyde in the presence of acetic acid and sodium cyanoborohydride to yield the final product 114 as a mixture of diastereomers. The diastereomers of 114 can be separated using a chiral SFC to yield chirally pure compounds. Similar compounds with differing substituents on the indole ring and other alkyl groups off the basic nitrogen can be prepared using a similar procedure.

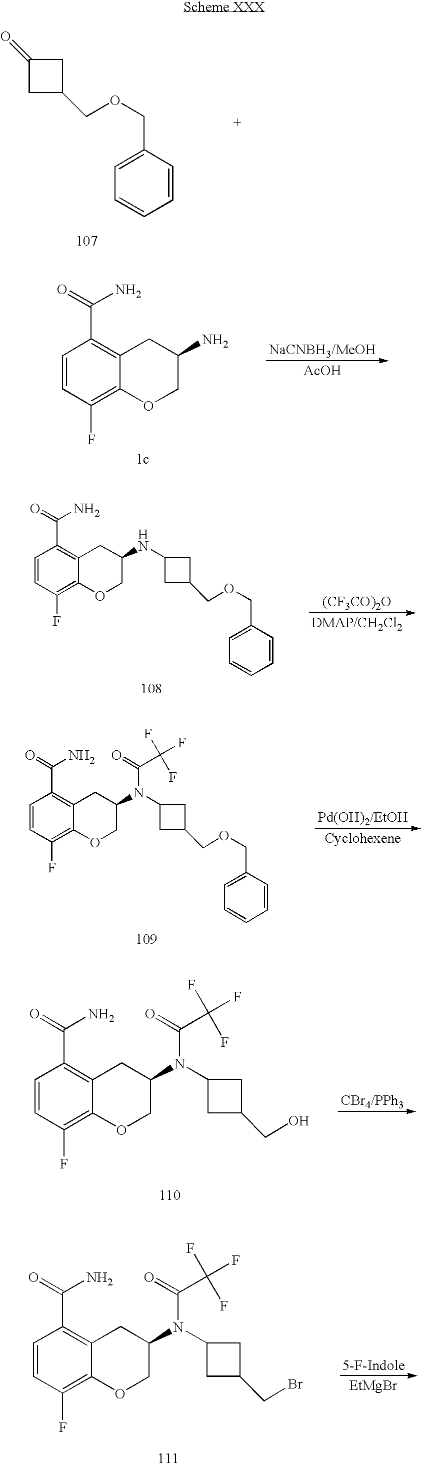

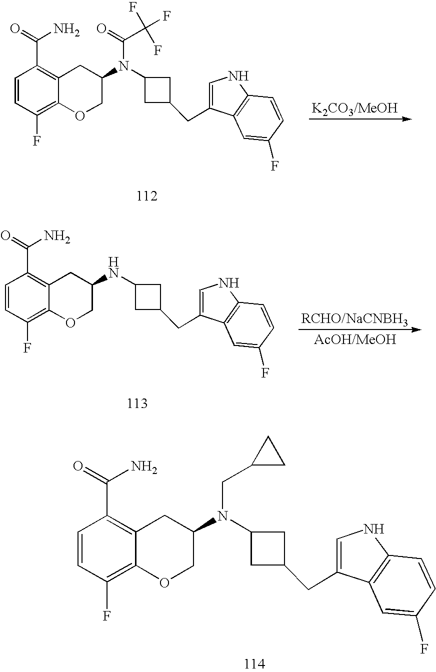

The terms “effective amount”, “therapeutically effective amount” and “effective dosage” as used herein, refer to the amount of a compound of formula I, Ia, Ib, Ic, Id, Ie, If, Ig, Ih, Ij, Ik or Im that, when administered to a patient, is effective to at least partially ameliorate a condition form which the patient is suspected to suffer. Such conditions include, but are not limited to, depression (including, but not limited to major depressive disorder, childhood depression and dysthymia), anxiety, panic disorder, post-traumatic stress disorder, premenstrual dysphoric disorder (also known as premenstrual syndrome), attention deficit disorder (with or without hyperactivity), obsessive compulsive disorder, social anxiety disorder, generalized anxiety disorder, obesity, eating disorders such as anorexia nervosa and bulimia nervosa, vasomotor flushing, cocaine and alcohol addiction, sexual dysfunction, cognitive deficits resulting from neurodegenerative disorders like Alzheimer's disease, and related illnesses.

Compounds of formula I have been found to act as serotonin reuptake inhibitors and to have an affinity for the 5-HT_{1A} reuptake transporter. They are therefore useful in the treatment of diseases affected by disorders of the serotonin affected neurological systems, including, but not limited to, depression (including, but not limited to major depressive disorder, childhood depression and dysthymia), anxiety, panic disorder, post-traumatic stress disorder, premenstrual dysphoric disorder (also known as premenstrual syndrome), attention deficit disorder (with or without hyperactivity), obsessive compulsive disorder, social anxiety disorder, generalized anxiety disorder, obesity, eating disorders such as anorexia nervosa and bulimia nervosa, vasomotor flushing, cocaine and alcohol addiction, sexual dysfunction, cognitive deficits resulting from neurodegenerative disorders like Alzheimer's disease, and related illnesses. The present invention thus provides pharmaceutical compositions comprising at least one compound of formula I; and optionally one or more pharmaceutically acceptable carrier, excipient, or diluents.

Examples of such carriers are well known to those skilled in the art and are prepared in accordance with acceptable pharmaceutical procedures, such as, for example, those described in Remington's Pharmaceutical Sciences, 17th edition, ed. Alfonoso R. Gennaro, Mack Publishing Company, Easton, Pa. (1985), which is incorporated herein by reference in its entirety. Pharmaceutically acceptable carriers are those that are compatible with the other ingredients in the formulation and biologically acceptable.

The compounds of this invention may be administered orally or parenterally, neat or in combination with conventional pharmaceutical carriers. Applicable solid carriers can include one or more substances which may also act as flavoring agents, lubricants, solubilizers, suspending agents, fillers, glidants, compression aids, binders or tablet-disintegrating agents or encapsulating materials. They are formulated in conventional manner, for example, in a manner similar to that used for known antihypertensive agents, diuretics and β-blocking agents. Oral formulations containing the active compounds of this invention may comprise any conventionally used oral forms, including tablets, capsules, buccal forms, troches, lozenges and oral liquids, suspensions or solutions. In powders, the carrier is a finely divided solid, which is an admixture with the finely divided active ingredient. In tablets, the active ingredient is mixed with a carrier having the necessary compression properties in suitable proportions and compacted in the shape and size desired. The powders and tablets preferably contain up to 99% of the active ingredient.

Capsules may contain mixtures of the active compound(s) with inert fillers and or diluents such as the pharmaceutically acceptable starches (e.g. corn, potato or tapioca starch), sugars, artificial sweetening agents, powdered celluloses, such as crystalline and microcrystalline celluloses, flours, gelatins, gums, etc.

Useful tablet formulations may be made by conventional compression, wet granulation or dry granulation methods and utilize pharmaceutically acceptable diluents, binding agents, lubricants, disintegrants, surface modifying agents (including surfactants), suspending or stabilizing agents, including, but not limited to, magnesium stearate, stearic acid, sodium lauryl sulfate, talc, sugars, lactose, dextrin, starch, gelatin, cellulose, methyl cellulose, microcrystalline cellulose, sodium carboxymethyl cellulose, carboxymethylcellulose calcium, polyvinylpyrrolidine, alginic acid, acacia gum, xanthan gum, sodium citrate, complex silicates, calcium carbonate, glycine, sucrose, sorbitol, dicalcium phosphate, calcium sulfate, lactose, kaolin, mannitol, sodium chloride, low melting waxes and ion exchange resins. Preferred surface modifying agents include nonionic and anionic surface modifying agents. Representative examples of surface modifying agents include, but are not limited to, poloxamer 188, benzalkonium chloride, calcium stearate, cetostearl alcohol, cetomacrogol emulsifying wax, sorbitan esters, colliodol silicon dioxide, phosphates, sodium dodecylsulfate, magnesium aluminum silicate, and triethanolamine. Oral formulations herein may utilize standard delay or time release formulations to alter the absorption of the active compound(s). The oral formulation may also consist of administering the active ingredient in water or fruit juice, containing appropriate solubilizers or emulisifiers as needed.

Liquid carriers may be used in preparing solutions, suspensions, emulsions, syrups and elixirs. The active ingredient of this invention can be dissolved or suspended in a pharmaceutically acceptable liquid carrier such as water, an organic solvent, a mixture of both or pharmaceutically acceptable oils or fat. The liquid carrier can contain other suitable pharmaceutical additives such as solubilizers, emulsifiers, buffers, preservatives, sweeteners, flavoring agents, suspending agents, thickening agents, colors, viscosity regulators, stabilizers or osmo-regulators. Suitable examples of liquid carriers for oral and parenteral administration include water (particularly containing additives as above, e.g. cellulose derivatives, preferably sodium carboxymethyl cellulose solution), alcohols (including monohydric alcohols and polyhydric alcohols, e.g. glycols) and their derivatives, and oils (e.g. fractionated coconut oil and arachis oil). For parenteral administration the carrier can also be an oily ester such as ethyl oleate and isopropyl myristate. Sterile liquid carriers are used in sterile liquid form compositions for parenteral administration. The liquid carrier for pressurized compositions can be halogenated hydrocarbon or other pharmaceutically acceptable propellant.

Liquid pharmaceutical compositions, which are sterile solutions or suspensions, can be utilized by, for example, intramuscular, intraperitoneal or subcutaneous injection. Sterile solutions can also be administered intravenously. Compositions for oral administration may be in either liquid or solid form.

Preferably the pharmaceutical composition is in unit dosage form, e.g. as tablets, capsules, powders, solutions, suspensions, emulsions, granules, or suppositories. In such form, the composition is sub-divided in unit dose containing appropriate quantities of the active ingredient; the unit dosage forms can be packaged compositions, for example, packeted powders, vials, ampoules, prefilled syringes or sachets containing liquids. The unit dosage form can be, for example, a capsule or tablet itself, or it can be the appropriate number of any such compositions in package form. Such unit dosage form may contain from 0.1 to 100 mg of a compound of the invention and preferably from 2 to 50 mg. Still further preferred unit dosage forms contain 5 to 25 mg of a compound of the present invention. The compounds of the present invention can be administered orally at a dose range of 0.01 to 100 mg/kg or preferably, at a dose range of 0.1 to 10 mg/kg. Such compositions may be administered from 1 to 6 times a day, more usually from 1 to 4 times a day.

When administered for the treatment or inhibition of a particular disease state or disorder, it is understood that the effective dosage may vary depending upon the particular compound utilized, the mode of administration, the condition, and severity thereof, of the condition being treated, as well as the various physical factors related to the individual being treated. In therapeutic application, compounds of the present invention are provided to a patient already suffering from a disease in an amount sufficient to cure or at least partially ameliorate the symptoms of the disease and its complications. An amount adequate to accomplish this is defined as a “therapeutically effective amount”. The dosage to be used in the treatment of a specific case must be subjectively determined by the attending physician. The variables involved include the specific condition and the size, age and response pattern of the patient. Effective administration of the compounds of this invention may be given at an oral dose of from about 0.1 mg/day to about 1000 mg/day. Preferably, administration will be from about 10 mg/day to about 600 mg/day, more preferably, a starting dose is about 5 mg/day with gradual increase in the daily dose to about 150 mg/day, to provide the desired dosage level in the human. Doses may be administered in a single dose or in two or more divided doses. The projected daily dosages are expected to vary with route of administration.

Such doses may be administered in any manner useful in directing the active compounds herein to the recipient's bloodstream, including orally, via implants, parentally (including intravenous, intraperitoneal, intraarticularly and subcutaneous injections), rectally, intranasally, topically, oculary (via eye drops), vaginally, and transdermally.

In some cases it may be desirable to administer the compounds directly to the airways in the form of an aerosol. For administration by intranasal or intrabrochial inhalation, the compounds of this invention may be formulated into an aqueous or partially aqueous solution.

The compounds of this invention may be administered parenterally or intraperitoneally. Solutions or suspensions of these active compounds as a free base or pharmaceutically acceptable salt may be prepared in water suitably mixed with a surfactant such as hydroxyl-propylcellulose. Dispersions may also be prepared in glycerol, liquid polyethylene glycols and mixtures thereof in oils. Under ordinary conditions of storage and use, these preparations contain a preservative to inhibit the growth of microorganisms.

The pharmaceutical forms suitable for injectable use include sterile aqueous solutions or dispersions and sterile powders for the extemporaneous preparation of sterile injectable solutions or dispersions. In all cases, the form mist be sterile and must be fluid to the extent that easy syringability exists. It must be stable under the conditions of manufacture and storage and must be preserved against the contaminating action of microorganisms such as bacteria and fungi. The carrier can be a solvent or dispersion medium containing, for example, water, ethanol, polyol (e.g., glycerol, propylene glycol and liquid polyethylene glycol), suitable mixtures thereof, and vegetable oils.

The compounds of this invention can be administered transdermally through the use of a transdermal patch. For the purposes of this disclosure, thransdermal administrations are understood to include all administrations across the surface of the body and the inner linings of bodily passages including epithelial and mucosal tissues. Such administrations may be carried out using the present compounds, or pharmaceutically acceptable salts thereof, in lotions, creams, foams, patches, suspensions, solutions, and suppositories (rectal and vaginal).

Transdermal administration may be accomplished through the use of a transdermal patch containing the active compound and a carrier that is inert to the active compound, is non-toxic to the skin, and allows delivery of the agent for systemic absorption into the blood stream via the skin. The carrier may take any number of forms such as creams and ointments, pastes, gels and occlusive devices. The creams and ointments may be viscous liquid or semisolid emulsions of either the oil-in-water or water-in-oil type. Pastes comprised of absorptive powders dispersed in petroleum or hydrophilic petroleum containing the active ingredient may also be suitable. A variety of occlusive devices may be used to release the active ingredient into the blood stream, such as a semi-permeable membrane covering a reservoir containing the active ingredient with or without a carrier, or a matrix containing the active ingredient. Other occlusive devices are known in the literature.

The compounds of this invention may be administered rectally or vaginally in the form of a conventional suppository. Suppository formulations may be made from traditional materials, including cocoa butter, with or without the addition of waxes to alter the suppository's melting point, and glycerin. Water soluble suppository bases, such as polyethylene glycols of various molecular weights, may also be used.

In certain embodiments, the present invention is directed to prodrugs of compounds of formula I, Ia, Ib, Ic, Id, Ie, If, Ig, Ih, Ij, Ik, or Im. Various forms of prodrugs are known in the art, for example, as discussed in, for example, Bundgaard, (ed.), Design of Prodrugs, Elsevier (1985); Widder, et al. (ed.), Methods in. Enzymology, vol. 4, Academic Press (1985); Krogsgaard-Larsen, et al. (ed.), “Design and Application of Prodrugs”, Textbook of Drug Design and Development, Chapter 5, 113-191 (1991), Bundgaard, et al., Journal of Drug Deliver reviews, 8:1-38 (1992), Bundgaard, J. of Pharmaceutical Sciences, 77:285 et seq. (1988); and Higuchi and Stella (eds.) Prodrugs as Novel Drug Delivery Systems, American Chemical Society (1975), each of which is incorporated by reference in its entirety.

The present invention further provides a compound of the invention for use as an active therapeutic substance. Compounds of the invention are of particular use in the treatment of diseases affected by disorders of serotonin.

The present invention further provides a method for treating depression (including, but not limited to major depressive disorder, childhood depression and dysthymia), anxiety, panic disorder, post-traumatic stress disorder, premenstrual dysphoric disorder (also known as premenstrual syndrome), attention deficit disorder (with or without hyperactivity), obsessive compulsive disorder, social anxiety disorder, generalized anxiety disorder, obesity, eating disorders such as anorexia nervosa and bulimia nervosa, vasomotor flushing, cocaine and alcohol addiction, sexual dysfunction, cognitive deficits resulting from neurodegenerative disorders like Alzheimer's disease, and related illnesses in mammals including man, which comprises administering to the afflicted mammal an effective amount of a compound or a pharmaceutical composition of the invention.

### EXAMPLES

Preparation of Intermediates

### Examples 1a and 1b

### Intermediate 4a—3-(5-fluoro-1H-indol-3-yl)propanal and Intermediate 4b—3-(6-fluoro-1H-indol-3-yl)propanal

To a solution of trifluoroacetic acid (3.2 ml, 41 mmol) and pyridine (6.7 ml, 83 mmol) in anhydrous DMSO (90 ml) and chlorobenzene (90 ml) was added 3-(5-fluoro-1H-indol-3-yl)propan-1-ol (4.0 g, 20.7 mmol) and then dicyclohexyl carbodiimide (25.6 g, 124 mmol). After stirring at room temperature for 16 hrs, the reaction mixture was diluted with H₂O, and extracted with methylene chloride (2×). The combined organic extracts were treated with brine, dried over anhydrous magnesium sulfate, filtered and concentrated. Chromatography-((4:1) Hexane-EtOAc) afforded 2.12 g (54%) of intermediate 4a, 3-(5-fluoro-1H-indol-3-yl)propanal, as a white solid: mp 79-80° C.; MS (ES) m/z 190.0 ([M−H]⁻). Anal. calculated for C₁₁H₁₀FNO; C, 69.10 H; 5.27 N; 7.33; Found: C, 68.91 H, 5.20 N, 7.11.

Intermediate 4b was prepared as described above for intermediate 4a (example 1a) using 3-(6-fluoro-1H-indol-3-yl)propan-1-ol (1 g, 5.17 mmol), trifluoroacetic acid (0.8 mL, 20.7 mmol), pyridine (1.8 mL, 20.7 mmol), benzene (29 mL), DMSO (29 mL) and dicyclohexylcarbodiimide (6.4 g, 31 mmol). Chromatography ((2:1) Hex-EtOAc) afforded 3-(6-fluoro-1H-indol-3-yl)propanal as a yellow-orange solid. The product was characterized by

¹HNMR.

### Example 2

### Intermediate 94—ethyl (2-bromo-4,6-difluorophenyl)carbamate

A solution of 2-bromo-4,6-difluoro-phenylamine (11.0 g, 53. mmol) in pyridine (45 mL) was cooled to 0° C. Ethyl chloroformate (7.7 mL, 80 mmol) was added at a rate such that the reaction temperature was maintained at less than 5° C. The resulting solution was stirred at 0° C. for 2 hours, then was allowed to warm to room temperature, and was filtered, then concentrated in vacuo. The residue was dissolved in 2:1 Et₂O/EtOAc (150 mL) and was washed successively with H₂O (3×50 mL), 2.5 N HCl (3×50 mL), saturated aqueous NaHCO₃ solution (3×50 mL), and brine (3×50 mL). The organic layer was dried over Na₂SO₄, filtered, and concentrated in vacuo. Purification by flash chromatography on silica gel (1:3 EtOAc:hexanes) afforded 8.56 g (58%) of the title product.

### Example 3

### Intermediate 95—ethyl{2,4-difluoro-6-[(trimethylsilyl)ethynyl]phenyl}carbamate

To a solution of (2-bromo-4,6-difluoro-phenyl)-carbamic acid ethyl ester (10 g, 35.7 mmol) in CH₃CN (120 mL) under N₂ was added PdCl₂(PPh₃)₂ (2.51 g, 3.57 mmol), CuI (170 mg, 0.893 mmol), Et₃N (9.76 mL), and ethynyltrimethylsilane (7.57 mL, 53.6 mmol). The reaction mixture was refluxed for 2 hours. After cooling to; room temperature, the mixture was filtered and concentrated in vacuo. The residue was dissolved in EtOAc (100 mL) and washed with H₂O (3×50 mL) and brine (3×50 mL).

The organic layer was dried over Na₂SO₄, filtered, and concentrated in vacuo. Purification by flash chromatography on silica gel (1:1 EtO'Ac:hexanes) afforded 9.93 g (94%) of the title compound.

### Example 4

### Intermediate 96—5,7-difluoro-1H-indole

To a solution of NaOEt (1.83 g, 26.9 mmol) in ethanol (35 mL) was added a solution of (2,4-difluoro-6-trimethylsilanylethynyl-phenyl)-carbamic acid ethyl ester (2 g, 6.73 mmol) in ethanol (10 mL). The reaction mixture was stirred at room temperature for 1 hour (until the disappearance of starting material by TLC), then refluxed for 1 hour. After cooling to room temperature, the reaction was concentrated in vacuo. The residue was dissolved in Et₂O (50 mL) and washed with H₂O (3×25 mL) and brine (3×25 mL). The ethereal solution was dried over Na₂SO₄, filtered, and concentrated in vacuo. Purification by flash chromatography on silica gel (1:4 EtOAc:hexanes) afforded 770 mg (75%) of the title compound.

### Example 5

### Intermediate 8b—4-(5,7-difluoro-1H-indol-3-yl)-butan-2-one

To a solution of 5,7-difluoro-1H-indole (770 mg, 5.03 mmol) in HOAc (3.41 mL) was added methylvinylketone (1.06 g, 1.26 mmol) and Ac₂O (1.14 mL). The reaction mixture was refluxed for 4 hours. After cooling to room temperature the reaction was treated with H₂O (5 mL). The aqueous mixture was extracted with EtOAc (3×5 mL). The combined organic layers were washed with H₂O (3×5 mL) and brine (3×5 mL), then were dried over Na₂SO₄, filtered, and concentrated in vacuo. Purification by flash chromatography on silica gel (1:4 EtOAc:hexanes) afforded 480 mg (43%) of the title compound.

### Example 6

### Intermediate 25c—Methyl 4-fluoro-3-hydroxybenzoate

To 4-fluoro-3-hydroxybenzoic acid (15 g, 0.096 mol) in anhydrous methanol (120 ml), under nitrogen at room temperature, was added trimethylorthoformate (18.4 ml, 0.168 mmol) and concentrated sulfuric acid (2.3 ml). The reaction mixture was stirred at 50° C. overnight. Half the solvent was removed in vacuo and the remaining solution poured into 500 ml of ice —H₂O. The product was then extracted with Et₂O (3×). The combined ether extracts were washed with H₂O (1×) followed by a cold solution of saturated sodium bicarbonate, treated with brine, dried over anhydrous magnesium sulfate, filtered and concentrated. 16.04 g (98%) of methyl 4-fluoro-3-hydroxybenzoate were isolated as an off-white solid: mp 89.5-91.5° C.; MS (ESI) m/z 169 ([M−H]⁻); Anal. calculated for C₈H₇FO₃; C, 56.48 H, 4.15; Found: C, 56.40 H, 3.94.

### Example 7

### Intermediate 26c—Methyl 4-fluoro-3-(prop-2-ynyloxy)benzoate

To methyl 4-fluoro-3-hydroxybenzoate (16 g, 0.094 mol) in anhydrous acetone (350 ml), under nitrogen at room temperature, was added propargyl bromide (20 ml, 80% w/toluene, 0.141 mol) and powdered potassium carbonate (26 g, 0.188 mol). The reaction mixture was stirred at room temperature overnight. The reaction mixture was then filtered and the precipitate washed thoroughly with acetone. The filtrate was concentrated. The residue was dissolved in ether and washed with H₂O (4×). The organic layer was treated with brine, dried over anhydrous magnesium sulfate, filtered and concentrated to generate 19.57 g (100%) of methyl 4-fluoro-3-(prop-2-ynyloxy)benzoate as a peach solid: mp 59.5-61.0° C.; MS (EI) m/z 208; Anal. calculated for C₁₁H₉FO₃; C, 63.46 H, 4.36; Found: C, 63.26H, 4.31.

### Example 8

### Intermediate 27c—Methyl 8-fluoro-2H-chromene-5-carboxylate

To methyl 4-fluoro-3-(prop-2-ynyloxy)benzoate (19.57 g, 0.094 mol) was added N,N-diethylaniline (125 ml). The reaction mixture was heated to 22° C. and kept at that temperature for 2 hrs. The reaction mixture was then cooled down to room temperature and diluted with ether. It was washed cautiously with 2N HCl. The aqueous extracts were then re-extracted with ether (2×). The combined Et₂O extracts were washed with H₂O (1×), treated with brine, dried over anhydrous magnesium sulfate, filtered and concentrated. Chromatography ((14:1) Hexane-EtOAc) afforded 15.66 g (80%) of methyl 8-fluoro-2H-chromene-5-carboxylate as a yellow solid: mp 72-74° C.; MS (EI) m/z 208; Anal. calculated for C, H₉FO₃; C, 63.46 H, 4.36; Found: C, 63.32 H, 4.49.

### Example 9

### Intermediate 28c—8-fluoro-2H-chromene-5-carboxylic acid

To methyl 8-fluoro-2H-chromene-5-carboxylate (15.66 g, 0.075 mol) in absolute ethyl alcohol (460 ml) was added a 2.5N NaOH/H₂O solution (42 ml, 0.105 mol). The reaction mixture was brought to reflux and kept under reflux for one hour. The reaction mixture was cooled down to room temperature and the solvent removed in vacuo. The yellow solid was dissolved in H₂O, treated with activated charcoal and filtered through Celite. The light colored liquid was washed once with Et₂O. The aqueous solution was made acidic with 2N HCl and the resulting slurry extracted with ethyl acetate (2×). The combined organic extracts were treated with brine, dried over anhydrous magnesium sulfate, filtered and concentrated to generate 14.16 g (97%) of 8-fluoro-2H-chromene-5-carboxylic acid as an off-white solid: mp 222-223.5° C.; MS (ESI) m/z 193 ([M−H]⁻); Anal. calculated for C₁₀H₇FO₃; C, 61.86 H, 3.63; Found: C, 61.62 H, 3.49.

### Example 10

### Intermediate 29c—8-fluoro-2H-chromene-5-carboxamide

To 8-fluoro-2H-chromene-5-carboxylic acid (14.16 g, 0.0729 mol) in anhydrous THF (350 ml), under nitrogen at room temperature, was added 1,1′-carbonyldiimidazole (17.6 g, 0.109 mol). The reaction mixture was stirred at room temperature for 3.25 hrs. Ammonia was then bubbled through the reaction mixture for a total of 1.25 hrs. A precipitate formed and it was filtered off. The filtrated was diluted with ethyl acetate and extracted with water. The organic layer was treated with brine, dried over anhydrous magnesium sulfate, filtered and concentrated. Upon dissolution in ethyl acetate, a precipitate formed that was filtered and washed with some ethyl acetate. 12.22 g (87%) of 8-fluoro-2H-chromene-5-carboxamide was isolated as a white solid. The filtrate was then chromatographed ((9:1) EtOAc-Hexane) to generate another 1.68 g (12%) of 8-fluoro-2H-chromene-5-carboxamide as a white solid: mp 189-190.5° C.; MS (EI) m/z 193; Anal. calculated for C₁₀H₈FNO₂; C, 62.18 H, 4.17 N, 7.25; Found: C, 61.85 H, 4.13 N, 7.13.

### Example 11

### Intermediate 30c—8-fluoro-3-nitro-2H-chromene-5-carboxamide

To 18-crown-6 (6 g, 22.8 mmol) in anhydrous THF (48 ml), under nitrogen at room temperature, was added potassium nitrite (2.65 g, 31.2 mmol). The reaction mixture was sonicated for 10 min. Iodine (8.7 g, 34.3 mmol) was added and sonication continued for another 30 min. To the reaction mixture was then added 8-fluoro-2H-chromene-5-carboxamide (2 g, 10.4 mmol) dissolved in anhydrous THF (19 ml)-dry pyridine (4.8 ml). Sonication was continued for 5 hrs and the reaction mixture stirred at room temperature overnight. Triethylamine (10 ml) was then added and the reaction mixture stirred for another 30 min. Silica gel was added to the flask and the reaction mixture concentrated. Chromatography ((3:1) EtOAc-Hexane) afforded 1.47 g (60%) of 8-fluoro-3-nitro-2H-chromene-5-carboxamide as a yellow solid: mp 225-227° C.; MS (ESI) m/z 237 ([M−H]⁻); Anal. calculated for C₁₀H₇FN₂O₄; C, 50.43 H, 2.96 N, 11.76; Found: C, 50.45 H, 3.04 N, 11.38.

### Example 12

### Intermediate 31c—8-fluoro-3-nitrochromane-5-carboxamide

To 8-fluoro-3-nitro-2H-chromene-5-carboxamide (3.73 g, 15.7 mmol) in chloroform (340 ml)-isopropanol (125 ml), under nitrogen at room temperature, was added silica, gel (11 g). To the slurry, was slowly added over a 15 min period, sodium borohydride (1.48 g, 39.2 mmol). After 30 min, the reaction mixture was quenched with acetic acid (28 ml) and stirred for another 30 min. The reaction mixture was then filtered and the silica gel washed thoroughly with methylene chloride. The filtrate was concentrated and the residue taken up in EtOAc/H₂O. The organic layer was separated, treated with brine, dried over anhydrous magnesium sulfate, filtered and concentrated. 3.65 g (97%) of 8-fluoro-3-nitrochromane-5-carboxamide was isolated as an off-white solid: mp 184-185.5° C.; MS (ESI) m/z 239 ([M−H]⁻); Anal. calculated for C₁₀H₉FN₂O₄; C, 50.01 H, 3.78 N, 11.66; Found: C, 50.35 H, 3.79 N, 11.36.

### Example 13

### Intermediate 1c—3-amino-8-fluorochromane-5-carboxamide

To 8-fluoro-3-nitrochromane-5-carboxamide (2 g, 8.3 mmol) in absolute ethyl alcohol (90 ml) was added THF (15 ml). The reaction mixture was then heated to 60 C to help solubilize the starting material and cooled down to 45° C. Wet Ra—Ni was added followed by, over a 30 min period, a solution of hydrazine hydrate (4.7 ml) in absolute ethyl alcohol (12 ml). The reaction mixture was kept at 45° C. for 1 hr. While still warm, the reaction mixture was filtered over Celite and the Ra—Ni washed thoroughly with hot EtOH. The filtrate was concentrated under vacuum. Chromatography ((4:1) CH₂Cl₂—MeOH (1% NH₄OH)) afforded 1.06 g (61%) of 3-amino-8-fluorochromane-5-carboxamide as an off-white solid: mp 229° C./dec; MS (ESI) m/z 211 ([M+H]⁺); Anal. calculated for C₁₀H₁₁FN₂O₂.HCl: C, 48.69 H, 4.90 N, 11.36; Found: C, 48.69 H, 5.05 N, 10.97.

### Example 14

### Intermediate 25d—Methyl 4-chloro-3-hydroxybenzoate

Commercial 2-chloro-5-trifluoromethyl-phenol (5 g, 25 mmol) was added to concentrated sulfuric acid (37 g, 375 mmol) under exclusion of moisture and heated under stirring to 100° C. The reaction mixture was stirred at this temperature for 1 hour, then cooled to ambient temperature and poured slowly into anhydrous methanol (300 ml). The obtained solution was refluxed for 3 hours, followed by stirring at ambient temperature overnight. The solvent was removed in vacuo to a final volume of −159 ml and then poured carefully into 10% aqueous sodium bicarbonate solution (300 ml). The product was extracted with ether (3×100 ml), the combined organic layers washed with brine (100 ml), dried over magnesium sulfate, filtered and evaporated to dryness to yield 4.2 g (90.5%) of methyl 4-chloro-3-hydroxybenzoate as an off-white solid: mp 100-1° C.; MS (APCI) m/z 187 ([M+H]⁺).

### Example 15

### Intermediate 26d—Methyl 4-chloro-3-(prop-2-ynyloxy)benzoate

To methyl 4-chloro-3-hydroxybenzoate (4 g, 21 mmol) in anhydrous acetone (100 ml), under nitrogen at room temperature, was added propargyl bromide (4.55 ml, 80% w/toluene, 32.1 mmole) and powdered potassium carbonate (5.9 g, 42.6 mmol). The reaction mixture was stirred at room temperature overnight, then filtered and the precipitate washed thoroughly with acetone. The filtrate was evaporated in vacuo and the residue was dissolved in ether and washed with H₂O (2×). The organic layer was treated with brine, dried over anhydrous magnesium sulfate, filtered and concentrated to generate 4.5 g (95.7%) of methyl 4-chloro-3-(prop-2-ynyloxy)benzoate as an off-white solid: mp 85-6° C.; MS (APCI) m/z 225 ([M+H]⁺).

### Example 16

### Intermediate 27d—Methyl 8-chloro-2H-chromene-5-carboxylate

To methyl 4-chloro-3-(prop-2-ynyloxy)benzoate (4.4 g, 19.5 mmole) was added N,N-diethylaniline (26 ml). The reaction mixture was heated to 220° C. and kept at that temperature for 2 hours after which it was cooled down to ambient temperature and diluted with ether. It was washed cautiously with 2N HCl. The aqueous extracts were then re-extracted with ether (2×). The combined ether extracts were washed with water (1×), treated with brine, dried over anhydrous magnesium sulfate, filtered and evaporated in vacuo. Chromatography [(95:5) Hexane-EtOAc] afforded 3.35 g (76%) of methyl 8-chloro-2H-chromene-5-carboxylate as yellow microcrystals: mp 49-50° C.; MS (EI) m/z 224 ([M+]).

### Example 17

### Intermediate 28d—8-Chloro-2H-chromene-5-carboxylic acid

To methyl 8-chloro-2H-chromene-5-carboxylate (3.3 g, 14.7 mmol) in absolute ethyl alcohol (130 ml) was added a 2.5N aqueous NaOH solution (8.228 ml, 20.5 mmol). The reaction mixture was brought to reflux and kept under reflux for one hour, cooled to ambient temperature and evaporated in vacuo. The residue was dissolved in water and extracted with ether. The aqueous solution was acidified with 2N HCl and the resulting slurry extracted with ethyl acetate (2×). The combined organic extracts were treated with brine, dried over anhydrous magnesium sulfate, filtered and evaporated in vacuo to generate 3 g (97%) of the desired 8-chloro-2H-chromene-5-carboxylic acid as a creamy yellow solid: mp 196-7° C.; MS (ES) m/z 209 ([M−H]⁻).

### Example 18

### Intermediate 29d—8-Chloro-2H-chromene-5-carboxamide

To 8-chloro-2H-chromene-5-carboxylic acid (2.99 g, 14.2 mmol) in anhydrous THF (70 ml), under nitrogen at room temperature, was added 1,1′-carbonyldiimidazole (3.438 g, 21.2 mmol). The reaction mixture was stirred at room temperature for 3 hours. Anhydrous ammonia gas was then bubbled through the reaction mixture, for 1.5 hours. A precipitate formed and it was filtered off. The filtrated was concentrated in vacuo and diluted with ethyl acetate and extracted with water (2×). The organic layer was treated with brine, dried over anhydrous magnesium sulfate, filtered and concentrated. Upon dissolution in ethyl acetate, a precipitate formed that was filtered and washed with some ethyl acetate. Upon drying 2.82 g (95%) of 8-chloro-2H-chromene-5-carboxamide was isolated as a yellow solid: mp 201-3° C.; MS (ES) m/z 208 ([M−H]⁻).

### Example 19

### Intermediate 30d—8-Chloro-3-nitro-2H-chromene-5-carboxamide

To 18-crown-6 (6.511 g, 24.63 mmol) in anhydrous THF (55 ml), under nitrogen at room temperature, was added potassium nitrite (2.86 g, 33.6 mmol). The reaction mixture was sonicated for 10 min. Iodine (9.38 g, 36.95 mmol) was added and sonication continued for another 30 min. To the reaction mixture was then added 8-chloro-2H-chromene-5-carboxamide (2.35 g, 11.2 mmol) dissolved in anhydrous THF (20 ml) and dry pyridine (5.2 ml). Sonication was continued for 4 hours and the reaction mixture stirred at room temperature overnight. Triethylamine (11.3 ml) was then added and the reaction mixture stirred for another 2 hours. Silica gel (10 g) was added to the flask and the reaction mixture evaporated in vacuo. The residue was chromatographed on silica gel (200 g). Elution with a solvent gradient (hexane/ethylacetate, 40% to 20%) afforded 1.55 g (60%) of the desired 8-chloro-3-nitro-2H-chromene-5-carboxamide as a yellow solid: mp 225-227° C.; MS (ESI) m/z 253/255 ([M+H]⁺).

### Example 20

### Intermediate 31d—8-Chloro-3-nitrochromane-5-carboxamide

To 8-chloro-3-nitro-2H-chromene-5-carboxamide (1.5 g, 5.89 mmol) in chloroform (128 ml) and isopropanol (47 ml), under nitrogen at room temperature, was added silica gel (4.2 g). To the slurry, was slowly added over a 15 min period, sodium borohydride (556 mg, 14.7 mmol). After 30 min, the reaction mixture was quenched with acetic acid (10.5 ml) and stirred for another 30 min. after which it was then filtered and the silica gel washed thoroughly with methylene chloride. The filtrate was evaporated in vacuo and the residue partitioned between ethyl acetate and water. The organic layer was separated, treated with brine, dried over anhydrous magnesium sulfate, filtered and concentrated. 1.28 g (85%) of 8-chloro-3-nitrochromane-5-carboxamide was isolated as a light beige solid: mp 193-4° C.; MS (ESI) m/z 255/257 ([M−H]⁻).

### Example 21

### Intermediate 1d—3-Amino-8-chlorochromane-5-carboxamide

To 8-chloro-3-nitrochromane-5-carboxamide (1.06 g, 4.13 mmol) in absolute ethyl alcohol (50 ml) was added some THF (9 ml). The reaction mixture was then heated to 60° C. to help solubilize the starting material and cooled down to 45° C. Wet Ra—Ni (3 small scoops) was added followed, over a 30 min period, by a solution of hydrazine hydrate (2.7 ml) in absolute ethyl alcohol (7 ml). The reaction mixture was kept at 45° C. for 1 hour. While still warm, the reaction mixture was filtered over Celite and the Ra—Ni washed thoroughly, with hot EtOH. The filtrate was evaporated in vacuo. The residue was flash chromatographed on silica gel using a solvent mixture of methanol/chloroform (1:4) with 1% ammonium hydroxide affording 0.78 g (83%) of 3-amino-8-chlorochromane-5-carboxamide as an off-white solid, mp 231-6° C.; MS (ES) m/z 225 ([M−H]⁻). It was then converted to the HCl salt by dissolving the free base in methanol, adding ethereal HCl and evaporating the solution in vacuo. The residue was crystallized from methanol/ether affording 3-amino-8-chlorochromane-5-carboxamide hydrochloride salt as off-white microcrystals: mp>250° C.; MS (ES) m/z 225.0 ([M−H]⁻).

### Example 22

### Intermediate 26e—methyl 3-(prop-2-yn-1-yloxy)benzoate

Methyl 3-hydroxybenzoate (131.4 mmole, 20 g) was dissolved in acetone (300 mL) and treated at once under stirring with propargyl bromide (80% in toluene, 197.1 mmole, 21.95 mL). The reaction mixture was cooled to 0° C., potassium carbonate (394.3 mmole, 54.5 g) added and the mixture stirred at ambient temperature for 20 h. The solids were filtered off and the filtrate evaporated. The residue was filtered through a plug of silica gel. Elution with 25% ethyl acetate/hexane furnished 23.5 g (94%) of the title compound as a yellow oil. MS (APPI) m/z 190.

### Example 23

### Intermediate 27e—methyl, 2H-chromene-5-carboxylate

A solution of methyl 3-(prop-2-yn-1-yloxy)benzoate (226.6 mmole, 43.1 g) in N,N-diethylaniline (500 mL) was heated to 250° C. for 3 h. After cooling the mixture to ambient temperature it was diluted with ether and washed repeatedly (8×300 mL) with 2N hydrochloric acid to remove the aniline. The separated organic layer was then washed consecutively with water and brine; the solution was dried over magnesium sulfate, filtered, and evaporated in vacuo. The residue was flash chromatographed on silica gel. Elution with 25% ethyl acetate/hexane gave 16.8 g (40%) of the title compound which was due to instability immediately converted to the 2H-chromene-5-carboxylic acid.

### Example 24

### Intermediate 28e—2H-chromene-5-carboxylic acid

A solution of methyl 2H-chromene-5-carboxylate (36.28 mmole, 6.9 g) in ethanol (50 mL) was treated at once with aqueous sodium hydroxide (2.5 N, 36 mL) and stirred at ambient temperature for 15 h. The mixture was then diluted with water (50 mL) and extracted with ether (2×60 mL). The separated aqueous layer was acidified with aqueous hydrochloric acid (6 M) to pH ˜2. The precipitated material was filtered, washed with water and dried in vacuo to yield 5.9 g (92%) of the desired compound as a white solid. MS (ES) m/z 175.1.

### Example 25

### Intermediate 29e—2H-chromene-5-carboxamide

N,N′-Carbonyldiimidazole (29.8 mmole, 483 mg) was added to a solution of 2H-chromene-5-carboxylic acid in tetrahydrofuran (40 mL) under stirring at ambient temperature. The solution was stirred for 3 h after which ammonia gas was introduced under stirring for 90 minutes. The precipitate was filtered and the filtrate was diluted with water (30 mL) and ethyl acetate (50 mL). The separated organic layer was washed with water, then brine after which it was dried over magnesium sulfate, filtered, and evaporated in vacuo. The residue was flash chromatographed on silica gel (10 g). Elution with 3% methanol in chloroform gave 3 g (86%) of the desired title compound as a white solid; mp 168-70° C. MS (ES) m/z 176.

### Example 26

### Intermediate 30e—3-nitro-2H-chromene-5-carboxamide

A solution of 2H-chromene-5-carboxamide (15.4 mmole, 2.7 g) was dissolved under stirring in tetrahydrofuran (40 mL). Ethylene glycol (1.5 mL) was added and the solution cooled to 0° C. followed by the addition of sodium nitrite (61.65 mmole, 4.25 g) and stirring continued for 30 minutes. Iodine (61.65 mmole, 1.56 g) was added and stirring continued for 3 h at 0° C. followed by ambient temperature for 26 h. The reaction mixture was cooled to 0° C. and then slowly treated with an aqueous sodium bisulfite solution (18%) until the reaction mixture remained clear in color. The precipitated product was filtered, washed consecutively with aqueous sodium bicarbonate (5%) and water. The yellow material was dried in high vacuum to yield 2.13 g (63%) of the title compound; mp 231-2° C. MS (EI) m/z 220.

### Example 27

### Intermediate 31e—3-nitrochromane-5-carboxamide

A solution of 3-nitro-2H-chromene-5-carboxamide (9.5 mmole, 2.1 g) in methanol (60 mL) was treated portionwise under stirring and dry nitrogen with sodium borohydride (30 mmole, 1.35 g) at ambient temperature. The reaction mixture was stirred at ambient temperature overnight, acetic acid (22 mL) was added and stirring continued for 30 minutes after which the mixture was concentrated in vacuo and the residue partitioned between water and ethyl acetate. The separated organic layer was washed with brine, dried over magnesium sulfate, filtered and evaporated to dryness in vacuo to yield 2 g (95%) of the desired compound as off-white micro crystals; mp 200-2° C. MS (ES) m/z 223.0.

### Example 28

### Intermediate 1e—3-aminochromane-5-carboxamide

A suspension of 3-nitrochromane-5-carboxamide (9 mmole, 2 g) in ethanol (130 mL) and tetrahydrofuran (40 mL) was heated to 60° C. (solution) and then cooled to −45° C. under stirring. Raney nickel (−6 spatula scoops) were added followed by the dropwise addition of a solution of hydrazine hydrate (5.9 mL) in ethanol (15.5 mL) over 20 minutes at C45°. The mixture was stirred 1 h at ˜45° C., and filtered warm through celite. The cake was washed with ethanol (10 mL) and the filtrate evaporated in vacuo to dryness furnishing 1.75 g (˜100%) of the title compound as waxy faintly green solid. MS (ES) m/z 193.1.

### Example 29

### Intermediate 83c—8-fluoro-N-methyl-2H-chromene-5-carboxamide

To 8-fluoro-2H-chromene-5-carboxylic acid (3.5 g, 0.018 mol) in anhydrous THF (100 mL), under nitrogen at room temperature, was added EDC (6.9 g, 0.036 mol), HOBt (4.86 g, 0.036 mol) and a 2M solution of methylamine in THF (36 mL, 0.072 mol). The reaction mixture was stirred at room temperature overnight. The reaction mixture was concentrated in vacuo and the residue taken up in CH₂Cl₂/H₂O. The organic layer was separated and the aqueous layer extracted one more time with CH₂Cl₂. The organic extracts were pooled, dried over anhydrous magnesium sulfate, filtered and concentrated. Chromatography ((3:1) EtOAc-Hex) afforded 3.69 g (99%) of desired product as a white solid: mp 150-153° C.; MS (ES) m/z 206.1.

### Example 30

### Intermediate 84c—8-fluoro-N-methyl-3-nitro-2H-chromene-5-carboxamide

To 8-fluoro-N-methyl-2H-chromene-5-carboxamide (3.58 g, 0.017 mol) in THF (105 mL)-ethylene glycol (15 mL), was added iodine (12.9 g, 0.051 mol) and sodium nitrite (3.56 g). The reaction mixture was stirred at room temperature overnight. The reaction mixture was concentrated, the residue taken up in ethyl acetate and extracted as follows: saturated sodium chloride (1×), 5% sodium bisulfite-saturated NaCl (1×), 5% sodium-bisulfite (2×), saturated NaCl (1×). The organic layer was then dried over anhydrous magnesium sulfate, filtered and concentrated. The residue was triturated with MeOH (2×) and 1.8 g (42%) of desired product was isolated as a yellow solid. The filtrate was chromatographed ((3:1) EtOAc-Hex) affording 1.2 gm of a mixture of unreacted starting material and desired product. This mixture was subjected to the same reaction conditions as above generating 0.75 g (18%) of desired product as a yellow solid. Its identity was confirmed by ¹HNMR.

### Example 31

### Intermediate 85c—8-fluoro-N-methyl-3-nitrochromane-5-carboxamide

To 8-fluoro-N-methyl-3-nitro-2H-chromene-5-carboxamide (2.55 g, 0.01 mol) in chloroform (190 mL)-isopropanol (60 mL), under nitrogen at room temperature, was added silica gel (7 g) and, slowly over a 10 min period, sodium borohydride (0.96 g, 0.025 mol). After 40 min, the reaction mixture was quenched with acetic acid (18 mL) and stirred for another 15 min. It was filtered and the silica washed thoroughly with CH₂Cl₂. The filtrate was concentrated in vacuo, the residue taken up in EtOAc/H₂O, the organic layer separated, treated with brine, dried over anhydrous magnesium sulfate, filtered and concentrated affording 2.54 g (98%) of desired product. Its identity was confirmed by ¹HNMR.

### Example 32

### Intermediate 86c—3-Amino-8-fluoro-N-methylchromane-5-carboxamide

To 8-fluoro-N-methyl-3-nitrochromane-5-carboxamide (2.54 g, 9.99 mmol) in absolute ethyl alcohol (10 mL) was added THF (17 mL). The reaction mixture was then heated to 45° C. and wet Ra—Ni was added followed by, over a 30 min period, hydrazine hydrate (5.7 mL) in absolute ethyl alcohol (15 mL). The reaction mixture was kept at 45° C. for 45 min. Same work up as described for example 13. Chromatography ((9:1) CH₂Cl₂—MeOH (1% NH₄OH)) afforded 1.35 g (60%) of desired product which was converted to the HCl salt to generate 3-amino-8-fluoro-N-methylchromane-5-carboxamide hydrochloride salt as a white solid: mp 131° C./dec; MS (ES) m/z 225.1.

### Example 33

### Intermediate 87—Methyl 4-fluoro-2-hydroxybenzoate

To 4-fluoro-2-hydroxybenzoic acid (5.33 g, 0.034 mol) in anhydrous methanol (26 mL), under nitrogen at room temperature, was added sulfuric acid (1.5 mL). The reaction mixture was brought to reflux and kept under reflux overnight. More sulfuric acid (2.5 mL) was added and the reaction mixture kept under reflux overnight. Half the solvent was removed in vacuo and the remaining solution poured over ice-H₂O. The product was then extracted with Et₂O (2×). The combined ether extracts were washed with a cold solution of saturated sodium bicarbonate, treated with brine, dried over anhydrous magnesium sulfate, filtered and concentrated. 5.49 g (95%) of methyl 4-fluoro-2-hydroxybenzoate were isolated as a white solid. Its identity was confirmed by ¹HNMR.

### Example 34

### Intermediate 88—Methyl 4-fluoro-2-(prop-2-yn-1-yloxy)benzoate

To methyl 4-fluoro-2-hydroxybenzoate (5.49 g, 0.032 mol) in anhydrous acetone (120 mL), under nitrogen at room temperature, was added propargyl bromide (7.2 mL, 80% w/toluene, 0.048 mol) and powdered potassium carbonate (8.8 g, 0.064 mol). The reaction mixture was stirred at room temperature overnight. Same work-up as for example 7. 6.45 g (97%) of methyl 4-fluoro-2-(prop-2-yn-1-yloxy)benzoate was isolated as a pale orange oil. Its identity was confirmed by ¹HNMR

### Example 35

### Intermediate 89—Methyl 5-fluoro-2H-chromene-8-carboxylate

To methyl 4-fluoro-2-(prop-2-yn-1-yloxy)benzoate (6.45 g, 0.031 mol) was added N,N-diethylaniline (40 mL). The reaction mixture was heated to 220° C. and kept at that temperature for 2 hrs. Same work up as described for example 8. Chromatography ((6:1) Hex-EtOAc) afforded 4.36 g (68%) of methyl 5-fluoro-2H-chromene-8-carboxylate as a yellow solid: mp 65-67° C.; MS (APPI) m/z 209.

### Example 36

### Intermediate 90—5-Fluoro-2H-chromene-8-carboxylic acid

To methyl 5-fluoro-2H-chromene-8-carboxylate (4.36 g, 0.021 mol) in absolute ethyl alcohol (130 mL) was added a 2.5N NaOH/H₂O solution (12 mL, 0.029 mol). The reaction mixture was brought to reflux and kept under reflux for 1.25 hrs. Same work up as described for example 9. No chromatography and 4 g (99%) of 5-fluoro-2H-chromene-8-carboxylic acid was isolated as a yellow solid: mp 187-189° C.; MS (ES) m/z 193.0.

### Example 37

### Intermediate 91—5-Fluoro-2H-chromene-8-carboxamide

To 5-fluoro-2H-chromene-8-carboxylic acid (4 g, 0.02 mol) in anhydrous THF (105 mL), under nitrogen at room temperature, was added 1,1′-carbonyldiimidazole (5.1 g, 0.03 mol). The reaction mixture was stirred at room temperature for 2.5 hrs. Ammonia was then bubbled through the reaction mixture for a total of 30 min. Same work up as described for example 10. 0.71 g (18%) of 5-fluoro-2H-chromene-8-carboxamide was isolated from filtration as a yellow solid. The filtrate was then chromatographed using (2:1) EtOAc-Hex followed by (3:1) EtOAc-Hex followed by (4:1) EtOAc-Hex to generate another 2.7 g (66%) of 5-fluoro-2H-chromene-8-carboxamide as yellow solid: mp 152-154° C.; MS (ES) m/z 194.0.

### Example 38

### Intermediate 92—5-Fluoro-3-nitro-2H-chromene-8-carboxamide

To 5-fluoro-2H-chromene-8-carboxamide (2.7 g, 0.014 mol) in anhydrous THF (80 mL)-ethylene glycol (14 mL), under nitrogen at 0° C., was added sodium nitrite (3.86 g, 0.056 mol) and the reaction mixture was stirred at 0° C. for 30 min. Iodine (14.2 g, 0.056 mol) was added over a 5 min period and the reaction mixture stirred at 0° C. for 30 min. The ice bath was removed and the reaction mixture stirred at room temperature overnight. The reaction mixture was concentrated, diluted with ethyl acetate and a sodium hydrogen sulfite solution (20 g/100 mL) was added until the solution turned yellow. The organic layer was separated, treated with brine, dried over anhydrous magnesium sulfate, filtered and concentrated. The reaction mixture was triturated with MeOH and the pale yellow precipitate filtered off, and washed with hexane. 1.1 g (31%) of 5-fluoro-3-nitro-2H-chromene-8-carboxamide was isolated as a pale yellow solid. The filtrate was concentrated to generate 1.73 g of product and unreacted starting material. This mixture was subjected to the same reaction conditions affording after chromatography ((3:1) EtOAc-Hex) 0.68 g (30%) of 5-fluoro-3-nitro-2H-chromene-8-carboxamide as a yellow solid. Its identity was confirmed by HNMR.

### Example 39

### Intermediate 93—5-Fluoro-3-nitrochromane-8-carboxamide

To 5-fluoro-3-nitro-2H-chromene-8-carboxamide (1.78 g, 7.47 mmol) in chloroform (150 mL)-isopropanol (60 mL), under nitrogen at room temperature, was added silica gel (5.1 g). To the slurry, was slowly added over a 10 min period, sodium borohydride (0.71 g, 18.7 mmol). After 30 min, the reaction mixture was quenched with acetic acid (15 mL) and stirred for another 10 min. Same work up as described for example 12. Chromatography ((1:1) acetone-Hex) afforded 1 g (55%) of 5-fluoro-3-nitrochromane-8-carboxamide as a white solid. Its identity was confirmed by ¹HNMR.

### Example 40

### Intermediate 1f—3-Amino-5-fluorochromane-8-carboxamide

To 5-fluoro-3-nitrochromane-8-carboxamide (1 g, 0.0042 mol) in absolute ethyl alcohol (45 mL) was added THF (7 mL). The reaction mixture was then heated to 55° C. to help solubilize the starting material and cooled down to 45° C. Wet Ra—Ni was added followed by, over a 10 min period, hydrazine hydrate (2.4 mL) in absolute ethyl alcohol (6 mL). The reaction mixture was kept at 45° C. for 20 min. Same work up as described for example 13. Chromatography ((9:1) CH₂Cl₂—MeOH (1% NH₄OH)) afforded 0.42 g (48%) of 3-amino-5-fluorochromane-8-carboxamide which was converted to the HCl salt to generate a white solid: mp 128° C./dec; MS (ES) m/z 211.1; Anal. Calcd for C10H11FN2O2; HCl; C, 48.69; H, 4.90; N, 11.36. Found: C, 48.61; H, 4.78; N, 10.70.

### Example 41

### Intermediate 32—5-methoxy-2H-chromene-3-carbonitrile

A mixture of 2-hydroxy-6-methoxybenzaldehyde (9.13 g, 0.06 mol), acrylonitrile (19.7 ml, 0.3 mol) and 1,4-diazabicyclo[2,2,2]octane (1.55 g, 0.0138 mol) were refluxed for 21 hrs. The reaction mixture was cooled down to room temperature, diluted with Et₂O, and washed with 1N NaOH followed by 1N HCl. The organic layer was dried over anhydrous magnesium sulfate, filtered and concentrated. Chromatography ((7:3) Hexane-EtOAc) afforded 5.78 g (52%) of 5-methoxy-2H-chromene-3-carbonitrile as a off-white solid: MS (APCI) m/z 187.

### Example 42

### Intermediate 33—5-methoxy-2H-chromene-3-carboxylic acid

A mixture of 5-methoxy-2H-chromene-3-carbonitrile (5.5 g, 0.029 mol) and 10% NaOH/H₂O (88 ml) was refluxed for 5 hrs. The reaction mixture was cooled down in an ice bath, and while on ice, was acidified with concentrated HCl. The precipitate was then filtered under vacuum and washed with hexane. The precipitate was dissolved in MeOH-toluene and concentrated several times under vacuum. Chromatography ((9:1) CH₂Cl₂—MeOH) afforded 5.86 g (98%) of 5-methoxy-2H-chromene-3-carboxylic acid as a slight yellow solid: MS (APCI) m/z 206.

### Example 43

### Intermediate 6—5-methoxy-2H-chromen-3(4H)-one

To 5-methoxy-2H-chromene-3-carboxylic acid (2 g, 9.7 mmol) in methylene chloride (20 ml), was added triethylamine (1.6 ml). To this mixture was added, dropwise over a 10 min period, diphenylphosphorylazide (2.2 ml, 10 mmol) in toluene (8 ml) while the mixture was heated slowly to distill the methylene chloride. When the reaction mixture reached 60° C., an additional 20 ml of toluene was added. At 85 C, the reaction mixture was refluxed for 1.5 hrs. To this mixture was then slowly added 6N HCl (16 ml) and refluxed for 2 hrs. The reaction mixture was cooled down to room temperature and the layers separated. The organic layer was washed with saturated sodium bicarbonate (2×) and H₂O (1×), dried over anhydrous sodium sulfate, filtered and concentrated. Chromatography ((4:1) Hexane-EtOAc) afforded 1.02 g (59%) of 5-methoxy-2H-chromen-3(4H)-one as a yellowish pale oil: MS (EI) m/z 178.

### Example 44

### Intermediate 35—1,7-dimethoxynaphthalen-2(1H)-one

To 1,7-dihydroxynaphthalene (10 g, 0.062 mol) in 2-butanone (125 ml), under nitrogen at room temperature, was added potassium carbonate (26 g, 0.187 mol) and iodomethane (11.6 ml, 0.187 mol). The reaction mixture was brought to reflux and kept under reflux overnight. The reaction mixture was then quenched with H₂O and extracted with methylene chloride (1×). The organic layer was then washed with 2N NaOH, dried over anhydrous magnesium sulfate, filtered and concentrated. Chromatography ((19:1) Hexane-EtOAc) afforded 9.45 g (80%) of 1,7-dimethoxynaphthalen-2(11H)-one as a yellow oil: MS m/z 188.

### Example 45

### Intermediate 7a—8-methoxy-3,4-dihydronaphthalen-2(1H)-one

1,7-dimethoxynaphthalen-2(1H)-one (8.0 g, 0.04 mol) was added to boiling absolute ethanol (200 ml) under mechanical stirring. Sodium (7.4 g, 0.3 mol) was added slowly. The resulting mixture was kept refluxing until all sodium had disappeared. The reaction mixture was cooled to 10° C., 2N HCl was added dropwise until a pH of 6 was obtained, and the reaction mixture refluxed for 1 hr. The solvent was removed under vacuum. Chromatography ((3:1) Hexane-EtOAc) afforded 4.2 g (60%) of 8-methoxy-3,4-dihydronaphthalen-2(1H)-one as an orange solid: mp 29-32° C.; Anal. calculated for C₁₁H₁₂O₂: C, 74.98; H, 6.86; Found: C, 74.92; H, 6.92.

### Example 46

### Intermediate 47—Diethyl[(5-fluoro-1H-indol-3-yl)methyl](methyl) malonate

A solution of 5-fluorogramine (15.5 g, 80.6 mmol) in acetonitrile (450 mL) was treated with diethyl methylmalonate (20.8 mL, 121 mmol) and tributylphosphine at reflux for 17 hours. The cooled reaction is concentrated under reduced pressure and dissolved in ethyl acetate (1.5L), washed with 1N aqueous HCl (500 mL), saturated aqueous NaCl (500 mL), dried over MgSO₄ and concentrated under reduced pressure to an orange oil. Chromatography ((3:1) hexane-EtOAc) afforded 16.5 g (64%) of desired product as an oil which solidifies on standing to a white solid: mp 76-77° C.

### Example 47

### Intermediate 48—[(5-fluoro-1H-indol-3-yl)methyl](methyl)malonic acid

A solution of diethyl[(5-fluoro-1H-indol-3-yl)methyl](methyl) malonate (15.7 g, 48.9 mmol) in ethanol (160 mL) was treated with 2.5N aqueous NaOH (80 mL, 200 mmol) and refluxed for 1.5 hours. The cooled solution is concentrated under reduced pressure to remove ethanol. The residue is acidified with concentrated HCl and extracted with ethyl acetate (3×500 mL). The combined ethyl acetate phases are washed with brine, dried over MgSO₄ and concentrated. This residue is triturated with methylene chloride/hexane and dried under vacuum to afford 12.1 g (93%) of desired product as a pinkish-white solid: mp 135-137° C./dec.

### Example 48

### Intermediate 49—(5-fluoro-1H-indol-3-yl)-2-methylpropan-1-ol

A suspension of [(5-fluoro-1H-indol-3-yl)methyl](methyl)malonic acid (12.3 g, 46.4 mmol) in bromobenzene (50 mL) was refluxed for 1.5 hours then concentrated under reduced pressure. The residue was triturated with methylene chloride/ hexane. Air drying afforded 8.86 g (86%) of 3-(5-fluoro-1H-indol-3-yl)-2-methylpropanoic acid as a tan solid: mp 112-113° C.; MS (ES) m/z 220.1.

A solution of 3-(5-fluoro-1H-indol-3-yl)-2-methylpropanoic acid (9.24 g, 41.8 mmol) in anhydrous tetrahydrofuran (30 mL) was chilled to 0° C. and treated with lithium aluminum hydride, 1N in tetrahydrofuran (50 ml, 50 mmol) at room temperature for 3 hours. Additional lithium aluminum hydride, 1N in tetrahydrofuran (34 mL, 34 mmol) was added and the reaction mixture stirred for an additional 2 hours. The reaction was quenched with ice water (100 mL) then diluted with saturated aqueous potassium sodium tartrate (200 mL) and extracted with ethyl acetate (3×400 mL). The combined organic phases were washed with saturated aqueous NaCl, dried over MgSO₄, and evaporated under reduced pressure affording the desired product 7.74 g (89%) as a viscous orange oil. The product was characterized by ¹HNMR.

### Example 49

### Intermediate 10—3-(5-fluoro-1H-indol-3-yl)-2-methylpropanal

A solution of pyridine (8.3 mL, 100 mmol) in anhydrous toluene (100 mL) was chilled to 0° C. and treated sequentially with trifluoroacetic acid (4.0 mL, 51 mmol), anhydrous DMSO (1100 mL), (5-fluoro-1H-indol-3-yl)-2-methylpropan-1-ol (7.06 g, 34.1 mmol) and dicyclohexylcarbodiimide (21.1 g, 102 mmol). It was stirred at room temperature for 5 hours. The reaction mixture was quenched with ice-water (200 mL) and stirred for 1 hour. A white precipitate was filtered out and removed. The resulting solution was extracted with ethyl acetate (3×300 mL). The combined organic phases were washed with saturated aqueous NaCl, dried over MgSO₄, and concentrated under reduced pressure. Chromatography ((85:15) hexane-EtOAc) afforded 5.45 g (78%) of desired product as a tan solid. The product was characterized by ¹HNMR.

### Example 50

### Intermediate 97—3-(3-bromopropyl)-5-fluoro-1H-indole

To 3-(5-fluoro-1H-indol-3-yl)propan-1-ol (7.1 g, 0.037 mol) in anhydrous dichloromethane (50 mL), under nitrogen at 0° C., was added carbon tetrabromide (18.2 g, 0.055 mol), and slowly, portionwise, over a 10 min period, triphenylphosphine (14.4 g, 0.055 mol). The reaction mixture was stirred at room temperature for 3.5 hrs. It was concentrated in vacuo. Chromatography ((3:1) Hex-EtOAc) followed by ((1:1) Hex-EtOAc) afforded 4.7 g (50%) of desired product. The product was characterized by ¹HNMR.

### Example 51

### Intermediate 98—5-fluoro-3-(3-isocyanopropyl)-1H-indole

To 3-(3-bromopropyl)-5-fluoro-1H-indole (4.6 g, 0.018 mol) in anhydrous DMF (25 mL), under nitrogen at room temperature, was added sodium cyanide (1.74 g, 0.036 mol). The reaction mixture was stirred at room temperature overnight. It was then quenched with water and extracted with ethyl acetate (2×). The organic extracts were pooled, washed with water (2×), treated with brine, dried over anhydrous magnesium sulfate, filtered and concentrated. Chromatography ((2:1) Hex-EtOAc) afforded 3.14 g (87%) of desired product as a pale orange oil. The product was characterized by ¹HNMR.

### Example 52

### Intermediate 99—4-(5-fluoro-1H-indol-3-yl)butanoic acid

To 5-fluoro-3-(3-isocyanopropyl)-1H-indole (11.7 g, 0.0635 mol) in ethanol (250 mL)-water (200 mL) was added potassium hydroxide pellets (85%, 140 g, 2.12 mmol). The reaction mixture was brought to reflux and kept under reflux for 16 hrs. The reaction mixture was cooled down to room temperature and poured over ice —H₂O. It was slowly neutralized with concentrated hydrochloric acid, the white solid filtered off and washed thoroughly with water. It was dried under vacuum and 11.1 g (86%) of desired product was isolated as a white solid. The product was characterized by ¹HNMR.

### Example 53

### Intermediate 100—Methyl 4-(5-fluoro-1H-indol-3-yl)butanoate

To 4-(5-fluoro-1H-indol-3-yl)butanoic acid (5 g, 0.023 mol) in methanol (100 mL), was added trimethylorthoformate (4.3 mL, 0.04 mol) and sulfuric acid (0.5 mL). The reaction mixture was stirred at 50° C. for 40 min. Half the solvent was removed under vacuum and the reaction mixture poured over ice-water. The product was extracted with diethyl ether (2×). The combined ether extracts were washed with water, treated with brine, dried over anhydrous magnesium sulfate, filtered and concentrated to generate 5.24 g (98%) of desired product. The product was characterized by ¹HNMR.

### Example 54

### Intermediate 101—tert-butyl 5-fluoro-3-(4-methoxy-4-oxobutyl)-1H-indole-1-carboxylate

To methyl 4-(5-fluoro-1H-indol-3-yl)butanoate (5.1 g, 0.022 mol) in anhydrous dichloromethane (250 mL), under nitrogen at room temperature, was added t-butyl dicarbonate (5.95 mL, 0.026 mol) and dimethylaminopyridine (3.16 g, 0.026 mol). The reaction mixture was stirred at room temperature for 2 hrs. It was diluted with dichloromethane and washed with water (2×). The organic layer was treated with brine, dried over anhydrous magnesium sulfate, filtered and concentrated. Chromatography ((4:1) Hex-EtOAc) afforded 6.77 g (94%) of desired product as a pale yellow solid. The product was characterized by ¹HNMR.

### Example 55

### Intermediate 102—4-[1-(tert-butoxycarbonyl)-5-fluoro-1H-indol-3-yl]butanoic acid

To tert-butyl 5-fluoro-3-(4-methoxy-4-oxobutyl)-1H-indole-1-carboxylate (6.77 g, 0.02 mol) in tetrahydrofuran (105 mL), was added 1N LiOH/H₂O (25 mL, 0.025 mol). The reaction mixture was stirred at room temperature overnight. It was then concentrated and the residue taken up in ethyl acetate. 5% Citric acid was added, the organic layer separated, washed once with water, treated with brine, dried over anhydrous magnesium sulfate, filtered and concentrated to generate 6.36 g (98%) of desired product as a yellow solid. The product was characterized by ¹HNMR.

### Example 56

### Intermediate 103a—ert-butyl 3-{4-[(4R)-4-benzyl-2-oxo-1,3-oxazolidin-3-yl]-4-oxobutyl}-5-fluoro-1H-indole-1-carboxylate

To 4-[1-(tert-butoxycarbonyl)-5-fluoro-1H-indol-3-yl]butanoic acid (0.71 g, 2.21 mmol) in anhydrous tetrahydrofuran (12.5 mL), under nitrogen at −78° C., was added triethylamine (0.32 mL, 2.32 mmol) and pivaloyl chloride (0.3 mL, 2.43 mmol). The reaction mixture was transferred to an ice bath (0° C.) and stirred for 1 hr. The slurry was then cooled down to −78° C., and added via a dropping funnel to the following solution:

To (R)-(+)-4-benzyl-2-oxazolidinone (0.39 g, 2.21 mmol) in anhydrous tetrahydrofuran (11 mL), under nitrogen at −78° C., was added dropwise (via syringe), a 2.5M solution of n-butyl lithium in hexanes (0.88 mL, 2.21 mmol). The reaction mixture was stirred at −78° C. for 1 hr. To this clear solution was then added the above mixture. The reaction mixture was allowed to warm gradually to room temperature and stirred overnight. The slurry was quenched with water and ethyl acetate was added. The organic layer was separated and the aqueous layer extracted once more with ethyl acetate. The organic extracts were pooled, washed with water (1×), treated with brine, dried over anhydrous magnesium sulfate, filtered and concentrated. Chromatography ((3:1) Hex-EtOAc) afforded 0.67 g (64%) of desired product as a white foamy solid. The product was characterized by ¹HNMR. [α]_{D}²⁵=−68.2° (c=1% solution, DMSO).

### Example 57

### Intermediate 103b—tert-butyl 3-{4-[(4S)-4-benzyl-2-oxo-1,3-oxazolidin-3-yl]-4-oxobutyl}-5-fluoro-1H-indole-1-carboxylate

This compound was prepared as described above for example 56 (intermediate 103a) using (S)-(−)-4-benzyl-2-oxazolidinone. Chromatography ((3:1) Hex-EtOAc) afforded 0.77 g (69%) of desired product as a white foamy solid. The product was characterized by ¹HNMR. [α]_{D}²⁵=+77.8° (c=1% solution, DMSO).

### Example 58

### Intermediate 104a—tert-butyl 3-{(3R)-4-[(4R)-4-benzyl-2-oxo-1,3-oxazolidin-3-yl]-3-methyl-4-oxobutyl}-5-fluoro-1H-indole-1-carboxylate

To a 1M solution of sodium bis(trimethylsilyl) amide in tetrahydrofuran (0.69 mL, 0.69 mmol) in anhydrous THF (3 mL), under nitrogen at −40° C., was added dropwise over a 8 min period (via syringe), tert-butyl 3-{4-[(4R)-4-benzyl-2-oxo-1,3-oxazolidin-3-yl]-4-oxobutyl}-5-fluoro-1H-indole-1-carboxylate (0.3 g, 0.62 mmol) in anhydrous THF (3 mL). The flask was rinsed with THF (0.75 mL) and the rinse added to the reaction mixture. It was then stirred at −40° C. for 45 min. Iodomethane (0.05 mL, 0.81 mmol) was added and the yellow reaction mixture kept at −40° C. for 2 hrs. The reaction mixture was then brought to −20° C. and quenched with saturated ammonium chloride. It was diluted with ethyl acetate-water, the organic layer separated and the aqueous layer extracted once more with ethyl acetate. The organic extracts were pooled, treated with brine, dried over anhydrous magnesium sulfate, filtered and concentrated. Chromatography ((4:1) Hex-EtOAc) afforded 0.14 g (45%) of desired product as a white solid. The product was characterized by ¹HNMR. [α]_{D}²⁵=−70.4° (c=1% solution, DMSO).

### Example 59

### Intermediate 104b—tert-butyl 3-{(3S)-4-[(4S)-4-benzyl-2-oxo-1,3-oxazolidin-3-yl]-3-methyl-4-oxobutyl}-5-fluoro-1H-indole-1-carboxylate

This compound was prepared as described above for example 58 (intermediate 104a) using tert-butyl 3-{4-[(4S)-4-benzyl-2-oxo-1,3-oxazolidin-3-yl]-4-oxobutyl}-5-fluoro-1H-indole-1-carboxylate as starting material. Chromatography ((4:1) Hex-EtOAc) afforded 0.15 g (49%) of desired product as a white solid. The product was characterized by ¹HNMR. [α]_{D}²⁵=+68.8° (c=1% solution, DMSO).

### Example 60

### Intermediate 105a—tert-butyl 5-fluoro-3-[(3R)-4-hydroxy-3-methylbutyl]-1H-indole-1-carboxylate

To tert-butyl 3-{(3R)-4-[(4R)-4-benzyl-2-oxo-1,3-oxazolidin-3-yl]-3-methyl-4-oxobutyl}-5-fluoro-1H-indole-1-carboxylate (0.188 g, 0.37 mmol) in anhydrous tetrahydrofuran (2 mL), at 0° C., was added water (0.0072 mL, 0.4 mmol) and dropwise a 2M solution of lithium borohydride in tetrahydrofuran (0.21 mL, 0.41 mmol). The reaction mixture was warmed up to room temperature and stirred for 2 hrs. The reaction mixture was then cooled down to 0° C. and quenched with 1N NaOH/H₂O (1.2 mL). The mixture was warmed up to room temperature and extracted with ethyl acetate (2×). The organic extracts were pooled, treated with brine, dried over anhydrous magnesium sulfate, filtered and concentrated. Chromatography ((3:1) Hex-EtOAc) afforded 0.09 g (77%) of desired product as a colorless gum. The product was characterized by ¹HNMR. [α]_{D}²⁵=+12.0° (c=1% solution, DMSO).

### Example 61

### Intermediate 105b—tert-butyl 5-fluoro-3-[(3S)-4-hydroxy-3-methylbutyl]-1H-indole-1-carboxylate

This compound was prepared as described above for example 60 (intermediate 105a) using tert-butyl 3-{(3S)-4-[(4S)-4-benzyl-2-oxo-1,3-oxazolidin-3-yl]-3-methyl-4-oxobutyl}-5-fluoro-1H-indole-1-carboxylate as starting material. Chromatography ((3:1) Hex-EtOAc) afforded 0.07 g (79%) of desired product as a gum. The product was characterized by ¹HNMR. [α]_{D}²⁵=−12.0° (c=1% solution, DMSO).

### Example 62

### Intermediate 55—1-[(2,2-diethoxyethyl)thio]-4-fluorobenzene

To 4-fluorothiophenol (10 g, 0.078 mol) in anhydrous acetone (100 ml), under nitrogen at room temperature, was added potassium carbonate (10.78 g, 0.078 mol). To the reaction mixture was slowly added bromoacetaldehyde diethyl acetal (10.8 ml, 0.072 mol). The reaction mixture was stirred at room temperature overnight. The potassium carbonate was filtered off and washed thoroughly with acetone. The filtrate was then concentrated and the oily residue diluted with H₂O and extracted with Et₂O. The Et₂O extracts were washed with 0.5 M KOH, H₂O and brine, dried over anhydrous magnesium sulfate, filtered and concentrated. Chromatography ((9.5:0.5) Hexane-EtOAc) afforded 17.8 g (93%) of 1-[(2,2-diethoxyethyl)thio]-4-fluorobenzene as a clear oil. The product was characterized by ¹HNMR.

### Example 63

### Intermediate 56—5-fluoro-1-benzothiophene

To a 3-neck 500 ml flask was introduced PPA (24 g) and anhydrous chlorobenzene (175 ml). The reaction mixture was mechanically stirred under nitrogen at reflux. 1-[(2,2-diethoxyethyl)thio]-4-fluorobenzene was then added over a 5 min period in 2 ml of chlorobenzene. Within 30 min the reaction mixture turned relatively dark and it was kept under reflux for 3 hrs. The reaction mixture was then cooled down to room temperature and the chlorobenzene layer decanted. The black tar was suspended in H₂O (200 ml) and stirred for about 30 min. The aqueous layer was extracted with CH₂Cl₂ (2×). The organic extracts were pooled with the chlorobenzene layer, treated with brine, dried over anhydrous magnesium sulfate, filtered and concentrated. Chromatography (Hexane) afforded 2 g (48%) of 5-fluoro-1-benzothiophene as a clear oil (very volatile). The product was characterized by ¹HNMR.

### Example 64

### Intermediate 11—3-chloro-1-(5-fluoro-1-benzothien-3-yl)propan-1-one

To AlCl₃ (1.82 g, 13.7 mmol) in anhydrous chloroform (40 ml), under nitrogen at 0 C, was added over a 1.25 hr period, a premixed solution of 5-fluoro-1-benzothiophene (2.32 g, 15.2 mmol) and 3-chloropropionyl chloride (1.74 ml, 18.2 mmol) in anhydrous chloroform (100 ml). The reaction mixture was then brought to room temperature and stirred overnight. The black reaction mixture was quenched with 1.5N HCl (330 ml), the organic layer treated with saturated sodium bicarbonate, H₂O and brine. It was dried over anhydrous magnesium sulfate, filtered and concentrated. Chromatography ((7:1) Hexane-EtOAc) afforded 0.44 g (46% based on recovered starting material) of 3-chloro-1-(5-fluoro-1-benzothien-3-yl)propan-1-one as a reddish solid. The product was characterized by ¹HNMR.

### Example 65

### Intermediate 58—2-(1-benzothien-3-yl)ethanol

To 1-benzothien-3-ylacetic acid (10 g, 0.052 mol) in anhydrous tetrahydrofuran (150 ml), under nitrogen at 0° C., was added dropwise a 1M solution of lithium aluminum hydride (57 ml, 0.057 mol) via an addition funnel. The reaction mixture was brought to room temperature and stirred overnight. The reaction mixture was then cooled to 0 C and slowly quenched with H₂O (20 ml). It was acidified to pH 4 with 1N HCl (70 ml) and concentrated to remove the THF. The aqueous mixture was extracted with ethyl acetate (3×) and the pooled organic extracts treated with saturated sodium bicarbonate, brine, dried over anhydrous magnesium sulfate, filtered and concentrated. Chromatography ((99:1) CH₂Cl₂—MeOH) afforded 8.35 g (90%) of 2-(1-benzothien-3-yl)ethanol as an orange oil. The product was characterized by ¹HNMR.

### Example 66

### Intermediate 59—2-(1-benzothien-3-yl)ethyl-4-methylbenzene sulfonate

To 2-(1-benzothien-3-yl)ethanol (8.35 g, 0.0468 mol) in anhydrous methylene chloride (140 ml), under nitrogen at 0° C., was added p-toluene sulfonyl chloride (9.82 g, 0.052 mol). Triethylamine (13 ml, 0.094 mol) was then slowly added and the reaction mixture stirred at room temperature over the weekend. The reaction mixture was washed with 1M KHSO₄ and the aqueous layer extracted with methylene chloride (2×). The organic extracts were combined, washed with saturated sodium bicarbonate, dried over anhydrous sodium sulfate, filtered and concentrated. Chromatography ((9:1) Hexane-EtOAc) afforded 14.7 g (94%) of 2-(1-benzothien-3-yl)ethyl-4-methylbenzene sulfonate. The product was characterized by ¹HNMR.

### Example 67

### Intermediate 60—3-(1-benzothien-3-yl)propanenitrile

To 2-(1-benzothien-3-yl)ethyl-4-methylbenzene sulfonate (14.7 g, 0.044 mol) in anhydrous dimethylformamide (50 ml), under nitrogen at room temperature, was added sodium cyanide (4.33 g, 0.088 mol). The reaction mixture was stirred at room temperature overnight. The reaction mixture was then poured into H₂O (200 ml) and extracted with ethyl acetate (3×). The organic extracts were combined, treated with brine, dried over anhydrous magnesium sulfate, filtered and concentrated. Chromatography ((4:1) Hexane-EtOAc) afforded 7.72 g (93%) of 3-(1-benzothien-3-yl)propanenitrile as an oil. The product was characterized by ¹HNMR.

### Example 68

### Intermediate 13—[3-(1-benzothien-3-yl)propyl]amine

3-(1-benzothien-3-yl)propanenitrile (0.44 g, 2.35 mmol) was dissolved in a solution of absolute ethanol (70 ml)-ammonium hydroxide (48 ml), and transferred to a Parr shaker bottle. After flushing with nitrogen, 5% Rh/Al catalyst (0.176 g, 40% by weight) was added and the reaction mixture hydrogenated at 50 psi overnight. The reaction mixture was filtered through Celite, washed with ethanol, and the filtrate concentrated. The remaining oil was dissolved in ethyl acetate and extracted with H₂O (2×). The organic extracts were dried over anhydrous magnesium sulfate, filtered and concentrated. The hydrogenation reaction was repeated one more time on the reaction mixture and worked up as described above. Chromatography ((9:1) CH₂Cl₂—MeOH (1% NH₄OH)) afforded 0.192 g (43%) of [3-(1-benzothien-3-yl)propyl]amine as an oil. The product was characterized by ¹HNMR.

### Example 69

### Intermediate 62—Methyl-1-benzofuran-3-yl acetate

1-benzofuran-3-(2H)-one (or 3-coumaranone) (7.25 g, 54.1 mmol) and (carbomethoxymethylene) triphenyl phosphorane (21.67 g, 64.86 mmol) in anhydrous toluene (225 ml) were refluxed for 5 days under nitrogen. The reaction mixture was cooled to room temperature and concentrated. Chromatography ((9:1) Hexane-EtOAc) afforded 8.45 g (82%) of methyl-1-benzofuran-3-yl acetate as a red liquid. The product was characterized by ¹HNMR.

### Example 70

### Intermediate 63—1-benzofuran-3-yl acetic acid

To methyl-1-benzofuran-3-yl acetate (10.8 g, 56.8 mmol) in absolute ethanol (350 ml) was added 2.5N NaOH (32 ml, 79.5 mmol) and the reaction mixture brought to reflux. After 30 min of reflux, the reaction mixture was cooled down to room temperature and concentrated. The red solid was dissolved in H₂O and acidified with 2N HCl. The precipitate that formed was dissolved in ethyl acetate. The aqueous layer was separated and extracted with ethyl acetate (2×). The organic extracts were combined, treated with brine, dried over anhydrous magnesium sulfate, filtered and concentrated. Chromatography ((4:1) CH₂Cl₂—MeOH (1% NH₄OH)) generated the 1-benzofuran-3-yl acetic acid ammonium salt. This salt was then converted back to the acid by extraction with 1N HCl and CH₂Cl₂. The organic layer was dried over anhydrous magnesium sulfate, filtered and concentrated to afford 9.31 g (93%) of 1-benzofuran-3-yl acetic acid as a peach solid. The product was characterized by ¹HNMR.

### Example 71

### Intermediate 64a—2-(1-benzofuran-3-yl)ethanol

The title compound was prepared by generally following the procedure as described above for Intermediate 58 (example 65) using 1-benzofuran-3-yl acetic acid (9.3 g, 52.8 mmol) and 1M lithium aluminum hydride solution (58 ml, 58.1 mmol) in anhydrous tetrahydrofuran (150 ml). Chromatography ((2:1) Hexane-EtOAc) afforded 7.56 g (88%) of 2-(1-benzofuran-3-yl)ethanol as a yellow oil. The product was characterized by ¹HNMR.

### Example 72

### Intermediate 12a—3-(2-bromoethyl)-1-benzofuran

To 2-(1-benzofuran-3-yl)ethanol (7.56 g, 46.6 mmol) in anhydrous methylene chloride (70 ml), under nitrogen at 0 C, was added carbon tetrabromide (23.19 g, 69.9 mmol). To the reaction mixture was then added triphenylphosphine (18.34 g, 69.9 mmol) dropwise over a 40 min period, after which all the starting material had disappeared. The reaction mixture was warmed up to room temperature and concentrated. Chromatography (Hexane) afforded 9.13 g (87%) of 3-(2-bromoethyl)-1-benzofuran as a clear oil. The product was characterized by ¹HNMR.

### Example 73

### Intermediate 65a—3-(1-benzofuran-3-yl)propanenitrile

The title compound was prepared by generally following the procedure as described above for Intermediate 60 (example 67) using 3-(2-bromoethyl)-1-benzofuran (9.13 g, 40.6 mmol) and sodium cyanide (3.98 g, 81.1 mmol) in anhydrous dimethylformamide (63 ml). Chromatography ((5:1) Hexane-EtOAc) afforded 6.29 g (91%) of 3-(1-benzofuran-3-yl)propanenitrile as a clear oil. The product was characterized by ¹HNMR.

### Example 74

### Intermediate 14—[3-(7-methoxy-1-benzofuran-3-yl)propyl]amine

The title compound was prepared by generally following the procedure as described above for Intermediate 13 (example 68) using 3-(1-benzofuran-3-yl)propanenitrile (0.32 g, 1.86 mmol), 5% Rh/Al (0.127 g (40% by weight)) in absolute ethanol (55 ml)-ammonium hydroxide (38 ml). Chromatography ((9:1) CH₂Cl₂—MeOH (1% NH₄OH)) afforded 0.26 g (80%) of [3-(7-methoxy-1-benzofuran-3-yl)propyl]amine as an oil. The product was characterized by ¹HNMR.

### Example 75

### Intermediate 63a—3-(1-benzofuran-3-yl)propanoic acid

To a solution of ethanol (100 ml) and H₂O (200 ml) chilled to 0° C. and treated with KOH, 85% pellets (87.2 g, 1.32 mol) was added 3-(1-benzofuran-3-yl)propanenitrile (5.65 g, 33.0 mmol) and the reaction mixture refluxed for 16 hrs. The reaction mixture was cooled down to room temperature and poured over ice water. It was then neutralized with conc. HCl (165 ml), and extracted with ethyl acetate (3×). The combined organic extracts were treated with brine, dried over anhydrous magnesium sulfate, filtered and concentrated to generate a yellowish semi-solid. This crude product was triturated with H₂O and the precipitate filtered affording 6.03 g (96%) of 3-(1-benzofuran-3-yl)propanoic acid as a white solid. The product was characterized by ¹HNMR.

### Example 76

### Intermediate 64b—3-(1-benzofuran-3-yl)propan-1-ol

The title compound was prepared by generally following the procedure as described above for Intermediate 58 (example 65) using 3-(1-benzofuran-3-yl)propanoic acid (6.0 g, 31.5 mmol) and 1M lithium aluminum hydride solution (35 ml, 35 mmol) in anhydrous tetrahydrofuran (100 ml). Chromatography ((49:1) CH₂Cl₂—MeOH) afforded 5.0 g (90%) of 3-(1-benzofuran-3-yl)propan-1-ol as a clear oil. The product was characterized by ¹HNMR.

### Example 77

### Intermediate 12b—3-(3-bromopropyl)-1-benzofuran

The title compound was prepared by generally following the procedure as described above for Intermediate 12a (example 72) using 3-(1-benzofuran-3-yl)propan-1-ol (5.0 g, 28.4 mmol), carbon tetrabromide (11.3 g, 34.1 mmol) and triphenyl phosphine (8.9 g, 34.1 mmol) in anhydrous THF (60 ml). Chromatography ((97:3) Hexane-EtOAc) afforded 6.41 g (94%) of 3-(3-bromopropyl)-1-benzofuran as a clear oil. The product was characterized by ¹HNMR.

### Example 78

### Intermediate 65b—4-(1-benzofuran-3-yl)butanenitrile

The title compound was prepared by generally following the procedure as described above for Intermediate 60 (example 67) using 3-(3-bromopropyl)-1-benzofuran (3.0 g, 12.5 mmol) and sodium cyanide (3.06 g, 62.5 mol) in anhydrous dimethylformamide (30 ml). From the extraction work-up, pure product was obtained affording 2.29 g (99%) of 4-(1-benzofuran-3-yl)butanenitrile as an amber oil. The product was characterized by ¹HNMR.

### Example 79

### Intermediate 63b—4-(1-benzofuran-3yl)butanoic acid

The title compound was prepared by generally following the procedure as described above for Intermediate 63a (example 75) using 4-(1-benzofuran-3-yl)butanenitrile (2.29 g, 12.4 mmol), KOH, 85% pellets (32.7 g, 0.496 mol) in ethanol (50 ml)-H₂O(80 ml). From trituration, 2.44 g (96%) of 4-(1-benzofuran-3yl)butanoic acid as a light-amber solid was isolated: mp 83-84° C.; MS (ES) m/z 203.1 ([M−H]⁻); Anal. calculated for C₁₂H₁₂O₃; C, 70.58; H, 5.92; N, 0.00; Found: C, 70.42; H, 5.98; N, 0.00.

### Example 80

### Intermediate 64c—4-(1-benzofuran-3-yl)butan-1-ol

The title compound was prepared by generally following the procedure as described above for Intermediate 58 (example 65) using 4-(1-benzofuran-3yl)butanoic acid (2.36 g, 11.6 mmol), 1M lithium aluminum hydride solution (14 ml, 14 mmol) in anhydrous tetrahydrofuran (35 ml). Chromatography ((49:1) CH₂Cl₂—MeOH) afforded 1.91 g (86%) of 4-(1-benzofuran-3-yl)butan-1-ol as an amber oil. The product was characterized by ¹HNMR.

### Example 81

### Intermediate 12c—3-(4-bromobutyl)-1-benzofuran

The title compound was prepared as described above for Intermediate 12a (example 72) using 4-(1-benzofuran-3-yl)butan-1-ol (1.9 g, 9.99 mmol), carbon tetrabromide (3.98 g, 12 mmol) and triphenylphosphine (3.15 g, 12 mmol) in anhydrous tetrahydrofuran (25 ml). Chromatography ((9:1) Hexane-EtOAc) afforded 2.21 g (87%) of 3-(4-bromobutyl)-1-benzofuran as an amber oil. The product was characterized by H

### Example 82

### Intermediate 67—5-Fluoro-1-(phenylsulfonyl)-1H-indole

To 5-fluoroindole (0.5 g, 3.7 mmol) in anhydrous tetrahydrofuran (8.5 ml), under nitrogen at −78° C., was added dropwise 2.5 M nBuLi/hexane (1.6 ml, 4.1 mmol) and the reaction mixture stirred for 40 min at −78 C. It was transferred to an ice bath and stirred for another 20 min. Benzenesulfonyl chloride (0.5 ml, 3.88 mmol) was then added dropwise and the reaction mixture slowly brought to room temperature, and stirred for 2.5 hrs. It was poured over 2% NaHCO₃ and extracted with diethyl ether. The organic extracts were washed with 2% NaHCO₃, water and brine, dried over anhydrous magnesium sulfate, filtered and concentrated under vacuum. Chromatography ((9:1) Hexane-EtOAc) afforded 1.05 g (100%) of the title compound as a white solid. The product was characterized by ¹HNMR.

### Example 83

### Intermediate 15—3-Chloro-1-[5-fluoro-1-(phenylsulfonyl)-1H-indol-3-yl]propan-1-one

To aluminum chloride (2.5 g, 19 mmol) in anhydrous dichloromethane (19 ml), under nitrogen at 0° C., was added dropwise 3-chloropropionyl chloride (1.8 ml, 19 mmol), and the reaction mixture stirred at 0° C. for 20 min. A solution of 5-fluoro-1-(phenylsulfonyl)-1H-indole (1.05 g, 3.8 mmol) in dichloromethane (19 ml) was added dropwise over a 15 min period. After 1.5 hrs, the reaction mixture was quenched with 1N HCl/H₂O, the organic layer separated and washed with saturated NaHCO₃, water and brine. It was dried over anhydrous magnesium sulfate, filtered and concentrated under vacuum. Chromatography ((3:1) Hexane-EtOAc) afforded 1.10 g (79%) of the title compound as an off-white solid. The product was characterized by ¹HNMR.

### Example 84

### Intermediate 50—8-fluoro-3-({3-[5-fluoro-1-(phenylsulfonyl)-1H-indol-3-yl]-3-oxopropyl}amino)chromane-5-carboxamide

To 3-amino-8-fluorochromane-5-carboxamide (0.4 g, 1.9 mmol) in anhydrous DMF (18 mL), under nitrogen at room temperature, was added K₂CO₃ (0.2 g, 1.46 mmol) and 3-chloro-1-[5-fluoro-1-(phenylsulfonyl)-1H-indol-3-yl]propan-1-one (0.53 g, 1.46 mmol) dissolved in anhydrous DMF (5 mL). The reaction mixture was stirred at room temperature overnight. The reaction mixture was concentrated and taken up in EtOAc/H₂O. The organic layer was separated and the aqueous layer extracted once more with EtOAc. The organic extracts were pooled, dried over magnesium sulfate, filtered and concentrated affording 0.8 g (100%) of desired product as a very insoluble pale yellow solid. Its identity was confirmed by ¹HNMR.

### Example 85

### Intermediate 51—8-fluoro-3-({3-[5-fluoro-1-(phenylsulfonyl)-1H-indol-3-yl]-3-hydroxypropyl}amino)chromane-5-carboxamide

To 8-fluoro-3-({3-[5-fluoro-1-(phenylsulfonyl)-1H-indol-3-yl]-3-oxopropyl}amino) chromane-5-carboxamide (0.18 g, 0.334 mmol) in CHCl₃ (8 mL)-iPrOH (2.2 mL), under nitrogen at room temperature, was added silica gel (0.225 g). To the slurry was then added sodium borohydride (0.064 g, 1.7 mmol) and the reaction mixture stirred at room temperature overnight. It was quenched with acetic acid, filtered, and the silica gel washed thoroughly with EtOAc/MeOH. The filtrate was then concentrated. Chromatography ((5:4:1) EtOAc-Hex-MeOH (1% NH₄OH)) afforded 0.12 g (68%) of desired product as a white solid. Its identity was confirmed by ¹HNMR.

### Example 86

### Intermediate 52—3-(cyclobutyl{3-[5-fluoro-1-(phenylsulfonyl)-1H-indol-3-yl]-3-hydroxypropyl}amino)-8-fluorochromane-5-carboxamide

To 8-fluoro-3-({3-[5-fluoro-1-(phenylsulfonyl)-1H-indol-3-yl]-3-hydroxypropyl}amino)chromane-5-carboxamide (0.12 g, 0.223 mmol) in anhydrous methanol (3 mL), under nitrogen at room temperature, was added cyclobutanone (0.42 mL, 0.558 mmol), acetic acid (0.26 mL, 0.535 mmol) and sodium cyanoborohydride (0.28 g, 0.446 mmol). The reaction mixture was stirred at room temperature overnight. More cyclobutanone (0.42 mL), acetic acid (0.026 mL) and sodium cyanoborohydride (0.028 g) were added. The reaction mixture was stirred at room temperature over the weekend. Chromatography ((6:3:1) Hex-EtOAc-MeOH (1% NH₄OH)) afforded 0.1 g (77%) of desired product as a white solid. Its identity was confirmed by ¹HNMR.

### Example 87

### Intermediate 68—5-[1-(5-fluoro-1H-indol-3-yl)ethyl]-2,2-dimethyl-1,3-dioxane-4,6-dione

To 5-fluoroindole (3.75 g, 27.7 mmol) in acetonitrile (55 ml), under nitrogen at room temperature, was added Meldrum's acid (3.99 g, 27.7 mmol) and acetaldehyde (3.1 ml, 54.4 mmol). The reaction mixture was stirred at room temperature overnight. The reaction mixture was then concentrated under vacuum to dryness. Chromatography ((4:1) Hexane-EtOAc) afforded 6.3 g (74%) of the title compound as a white solid: mp 129-130 C; MS (ESI) m/z 304 ([M−H]⁻); Anal. Calculated for C₁₆H₁₆FNO₄.0.30 H₂O; C, 61.85; H, 5.39; N, 4.51; Found: C, 61.51; H, 4.85; N, 4.23.

### Example 88

### Intermediate 69—Ethyl 3-(5-fluoro-1H-indol-3-yl) butanoate

To 5-[1-(5-fluoro-1H-indol-3-yl)ethyl]-2,2-dimethyl-1,3-dioxane-4,6-dione (6.3 g, 20.6 mmol) in absolute ethanol (10 ml)-pyridine (50 ml), was added Cu° powder (0.8 g). The reaction mixture was heated to 105 C and stirred at that temperature for 1 hour. The reaction mixture was concentrated under vacuum, the residue dissolved in ethyl acetate and the copper solids filtered off. The organic layer was washed with 2N HCl, water and saturated NaHCO₃, dried over anhydrous magnesium sulfate, filtered and concentrated. Chromatography (15% EtOAc in hexane) afforded 2.91 g (49%) of the title compound as a red semi-solid. The product was characterized by ¹HNMR.

The enantiomers of ethyl 3-(5-fluoro-1H-indol-3-yl) butanoate (3.7 g) were separated by chiral HPLC and isolated generating 1.62 g of ethyl (3S)-3-(5-fluoro-1H-indol-3-yl)butanoate as an amber gum and 1.61 g of ethyl (3R)-3-(5-fluoro-1H-indol-3-yl)butanoate as an amber gum. The products were characterized by ¹HNMR.

### Example 89

### Intermediate 70—3-(5-fluoro-1H-indol-3-yl) butan-1-ol

To ethyl (3S)-3-(5-fluoro-1H-indol-3-yl) butanoate (1.59 g, 6.38 mmol) in anhydrous tetrahydrofuran (30 ml), under nitrogen at 0° C., was added dropwise a 1M solution of lithium aluminum hydride in THF (19.1 ml, 19.1 mmol). The reaction mixture was stirred at room temperature for 2 hours. The reaction mixture was then poured into ice water and ethyl acetate was added. It was filtered through Celite. The organic layer was separated and the aqueous phase extracted with EtOAc (2×). The combined organic extracts were washed with brine, dried over anhydrous magnesium sulfate, filtered and concentrated. 1.24 g (94%) of (3S)-3-(5-fluoro-1H-indol-3-yl)butan-1-ol was isolated as a clear gum. The product was characterized by ¹HNMR.

The same procedure was used for the preparation of (3R)-3-(5-fluoro-1H-indol-3-yl) butan-1-ol and 1.30 g (98%) of the title compound was isolated as a slightly grayish gum. The product was characterized by ¹HNMR.

### Example 90

### Intermediate 16—3-(5-fluoro-1H-indol-3-yl)butanal

This compound was prepared by following the procedure as described above for Intermediate 4a (example 1a) using (3S)-3-(5-fluoro-1H-indol-3-yl)butan-1-ol (1.24 g, 5.98 mmol), trifluoroacetic acid (0.93 ml, 12 mmol), pyridine (1.93 ml, 23.9 mmol), chlorobenzene (25 ml), DMSO (25 ml) and dicyclohexylcarbodiiimide (4.93 g, 23.9 mmol). Chromatography ((4:1) Hexane-EtOAc) afforded 1.1 g (90%) of (3S)-3-(5-fluoro-1H-indol-3-yl) butanal as an amber semi-solid. The product was characterized by ¹HNMR.

The same procedure was used for the preparation of (3R)-3-(5-fluoro-1H-indol-3-yl) butanal using (3R)-3-(5-fluoro-1H-indol-3-yl) butan-1-ol (1.30 g, 6.27 mmol), trifluoroacetic acid (0.97 ml, 12.5 mmol), pyridine (2.03 ml, 25.1 mmol), chlorobenzene (25 ml), DMSO (25 ml) and dicyclohexylcarbodiiimide (5.18 g, 25.1 mmol). Chromatography ((4:1) Hexane-EtOAc) afforded 1.08 g (84%) of (3R)-3-(5-fluoro-1H-indol-3-yl)butanal as an amber semi-solid. The product was characterized by ¹HNMR.

### Example 91

### Intermediate 72—3-(5,7-difluoro-1H-indol-3-yl)-propan-1-ol

A mixture of commercial 2,4-difluorophenylhydrazine HCl (10 g, 55.3 mmol) and 3,4-dihydropyran (4,652 g, 55.3 mmol) in water (50 ml) and dioxane (200 ml) was refluxed for 16 hours. After cooling to ambient temperature the mixture was diluted with ethyl acetate. The aqueous layer was separated and re-extracted with ethyl acetate. The combined organic layer was washed with brine, dried over magnesium sulfate, filtered and evaporated in vacuo to dryness. The residue was subjected to flash column chromatography on silica gel (400 g). Elution with ethyl acetate/hexane (2:3) afforded 8.4 g (72%) of the desired product as an amber oil. MS (ES) m/z 210.1 ([M−H]⁻).

### Example 92

### Intermediate 73—3-(5,7-Difluoro-1H-indol-3-yl)-propionaldehyde

Pyridine (6.33 g, 8 mmol) was added to benzene (90 ml). Under stirring and at ambient temperature trifluoroacetic acid (4.56 g, 40 mmol) was added, followed by DMSO (90 ml), 3-(5,7-difluoro-1H-indol-3-yl)-propan-1-ol (4.22 g, 20 mmol) and DCC (24.76 g, 120 mmol). The reaction mixture was stirred at ambient temperature for 24 hours after which water (200 ml) was added and stirring continued for 2 hours. The crude product was extracted with chloroform (2×150 ml) and the combined organic layer dried over magnesium sulfate, filtered and evaporated to dryness in vacuo. The residue was flash chromatographed on silica gel (400 g). Elution with a solvent gradient of 20 to 25% ethyl acetate/hexane afforded ˜2.5 g (˜60%) of the desired compound as an amber oil. MS (ES) m/z 208.1 ([M−H]⁻).

### Example 93

### Intermediate 76a—Ethyl 6-fluoro-2,3,4,9-tetrahydro-1H-carbazole-3-carboxylate

4-Cyclohexanonecarboxylic acid ethyl ester (25 g, 0.14 mol) and 4-fluorophenyhydrazine hydrochloride (22.5 g, 0.13 mol) were dissolved in ethanol (450 mL) and heated under reflux for 16 hours. After cooling, the white solid was filtered off and the solvent removed under reduced pressure. After partitioning the residue between water and ethyl acetate, the organic portion was separated, dried (MgSO₄) and evaporated under reduced pressure to generate 35.5 g (93%) of desired product which was recrystallized from heptane. mp: 115-117° C. MS: [M+H]+@m/e=262 [Lit. ref.: Block, M. H., et al. J. Med. Chem. 2002, 45, 3509].

### Example 94

### Intermediate 77a—(6-fluoro-2,3,4,9-tetrahydro-1H-carbazol-3-yl)methanol

Lithium aluminum hydride (800 mg) was added portionwise to a solution of ethyl 6-fluoro-2,3,4,9-tetrahydro-1H-carbazole-3-carboxylate (5.77 g, 22.1 mmol) in dry THF (100 mL). The mixture was stirred at ambient temperature under nitrogen for 16 hours, followed by quenching with the addition of an aqueous Rochelle salt solution. The reaction mixture was diluted with ether and the phases were separated. The aqueous phase was extracted once with ether and the ether layers were combined, dried (MgSO₄), and evaporated to give a residue. Chromatography ((2:1) Hex-EtOAc) afforded 3.90 g (80%) of desired product: mp: 107-109° C. MS: [M−H]−=218.1.

### Example 95

### Intermediate 78a—6-fluoro-2,3,4,9-tetrahydro-1H-carbazole-3-carbaldehyde

Dess-Martin periodinane (7.37 g 17.4 mmol) was added portionwise to a stirred solution/suspension of (6-fluoro-2,3,4,9-tetrahydro-1H-carbazol-3-yl) methanol (2.64 g, 11.8 mmole) in dichloromethane (120 mL). The alcohol completely dissolved after the Dess-Martin reagent was added. The reaction mixture was stirred at ambient temperature for 30 minutes, then quenched with ethanol. The reaction mixture was diluted with ether (860 mL) and washed twice with saturated aqueous sodium bicarbonate (550 mL) followed by 5% sodium thiosulfate pentahydrate. After washing with brine and drying (MgSO₄), the solvent was evaporated. Chromatography ((4:1) hex-EtOAc) afforded 1.2 g (47%) of desired product: mp: 96-98° C. MS: [M−H]−@m/z=216.1.

### Example 96

### Intermediate 116a—1-(4-Bromo-butyl)-4-fluoro-1H-indole

To a solution of the appropriate indole (1 g) in 20 mL of dimethylformamide, was added sodium hydride (60% in mineral oil, 8.14 mmol). The solution was stirred for 1-2 hours and then treated with 1,4 dibromobutane (2.66 mL, 22 mmol). The mixture was stirred for 45 minutes to 2 hours, quenched with 20 mL of water and extracted with ethyl acetate. The combined extracts were dried over anhydrous magnesium sulfate, and concentrated to a clear oil.

Purification by HPLC (Chromolith Monolith, 0.46×10 cm column, sample dissolved in dimethylsulfoxide, gradient acetonitrile/water (0.1% trifluoroacetic acid), 254 nm detection); Rt=2.68 generated a clear oil (72% yield). MS [(+)ESI, m/z]: 269.1, 271.1 [M+H]⁺.

### Example 97

### Intermediate 116b—1-(4-Bromo-butyl)-5-fluoro-1H-indole

This compound was prepared by generally following the procedure of example 96. Obtained as a clear oil (69% yield). MS [(+)ESI, m/z]: 269.1, 271.1 [M+H]+. HPLC (Chromolith Monolith, 0.46×10 cm column, sample dissolved in dimethylsulfoxide, gradient acetonitrile/water (0.1% trifluoroacetic acid), 254 nm detection); Rt=2.65.

### Example 98

### Intermediate 116c—1-(4-Bromo-butyl)-6-fluoro-1H-indole

This compound was prepared by generally following the procedure of example 96. Obtained as a clear oil (67.5% yield). MS [(+)ESI, m/z]: 269.0, 271.1 [M+H]⁺ HPLC (Chromolith Monolith, 0.46×10 cm column, sample dissolved in dimethylsulfoxide, gradient acetonitrile/water (0.1% trifluoroacetic acid, 254 nm detection); Rt=2.66.

### Example 99

### Intermediate 116d—1-(4-Bromo-butyl)-7-fluoro-1H-indole

This compound was prepared by generally following the procedure of example 96. Obtained as a clear oil (44% yield) by prep HPLC (Primesphere Silica, 5×25 cm column, flow rate 95 mL/min, sample dissolved in hexane, mobile phase: 5% ethyl acetate in hexane). MS [(+)ESI, m/z]: 270.0, 272.0 [M+H]⁺. HPLC (Chromolith Monolith, 0.46×10 cm column, sample dissolved in dimethylsulfoxide, gradient acetonitrile/water (0.1% trifluoroacetic acid, 254 nm detection); Rt=2.712.

### Example 100

### Intermediate 117a—8-Fluoro-3-[4-(4-fluoro-indol-1-yl)-butylamino]-chroman-5-carboxylic Acid Amide

To a solution of the appropriate N-(4-bromobutyl)indoles of examples 96-99 (4 mmole) in dimethylsulfoxide (20 mL) was added 3-amino-8-fluoro-chroman-5-carboxylic acid amide (1 equivalent) followed by N,N′-diisopropylethyl amine (Hünig's base, 1.2 equivalents). The reaction mixture was stirred under nitrogen at 85° C. for 5 hours and then overnight at room temperature, diluted with ethyl acetate and washed with aqueous sodium bicarbonate. The aqueous phase was extracted with ethyl acetate (1×) and the pooled organic extracts were dried with anhydrous magnesium sulfate and evaporated to dryness. Purification was carried out by flash chromatography using a Biotage Quad 12/25 (Dyax Corp) with KP Sil 32-63 mM, 60 Å cartridges and the crude product was preabsorbed. Elution with a gradient from 100% dichloromethane to 4% methanolic ammonia in dichloromethane provided the title products. Obtained as a pale yellow foam (63.5% yield). MS [(+)ESI, m/z]: 400.17 [M+H]⁺. MS [(−)ESI, m/z]: 398.2 [M−H]⁻. HPLC (Chromolith Monolith, 0.46×10 cm column, sample dissolved in ethanol, acetonitrile/water (0.1% trifluoroacetic acid) gradient, 254 nm detection): Rₜ=1.7 min

### Example 101

### Intermediate 117b—8-Fluoro-3-[4-(5-fluoro-indol-1-yl)-butylamino]-chroman-5-carboxylic acid amide

This compound was prepared by generally following the procedure of example 100. Obtained as a pale yellow foam (58% yield). MS [(+) ESI, m/z]: 400.2 [M+H]⁺. MS [(−)ESI, m/z]: 398.2 [M−H]⁻. HPLC (Chromolith Monolith, 0.46×10 cm column, sample dissolved in dimethylsulfoxide, acetonitrile/water (0.1% trifluoroacetic acid) gradient, 254 nm detection): Rₜ=1.7 min.

### Example 102

### Intermediate 117c—8-Fluoro-3-[4-(6-fluoro-indol-1-yl)-butylamino]-chroman-5-carboxylic acid amide

This compound was prepared by generally following the procedure of example 100. Obtained as a white solid (60% yield), m.p. 146-148° C. MS [(+)ESI, m/z]: 400.2 [M+H]⁺. MS [(−)ESI, m/z]: 398.2 [M−H]⁻. HPLC (Chromolith Monolith, 0.46×10 cm column, sample dissolved in acetonitrile, acetonitrile/water (0.1% trifluoroacetic acid) gradient, 254 nm detection): 1.69 min.

### Example 103

### Intermediate 117d—8-Fluoro-3-{[4-(7-fluoro-1H-indol-1-yl)butyl]amino}chromane-5-carboxamide hydrochloride salt

This compound was prepared by generally following the procedure of example 100. The free base was obtained as a white solid, m.p. 166-168° C. MS [(+)ESI, m/z]: 400.2 [M+H]⁺. MS [(−)ESI, m/z]: 398.2 [M−H]⁻. HPLC (Chromolith Monolith, 0.46×10 cm column, sample dissolved in ethanol, acetonitrile/water (0.1% trifluoroacetic acid) gradient, 254 nm detection): Rₜ=1.69 min.

The hydrochloride salt was obtained as an off-white amorphous solid by the addition of 1 equivalent of 1N hydrochloric acid in diethyl ether to an ethyl acetate/methanol solution of the free base. MS [(+)ESI, m/z]: 400.1 [M+H]⁺. MS [(−)ESI, m/z]: 398.1 [M−H]⁻. HPLC (Chromolith Monolith, 0.46×10 cm column, sample dissolved in dimethylsulfoxide, acetonitrile/water (0.1% trifluoroacetic acid) gradient, 254 nm detection): Rₜ=1.70 min.

### Example 104

### Intermediate 107—3-[(benzyloxy)methyl]cyclobutanone

3-[(benzyloxy)methyl]cyclobutanone was prepared according to the procedure described by T. Rammeloo and C. V. Stevens, Chem. Comm., 2002, 250-251.

### Example 105

### Intermediate 108-(3R)-3-[{(3-benzyloxy)methyl]-cyclobutyl}amino)-8-fluorochromane-5-carboxamide

(3R)-3-amino-8-fluorochromane-5-carboxamide was dissolved in methanol and 3-[(benzyloxy)methyl]cyclobutanone (1.25 eq.) was added followed by sodium cyanoborohydride (2.1 eq.) and acetic acid (2.4 eq.). The reaction was stirred at room temperature until completion and subject to workup and purification to yield pure (3R)-3-[{(3-benzyloxy)methyl]-cyclobutyl}amino)-8-fluorochromane-5-carboxamide as a mixture of diastereomers. MS (ES, m/z) calcd. For C₂₂H₂₅FN₂O₃ (M⁺) 384.1850, found 385.1 (M+H⁺).

### Example 106

### Intermediate 109—(3R)-3-[{3-[(benzyloxy)methyl]cyclobutyl}(trifluoroacetyl)amino]-8-fluorochromane-5-carboxamide

To a solution of (3R)-3-[{(3-benzyloxy)methyl]-cyclobutyl}amino)-8-fluorochromane-5-carboxamide in CH₂Cl₂, DMAP (1.2 eq.) was added followed by trifluoroacetic anhydride (1.2 eq.). After reaction was complete, the reaction was diluted with additional CH₂Cl₂ and washed with water, 0.1N HCl soln. and saturated brine soln. successively. The organic layer was dried with MgSO₄ and filtered and concentrated to give the title compound as a mixture of diastereomers. ¹H NMR consistent.

### Example 107

### Intermediate 110—(3R)-8-fluoro-3-[[3-(hydroxymethyl)cyclobutyl](trifluoroacetyl)amino]chromane-5-carboxamide

Treatment of (3R)-3-[{3-[(benzyloxy)methyl]cyclobutyl}(trifluoroacetyl)amino]-8-fluorochromane-5-carboxamide with Pd(OH)₂ and cyclohexene in refluxing ethanol yields the title compound as a mixture of diastereomers.

### Example 108

### Intermediate 111—(3R)-3-[[3-(bromomethyl)cyclobutyl](trifluoroacetyl)amino]-8-fluorochromane-5-carboxamide

Treatment of (3R)-8-fluoro-3-[[3-(hydroxymethyl)cyclobutyl](trifluoroacetyl)amino]chromane-5-carboxamide with carbon tetrabromide and triphenylphosphine yields the title compound as a mixture of diastereomers.

Preparation of Compounds of the Invention

### Example 109

### 8-fluoro-3-{[3-(5-fluoro-1H-indol-3-yl)propyl]amino}chromane-5-carboxamide (“Compound 1”)

A solution of 3-amino-8-fluorochromane-5-carboxamide (0.81 g, 3.8 mmol), 3-(3-bromopropyl)-5-fluoro-1H-indole (0.55 g, 2.1 mmol), and triethylamine (0.60 ml, 4.2 mmol) in anhydrous dimethylsulfoxide (20 ml) was stirred at 90 C for 9.5 hrs. The reaction mixture was cooled down to room temperature, diluted with ethyl acetate, and extracted a few times with water. The aqueous layer was back extracted once with ethyl acetate. The organic layer was treated with brine, dried over anhydrous magnesium sulfate, filtered and concentrated under vacuum. Chromatography ((5:4:1) EtOAc-Hexane-MeOH (1% NH₄OH)) afforded 0.48 g (60%) of the title compound as a peach solid.

It was converted to the HCl salt by dissolution in ethyl acetate and addition of 1M HCl/Et₂O solution (1.2 eq) to generate 8-fluoro-3-{[3-(5-fluoro-1H-indol-3-yl)propyl]amino}chromane-5-carboxamide hydrochloride salt as an off-white solid: mp 122° C./dec; MS (ESI) m/z 384 ([M−H]⁻); Anal. calculated for C₂₁H₂₁F₂N₃O₂.1.20 HCl; C, 58.77; H, 5.21; N, 9.79; Found: C, 58.82; H, 5.23; N, 9.73.

### Examples 109a and 109b

### (+)-8-fluoro-3-{[3-(5-fluoro-1H-indol-3-yl)propyl]amino}chromane-5-carboxamide (“Compound 1a”) and (−)-8-fluoro-3-{[3-(5-fluoro-1H-indol-3-yl)propyl]amino}chromane-5-carboxamide (“Compound 1b”)

The enantiomers of 8-fluoro-3-{[3-(5-fluoro-1H-indol-3-yl)propyl]amino}chromane-5-carboxamide (example 109) were separated by chiral HPLC, isolated and converted to the HCl salt as described above for the racemate, generating the following products:

(+)-8-fluoro-3-{[3-(5-fluoro-1H-indol-3-yl)propyl]amino chromane-5-carboxamide hydrochloride salt as a white solid: mp 89° C./dec; [α]D²⁵=+19.8° (c=1% SOLUTION, DMSO); MS (ES) m/z 384.2 ([M−H]⁻); Anal. Calculated for C₂₁H₂₁F₂N₃O₂.HCl.1.20 H₂O; C, 56.87; H, 5.55; N, 9.47. Found: C, 56.79; H, 5.60; N, 10.06.

(−)-8-fluoro-3-{[3-(5-fluoro-1H-indol-3-yl)propyl]amino}chromane-5-carboxamide hydrochloride salt as a white solid: mp 87° C./dec; [α_{D}²⁵=−16.0° (c=1%, DMSO); MS (ES) m/z 386.1 ([M+H]⁺); Anal. Calculated for C₂₁H₂₁F₂N₃O₂.HCl.1.80 H₂O; C, 55.52; H, 5.68; N, 9.25. Found: C, 54.86; H, 6.23; N, 10.13.

### Example 110

### 8-fluoro-3-{[3-(5-fluoro-1H-indol-3-yl)propyl](propyl)amino}chromane-5-carboxamide (“Compound 2”)

To 8-fluoro-3-{[3-(5-fluoro-1H-indol-3-yl)propyl]amino}chromane-5-carboxamide (0.12 g, 0.31 mmol) in anhydrous methanol (5.2 ml), under nitrogen at room temperature, was added propionaldehyde (0.025 ml, 0.341 mmol), acetic acid (0.042 ml, 0.744 mmol) and sodium cyanoborohydride (0.039 g, 0.62 mmol). The reaction mixture was stirred at room temperature overnight. The reaction mixture was then quenched with 1N NaOH/H₂O and concentrated under vacuum to get rid of methanol. The residue was taken up in CH₂Cl₂/H₂O, extracted with methylene chloride (3×), and the organic layer treated with brine, dried over anhydrous magnesium sulfate, filtered and concentrated under vacuum. Chromatography ((5:4:1) EtOAc-Hexane-MeOH (1% NF₄OH)) afforded 0.12 g (90%) of the title compound as a sticky gum.

It was converted to the HCl salt by dissolution in ethyl acetate and addition of 1M HCl/Et₂O solution (1.2 eq) to generate 8-fluoro-3-{[3-(5-fluoro-1H-indol-3-yl)propyl](propyl)amino}chromane-5-carboxamide hydrochloride salt as a pale yellow solid: mp 125° C./dec; MS (ESI) m/z 426 ([M−H]⁻); Anal. calculated for C₂₄H₂₇F₂N₃O₂.HCl.0.20 H₂O; C, 61.65; H, 6.12; N, 8.99; Found: C, 61.61; H, 6.10; N, 8.87.

### Examples 110a and 110b

### (−)-8-fluoro-3-[[3-(5-fluoro-1H-indol-3-yl)propyl](propyl)amino]-3,4-dihydro-2H-chromene-5-carboxamide (“Compound 2a”) and (+)-8-fluoro-3-[[3-(5-fluoro-1H-indol-3-yl)propyl](propyl)amino]-3,4-dihydro-2H-chromene-5-carboxamide (“Compound 2b”)

The enantiomers of 8-fluoro-3-{[3-(5-fluoro-1H-indol-3-yl)propyl](propyl) amino}chromane-5-carboxamide (example 110) were separated by chiral HPLC, isolated, and converted to the HCl salt as described above for the racemate, generating the following products:

(−)-8-fluoro-3-[[3-(5-fluoro-1H-indol-3-yl)propyl](propyl)amino]-3,4-dihydro-2H-chromene-5-carboxamide hydrochloride salt as a white solid: mp 126° C./dec; [α]_{D}²⁵=−31.49° (c=1% SOLUTION, DMSO); MS (ESI) m/z 428 ([M+H]⁺); Anal. calculated for C₂₄H₂₇F₂N₃O₂.1.20 HCl.0.25 H₂O; C, 60.59; H, 6.08; N, 8.83; Found: C, 60.50; H, 5.96; N, 8.64.

(+)-8-fluoro-3-[[3-(5-fluoro-1H-indol-3-yl)propyl](propyl)amino]-3,4-dihydro-2H-chromene-5-carboxamide hydrochloride salt as a white solid: mp 126° C./dec; [α]_{D}²⁵=+30.67° (c=1% SOLUTION, DMSO); MS (ESI) m/z 426 ([M−H]⁻); Anal. calculated for C₂₄H₂₇F₂N₃O₂.1.20 HCl.0.20 H₂O; C, 60.71; H, 6.07; N, 8.85; Found: C, 60.69; H, 5.85; N, 8.65.

### Example 111

### 3-{ethyl[3-(5-fluoro-1H-indol-3-yl)propyl]amino}-8-fluoro-3,4-dihydro-2H-chromene-5-carboxamide (“Compound 3”)

This compound was prepared by generally following the procedure as described above for example 110 using 8-fluoro-3-{[3-(5-fluoro-1H-indol-3-yl)propyl]amino}chromane-5-carboxamide (0.24 g, 0.61 mmol), acetaldehyde (0.037 ml, 0.668 mmol), acetic acid (0.086 ml, 1.46 mmol) and sodium cyanoborohydride (0.076 g, 1.21 mmol) in anhydrous methanol (10 ml). Chromatography ((5:4:1) EtOAc-Hexane-MeOH (1% NH₄OH)) afforded 0.23 g (91%) of the title compound as a foamy solid.

### Examples 111a and 111b

### (−)-3-{ethyl[3-(5-fluoro-1H-indol-3-yl)propyl]amino}-8-fluoro-3,4-dihydro-2H-chromene-5-carboxamide (“Compound 3a”) and (+)-3-{ethyl[3-(5-fluoro-1H-indol-3-yl)propyl]amino}-8-fluoro-3,4-dihydro-2H-chromene-5-carboxamide (“Compound 3b”)

The enantiomers of 3-{ethyl[3-(5-fluoro-1H-indol-3-yl)propyl]amino}-8-fluoro-3,4-dihydro-2H-chromene-5-carboxamide (example 111) were separated by chiral HPLC, isolated, and converted to the HCl salt as described above for example 110, generating the following products:

(−)-3-{ethyl[3-(5-fluoro-1H-indol-3-yl)propyl]amino}-8-fluoro-3,4-dihydro-2H-chromene-5-carboxamide hydrochloride salt as an off-white solid: mp dec/118.0° C.; [α]_{D}²⁵=−29.30° (c=1% SOLUTION, DMSO); MS (ESI) m/z 414 ([M+H]⁺); Anal. calculated for C₂₃H₂₅F₂N₃O₂.1.10 HCl.0.85 H₂O; C, 58.92; H, 5.98; N, 8.96; Found: C, 58.99; H, 6.08; N, 8.81.

(+)-3-{ethyl[3-(5-fluoro-1H-indol-3-yl)propyl]amino}-8-fluoro-3,4-dihydro-2H-chromene-5-carboxamide hydrochloride salt as an off-white solid: mp dec/102.0° C.; [α]_{D}²⁵=+26.48° (c=1% SOLUTION, DMSO); MS (ESI) m/z 414 ([M+H]⁺); Anal. calculated for C₂₃H₂₅F₂N₃O₂.1.10 HCl.0.70 H₂O; C, 59.26; H, 5.95; N, 9.01; Found: C, 59.21; H, 6.08; N, 8.82.

### Example 112

### 3-{cyclobutyl[3-(5-fluoro-1H-indol-3-yl)propyl]amino}-8-fluorochromane-5-carboxamide (“Compound 4”)

To 8-fluoro-3-{[3-(5-fluoro-1H-indol-3-yl)propyl]amino}chromane-5-carboxamide (0.14 g, 0.35 mmol) in anhydrous methanol (6 ml), under nitrogen at room temperature, was added cyclobutanone (0.070 ml, 0.876 mmol), acetic acid (0.050 ml, 0.84 mmol) and sodium cyanoborohydride (0.044 g, 0.70 mmol). The reaction mixture was stirred at room temperature overnight. More cyclobutanone (0.026 ml), acetic acid (0.21 ml) and sodium cyanoborohydride (0.22 g) were added after 24 hrs and 48 hrs at which time the reaction went to completion. The reaction mixture was then quenched with 1N NaOH(H₂O and concentrated under vacuum to get rid of methanol. The residue was taken up in CH₂Cl₂/H₂O, extracted with methylene chloride (3×), and the organic layer treated with brine, dried over anhydrous magnesium sulfate, filtered and concentrated under vacuum. Chromatography ((5:4:1) EtOAc-Hexane-MeOH (1% NH₄OH)) afforded 0.12 g (78%) of the title compound as a sticky gum.

It was converted to the HCl salt by dissolution in ethyl acetate and addition of 1M HCl/Et₂O solution (1.2 eq) to generate 3-{cyclobutyl[3-(5-fluoro-1H-indol-3-yl)propyl]amino}-8-fluorochromane-5-carboxamide hydrochloride salt as an off-white solid: mp dec/109° C.; MS (ES) m/z 438.2 ([M−H]⁻); Anal. calculated for C₂₅H₂₇F₂N₃O₂.1.10 HCl.0.50H₂O; C, 61.45; H, 6.00; N, 8.60; Found: C, 61.46; H, 5.96; N, 8.37.

### Examples 112a and 112b

### (+)-3-{cyclobutyl[3-(5-fluoro-1H-indol-3-yl)propyl]amino}-8-fluorochromane-5-carboxamide (“Compound 4a”) and (−)-3-{cyclobutyl[3-(5-fluoro-1H-indol-3-yl)propyl]amino}-8-fluorochromane-5-carboxamide (“Compound 4b”)

The enantiomers of 3-{cyclobutyl[3-(5-fluoro-1H-indol-3-yl)propyl]amino}-8-fluorochromane-5-carboxamide (example 112) were separated by chiral HPLC, isolated, and converted to the HCl salt as described above for the racemate, generating the following products:

(+)-3-{cyclobutyl[3-(5-fluoro-1H-indol-3-yl)propyl]amino}-8-fluorochromane-5-carboxamide hydrochloride salt as a white solid: mp 129° C./dec; [α]_{D}²⁵=+27.56° (c=1% SOLUTION, DMSO); MS (ES) m/z 438.2 ([M−H]⁻); Anal. calculated for C₂₅H₂₇F₂N₃O₂.HCl.0.50 H₂O; C, 61.92; H, 6.03; N, 8.66; Found: C, 61.92; H, 6.09; N, 8.38.

(−)-3-{cyclobutyl[3-(5-fluoro-1H-indol-3-yl)propyl]amino}-8-fluorochromane-5-carboxamide hydrochloride salt as a white solid: mp 129° C./dec; [α]_{D}²⁵=26.76° (c=1% SOLUTION, DMSO); MS (ES) m/z 440.1 ([M+H]⁺); Anal. calculated for C₂₅H₂₇F₂N₃O₂.HCl.0.40 H₂O; C, 62.15; H, 6.01; N, 8.70; Found: C, 62.09; H, 5.99; N, 8.45.

### Example 113

### 3-{(cyclopropylmethyl)[3-(5-fluoro-1H-indol-3-yl)propyl]amino}-8-fluorochromane-5-carboxamide (“Compound 5”)

This compound was prepared by generally following the procedure as described above for example 10 using 8-fluoro-3-{[3-(5-fluoro-1H-indol-3-yl)propyl]amino}chromane-5-carboxamide (0.13 g, 0.34 mmol), cyclopropanecarboxaldehyde (0.035 ml, 0.472 mmol), acetic acid (0.046 ml, 0.809 mmol) and sodium cyanoborohydride (0.042 g, 0.674 mmol) in anhydrous methanol (5.8 ml). Chromatography ((5:4:1) EtOAc-Hexane-MeOH (1% NH₄OH)) afforded 0.13 g (89%) of the title compound as a gum which was converted to the HCl salt to generate 3-{(cyclopropylmethyl)[3-(5-fluoro-1H-indol-3-yl)propyl]amino}-8-fluorochromane-5-carboxamide hydrochloride salt as a white solid: mp 121° C./dec; MS (ES) m/z 440.0 ([M+H]⁺); Anal. calculated for C₂₅H₂₇F₂N₃O₂.HCl.0.25 H₂O; C, 62.50; H, 5.98; N, 8.75; Found: C, 62.49; H, 6.11; N, 8.63.

### Examples 113a and 113b

### (−)-3-{(cyclopropylmethyl)[3-(5-fluoro-1H-indol-3-yl)propyl]amino}-8-fluorochromane-5-carboxamide (“Compound 5a”) and (+)-3-{(cyclopropylmethyl)[3-(5-fluoro-1H-indol-3-yl)propyl]amino}-8-fluorochromane-5-carboxamide (“Compound 5b”)

The enantiomers of 8-fluoro-3-{[3-(5-fluoro-1H-indol-3-yl)propyl]amino}chromane-5-carboxamide (example 109) were separated by chiral HPLC and isolated. Each enantiomer was subjected to the reaction conditions as described above for example 113 (the racemate) and converted to the HCl salt, generating the following products:

(−)-3-{(cyclopropylmethyl)[3-(5-fluoro-1H-indol-3-yl)propyl]amino}-8-fluorochromane-5-carboxamide hydrochloride salt as a white solid: mp 123° C./dec; [α]_{D}²⁵=−29.4° (c=1% SOLUTION, DMSO); MS (ES) m/z 440.1 ([M+H]⁺); Anal. calculated for C₂₅H₂₇F₂N₃O₂.1.10.HCl: C, 62.61; H, 5.91; N, 8.76; Found: C, 62.49; H, 5.79; N, 8.74.

(+)-3-{(cyclopropylmethyl)[3-(5-fluoro-1H-indol-3-yl)propyl]amino}-8-fluorochromane-5-carboxamide hydrochloride salt as a white solid: mp 123° C./dec; [α]_{D}²⁵=+32.4° (c=1% SOLUTION, DMSO); MS (ES) m/z 440.1 ([M+H]⁺); Anal. calculated for C₂₅H₂₇F₂N₃O₂.1.10 HCl.0.10 H₂O; C, 62.37; H, 5.93; N, 8.73; Found: C, 62.33; H, 5.89; N, 8.61.

### Example 114

### 3-{(1-cyclopropylethyl)[3-(5-fluoro-1H-indol-3-yl)propyl]amino}-8-fluorochromane-5-carboxamide (“Compound 6”)

To 3-amino-8-fluorochromane-5-carboxamide (0.075 g, 0.357 mmol) in anhydrous methanol (2 ml), under nitrogen at room temperature, was added cyclopropylmethylketone (0.18 ml, 1.78 mmol), acetic acid (0.048 ml, 0.857 mmol) and sodium cyanoborohydride (0.045 g, 0.714 mmol). The reaction mixture was stirred at room temperature overnight. The reaction mixture was quenched with 1N NaOH/H₂O and concentrated under vacuum to get rid of methanol. The residue was taken up in EtOAc/H₂O, extracted with ethyl acetate (2×), and the organic layer treated with brine, dried over anhydrous magnesium sulfate, filtered and concentrated. Chromatography ((5:4:1) EtOAc-Hexane-MeOH (1% NH₄OH)) afforded 0.089 g (90%) of 3-[(1-cyclopropylethyl)amino]-8-fluorochromane-5-carboxamide as a white solid: MS (ES) m/z 279 ([M+H]⁺).

To 3-[(1-cyclopropylethyl)amino]-8-fluorochromane-5-carboxamide (0.089 g, 0.32 mmol) in anhydrous methanol (5 ml), under nitrogen at room temperature, was added 3-(5-fluoro-1H-indol-3-yl)propanal (0.064 g, 0.336 mmol), acetic acid (0.04 ml, 0.768 mmol) and sodium cyanoborohydride (0.04 g, 0.64 mmol). The reaction mixture was stirred at room temperature overnight and worked up as described above. Chromatography ((5:4:1) EtOAc-Hexane-MeOH (1% NHiOH)) afforded 0.06 g (42%) of the title compound as a gum, which was then converted to the HCl salt as described above for example 109 generating 3-{(1-cyclopropylethyl)[3-(5-fluoro-1H-indol-3-yl)propyl]amino}-8-fluorochromane-5-carboxamide hydrochloride salt as an off-white solid: mp 124° C./dec; MS (ES) m/z 452.4 ([M−H]⁻); Anal. Calculated for C₂₆H₂₉F₂N₃O₂.HCl: C, 63.73; H, 6.17; N, 8.58. Found: C, 63.49; H, 5.96; N, 8.27.

### Example 115

### 8-chloro-3-{cyclobutyl[3-(5-fluoro-1H-indol-3-yl)propyl]amino}chromane-5-carboxamide (“Compound 7”)

A solution of 3-amino-8-chlorochromane-5-carboxamide HCl (450 mg, 1.71 mmol) in methanol (30 ml) was treated at ambient temperature under dry nitrogen with 3-(5-fluoro-1H-indol-3-yl)-propionaldehyde (345 mg, 1.8 mmol) followed by acetic acid (0.11 ml) and sodium cyanoborohydride (217 mg, 3.46 mmol). The reaction mixture was stirred at ambient temperature overnight, quenched with 1N sodium hydroxide and evaporated in vacuo. The residue was partitioned between ethyl acetate and water. The organic layer was extracted with 1N hydrochloric acid and the separated aqueous phase basified with 2N sodium hydroxide. The product was extracted with ethyl acetate (2×). The combined organic extracts were washed with brine, dried over magnesium sulfate, filtered and the filtrate evaporated to dryness in vacuo. The residue was flash chromatographed on silica gel. Elution with a solvent gradient of 50% ethylacetate/40% hexane/5 to 10% methanol and 1% ammonium hydroxide afforded 370 mg (54%) of 8-chloro-3-{[3-(5-fluoro-1H-indol-3-yl)-propyl]-amino}-chroman-5-carboxamide as a dense white foam. MS (ES) m/z 400 ([M−H]⁻).

To 8-chloro-3-{[3-(5-fluoro-1H-indol-3-yl)propyl]amino}chromane-5-carboxamide (0.13 g, 0.3 mmol) in anhydrous methanol (5 ml), under nitrogen at room temperature, was added cyclobutanone (56 mg, 0.8 mmol), acetic acid (0.05 ml, 0.84 mmol) and sodium cyanoborohydride (41 mg, 0.65 mmol). The reaction mixture was stirred at room temperature overnight. More cyclobutanone (56 mg, 0.8 mmole), acetic acid (0.05 ml, 0.84 mmol) and sodium cyanoborohydride (41 mg, 0.65 mmol) were added after 24 hours and 48 hours at which time the reaction went to completion. The reaction mixture was then quenched with 1N aqueous sodium hydroxide and evaporated in vacuo. The residue was partitioned between water and ethyl acetate, the separated organic layer treated with brine, dried over anhydrous magnesium sulfate, filtered and evaporated in vacuo. The residue was chromatographed on silica gel. Elution with 5% methanol in ethyl acetate afforded 0.13 g (89%) of the title compound as a dense colorless oil. MS (ES) m/z 456 ([M+H]⁺).

### Example 116

### 3-{cyclobutyl[3-(5-fluoro-1H-indol-3-yl)-3-oxopropyl]amino}-8-fluorochromane-5-carboxamide (“Compound 8”)

To 3-amino-8-fluorochromane-5-carboxamide (0.087 g, 0.412 mmol) in anhydrous dimethylformamide (4 ml), under nitrogen at room temperature, was added potassium carbonate (0.044 g, 0.317 mmol) and 3-chloro-1-[5-fluoro-1-(phenylsulfonyl)-1H-indol-3-yl]propan-1-one (0.12 g, 0.317 mmol) dissolved in DMF (2.5 ml). The reaction mixture was stirred at room temperature overnight. The reaction mixture was then diluted with EtOAc/H₂O. The organic layer was separated and the aqueous layer extracted with ethyl acetate (2×). The organic extracts were pooled, dried over anhydrous magnesium sulfate, filtered and concentrated to generate 0.18 g (100%) of 3-[3-(1-benzenesulfonyl-5-fluoro-1H-indol-3-yl)-3-oxo-propylamino]-8-fluoro-chroman-5-carboxylic acid amide as a yellow solid. The product was characterized by ¹HNMR and used without further purification in the next step.

To 3-[3-(1-benzenesulfonyl-5-fluoro-1H-indol-3-yl)-3-oxo-propylamino]-8-fluoro-chroman-5-carboxylic acid amide (0.18 g, 0.342 mmol) in methanol (5.6 ml)-water (1.4 ml) was added potassium carbonate (0.12 g, 0.855 mmol). The reaction mixture was brought to reflux and kept under reflux for one hour. The reaction mixture was then concentrated and the residue taken up in EtOAc/H₂O. The organic layer was separated, dried over anhydrous magnesium sulfate, filtered and concentrated. Chromatography ((5:4:1) EtOAc-Hexane-MeOH (1% NH₄OH)) afforded 0.076 g (56%) of 8-fluoro-3-[3-(5-fluoro-1H-indol-3-yl)-3-oxo-propylamino]-chroman-5-carboxylic acid amide as a white solid. MS (ES) m/z 400 ([M+H]⁺); MS (ES) m/z 398 ([M+H]⁻).

To 8-fluoro-3-[3-(5-fluoro-1H-indol-3-yl)-3-oxo-propylamino]-chroman-5-carboxylic acid amide (0.076 g, 0.19 mmol) in anhydrous methanol (2.3 ml), under nitrogen at room temperature, was added cyclobutanone (0.035 ml, 0.475 mmol), acetic acid (0.022 ml, 0.456 mmol) and sodium cyanoborohydride (0.024 g, 0.38 mmol). The reaction mixture was stirred at room temperature overnight. After 24 hrs, more cyclobutanone (0.035 ml), acetic acid (0.022 ml) and sodium cyanoborohydride (0.024 g) were added, and the reaction mixture stirred at room temperature for another 24 hrs. Same work up as described above for example 112. Chromatography ((5:4:1) EtOAc-Hexane-MeOH (1% NH₄OH)) afforded 0.036 g (42%) of the title compound as a white solid, which was then converted to the HCl salt as described above for example 110 generating 3-{cyclobutyl[3-(5-fluoro-1H-indol-3-yl)-3-oxopropyl]amino}-8-fluorochromane-5-carboxamide hydrochloride salt as a white solid: mp 139° C./dec; MS (ES) m/z 454.3 ([M+H]⁺); MS (ES) m/z 476.3 ([M+NA]⁺); Anal. Calculated for C₂₅H₂₅F₂N₃O₃.1.20 HCl.0.50 H₂O; C, 59.31; H, 5.42; N, 8.30. Found: C, 58.96; H, 5.43; N, 8.20.

### Examples 117a and 117b: (−)-3-{cyclobutyl[3-(5,7-difluoro-1H-indol-3-yl)propyl]amino}-8-fluorochromane-5-carboxamide (“Compound 9a”) and (+)-3-{cyclobutyl[3-(5,7-difluoro-1H-indol-3-yl)propyl]amino}-8-fluorochromane-5-carboxamide (“Compound 9b”)

A solution of 3-amino-8-fluorochromane-5-carboxamide (370 mg, 1.76 mmol) in methanol (30 ml) was treated at ambient temperature under dry nitrogen with 3-(5,7-difluoro-1H-indol-3-yl)-propionaldehyde (387 mg, 1.85 mmol) followed by acetic acid (0.11 ml) and sodium cyanoborohydride (220 mg, 3.5 mmol). The reaction mixture was stirred at ambient temperature overnight. The reaction was quenched with 0.1 N sodium hydroxide, evaporated in vacuo and the residue partitioned between water and ethyl acetate. The separated organic layer was washed with brine, dried over magnesium sulfate, filtered and evaporated to dryness affording 705 mg (99%) of the desired racemic compound as an off-white foam. MS (ES) m/z 404.1 ([M+H]+).

The enantiomers of 3-[3-(5,7-difluoro-1H-indol-3-yl)-propylamino]-8-fluoro-chroman-5-carboxylic acid amide were separated by chiral HPLC and isolated generating the following products:
(+)-3-[3-(5,7-difluoro-1H-indol-3-yl)-propylamino]-8-fluoro-chroman-5-carboxylic acid amide as a white foam; MS (ES) m/z 404.2 ([M+H]⁺);
(−)-3-[3-(5,7-difluoro-1H-indol-3-yl)-propylamino]-8-fluoro-chroman-5-carboxylic acid amide as a white foam; MS (ES) m/z 402.3 ([M−H]⁻).

A solution of the starting chiral 3-[3-(5,7-difluoro-1H-indol-3-yl)-propylamino]-8-fluoro-chroman-5-carboxylic acid amide (0.5 mmol) in methanol (8 ml) was treated 3 times in 10 hour intervals under dry nitrogen at ambient temperature with cyclobutanone (1.25 mmol), acetic acid (0.08 ml) and sodium cyanoborohydride (1.01 mmol). The reaction mixture was basified with 1N sodium hydroxide, the methanol removed in vacuo and the residue partitioned between water and ethyl acetate. The organic layer was separated, washed with brine, dried over magnesium sulfate, filtered and evaporated in vacuo to dryness. The residue was flash chromatographed on silica gel (20 g). Elution with 1% methanol in chloroform afforded the title compounds in over 70% yield. MS for both compounds (ES) m/z 458.2 ([M+H]⁺).

### Example 118

### Methyl 8-fluoro-3-{[3-(5-fluoro-1H-indol-3-yl)propyl]amino}chromane-5-carboxylate (“Compound 10”)

To methyl 3-amino-8-fluorochromane-5-carboxylate (0.28 g, 1.25 mmol) in anhydrous methanol (20 ml), under nitrogen at room temperature, was added 3-(5-fluoro-1H-indol-3-yl)propanal (0.25 g, 1.31 mmol), acetic acid (0.16 ml, 3 mmol) and sodium cyanoborohydride (0.157 g, 2.5 mmol). The reaction mixture was stirred at room temperature overnight. Same work up as described above for example 110. Chromatography ((6:3:1) Hexane-EtOAc-MeOH (1% NH₄OH)) afforded 0.457 g (91%) of the title compound as a white solid, which was then converted to the HCl salt as described above for example 110 generating methyl 8-fluoro-3-{[3-(5-fluoro-1H-indol-3-yl)propyl]amino}chromane-5-carboxylate hydrochloride salt as a white solid: mp 219° C./dec; MS (ES) m/z 401.2 ([M+H]⁺); Anal. Calculated for C₂₂H₂₂F₂N₂O₃.HCl: C, 60.48; H, 5.31; N, 6.41; Found: C, 60.20; H, 4.85; N, 6.16.

### Example 119

### Methyl 3-{cyclobutyl[3-(5-fluoro-1H-indol-3-yl)propyl]amino}-8-fluorochromane-5-carboxylate (“Compound 11”)

This compound was prepared by generally following the procedure as described above for example 112 (compound 4) using methyl 8-fluoro-3-{[3-(5-fluoro-1H-indol-3-yl)propyl]amino}chromane-5-carboxylate (0.36 g, 0.899 mmol), cyclobutanone (0.17 ml, 2.25 mmol), acetic acid (0.11 ml, 2.16 mmol) and sodium cyanoborohydride (0.113 g, 1.798 mmol) in anhydrous methanol (11 ml). After 24 hrs, more cyclobutanone (0.17 ml), acetic acid (0.11 ml) and sodium cyanoborohydride (0.113 g) were added, and the reaction mixture stirred at room temperature for another night. Chromatography ((2:1) Hexane-EtOAc) afforded 0.33 g (81%) of the title compound as a gummy solid, which was then converted to the HCl salt as described above for example 112 generating methyl 3-{cyclobutyl[3-(5-fluoro-1H-indol-3-yl)propyl]amino}-8-fluorochromane-5-carboxylate hydrochloride salt as a white solid: mp 110° C./dec; MS (ES) m/z 455.2 ([M+H]⁺); Anal. Calculated for C₂₆H₂₈F₂N₂O₃.HCl: C, 63.61; H, 5.95; N, 5.71. Found: C, 63.43; H, 5.82; N, 5.69.

### Example 120

### 8-fluoro-3-[[3-(5-fluoro-1H-indol-3-yl)-1-methylpropyl]amino]-chromane-5-carboxamide (“Compound 12”)

To a slurry of 3-amino-8-fluorochromane-5-carboxamide (0.59 g, 2.79 mmol) in anhydrous 1,2-dichloroethane (25 ml), under nitrogen at room temperature, was added 4-(5-fluoro-1H-indol-3-yl)-butan-2-one (0.57 g, 2.79 mmol), acetic acid (0.29 ml, 5.58 mmol) and sodium triacetoxyborohydride (0.83 g, 3.91 mmol). The reaction mixture was stirred at room temperature overnight. The reaction mixture was quenched with 1N NaOH/H₂O and extracted with methylene chloride followed by ethyl acetate. The organic extracts were then treated with brine, dried over magnesium sulfate, filtered and concentrated under vacuum. Chromatography ((14:1) CH₂Cl₂—MeOH (1% NH₄OH)) afforded 0.82 g (74%) of 8-fluoro-3-{[3-(5-fluoro-1H-indol-3-yl)-1-methylpropyl]amino}chromane-5-carboxamide as a white solid: MS (ESI) m/z 400 ([M+H]⁺).

### Examples 120a, 120b, 120c and 120d

### Isomers 1, 2, 3 and 4 of 8-fluoro-3-[[3-(5-fluoro-1H-indol-3-yl)-1-methylpropyl](propyl)amino]-3,4-dihydro-2H-chromene-5-carboxamide (“Compounds 12a, 12b, 12c and 12d”)

The diastereomers and enantiomers of 8-fluoro-3-{[3-(5-fluoro-1H-indol-3-yl)-1-methylpropyl]amino}chromane-5-carboxamide (example 120) were separated by HPLC, and isolated.

Isomer 1 of compound 12 was converted to the title compound as described above for example 110 using 8-fluoro-3-{[3-(5-fluoro-1H-indol-3-yl)-1-methylpropyl]amino}chromane-5-carboxamide (0.09 g, 0.225 mmol), propionaldehyde (0.097 ml, 1.35 mmol), acetic acid (0.031 ml, 0.54 mmol) and sodium cyanoborohydride (0.28 g, 0.45 mmol) in anhydrous methanol (3.7 ml). Chromatography ((6:3:1) Hexane-EtOAc-MeOH (1% NH₄OH)) afforded 0.114 g (100%) of colorless gum which was converted to the HCl salt to generate (−)-8-fluoro-3-[3-(5-fluoro-1H-indol-3-yl)-1-methylpropyl](propyl)amino]-3,4-dihydro-2H-chromene-5-carboxamide hydrochloride salt (compound 12a) as a white solid: mp 133° C./dec; [α]_{D}²⁵=−22.180 (c=1% SOLUTION, DMSO); MS (ESI) m/z 442 ([M+H]⁺); Anal. calculated for C₂₅H₂₉F₂N₃O₂.HCl.0.60 H₂O; C, 61.43; H, 6.43; N, 8.60; Found: C, 61.42; H, 6.52; N, 8.44.

Isomer 2 of compound 12 was converted to the title compound as described above for isomer 1 using 8-fluoro-3-{[3-(5-fluoro-1H-indol-3-yl)-1-methylpropyl]amino}chromane-5-carboxamide (0.10 g, 0.25 mmol), propionaldehyde (0.108 ml, 1.5 mmol), acetic acid (0.035 ml, 0.6 mmol) and sodium cyanoborohydride (0.031 g, 0.5 mmol) in anhydrous methanol (4.1 ml). Chromatography ((6:3:1) Hexane-EtOAc-MeOH (1% NH₄OH)) afforded 0.11 g (96%) of colorless gum which was converted to the HCl salt to generate (+)-8-fluoro-3-[3-(5-fluoro-1H-indol-3-yl)-1-methylpropyl](propyl)amino]-3,4-dihydro-2H-chromene-5-carboxamide hydrochloride salt (compound 12b) as a white solid: mp 133° C./dec; [α]_{D}²⁵=+23.44° (c=1% SOLUTION, DMSO); MS (ESI) m/z 442 ([M+H]⁺); Anal. calculated for C₂₅H₂₉F₂N₃O₂.HCl.0.60 H₂O; C, 61.43; H, 6.43; N, 8.60; Found: C, 61.35; H, 6.48; N, 8.46.

Isomer 3 of compound 12 was converted to the title compound as described above for isomer 1 using 8-fluoro-3-{[3-(5-fluoro-1H-indol-3-yl)-1-methylpropyl]amino}chromane-5-carboxamide (0.09 g, 0.225 mmol), propionaldehyde (0.097 ml, 1.35 mmol), acetic acid (0.031 ml, 0.54 mmol) and sodium cyanoborohydride (0.028 g, 0.45 mmol) in anhydrous methanol (3.7 ml). Chromatography ((6:3:1) Hexane-EtOAc-MeOH (1% NH₄OH)) afforded 0.093 g (94%) of colorless gum which was converted to the HCl salt to generate (−)-8-fluoro-3-[3-(5-fluoro-1H-indol-3-yl)-1-methylpropyl](propyl)amino]-3,4-dihydro-2H-chromene-5-carboxamide hydrochloride salt (compound 12c) as a white solid: mp 133° C./dec; [α]_{D}²⁵=−67.48° (c=1% SOLUTION, DMSO); MS (ESI) m/z 442 ([M+H]⁺); Anal. calculated for C₂₅H₂₉F₂N₃O₂.HCl.0.60 H₂O; C, 61.43; H, 6.43; N, 8.60; Found: C, 61.46; H, 6.44; N, 8.54.

Isomer 4 of compound 12 was converted to the title compound as described above for isomer 1 using 8-fluoro-3-{[3-(5-fluoro-1H-indol-3-yl)-1-methylpropyl]amino}chromane-5-carboxamide (0.09 g, 0.225 mmol), propionaldehyde (0.097 ml, 1.35 mmol), acetic acid (0.031 ml, 0.54 mmol) and sodium cyanoborohydride (0.028 g, 0.45 mmol) in anhydrous methanol (3.7 ml). Chromatography ((6:3:1) Hexane-EtOAc-MeOH (1% NH₄OH)) afforded 0.095 g (95%) of colorless gum which was converted to the HCl salt to generate (+)-8-fluoro-3-[3-(5-fluoro-1H-indol-3-yl)-1-methylpropyl](propyl)amino]-3,4-dihydro-2H-chromene-5-carboxamide hydrochloride salt (compound 12d) as a white solid: mp 133° C./dec; [α]_{D}²⁵=+61.36° (c=1% SOLUTION, DMSO); MS (ESI) m/z 442 ([M+H]⁺); Anal. calculated for C₂₅H₂₉F₂N₃O₂.HCl.0.60 H₂O; C, 61.43; H, 6.43; N, 8.60; Found: C, 61.42; H, 6.31; N, 8.44.

### Example 121

### 3-{[3-(5-cyano-1H-indol-3-yl)-1-methylpropyl]amino}-8-fluoro chroman-5-carboxamide (“Compound 13”)

3-{[3-(5-cyano-1H-indol-3-yl)-1-methylpropyl]amino}-8-fluorochroman-5-carboxamide was prepared as described above for example 120 using 3-amino-8-fluorochromane-5-carboxamide (0.50 g, 2.38 mmol), 3-(3-oxobutyl)-1H-indole-5-carbonitrile (0.51 g, 2.38 mmol), acetic acid (0.25 ml, 4.76 mmol) and sodium triacetoxyborohydride (0.71 g, 3.33 mmol) in anhydrous 1,2-dichloroethane (20 ml). Chromatography ((14:1) CH₂Cl₂—MeOH (1% NH₄OH)) afforded 0.70 g (72%) of 3-{[3-(5-cyano-1H-indol-3-yl)-1-methylpropyl]amino}-8-fluorochroman-5-carboxamide as a white solid: MS (ESI) m/z 407 ([M+H]⁺).

### Examples 121a, 121b, 121c, and 121d

### Isomers 1, 2, 3, and 4 of 3-[[3-(5-cyano-1H-indol-3-yl)-1-methylpropyl](propyl)amino]-8-fluoro-3,4-dihydro-2H-chromene-5-carboxamide—(“Compounds 13a, 13b, 13c and 13d”)

The diastereomers and enantiomers of 3-{[3-(5-cyano-1H-indol-3-yl)-1-methylpropyl]amino}-8-fluorochroman-5-carboxamide (example 121) were separated by HPLC, and isolated.

Isomer 1 of compound 13 was converted to the title compound as described above for example 110 using 3-{[3-(5-cyano-1H-indol-3-yl)-1-methylpropyl]amino}-8-fluorochroman-5-carboxamide (0.125 g, 0.307 mmol), propionaldehyde (0.133 ml, 1.84 mmol), acetic acid (0.042 ml, 0.734 mmol) and sodium cyanoborohydride (0.038 g, 0.614 mmol) in anhydrous methanol (5 ml). Chromatography ((6:3:1) Hexane-EtOAc-MeOH (1% NH₄OH)) afforded 0.107 g (78%) of a white solid which was converted to the HCl salt to generate (−)-3-[[3-(5-cyano-1H-indol-3-yl)-1-methylpropyl](propyl)amino]-8-fluoro-3,4-dihydro-2H-chromene-5-carboxamide hydrochloride salt (compound 13a) as a white solid: mp 140° C./dec; [α]_{D}²⁵=−10.24° (c=1% SOLUTION, DMSO); MS (ES) m/z 447.2 ([M−H]⁻); Anal. calculated for C₂₆H₂₉FN₄O₂.1.10 HCl.0.50 H₂O; C, 62.75; H, 6.30; N, 11.26; Found: C, 62.90; H, 6.43; N, 11.10.

Isomer 2 of compound 13 was converted to the title compound as described above for isomer 1 using 3-{[3-(5-cyano-1H-indol-3-yl)-1-methylpropyl]amino}-8-fluorochroman-5-carboxamide (0.095 g, 0.234 mmol), propionaldehyde (0.101 ml, 1.40 mmol), acetic acid (0.032 ml, 0.562 mmol) and sodium cyanoborohydride (0.029 g, 0.468 mmol) in anhydrous methanol (3.8 ml). Chromatography ((6:3:1) Hexane-EtOAc-MeOH (1% NH₄OH)) afforded 0.087 g (82%) of white solid which was converted to the HCl salt to generate (+)-3-[[3-(5-cyano-1H-indol-3-yl)-1-methylpropyl](propyl)amino]-8-fluoro-3,4-dihydro-2H-chromene-5-carboxamide hydrochloride salt (compound 13b) as a white solid: mp 143° C./dec; [α]_{D}²⁵=−11.62° (c=1% SOLUTION, DMSO); MS (ES) m/z 447.2 ([M−H]⁻); Anal. calculated for C₂₆H₂₉FN₄O₂.1.10 HCl.0.50 H₂O; C, 62.75; H, 6.30; N, 11.26; Found: C, 62.82; H, 6.29; N, 11.13.

Isomer 3 of compound 13 was converted to the title compound as described above for isomer 1 using 3-{[3-(5-cyano-1H-indol-3-yl)-1-methylpropyl]amino}-8-fluorochroman-5-carboxamide (0.115 g, 0.283 mmol), propionaldehyde (0.123 ml, 1.70 mmol), acetic acid (0.039 ml, 0.679 mmol) and sodium cyanoborohydride (0.036 g, 0.566 mmol) in anhydrous methanol (4.6 ml). Chromatography ((6:3:1) Hexane-EtOAc-MeOH (1% NH₄OH)) afforded 0.112 g (88%) of white solid which was converted to the HCl salt to generate (−)-3-[[3-(5-cyano-1H-indol-3-yl)-1-methylpropyl](propyl)amino]-8-fluoro-3,4-dihydro-2H-chromene-5-carboxamide hydrochloride salt (compound 13c) as a white solid: mp 145° C./dec; [α]_{D}²⁵=−60.58° (c=1% SOLUTION, DMSO); MS (ES) m/z 447.2 ([M−H]⁻); Anal. calculated for C₂₆H₂₉FN₄O₂.1.10 HCl.0.30 H₂O; C, 63.21; H, 6.26; N, 11.34; Found: C, 63.15; H, 6.16; N, 11.18.

Isomer 4 of compound 13 was converted to the title compound as described above for isomer 1 using 3-{[3-(5-cyano-1H-indol-3-yl)-1-methylpropyl]amino}-8-fluorochroman-5-carboxamide (0.116 g, 0.285 mmol), propionaldehyde (0.123 ml, 1.70 mmol), acetic acid (0.039 ml, 0.679 mmol) and sodium cyanoborohydride (0.036 g, 0.566 mmol) in anhydrous methanol (4.6 ml). Chromatography ((6:3:1) Hexane-EtOAc-MeOH (1% NH₄OH)) afforded 0.103 g (80%) of white solid which was converted to the HCl salt to generate (+)-3-[[3-(5-cyano-1H-indol-3-yl)-1-methylpropyl](propyl)amino]-8-fluoro-3,4-dihydro-2H-chromene-5-carboxamide hydrochloride salt (compound 13d) as a white solid: mp 145° C./dec; [α]_{D}²⁵=+59.38° (c=1% SOLUTION, DMSO); MS (ES) m/z 447.2 ([M−H]⁻); Anal. calculated for C₂₆H₂₉FN₄O₂.1.20 HCl.0.25 H₂O; C, 62.86; H, 6.23; N, 11.28; Found: C, 62.79; H, 6.10; N, 11.20.

### Example 122

### 8-fluoro-3-{[4-(5-fluoro-1H-indol-3-yl)-1-methylbutyl]amino}chromane-5-carboxamide (“Compound 14”)

8-fluoro-3-{[4-(5-fluoro-1H-indol-3-yl)-1-methylbutyl]amino}chromane-5-carboxamide was prepared as described above for example 120 using 3-amino-8-fluorochromane-5-carboxamide (0.53 g, 2.52 mmol), 5-(5-fluoro-1H-indol-3-yl)pentan-2-one (0.553 g, 2.52 mmol), glacial acetic acid (0.3 ml) and sodium triacetoxyborohydride (0.80 g, 3.78 mmol) in anhydrous 1,2-dichloroethane (25 ml). Chromatography ((19:1) CH₂Cl₂—MeOH (5% NH₄OH) afforded 0.787 g (75%) of 8-fluoro-3-{[4-(5-fluoro-1H-indol-3-yl)-1-methylbutyl]amino}chromane-5-carboxamide as a white solid. This product was characterized by HNMR

### Example 122a and 122b

### (+)-8-fluoro-3-[[4-(5-fluoro-1H-indol-3-yl)-1-methylbutyl](propyl)amino]chromane-5-carboxamide (“Compound 14a”) and (−)-8-fluoro-3-[[4-(5-fluoro-1H-indol-3-yl)-1-methylbutyl](propyl)amino]chromane-5-carboxamide (“Compound 14b”)

The diastereomers and enantiomers of 8-fluoro-3-{[4-(5-fluoro-1H-indol-3-yl)-1-methylbutyl]amino}chromane-5-carboxamide (example 122) were separated by HPLC, and isolated.

Isomer 1 was converted to the title compound as described above for example 110 using 8-fluoro-3-{[4-(5-fluoro-1H-indol-3-yl)-1-methylbutyl]amino}chromane-5-carboxamide (0.09 g, 0.22 mmol), propionaldehyde (0.08 ml, 1.1 mmol), glacial acetic acid (0.05 ml) and sodium cyanoborohydride (0.035 g, 0.56 mmol) in anhydrous methanol (3.5 ml). Chromatography ((1:1) Hexane-EtOAc) afforded 0.067 g (68%) of a clear gum which was converted to the HCl salt to generate (+)-8-fluoro-3-[[4-(5-fluoro-1H-indol-3-yl)-1-methylbutyl](propyl)amino]chromane-5-carboxamide hydrochloride salt as a white solid: mp 148-150° C.; [α]_{D}²⁵=+35.44° (c=1% SOLUTION, DMSO); MS (ES) m/z 456.1 ([M+H]⁺); Anal. calculated for C₂₆H₃₁F₂N₃O₂.HCl.0.30 H₂O; C, 62.78; H, 6.61; N, 8.45; Found: C, 62.73; H, 6.64; N, 8.36.

Isomer 2 was converted to the title compound as described above for example 110 using 8-fluoro-3-{[4-(5-fluoro-1H-indol-3-yl)-1-methylbutyl]amino}chromane-5-carboxamide (0.89 g, 0.22 mmol), propionaldehyde (0.08 ml, 1.1 mmol), glacial acetic acid (0.05 ml) and sodium cyanoborohydride (0.035 g, 0.56 mmol) in anhydrous methanol (3.5 ml). After stirring at room temperature for 16 hrs, additional reagents were added: propionaldehyde (0.08 ml), glacial acetic acid (0.05 ml) and sodium cyanoborohydride (0.035 g). Stirring was continued at room temperature for an additional 66 hrs. Chromatography ((1:1) Hexane-EtOAc) afforded 0.087 g (86%) of a clear gum which was converted to the HCl salt to generate (−)-8-fluoro-3-[[4-(5-fluoro-1H-indol-3-yl)-1-methylbutyl](propyl)amino]chromane-5-carboxamide hydrochloride salt as a white solid: mp 147-150° C.; [α]_{D}²⁵=−40.32° (c=1% SOLUTION, DMSO); MS (ES) m/z 456.1 ([M+H]⁺); Anal. calculated for C₂₆H₃₁F₂N₃O₂.HCl.0.30 H₂O; C, 62.78; H, 6.61; N, 8.45; Found: C, 62.51; H, 6.66; N, 8.37.

### Examples 123a and 123b

### (−)-3-{(cyclopropylmethyl)[3-(5-fluoro-1H-indol-3-yl)-1-methylpropyl]amino}-8-fluorochromane-5-carboxamide (“Compound 15a”) and (+)-3-{(cyclopropylmethyl)[3-(5-fluoro-1H-indol-3-yl)-1-methylpropyl]amino}-8-fluorochromane-5-carboxamide (“Compound 15b”)

Isomer 1 of compound 12 was converted to the title compound as described above for example 113 using 8-fluoro-3-{[3-(5-fluoro-1H-indol-3-yl)-1-methylpropyl]amino}chromane-5-carboxamide (0.078 g, 0.195 mmol), cyclopropanecarboxaldehyde (0.087 ml, 1.17 mmol), acetic acid (0.027 ml, 0.468 mmol) and sodium cyanoborohydride (0.025 g, 0.39 mmol) in anhydrous methanol (3.5 ml). Chromatography ((5:4:1) EtOAc-hexane-MeOH (1% NH₄OH)) afforded 0.08 g (90%) of gum which was converted to the HCl salt to generate (−)-3-{(cyclopropylmethyl)[3-(5-fluoro-1H-indol-3-yl)-1-methylpropyl]amino}-8-fluorochromane-5-carboxamide hydrochloride salt (compound 15a) as a white solid: mp 135° C./dec; [α]_{D}²⁵=−17.00° (c=1% SOLUTION, DMSO); MS (ES) m/z 454.2 ([M+H]⁺); Anal. Calcd for C₂₆H₂₉F₂N₃O₂.HCl: C, 63.73; H, 6.17; N, 8.58. Found: C, 63.39; H, 6.28; N, 8.39.

Isomer 2 of compound 12 was converted to the title compound as described above for example 113 using 8-fluoro-3-{[3-(5-fluoro-1H-indol-3-yl)-1-methylpropyl]amino}chromane-5-carboxamide (0.073 g, 0.183 mmol), cyclopropanecarboxaldehyde (0.082 ml, 1.10 mmol), acetic acid (0.025 ml, 0.439 mmol) and sodium cyanoborohydride (0.023 g, 0.366 mmol) in anhydrous methanol (3.3 ml). Chromatography ((5:4:1) EtOAc-hexane-MeOH (1% NH₄OH)) afforded 0.075 g (90%) of gum which was converted to the HCl salt to generate (+)-3-{(cyclopropylmethyl)[3-(5-fluoro-1H-indol-3-yl)-1-methylpropyl]amino}-8-fluorochromane-5-carboxamide hydrochloride salt (compound 15b) as a white solid: mp 135° C./dec; [α]_{D}²⁵=+15.4° (c=1% SOLUTION, DMSO); MS (ES) m/z 452.2 ([M−H]⁻); Anal. Calculated for C₂₆H₂₉F₂N₃O₂.HCl.0.60 H₂O; C, 62.36; H, 6.28; N, 8.39. Found: C, 62.25; H, 6.37; N, 8.27.

### Examples 124a and 124b

### (−)-3-{ethyl[3-(5-fluoro-1H-indol-3-yl)-1-methylpropyl]amino}-8-fluorochromane-5-carboxamide (“Compound 16a”) and (+)-3-{ethyl[3-(5-fluoro-1H-indol-3-yl)-1-methylpropyl]amino}-8-fluorochromane-5-carboxamide (“Compound 16b”)

Isomer 1 of compound 12 was converted to the title compound as described above for example 111 using 8-fluoro-3-{[3-(5-fluoro-1H-indol-3-yl)-1-methylpropyl]amino}chromane-5-carboxamide (0.078 g, 0.195 mmol), acetaldehyde (0.065 ml, 1.17 mmol), acetic acid (0.027 ml, 0.468 mmol) and sodium cyanoborohydride (0.025 g, 0.39 mmol) in anhydrous methanol (3.5 ml). Chromatography ((5:4:1) EtOAc-hexane-MeOH (1% NH₄OH)) afforded 0.065 g (78%) of gum which was converted to the HCl salt to generate (−)-3-{ethyl[3-(5-fluoro-1H-indol-3-yl)-1-methylpropyl]amino}-8-fluorochromane-5-carboxamide hydrochloride salt (compound 16a) as a white solid: mp 132° C./dec; [O]D25=18.00° (c=1% SOLUTION, DMSO); MS (ES) mn/z 426.1 ([M−H]⁻); MS (ES) m/z 486.2 ([M+CH3COO]⁻); Anal. Calculated for C₂₄H₂₇F₂N₃O₂.HCl.0.50 H₂O; C, 60.95; H, 6.18; N, 8.88. Found: C, 60.82; H, 6.23; N, 8.70.

Isomer 2 of compound 12 was converted to the title compound as described above for example 111 using 8-fluoro-3-{[3-(5-fluoro-1H-indol-3-yl)-1-methylpropyl]amino}chromane-5-carboxamide (0.073 g, 0.183 mmol), acetaldehyde (0.082 ml, 1.46 mmol), acetic acid (0.025 ml, 0.439 mmol) and sodium cyanoborohydride (0.023 g, 0.366 mmol) in anhydrous methanol (3.3 ml). Chromatography ((5:4:1) EtOAc-hexane-MeOH (1% NH₄OH)) afforded 0.068 g (87%) of gum which was converted to the HCl salt to generate (+)-3-{ethyl[3-(5-fluoro-1H-indol-3-yl)-1-methylpropyl]amino}-8-fluorochromane-5-carboxamide hydrochloride salt (compound 16b) as a white solid: mp 135° C./dec; [α]_{D}²⁵=+17.4° (c=1% SOLUTION, DMSO); MS (ES) m/z 426.2 ([M−H]⁻); Anal. Calculated for C₂₄H₂₇F₂N₃O₂.HCl.0.25 H₂O; C, 61.53; H, 6.13; N, 8.97. Found: C, 61.53; H, 6.05; N, 8.85.

### Example 125

### 8-fluoro-3-[[3-(5-fluoro-1-benzothien-3-yl)-3-hydroxypropyl](propyl)amino] chromane-5-carboxamide (“Compound 17”)

To a slurry of 3-amino-8-fluorochromane-5-carboxamide (0.20 g, 0.951 mmol) in anhydrous 1,2-dichloroethane (15 ml), under nitrogen at room temperature, was added propionaldehyde (0.076 ml, 1.05 mmol), acetic acid (0.1 ml, 1.90 mmol) and sodium triacetoxyborohydride (0.28 g, 1.33 mmol). The reaction mixture was stirred at room temperature for 2.5 hrs. The reaction mixture was quenched with 1N NaOH/H₂O and extracted with methylene chloride. The organic extracts were then treated with brine, dried over anhydrous magnesium sulfate, filtered and concentrated. Chromatography ((6:3:1) Hexane-EtOAc-MeOH (1% NH₄OH)) afforded 0.11 g (46%) of 8-fluoro-3-(propylamino)chromane-5-carboxamide as a white solid: MS (ESI) m/z 253 ([M+H]⁺).

To 8-fluoro-3-(propylamino)chromane-5-carboxamide (0.11 g, 0.436 mmol) in anhydrous THF (2 ml), under nitrogen at room temperature, was added potassium carbonate (0.06 g, 0.436 mmol) and 3-chloro-1-(5-fluoro-1-benzothien-3-yl)propan-1-one (0.11 g, 0.452 mmol) dissolved in 1.5 ml of anhydrous THF. The reaction mixture was first stirred at room temperature for 24 hrs followed by 50 C for another 24 hrs. The reaction mixture was then cooled down to room temperature, concentrated, and the residue taken up in EtOAc/H₂O. The aqueous layer was extracted with EtOAc (2×). The organic extracts were treated with brine, dried over anhydrous magnesium sulfate, filtered and concentrated. Chromatography ((6:3:1) Hexane-EtOAc-MeOH (1% NH₄OH)) afforded 0.12 g (60%) of 8-fluoro-3-{[3-(5-fluoro-benzothien-3-yl)-3-oxo-propyl](propyl)amino}-chromane-5-carboxamide as a gummy solid. The product was characterized by ¹HNMR.

To 8-fluoro-3-{[3-(5-fluoro-benzothien-3-yl)-3-oxo-propyl](propyl)amino}-chromane-5-carboxamide (0.11 g, 0.24 mmol) in anhydrous methanol (3 ml), was added sodium borohydride (0.046 g, 1.2 mmol) at 0 C. After 45 min, the reaction mixture was poured over water and stirred overnight. The slurry was then extracted with EtOAc (2×). The organic extracts were dried over anhydrous magnesium sulfate, filtered and concentrated. Chromatography ((6:3:1) Hexane-EtOAc-MeOH (1% NH₄OH)) afforded 0.08 g (72%) of the title compound as a white solid which was converted to the HCl salt to generate 8-fluoro-3-[[3-(5-fluoro-1-benzothien-3-yl)-3-hydroxypropyl](propyl)amino]chromane-5-carboxamide hydrochloride salt as a white solid: mp 117° C./dec; MS (ESI) m/z 461 ([M+H]⁺); Anal. calculated for C₂₄H₂₆F₂N₂O₃S.HCl: C, 58.00; H, 5.48; N, 5.64; Found: C, 57.79; H, 5.49; N, 5.29.

### Example 126

### N-[3-(1-benzothien-3-yl)propyl]-N-ethyl-5-methoxychroman-3-amine (“Compound 18”)

To [3-(1-benzothien-3-yl)propyl]amine (0.19 g, 1.0 mmol) in anhydrous 1,2-dichloroethane (6 ml), under nitrogen at room temperature, was added 5-methoxy-2H-chromen-3(4H)-one (0.2 g, 1.1 mmol), acetic acid (0.14 ml, 2.3 mmol) and sodium triacetoxyborohydride (0.3 g, 1.4 mmol). The reaction mixture was stirred at room temperature overnight. The reaction was quenched with 1N NaOH/H₂O and extracted with methylene chloride (3×). The organic layer was treated with brine, dried over anhydrous sodium sulfate, filtered and concentrated. Chromatography ((2:1) Hexane-EtOAc (extracted with 1% NH₄OH)) afforded 0.16 g (45%) of N-[3-(1-benzothien-3-yl)propyl]-5-methoxychroman-3-amine as a gum: MS (ESI) m/z 354 ([M+H⁺).

The title compound was prepared by generally following the procedure as described above for example 110 using N-[3-(1-benzothien-3-yl)propyl]-5-methoxychroman-3-amine (0.11 g, 0.3 mmol), acetaldehyde (0.018 ml, 0.33 mmol), acetic acid (0.042 ml, 0.72 mmol) and sodium cyanoborohydride (0.037 g, 0.6 mmol) in anhydrous methanol (7 ml). Chromatography ((6:1) Hexane-EtOAc (extracted with 1% NH₄OH)) afforded 0.077 g (68%) of the title compound as a gum which was converted to the HCl salt to generate N-[3-(1-benzothien-3-yl)propyl]-N-ethyl-5-methoxychroman-3-amine hydrochloride salt as a off-white solid: mp dec/59.0° C.; MS (ESI) m/z 382 ([M+H]⁺); Anal. calculated for C₂₃H₂₇NO₂S.1.10 HCl. 0.30 H₂.0.25 C₄H₈O₂; C, 64.19; H, 6.89; N, 3.12; Found: C, 64.18; H, 6.96; N, 3.11.

### Example 127

### N-[3-(5-fluoro-1-benzothien-3-yl)propyl]-5-methoxy-N-propylchroman-3-amine (“Compound 19”)

N-(5-methoxy-3,4-dihydro-2H-chromen-3-yl)-N-propylamine was prepared by generally following the procedure as described above for example 125 using (5-methoxy-3,4-dihydro-2H-chromen-3-yl)amine (0.2 g, 1.12 mmol), propionaldehyde (0.088 ml, 1.23 mmol), acetic acid (0.12 ml, 2.23 mmol) and sodium triacetoxyborohydride (0.33 g, 1.56 mmol) in anhydrous 1,2-dichloroethane (10 ml). Chromatography ((7:3) EtOAc-Hexane) afforded 0.16 g (65%) of N-(5-methoxy-3,4-dihydro-2H-chromen-3-yl)-N-propylamine as a gum: MS (ESI) m/z 222 ([M+H]⁺).

1-(5-fluoro-1-benzothien-3-yl)-3-[(5-methoxy-3,4-dihydro-2H-chromen-3-yl) (propyl)amino]propan-1-one was prepared by generally following the procedure as described above for example 125 using N-(5-methoxy-3,4-dihydro-2H-chromen-3-yl)-N-propylamine (0.20 g, 0.904 mmol), 3-chloro-1-(5-fluoro-1-benzothien-3-yl)propan-1-one (0.22 g, 0.904 mmol) dissolved in 2 ml of anhydrous THF and potassium carbonate (0.125 g, 0.904 mmol) in anhydrous THF (4 ml). Chromatography ((6:1) Hexane-EtOAc) afforded 0.19 g (49%) of 1-(5-fluoro-1-benzothien-3-yl)-3-[(5-methoxy-3,4-dihydro-2H-chromen-3-yl) (propyl)amino]propan-1-one as a gummy solid: MS (ESI) m/z 428 ([M+H]⁺).

1-(5-fluoro-1-benzothien-3-yl)-3-[(5-methoxy-3,4-dihydro-2H-chromen-3-yl) (propyl)amino]propan-1-ol was prepared by generally following the procedure as described above for example 125 using 1-(5-fluoro-1-benzothien-3-yl)-3-[(5-methoxy-3,4-dihydro-2H-chromen-3-yl)(propyl)amino]propan-1-one (0.19 g, 0.444 mmol), sodium borohydride (0.25 g, 6.66 mmol) in anhydrous methanol (5 ml) at 0° C. Chromatography ((3:1) Hexane-EtOAc) afforded 0.15 g (79%) of 1-(5-fluoro-1-benzothien-3-yl)-3-[(5-methoxy-3,4-dihydro-2H-chromen-3-yl)(propyl)amino]propan-1-ol as a gum: MS (ESI) m/z 430 ([M+H]⁺).

To 1-(5-fluoro-1-benzothien-3-yl)-3-[(5-methoxy-3,4-dihydro-2H-chromen-3-yl)(propyl)amino]propan-1-ol (0.31 g, 0.722 mmol) in anhydrous methylene chloride (2.8 ml), under nitrogen at room temperature, was added triethylsilane (0.13 ml, 0.80 mmol) and trifluoroacetic acid (0.56 ml, 7.22 mmol). The reaction mixture was stirred at room temperature overnight. The reaction mixture was concentrated, the residue taken up in CH₂Cl₂ and saturated sodium bicarbonate. The organic layer was separated, treated with brine, dried over anhydrous magnesium sulfate, filtered and concentrated. Chromatography ((9:1) Hexane-EtOAc) afforded 0.068 g (23%) of the title compound as a gum which was converted to the HCl salt to generate N-[3-(5-fluoro-1-benzothien-3-yl)propyl]-5-methoxy-N-propylchroman-3-amine hydrochloride salt as a white solid: mp 78° C./dec; MS (ESI) m/z 414 ([M+H]⁺); Anal. calculated for C₂₄H₂₈FNO₂S 1.30 HCl; C, 62.54; H, 6.41; N, 3.04; Found: C, 62.68; H, 6.44; N, 2.91.

### Example 128

### 3-[[3-(1-benzofuran-3-yl)propyl](propyl)amino]-8-fluorochromane-5-carboxamide (“Compound 20”)

3-{[3-(1-benzofuran-3-yl)propyl]amino}-8-fluorochromane-5-carboxamide was prepared by generally following the procedure as described above for example 109 using 3-(2-bromopropyl)-1-benzofuran (0.165 g, 0.785 mmol), 3-amino-8-fluorochromane-5-carboxamide (0.165 g, 0.785 mmol) and triethylamine (0.22 ml, 1.54 mmol) in anhydrous DMSO (5 ml) at 95° C. for 16 hrs. Chromatography ((49:1) CH₂Cl₂—MeOH (5% NH₄OH)) afforded 0.121 g (42%) of 3-{[3-(1-benzofuran-3-yl)propyl]amino}-8-fluorochromane-5-carboxamide as a light brown gum. The product was characterized by ¹HNMR.

The title compound was prepared by generally following the procedure as described above for example 110 using 3-{[3-(1-benzofuran-3-yl)propyl]amino}-8-fluorochromane-5-carboxamide (0.12 g, 0.33 mmol), propionaldehyde (0.12 ml, 1.65 mmol), acetic acid (0.06 ml, 1.05 mmol) and sodium cyanoborohydride (52 mg, 0.83 mmol) in anhydrous methanol (4 ml). Chromatography ((99:1) CH₂Cl₂—MeOH (5% NH₄OH)) afforded 0.116 g (86%) of the title compound as a tan gum which was converted to the HCl salt to generate 3-[[3-(1-benzofuran-3-yl)propyl](propyl)amino]-8-fluorochromane-5-carboxamide hydrochloride salt as a light-amber solid: mp 114-118° C. (melts with decomposition); MS (ES) m/z 411.04 ([M+H]⁺); Anal. calculated for C₂₄H₂₇FN₂O₃.HCl.0.60 H₂O; C, 62.97; H, 6.43; N, 6.12; Found: C, 62.61; H, 6.40; N, 5.88.

### Example 129

### N-[3-(1-benzofuran-3-yl)propyl]-N-ethyl-5-methoxychroman-3-amine (“Compound 21”)

N-[3-(1-benzofuran-3-yl)propyl]-5-methoxychroman-3-amine was prepared by generally following the procedure as described above for example 126 using [3-(1-benzofuran-3-yl)propyl]amine (0.26 g, 1.47 mmol), 5-methoxy-2H-chromen-3(4H)-one (0.29 g, 1.61 mmol), acetic acid (0.20 ml, 3.37 mmol) and sodium triacetoxyborohydride (0.435 g, 2.05 mmol) in anhydrous 1,2-dichloroethane (9 ml). Chromatography ((2:1) Hexane-EtOAc (extracted with 1% NH₄OH)) afforded 0.287 g (58%) of N-[3-(1-benzofuran-3-yl)propyl]-5-methoxychroman-3-amine as a gum: MS (ESI) m/z 338 ([M+H⁺).

The title compound was prepared by generally following the procedure as described above for example 110 using N-[3-(1-benzofuran-3-yl)propyl]-5-methoxychroman-3-amine (0.166 g, 0.49 mmol), acetaldehyde (0.030 ml, 0.54 mmol), acetic acid (0.069 ml, 1.18 mmol) and sodium cyanoborohydride (0.062 g, 0.984 mmol) in anhydrous methanol (8 ml). Chromatography ((3:1) Hexane-EtOAc (extracted with 1% NH₄OH)) afforded 0.15 g (83%) of the title compound as an oil which was converted to the HCl salt to generate N-[3-(1-benzofuran-3-yl)propyl]-N-ethyl-5-methoxychroman-3-amine hydrochloride salt as a white solid: mp dec/77.0° C.; MS (ES) m/z 366.1 ([M+H]⁺); Anal. calculated for C₂₃H₂₇NO₃.HCl.0.50 H₂O.0.20 C₄H₈O₂: C, 66.70; H, 7.20; N, 3.27; Found: C, 66.81; H, 7.22; N, 3.25.

### Example 130

### N-[4-(1-benzofuran-3-yl)butyl]-N-ethyl-N-(5-methoxy-3,4-dihydro-2H-chromen-3-yl)amine (“Compound 22”)

N-[4-(1-benzofuran-3-yl)butyl]-5-methoxychroman-3-amine was prepared by generally following the procedure as described above for example 109 using 3-(4-bromobutyl)-1-benzofuran (0.648 g, 2.56 mmol), 8-fluoro-5-methoxy-3,4-dihydro-2H-chroman-3-yl)amine (0.62 g, 3.46 mmol) and triethylamine (0.71 ml, 5.1 mmol) in anhydrous DMSO (24 ml). Chromatography ((99:1) CH₂Cl₂—MeOH (5% NH₄OH)) afforded 0.625 g (51%) of N-[4-(1-benzofuran-3-yl)butyl]-5-methoxychroman-3-amine as brown gum. The product was characterized by ¹HNMR.

The title compound was prepared by generally following the procedure as described above for example 110 using N-[4-(1-benzofuran-3-yl)butyl]-5-methoxychroman-3-amine (0.125 g, 0.36 mmol), acetaldehyde (0.05 ml, 0.89 mmol), acetic acid (0.07 ml, 1.2 mmol) and sodium cyanoborohydride (0.057 g, 0.90 mmol) in anhydrous methanol (4 ml). Chromatography ((4:1) Hexane-EtOAc)) afforded 0.094 g (%) of the title compound as a clear gum which was converted to the HCl salt to generate N-[4-(1-benzofuran-3-yl)butyl]-N-ethyl-N-(5-methoxy-3,4-dihydro-2H-chromen-3-yl)amine hdyrochloride salt as a off-white solid: mp 152-154° C.; MS (ES) m/z 380.08 ([M+H]⁺); Anal. calculated for C₂₄H₂₉NO₃.HCl.0.40 H₂O: C, 68.12; H, 7.34; N, 3.31; Found: C, 59.97; H, 5.96; N, 2.81.

### Example 131

### [3-(5-fluoro-1H-indol-3-yl)propyl]-N-(5-methoxy-chroman-3-yl)propylamine (“Compound 23”)

[3-(5-fluoro-1H-indol-3-yl)-propyl]-(5-methoxy-chroman-3-yl)-amine was prepared by generally following the procedure as described above for example 109 using (5-methoxy-3,4-dihydro-2H-chromen-3-yl)amine (0.6 g, 2.8 mmol), 3-(3-bromopropyl)-5-fluoro-1H-indole (0.72 g, 1.8 mmol) and triethylamine (0.53 ml, 3.6 mmol) in anhydrous dimethylsulfoxide (20 ml) at 80° C. for 10 hrs. Chromatography ((19.1) CH₂Cl₂—MeOH) afforded 0.64 g (100%) of [3-(5-Fluoro-1H-indol-3-yl)-propyl]-(5-methoxy-chroman-3-yl)-amine as a yellow oil. The oxalate salt was prepared in tetrahydrofuran to generate [3-(5-fluoro-1H-indol-3-yl)-propyl]-(5-methoxy-chroman-3-yl)-amine oxalate salt as a white solid: mp 133-136° C.; MS (ESI) m/z 355 ([M+H]⁺); Anal. calculated for 1.00 C₂₁H₂₃FN₂O₂+1.00 C₂H₂O₄; C, 62.11; H, 5.67; N, 6.30; Found: C, 62.03; H, 5.79; N, 6.12.

The title compound was prepared by generally following the procedure as described above for example 110 using [3-(5-fluoro-1H-indol-3-yl)-propyl]-(5-methoxy-chroman-3-yl)-amine (0.4 g, 1.13 mmol), propionaldehyde (0.84 ml, 11.3 mmol), acetic acid (0.065 ml, 1.13 mmol) and sodium cyanoborohydride (0.134 g, 2.0 mmol) in anhydrous methanol (20 ml). Chromatography ((49:1) CH₂Cl₂—MeOH) afforded 0.44 g (98%) of the title compound as a yellow oil which was converted to the HCl salt to generate [3-(5-fluoro-1H-indol-3-yl)propyl]-N-(5-methoxy-chroman-3-yl)propylamine hydrochloride salt as a off-white solid: mp 117-120° C.; MS (ESI) m/z 397 ([M+H]⁺); Anal. calculated for C₂₄H₂₉FN₂O₂.HCl.0.50H₂O; C, 65.22; H, 7.07; N, 6.34; Found: C, 65.57; H, 7.12; N, 5.89.

### Example 132

### [3-(5-fluoro-1H-indol-3-yl)-propyl]-((3R)-5-methoxychroman-3-yl)propylamine (“Compound 24”)

The title compound was prepared by generally following the procedure as described above for example 110 using (3R)-N-[3-(5-fluoro-1H-indol-3-yl)-propyl]-5-methoxy-chroman-3-amine (0.1 g, 0.28 mmol), propionaldehyde (0.2 ml, 2.8 mmol), acetic acid (0.016 ml, 0.28 mmol) and sodium cyanoborohydride (0.032 g, 0.50 mmol) in anhydrous methanol (20 ml). Chromatography ((49:1) CH₂Cl₂—MeOH afforded 0.1 g (81%) of the title compound as a yellow oil which was converted to the HCl salt to generate [3-(5-fluoro-1H-indol-3-yl)-propyl]-((3R)-5-methoxychroman-3-yl)propylamine hydrochloride salt as a white solid: mp>90° C.; [α]_{D}²⁵=−8.06° (c=8.808, EtOH); MS (APCI) m/z 397 ([M+H]⁺); Anal. calculated for C₂₄H₂₉FN₂O₂.HCl.0.50 H₂O: C, 65.22; H, 7.07; N, 6.34; Found: C, 64.94; H, 7.15; N, 6.20.

### Example 133

### [3-(5-fluoro-1H-indol-3-yl)-propyl]-((3S)-5-methoxychroman-3-yl)propylamine (“Compound 25”)

The title compound was prepared by generally following the procedure as described for example 132 using (3S)-N-[3-(5-fluoro-1H-indol-3-yl)-propyl]-5-methoxy-chroman-3-amine (0.15 g, 0.42 mmol), propionaldehyde (0.0.31 ml, 4.2 mmol), acetic acid (0.02 ml, 0.42 mmol) and sodium cyanoborohydride (0.05 g, 0.76 mmol) in anhydrous methanol (20 ml). Chromatography ((49:1) CH₂Cl₂—MeOH afforded 0.12 g (72%) of the title compound as a light brown foam which was converted to the HCl salt to generate [3-(5-fluoro-1H-indol-3-yl)-propyl]-((3S)-5-methoxychroman-3-yl)propylamine hydrochloride salt as a white solid: mp>100° C.; [α]_{D}²⁵=+8.47° (c=8.032, EtOH); Anal. calculated for C₂₄H₂₉FN₂O₂+1.00 HCl+0.50 H₂O: C, 65.22; H, 7.07; N, 6.34; Found: C, 64.99; H, 7.23; N, 6.21.

### Example 134

### [3-(5-fluoro-1H-indol-3-yl)-propyl]-(8-fluoro-5-methoxychroman-3-yl)propylamine (“Compound 26”)

N-[3-(5-fluoro-1H-indol-3-yl)propyl]-N-(8-fluoro-5-methoxy-3,4-dihydro-2H-chromen-3-yl)amine was prepared by generally following the procedure as described above for example 109 using (8-fluoro-5-methoxy-3,4-dihydro-2H-chromen-3-yl)amine (0.2 g, 1.02 mmol), 3-(3-bromopropyl)-5-fluoro-1H-indole (0.25 g, 0.97 mmol), and triethylamine (0.3 ml, 2.1 mmol) in anhydrous DMSO (10 ml) at 90° C. for 3 hrs. Chromatography ((3:1) MeOH-EtOAc) afforded 0.184 g (40%) of N-[3-(5-fluoro-1H-indol-3-yl)propyl]-N-(8-fluoro-5-methoxy-3,4-dihydro-2H-chromen-3-yl)amine: MS (ESI) m/z 373 ([M+H]⁺).

The title compound was prepared by generally following the procedure as described above for example 110 using N-[3-(5-fluoro-1H-indol-3-yl)propyl]-N-(8-fluoro-5-methoxy-3,4-dihydro-2H-chromen-3-yl)amine (0.155 g, 0.42 mmol), propionaldehyde (0.32 ml, 4.2 mmol), acetic acid (2-3 drops) and sodium cyanoborohydride (0.06 g, 0.954 mmol) in anhydrous methanol (10 ml). Chromatography ((4:1) EtOAc-Hexane) afforded 0.168 g (96%) of the title compound which was converted to the HCl salt to generate [3-(5-fluoro-1H-indol-3-yl)-propyl]-(8-fluoro-5-methoxychroman-3-yl)propylamine hydrochloride salt as a white solid: mp BROAD; MS (ESI) m/z [M+H]+415; Anal. calculated for C₂₄H₂₈F₂N₂O₂.HCl.0.75 H₂O: C, 62.06; H, 6.62; N, 6.03; Found: C, 62.22; H, 6.30; N, 5.92.

### Example 135

### (3S)-8-fluoro-N-[3-(5-fluoro-1H-indol-3-yl)propyl]-5-methoxy-N-propylchroman-3-amine (“Compound 27”)

The enantiomers of (8-fluoro-5-methoxy-3,4-dihydro-2H-chromen-3-yl)amine were separated by chiral resolution as described extensively in the literature.

The title compound was prepared by generally following the procedure as described above for example 110 using (3S)-8-fluoro-N-[3-(5-fluoro-1H-indol-3-yl)propyl]-5-methoxychroman-3-amine (0.115 g, 0.308 mmol), propionaldehyde (0.2 ml, 2.62 mmol), acetic acid (0.1 ml, 1.77 mmol) and sodium cyanoborohydride (0.07 g, 1.11 mmol) in anhydrous methanol (10 ml). Chromatography ((4:1) EtOAc-Hexane) afforded 0.12 g (93%) of the title compound which was converted to the HCl to generate (3S)-8-fluoro-N-[3-(5-fluoro-1H-indol-3-yl)propyl]-5-methoxy-N-propylchroman-3-amine hydrochloride salt as a white solid: mp Broad; [α]_{D}²⁵=−20.48° (c=8.300, MeOH); Anal. calculated for C₂₄H₂₈F₂N₂O₂.HCl: C, 63.92; H, 6.48; N, 6.21; Found: C, 63.57; H, 6.41; N, 6.11.

### Example 136

### (3R)-8-fluoro-N-[3-(5-fluoro-1H-indol-3-yl)propyl]-5-methoxy-N-propylchroman-3-amine (“Compound 28”)

The title compound was prepared by generally following the procedure as described above for example 110 using (3R)-8-fluoro-N-[3-(5-fluoro-1H-indol-3-yl)propyl]-5-methoxychroman-3-amine (0.13 g, 0.35 mmol), propionaldehyde (0.35 ml, 4.85 mmol), acetic acid (0.1 ml, 1.77 mmol) and sodium cyanoborohydride (0.06 g, 0.95 mmol) in anhydrous methanol (10 ml). Chromatography ((4:1) EtOAc-Hexane) afforded 0.11 g (69%) of the title compound which was converted to the HCl salt to generate (3R)-8-fluoro-N-[3-(5-fluoro-1H-indol-3-yl)propyl]-5-methoxy-N-propylchroman-3-amine hydrochloride salt as a white solid: mp Broad; [α]_{D}²⁵=+18.8° (c=5.13 MG/0.513 ML, MeOH); MS (APCI) m/z 415 ([M+H]⁺); Anal. calculated for C₂₄H₂₈F₂N₂O₂.HCl: C, 63.92; H, 6.48; N, 6.21; Found: C, 63.63; H, 6.44; N, 5.91.

### Example 137

### N-[2-(5-fluoro-1H-indol-3-yl)ethyl]-5-methoxy-N-propylchroman-3-amine (“Compound 29”)

N-[2-(5-fluoro-1H-indol-3-yl)ethyl]-(5-methoxychroman-3-yl)amine was prepared by generally following the procedure as described above for example 109 using (5-methoxy-3,4-dihydro-2H-chromen-3-yl)amine (0.466 g, 2.6 mmol), 3-(2-bromoethyl)-5-fluoro-1H-indole (0.35 g, 1.45 mmol) and triethylamine (0.4 ml, 2.89 mmol) in anhydrous DMSO (20 ml) at 90 C for 9 hrs. Chromatography ((3:1) EtOAc-Hexane (extracted with 1% NH₄OH)) afforded 0.11 g (23%) of N-[2-(5-fluoro-1H-indol-3-yl)ethyl]-(5-methoxychroman-3-yl)amine as a gum: MS (ESI) m/z 341 ([M+H]⁺).

The title compound was prepared by generally following the procedure as described above for example 110 using N-[2-(5-fluoro-1H-indol-3-yl)ethyl]-(5-methoxychroman-3-yl)amine (0.158 g, 0.464 mmol), propionaldehyde (0.03 ml, 0.464 mmol), acetic acid (0.07 ml) and sodium cyanoborohydride (0.58 g, 0.928 mmol) in anhydrous methanol (10 ml). Chromatography ((3:1) Hexane-EtOAc (extracted with 1% NH₄OH)) afforded 0.128 g (72%) of the title compound as a gum which was converted to the HCl salt to generate N-[2-(5-fluoro-1H-indol-3-yl)ethyl]-5-methoxy-N-propylchroman-3-amine hydrochloride salt as a white solid: mp dec/74.2° C.; MS (ESI) m/z 383 ([M+H]⁺); Anal. calculated for C₂₃H₂₇FN₂O₂.1.10 HCl.0.20 C₄H₈O₂.0.15 H₂O: C, 64.54; H, 6.83; N, 6.32; Found: C, 64.56; H, 7.02; N, 6.30.

### Example 138

### N-[4-(5-fluoro-1H-indol-3-yl)butyl]-5-methoxy-N-propylchroman-3-amine (“Compound 30”)

N-[4-(5-fluoro-1H-indol-3-yl)butyl]-5-methoxychroman-3-amine was prepared by generally following the procedure as described above for example 126 using [4-(5 fluoro-1H-indol-3-yl)butyl]amine (0.556 g, 2.70 mmol), 5-methoxy-2H-chromen-3(4H)-one (0.48 g, 2.70 mmol), acetic acid (0.36 ml, 6.21 mmol) and sodium triacetoxyborohydride (0.901 g, 3.78 mmol) in anhydrous 1,2,-dichloroethane (14 ml). Chromatography ((1:1) Hexane-EtOAc (extracted with 2% NH₄OH)) afforded 0.826 g (83%) of N-[4-(5-fluoro-1H-indol-3-yl)butyl]-5-methoxychroman-3-amine as a gum: MS (ESI) m/z 369 ([M+H]⁺).

The title compound was prepared by generally following the procedure as described above for example 110 using N-[4-(5-fluoro-1H-indol-3-yl)butyl]-5-methoxychroman-3-amine (0.2 g, 0.543 mmol), propionaldehyde (0.04 ml, 0.543 mmol), acetic acid (0.08 ml, 1.3 mmol) and sodium cyanoborohydride (0.068 g, 1.086 mmol) in anhydrous methanol (7 ml). Chromatography ((3:1) Hexane-EtOAc (extracted with 1% NH₄OH)) afforded 0.157 g (70%) of the title compound as a gum which was converted to the HCl salt to generate N-[4-(5-fluoro-1H-indol-3-yl)butyl]-5-methoxy-N-propylchroman-3-amine hydrochloride salt as a off-white solid: mp Dec/68.5° C.; MS (ESI) m/z 411 ([M+H]⁺); Anal. calculated for C₂₅H₃₁FN₂O₂.1.10 HCl.0.25 H₂O: C, 65.97; H, 7.22; N, 6.15; Found: C, 65.89; H, 7.09; N, 6.10.

### Example 139

### N-ethyl-N-[3-(5-fluoro-1H-indol-3-yl)propyl]-5-methoxychroman-3-amine (“Compound 31”)

[3-(5-fluoro-1H-indol-3-yl)-propyl]-(5-methoxy-chroman-3-yl)-amine was prepared by generally following the procedure as described above for example 109 using (5-methoxy-3,4-dihydro-2H-chromen-3-yl)amine (0.764 g, 4.27 mmol), 3-(3-bromopropyl)-5-fluoro-1H-indole (0.52 g, 2.03 mmol) and triethylamine (0.57 ml, 4.06 mmol) in anhydrous DMSO (19 ml) at 90° C. for 9 hrs. Chromatography ((7:2:1) Hexane-EtOAc-MeOH (1% NH₄OH)) afforded 0.476 g (66%) of [3-(5-fluoro-1H-indol-3-yl)-propyl]-(5-methoxy-chroman-3-yl)-amine as a brown solid: MS (ESI) m/z 355 ([M+H]⁺).

The title compound was prepared by generally following the procedure as described for example 110 using [3-(5-fluoro-1H-indol-3-yl)-propyl]-(5-methoxy-chroman-3-yl)-amine (0.238 g, 0.672 mmol), acetaldehyde (0.024 ml, 0.681 mmol), acetic acid (0.084 ml, 1.49 mmol) and sodium cyanoborohydride (0.078 g, 1.24 mmol) in anhydrous methanol (10 ml). Chromatography ((8:1:1) Hexane-EtOAc-MeOH)) afforded 0.18 g (70%) of the title compound as a gum which was converted to the HCl salt to generate N-ethyl-N-[3-(5-fluoro-1H-indol-3-yl)propyl]-5-methoxychroman-3-amine hydrochloride salt as a off-white solid: mp 91.2° C./dec; MS (ESI) m/z 381 ([M−H]⁻); Anal. calculated for C₂₃H₂₇FN₂O₂.1.30.HCl 0.30 C₄H₈O₂: C, 63.70; H, 6.78; N, 6.14; Found: C, 63.55; H, 6.95; N, 6.03.

### Example 140

### N-ethyl-N-[4-(5-fluoro-1H-indol-3-yl)butyl]-5-methoxychroman-3-amine (“Compound 32”)

The title compound was prepared as described above for example 110 using N-[4-(5-fluoro-1H-indol-3-yl)butyl]-5-methoxychroman-3-amine (0.16 g, 0.434 mmol), acetaldehyde (0.027 ml, 0.477 mmol), acetic acid (0.061 ml, 1.04 mmol) and sodium cyanoborohydride (0.055 g, 0.868 mmol) in anhydrous methanol (7 ml). Chromatography ((2:1) Hexane-EtOAc (extracted with 1% NH₄OH)) afforded 0.127 g (74%) of the title compound as a gum which was converted to the HCl salt to generate N-ethyl-N-[4-(5-fluoro-1H-indol-3-yl)butyl]-5-methoxychroman-3-amine hydrochloride salt as off-white solid: mp dec/59.4° C.; MS (ESI) m/z 397 ([M+H]⁺); Anal. calculated for C₂₄H₂₉FN₂O₂.1.10 HCl.0.40 H₂O.0.30 C₄H₈O₂: C, 64.37; H, 7.14; N, 5.96; Found: C, 64.30; H, 7.35; N, 5.98.

### Example 141

### N-[3-(5-fluoro-1H-indol-3-yl)propyl]-5-methoxy-N-methylchroman-3-amine (“Compound 33”)

The title compound was prepared by generally following the procedure as described above for example 110 using [3-(5-fluoro-1H-indol-3-yl)-propyl]-(5-methoxy-chroman-3-yl)-amine (0.311 g, 0.877 mmol), 37% formaldehyde in water (0.7 ml, 8.77 mmol), acetic acid (0.12 ml, 2.10 mmol) and sodium cyanoborohydride (0.11 g, 1.75 mmol) in anhydrous methanol (10 ml). Chromatography ((6.5:2.5:1) Hexane-EtOAc-MeOH (1% NH₄OH)) afforded 0.05 g (16%) of the title compound as a gum which was converted to the HCl salt to generate N-[3-(5-fluoro-1H-indol-3-yl)propyl]-5-methoxy-N-methylchroman-3-amine hydrochloride salt as a off-white solid: mp dec/69.6° C.; MS (ESI) m/z 369 ([M+H]⁺); Anal. -calculated for C₂₂H₂₅FN₂O₂.1.10 HCl.0.30 C₄H₈O₂.0.30 H₂O: C, 63.27; H, 6.66; N, 6.36; Found: C, 63.18; H, 6.43; N, 6.27.

### Example 142

### N-cyclobutyl-N-[3-(5-fluoro-1H-indol-3-yl)propyl]-5-methoxychroman-3-amine (“Compound 34”)

The title compound was prepared by generally following the procedure as described for example 112 using [3-(5-fluoro-1H-indol-3-yl)-propyl]-(5-methoxy-chroman-3-yl)-amine (0.24 g, 0.677 mmol), cyclobutanone (0.12 ml, 1.56 mmol), acetic acid (0.085 ml, 1.5 mmol) and sodium cyanoborohydride (0.078 g, 1.25 mmol) in anhydrous methanol (11 ml). After 24 hrs, more cyclobutanone (0.09 ml), acetic acid (0.035 ml) and sodium cyanoborohydride (0.039 g) was added. Chromatography ((9:1:1) Hexane-EtOAc-MeOH (1% NH₄OH)) afforded 0.174 g (65%) of the title compound as an oil which was converted to the HCl salt to generate N-cyclobutyl-N-[3-(5-fluoro-1H-indol-3-yl)propyl]-5-methoxychroman-3-amine hydrochloride salt as a off-white solid: mp 70.5° C./dec; MS (ESI) m/z 407 ([M−H]⁻); Anal. calculated for C₂₅H₂₉FN₂O₂.1.20 HCl.0.40 C₄H₈O₂: C, 65.54; H, 6.91; N, 5.75; Found: C, 65.55; H, 7.14; N, 5.69.

### Example 143

### (3R)-N-cyclobutyl-N-[3-(5-fluoro-1H-indol-3-yl)propyl]-5-methoxy-3,4-dihydro-2H-chromen-3-amine (“Compound 35”)

(3R)-N-[3-(5-fluoro-1H-indol-3-yl)-propyl]-(5-methoxy-chroman-3-yl)-amine was prepared by generally following the procedure as described above for example 109 using (3R)-[5-methoxy-3,4-dihydro-2H-chromen-3-yl]amine (0.19 g, 1.06 mmol), 3-(3-bromopropyl)-5-fluoro-1H-indole (0.20 g, 0.78 mmol) and triethylamine (0.22 ml, 1.56 mmol) in anhydrous DMSO (7 ml) at 90 C for 10 hrs. Chromatography ((6:3:1) Hexane-EtOAc-MeOH (1% NH₄OH)) afforded 0.196 g (71%) of (3R)-N-[3-(5-fluoro-1H-indol-3-yl)-propyl]-(5-methoxy-chroman-3-yl)-amine as an orange and gummy solid. The product is characterized by ¹HNMR.

The title compound was prepared by generally following the procedure as described above for example 112 using (3R)-N-[3-(5-fluoro-1H-indol-3-yl)-propyl]-(5-methoxy-chroman-3-yl)-amine (0.195 g, 0.55 mmol), cyclobutanone (0.10 ml, 1.375 mmol), acetic acid (0.068 ml, 1.32 mmol) and sodium cyanoborohydride (0.069 g, 1.1 mmol) in anhydrous MeOH (8 ml). After overnight stirring at room temperature, more cyclobutanone (0.1 ml), acetic acid (0.068 ml) and sodium cyanoborohydride (0.069 g) were added, and the reaction mixture stirred at room temperature over the weekend. Chromatography ((1:1) Hexane-EtOAc) afforded 0.154 g (69%) of the title compound as a gummy solid which was converted to the HCl salt to generate (3R)-N-cyclobutyl-N-[3-(5-fluoro-1H-indol-3-yl)propyl]-5-methoxy-3,4-dihydro-2H-chromen-3-amine hydrochloride as a off-white solid: mp 115° C./dec; [α]_{D}²⁵−33.02° (c=1% SOLUTION, DMSO); MS (ES) m/z 409.2 ([M+H]⁺); Anal. calculated for C₂₅H₂₉FN₂O₂.HCl.0.50 H₂O: C, 66.14; H, 6.88; N, 6.17; Found: C, 66.04; H, 6.87; N, 5.96.

### Example 144

### N-cyclobutyl-N-[4-(5-fluoro-1H-indol-3-yl)butyl]-N-(5-methoxy-3,4-dihydro-2H-chromen-3-yl)amine (“Compound 36”)

The title compound was prepared by generally following the procedure as described above for example 143 using N-[4-(5-fluoro-1H-indol-3-yl)butyl]-5-methoxychroman-3-amine (0.145 g, 0.394 mmol), cyclobutanone (0.074 ml, 0.985 mmol), acetic acid (0.049 ml, 0.946 mmol) and sodium cyanoborohydride (0.05 g, 0.788 mmol) in anhydrous methanol (5.7 ml). After 24 and 48 hrs, more cyclobutanone (0.074 ml each time), acetic acid (0.049 ml each time) and sodium cyanoborohydride (0.05 g each time) was added. Chromatography ((1:1) Hexane-EtOAc) afforded 0.137 g (83%) of the title compound as a clear gum which was converted to the HCl salt to generate N-cyclobutyl-N-[4-(5-fluoro-1H-indol-3-yl)butyl]-N-(5-methoxy-3,4-dihydro-2H-chromen-3-yl)amine hydrochloride salt as a white solid: mp 89° C./dec; MS (ES) m/z 423.19 ([M+H]⁺); Anal. calculated for C₂₆H₃₁FN₂O₂.HCl.0.50 H₂O: C, 66.73; H, 7.11; N, 5.99; Found: C, 66.66; H, 7.14; N, 5.80.

### Example 145

### N-(cyclopropylmethyl)-N-[3-(5-fluoro-1H-indol-3-yl)propyl]-N-(5-methoxy-3,4-dihydro-2H-chromen-3-yl)amine (“Compound 37”)

The title compound was prepared by generally following the procedure as described above for example 110 using N-[3-(5-fluoro-1H-indol-3-yl)-propyl]-(5-methoxy-chroman-3-yl)-amine (0.12 g, 0.338 mmol), cyclopropane carboxaldehyde (0.028 ml, 0.372 mmol), acetic acid (0.046 ml, 0.811 mmol) and sodium cyanoborohydride (0.042 g, 0.676 mmol) in anhydrous methanol (5.7 ml). Chromatography ((6:3.5:0.5) Hexane-EtOAc-MeOH (1% NH₄OH)) afforded 0.103 g (75%) of the title compound as a gum which was converted to the HCl salt to generate N-(cyclopropylmethyl)-N-[3-(5-fluoro-1H-indol-3-yl)propyl]-N-(5-methoxy-3,4-dihydro-2H-chromen-3-yl)amine hydrochloride salt as a off-white solid: mp 114° C./dec; MS (ES) m/z 407.2 ([M−H]⁻); Anal. calculated for C₂₅H₂₉FN₂O₂.1.10 HCl.0.20H₂O: C, 66.40; H, 6.80; N, 6.19; Found: C, 66.47; H, 6.84; N, 6.14.

### Example 146

### N-(cyclopropylmethyl)-N-[3-(5-fluoro-1-methyl-1H-indol-3-yl)propyl]-N-(5-methoxy-3,4-dihydro-2H-chromen-3-yl)amine (“Compound 38”)

To sodium hydride (60% dispersion in mineral oil, 0.014 g, 0.322 mmol) in anhydrous THF (5 ml), under nitrogen at room temperature was added N-(cyclopropylmethyl)-N-[3-(5-fluoro-1H-indol-3-yl)propyl]-N-(5-methoxy-3,4-dihydro-2H-chromen-3-yl)amine (0.12 g, 0.291 mmol) in anhydrous THF (5 ml). The reaction mixture was stirred at room temperature for 30 min. Iodomethane (0.022 ml, 0.352 mmol) was added and the reaction mixture stirred at room temperature overnight. The reaction mixture was then quenched with water and extracted with ethyl acetate. The organic layer was treated with brine, dried over anhydrous magnesium sulfate, filtered and concentrated. Chromatography ((1:1) Hexane-EtOAc) afforded 0.094 g (76%) of the title compound as a colorless gum which was converted to the HCl salt to generate N-(cyclopropylmethyl)-N-[3-(5-fluoro-1-methyl-1H-indol-3-yl)propyl]-N-(5-methoxy-3,4-dihydro-2H-chromen-3-yl)amine hydrochloride salt as a white solid: mp 86° C./dec; MS (ES) m/z 423.0 ([M+H]⁺); Anal. calculated for C₂₆H₃₁FN₂O₂.1.20 HCl: C, 66.97; H, 6.96; N, 6.01; Found: C, 67.01; H, 6.85; N, 5.85.

### Example 147

### N-cyclopentyl-N-[3-(5-fluoro-1H-indol-3-yl)propyl]-N-(5-methoxy-3,4-dihydro-2H-chromen-3-yl)amine (“Compound 39”)

The title compound was prepared by generally, following the procedure as described above for example 110 using N-[3-(5-fluoro-1H-indol-3-yl)-propyl]-(5-methoxy-chroman-3-yl)-amine (0.095 g, 0.268 mmol), cyclopentanone (0.059 ml, 0.67 mmol), acetic acid (0.033 ml, 0.643 mmol) and sodium cyanoborohydride (0.034 g, 0.536 mmol) in anhydrous MeOH (4 ml). At 24 hrs, 48 hrs and 72 hrs, more cyclopentanone (0.1 ml each time), acetic acid (0.07 ml) and sodium cyanoborohydride (0.07 g) were added. Chromatography ((1:1) Hexane-EtOAc) afforded 0.042 g (37%) of the title compound as a gum which was converted to the HCl salt to generate N-cyclopentyl-N-[3-(5-fluoro-1H-indol-3-yl)propyl]-N-(5-methoxy-3,4-dihydro-2H-chromen-3-yl)amine hydrochloride salt as a white solid: mp 112° C./dec; MS (ES) m/z 421.2 ([M−H]⁻); Anal. calculated for C₂₆H₃₁FN₂O₂.HCl.0.50 H₂O: C, 66.73; H, 7.11; N, 5.99; Found: C, 66.62; H, 7.09; N, 5.63.

### Example 148

### N-[3-(5-fluoro-1H-indol-3-yl)propyl]-N-isopropyl-N-(5-methoxy-3,4-dihydro-2H-chromen-3-yl)amine (“Compound 40”)

N-isopropyl-5-methoxychroman-3-amine was prepared by generally following the procedure as described above for example 110 using [5-methoxy-3,4-dihydro-2H-chromen-3-yl]amine (0.30 g, 1.67 mmol), acetone (1.23 ml, 16.7 mmol), acetic acid (0.23 ml, 4 mmol) and sodium cyanoborohydride (0.21 g, 3.34 mmol) in anhydrous methanol (6 ml). Chromatography ((7:3) Hexane-EtOAc) afforded 0.318 g (86%) of N-isopropyl-5-methoxychroman-3-amine as a clear oil. The product was characterized by ¹HNMR.

The title compound was prepared by generally following the procedure as described above for example 110 using N-isopropyl-5-methoxychroman-3-amine (0.10 g, 0.45 mmol), 3-(5-fluoro-1H-indol-3-yl)propanal (0.172 g, 0.9 mmol), acetic acid (0.062 ml, 1.08 mmol) and sodium cyanoborohydride (0.056 g, 0.9 mmol) in anhydrous methanol (7.5 ml). Chromatography ((3:1) Hexane-EtOAc followed by (1:1) Hexane-EtOAc) afforded 0.057 g (32%) of the title compound as a gum which was converted to the HCl salt to generate N-[3-(5-fluoro-1H-indol-3-yl)propyl]-N-isopropyl-N-(5-methoxy-3,4-dihydro-2H-chromen-3-yl)amine hydrochloride salt as a pale yellow solid: mp 105° C./dec; MS (ES) Hvz 395.13 ([M−H]⁻); Anal. calculated for C₂₄H₂₉FN₂O₂.1.10 HCl: C, 66.02; H, 6.95; N, 6.42; Found: C, 65.98; H, 6.97; N, 6.31.

### Example 149

### N-cyclopropyl-N-[3-(5-fluoro-1H-indol-3-yl)propyl]-N-(5-methoxy-3,4-dihydro-2H-chromen-3-yl)amine (“Compound 41”)

To 5-methoxy-2H-chromen-3(4H)-one (0.13 g, 0.73 mmol) in anhydrous 1,2-dichloroethane (3.5 ml), under nitrogen at room temperature, was added cyclopropylamine (0.051 ml, 0.73 mmol), acetic acid (0.083 ml, 1.46 mmol) and sodium triacetoxyborohydride (0.216 g, 1.02 mmol). The reaction mixture was stirred at room temperature for 2 hrs. The reaction mixture was then quenched with 1N NaOH/H₂O and diluted with dichloromethane. The organic layer was separated, treated with brine, dried over anhydrous magnesium sulfate, filtered and concentrated. Chromatography ((1:1) Hexane-EtOAc) afforded 0.076 g (48%) of N-cyclopropyl-N-cyclopropyl-N-[5-methoxy-3,4-dihydro-2H-chromen-3-yl)amine as a gum.

To N-cyclopropyl-N-[5-methoxy-3,4-dihydro-2H-chromen-3-yl)amine (0.076 g, 0.346 mmol) in anhydrous methanol (5.6 ml), under nitrogen at room temperature, was added 3-(5-fluoro-1H-indol-3-yl)propanal (0.079 g, 0.415 mmol), acetic acid (0.048 ml, 0.83 mmol) and sodium cyanoborohydride (0.043 g, 0.692 mmol). The reaction mixture was stirred at room temperature overnight and worked up as described above for example 110. Chromatography ((3:1) Hexane-EtOAc) afforded 0.113 g (82%) of the title compound which was converted to the HCl salt to generate N-cyclopropyl-N-[3-(5-fluoro-1H-indol-3-yl)propyl]-N-(5-methoxy-3,4-dihydro-2H-chromen-3-yl)amine hydrochloride salt as an off-white solid: mp 115° C./dec; MS (ES) m/z 395.1 ([M+H]⁺); Anal. Calculated for C₂₄H₂₇FN₂O₂.1.10 HCl: C, 66.33; H, 6.52; N, 6.45. Found: C, 66.16; H, 6.41; N, 6.25.

### Example 150

### N-(cyclobutylmethyl)-N-[3-(5-fluoro-1H-indol-3-yl)propyl]-N-(5-methoxy-3,4-dihydro-2H-chromen-3-yl)amine (“Compound 42”)

To (5-methoxy-3,4-dihydro-2H-chromen-3-yl)amine (0.20 g, 1.12 mmol) in anhydrous dimethylsulfoxide (6 ml), under nitrogen at room temperature, was added cyclobutylmethyl bromide (0.096 ml, 0.86 mmol) and triethylamine (0.24 ml, 1.72 mmol). The reaction mixture was brought to 90° C. and kept at that temperature for 9 hrs. The reaction mixture was then cooled down to room temperature and diluted with EtOAc/H₂O. The organic layer was separated, treated with brine, dried over anhydrous magnesium sulfate, filtered and concentrated. Chromatography ((1:1) Hexane-EtOAc) afforded 0.066 g (24%) of N-(cyclobutylmethyl)-N-(5-methoxy-3,4-dihydro-2H-chromen-3-yl) amine as a gum. MS (ES) nvz 248 ([M+H]⁺). The product was also characterized by ¹HNMR.

To N-(cyclobutylmethyl)-N-(5-methoxy-3,4-dihydro-2H-chromen-3-yl) amine (0.152 g, 0.614 mmol) in anhydrous methanol (10 ml), under nitrogen at room temperature, was added 3-(5-fluoro-1H-indol-3-yl)propanal (0.14 g, 0.736 mmol), acetic acid (0.085 ml, 1.47 mmol) and sodium cyanoborohydride (0.077 g, 1.23 mmol). The reaction mixture was stirred at room temperature overnight and worked up as described above for example 110. Chromatography ((3:1) Hexane-EtOAc) afforded 0.198 g (76%) of the title compound which was converted to the HCl salt to generate N-(cyclobutylmethyl)-N-[3-(5-fluoro-1H-indol-3-yl)propyl]-N-(5-methoxy-3,4-dihydro-2H-chromen-3-yl)amine hydrochloride salt as a white solid: mp 115° C./dec; MS (ES) m/z 423.1 ([M+H]⁺); Anal. Calculated for C₂₆H₃₁FN₂O₂.1.10 HCl: C, 67.50; H, 6.99; N, 6.05. Found: C, 67.37; H, 6.63; N, 5.95.

### Example 151

### N-(cyclopropylmethyl)-N-[3-(1H-indol-3-yl)propyl]-N-(5-methoxy-3,4-dihydro-2H-chromen-3-yl)amine (“Compound 43”)

N-[3-(1H-indol-3-yl)propyl]-5-methoxy-chroman-3-yl)-amine was prepared by generally following the procedure as described above for example 149 using (5-methoxy-3,4-dihydro-2H-chromen-3-yl)amine (0.244 g, 1.36 mmol), 3-(1H-indol-3-yl)propanal (0.26 g, 1.5 mmol), acetic acid (0.2 ml, 3.4 mmol) and sodium cyanoborohydride (0.17 g, 2.72 mmol) in anhydrous methanol (13 ml). Chromatography (2% MeOH in CH₂Cl₂ (5% NH₄OH)) afforded 0.21 g (45%) of N-[3-(1H-indol-3-yl)propyl]-5-methoxy-chroman-3-yl)-amine as a white solid. The product was characterized by ¹HNMR.

The title compound was prepared by generally following the procedure as described above for example 110 using N-[3-(1H-indol-3-yl)propyl]-5-methoxy-chroman-3-yl)-amine (0.103 g, 0.306 mmol), cyclopropane carboxaldehyde (0.11 ml, 1.47 mmol), acetic acid (0.05 ml, 0.9 mmol) and sodium cyanoborohydride (0.048 g, 0.76 mmol) in anhydrous methanol (4 ml). Chromatography (1% MeOH in CH₂Cl₂ (5% NH₄OH)) afforded 0.11 g (94%) of the title compound which was converted to the HCl salt to generate N-(cyclopropylmethyl)-N-[3-(1H-indol-3-yl)propyl]-N-(5-methoxy-3,4-dihydro-2H-chromen-3-yl)amine hydrochloride salt as a white solid: mp 107-110° C.; MS (ES) m/z 390.2 ([M+H]⁺); Anal. Calculated for C₂₅H₃₀N₂O₂.HCl.0.80 H₂O: C, 68.03; H, 7.44; N, 6.35. Found: C, 67.90; H, 6.71; N, 6.27.

### Example 152

### N-cyclobutyl-N-[3-(1H-indol-3-yl)propyl]-N-(5-methoxy-3,4-dihydro-2H-chromen-3-yl)amine (“Compound 44”)

The title compound was prepared as described above for example 112 using N-[3-(1H-indol-3-yl)propyl]-5-methoxy-chroman-3-yl)-amine (0.102 g, 0.303 mmol), cyclobutanone (0.081 ml, 1.08 mmol), acetic acid (0.05 ml, 0.87 mmol) and sodium cyanoborohydride (0.048 g, 0.76 mmol) in anhydrous methanol (3.5 ml). After overnight stirring at room temperature, more cyclobutanone (0.081 ml), acetic acid (0.05 ml) and sodium cyanoborohydride (0.048 g) were added, and the reaction mixture stirred at room temperature for another night. Chromatography (1% MeOH in CH₂Cl₂ (5% NH₄OH)) afforded 0.056 g (46%) of the title compound which was converted to the HCl salt to generate N-cyclobutyl-N-[3-(1H-indol-3-yl)propyl]-N-(5-methoxy-3,4-dihydro-2H-chromen-3-yl)amine hydrochloride salt as a white solid: mp 113-117° C.; MS (ES) m/z 391.1 ([M+H]⁺); Anal. Calculated for C₂₅H₃₀N₂O₂.HCl.0.50 H₂O: C, 68.87; H, 7.40; N, 6.43. Found: C, 68.77; H, 7.26; N, 6.42.

### Example 153

### 3-{3-[(cyclopropylmethyl)(5-methoxy-3,4-dihydro-2H-chromen-3-yl)amino]propyl}-1H-indole-5-carbonitrile (“Compound 45”)

3-{3-[(5-methoxy-3,4-dihydro-2H-chromen-3-yl)amino]propyl}-1H-indole-5-carbonitrile was prepared by generally following the procedure as described above for example 150 using (5-methoxy-3,4-dihydro-2H-chromen-3-yl)amine (0.35 g, 1.95 mmol), 3-(3-bromopropyl)-1H-indole-5-carbonitrile (0.513 g, 1.95 mmol), triethylamine (0.68 ml, 4.8 mmol) in anhydrous dimethylsulfoxide (12 ml) at 90° C. for 16 hrs. Chromatography (2% MeOH in CH₂Cl₂ (5% NH₄OH)) afforded 0.34 g (48%) of 3-{3-[(5-methoxy-3,4-dihydro-2H-chromen-3-yl)amino]propyl}-1H-indole-5-carbonitrile as a clear gum. The product was characterized by ¹HNMR.

The title compound was prepared by generally following the procedure as described above for example 110 using 3-{3-[(5-methoxy-3,4-dihydro-2H-chromen-3-yl)amino]propyl}-1H-indole-5-carbonitrile (0.102 g, 0.282 mmol), cyclopropane carboxaldehyde (0.105 ml, 1.40 mmol), acetic acid (0.05 ml, 0.87 mmol) and sodium cyanoborohydride (0.044 g, 0.70 mmol) in anhydrous methanol (3.5 ml). Chromatography (1% MeOH in CH₂Cl₂ (5% NH₄OH)) afforded 0.07 g (60%) of the title compound which was converted to the HCl salt to generate 3-{3-[(cyclopropylmethyl)(5-methoxy-3,4-dihydro-2H-chromen-3-yl)amino]propyl}-1H-indole-5-carbonitrile hydrochloride salt as a white solid: mp 123-127° C. (melts with decomposition); MS (ES) m/z 416.1 ([M+H]⁺); Anal. Calculated for C₂₆H₂₉N₃O₂.HCl.0.60 H₂O: C, 67.48; H, 6.80; N, 9.08. Found: C, 67.40; H, 6.29; N, 8.74.

### Example 154

### 3-{3-[cyclobutyl(5-methoxy-3,4-dihydro-2H-chromen-3-yl)amino]propyl}-1H-indole-5-carbonitrile (“Compound 46”)

The title compound was prepared as described above for example 112 using 3-{3-[(5-methoxy-3,4-dihydro-2H-chromen-3-yl)amino]propyl}-1H-indole-5-carbonitrile (0.14 g, 0.387 mmol), cyclobutanone (0.10 ml, 1.50 mmol), acetic acid (0.06 ml, 1.05 mmol) and sodium cyanoborohydride (0.061 g, 0.97 mmol) in anhydrous methanol (4.5 ml). After overnight stirring at room temperature, more cyclobutanone (0.10 ml), acetic acid (0.06 ml) and sodium cyanoborohydride (0.061 g) were added, and the reaction mixture stirred at room temperature for another night. Chromatography (1% MeOH in CH₂Cl₂ (5% NH₄OH)) afforded 0.13 g (81%) of the title compound which was converted to the HCl salt to generate 3-{3-[cyclobutyl(5-methoxy-3′,4-dihydro-2H-chromen-3-yl)amino]propyl}-1H-indole-5-carbonitrile hydrochloride salt as a white solid: mp 133-137° C.; MS (ES) m/z 414.2 ([M−H]⁻); Anal. Calculated for C₂₆H₂₉N₃O₂.HCl.0.60 H₂O: C, 67.48; H, 6.80; N, 9.08. Found: C, 67.41; H. 6.70; N, 8.77.

### Example 155

### N-[3-(5-fluoro-1H-indol-3-yl)propyl]-N-(8-methoxy-1,2,3,4-tetrahydronaphthalen-2-yl)-N-propylamine (“Compound 47”)

[3-(5-(Fluoro-1H-indol-3-yl)-propyl]-(8-methoxy-1,2,3,4-tetrahydronaphthalen-2-yl)-amine was prepared by generally following the procedure as described above for example 126 using 8-methoxy-3,4-dihydro-1H-naphthalen-2-one (1.02 g, 5. mmol), 3-(5-fluoro-1H-indol-3-yl)propylamine (1.1 g, 5.8 mmol), acetic acid (0.67 ml, 1.2 mol) and sodium triacetoxyborohydride (1.84 g, 0.87 mmol) in anhydrous 1,2-dichloroethane (40 ml). Chromatography ((9:1) CH₂Cl₂—MeOH) afforded 1.52 g of [3-(5-(Fluoro-1H-indol-3-yl)-propyl]-(8-methoxy-1,2,3,4-tetrahydronaphthalen-2-yl)-amine as a off-white solid: mp 152-153° C. The oxalate salt was prepared in ethanol and collected as a white solid: mp 103° C. decomposed; Anal. calculated for C₂₂H₂₅FN₂O C₂H₂O₄.0.25.H₂O: C, 64.49; H, 6.20; N, 6.27; Found: C, 64.16; H, 6.16; N, 6.12

The title compound was prepared by generally following the procedure as described above for example 110 using [3-(5-(fluoro-1H-indol-3-yl)-propyl]-(8-methoxy-1,2,3,4-tetrahydronaphthalen-2-yl)-amine (0.6 g, 1.7 mmol), propionaldehyde (1.23 ml, 17 mmol), acetic acid (0.02 ml, 0.42 mmol) and sodium cyanoborohydride (0.05 g, 0.76 mmol) in anhydrous methanol (40 ml). Chromatography ((49:1) CH₂Cl₂—MeOH) afforded 0.12 g of the title compound as a light brown foam which was converted to the HCl salt to generate N-[3-(5-fluoro-1H-indol-3-yl)propyl]-N-(8-methoxy-1,2,3,4-tetrahydronaphthalen-2-yl)-N-propylamine hydrochloride salt as a light yellow solid: mp decomposed at 97.6° C.; MS (APCI) m/z 395 ([M+H]⁺); Anal. calculated for C₂₅H₃₁FN₂O.HCl.0.75 H₂O: C, 67.55; H, 7.60; N, 6.30; Found: C, 67.72; H, 7.50; N, 6.05.

### Examples 155a and 155b

### (−)-N-[3-(5-fluoro-1H-indol-3-yl)propyl]-8-methoxy-N-propyl-1,2,3,4-tetrahydronaphthalen-2-amine (“Compound 47a”) and (2R)-N-[3-(5-fluoro-1H-indol-3-yl)propyl]-8-methoxy-N-propyl-1,2,3,4-tetrahyoronaphthalen-2-amine (“Compound 47b”)

The enantiomers of N-[3-(5-fluoro-1H-indol-3-yl)propyl]-N-(8-methoxy-1,2,3,4-tetrahydronaphthalen-2-yl)-N-propylamine were separated by chiral HPLC, isolated, and converted to the HCl salt as described above for the racemate, generating the following products:

(−)-N-[3-(5-fluoro-1H-indol-3-yl)propyl]-8-methoxy-N-propyl-1,2,3,4-tetrahydronaphthalen-2-amine hydrochloride salt as a off-white solid: mp 107° C. decomposed; [α]_{D}²⁵=−61.7° (c=5.3MG/0.53ML, DMSO); MS (ESI) m/z 395 ([M+H]⁺); Anal. calculated for C₂₅H₃₁FN₂O.HCl.0.50 H₂O: C, 68.24; H, 7.56; N, 6.37; Found: C, 67.96; H, 7.62; N, 6.23.

(2R)-N-[3-(5-fluoro-1H-indol-3-yl)propyl]-8-methoxy-N-propyl-1,2,3,4-tetrahydronaphthalen-2-amine hydrochloride salt as a off-white solid: mp 110° C. decomposed; [α]_{D}²⁵=+57.6° (c=5.6MG/0.56ML, DMSO); MS (ESI) m/z 395 ([M+H]⁺); Anal. calculated for C₂₅H₃₁FN₂O.HCl.0.75 H₂O: C, 67.55H, 7.60 N, 6.30; Found: C, 67.52H, 7.57 N, 6.08.

### Example 156

### N-[2-(5-fluoro-1H-indol-3-yl)ethyl]-N-(8-methoxy-1,2,3,4-tetrahydronaphthalen-2-yl)-N-propylamine (“Compound 48”)

[2-(5-(fluoro-1H-indol-3-yl)-ethyl]-(8-methoxy-1,2,3,4-tetrahydronaphthalen-2-yl)-amine was prepared by generally following the procedure as described above for example 126 using 8-methoxy-3,4-dihydro-1H-naphthalen-2-one (0.52 g, 2.8 mmol), 2-(5-fluoro-1H-indol-3-yl)ethylamine (0.5 g, 2.8 mmol), acetic acid (0.49 ml, 8.4 mmol) and sodium triacetoxyborohydride (0.89 g, 4.2 mmol) in anhydrous 1,2-dichloroethane (30 ml). Chromatography ((9:1) CH₂Cl₂—MeOH) afforded 0.83 g (87%) of [2-(5-(fluoro-1H-indol-3-yl)-ethyl]-(8-methoxy-1,2,3,4-tetrahydro-naphthalene-2-yl)-amine as a light brown oil. The oxalate salt was prepared in tetrahydrofuran and collected as an off-white solid: mp 227-229° C.; Anal. calculated for C₂₁H₂₃FN₂O.C₂H₂O₄.0.25 H₂O: C, 63.81H, 5.94 N, 6.47; Found: C, 63.96H, 5.84 N, 6.41.

The title compound was prepared by generally following the procedure as described above for example 110 using [2-(5-(fluoro-1H-indol-3-yl)-ethyl]-(8-methoxy-1,2,3,4-tetrahydronaphthalen-2-yl)-amine (0.2 g, 0.6 mmol), propionaldehyde (0.43 ml, 6 mmol), acetic acid (0.03 ml, 0.6 mmol) and sodium cyanoborohydride (0.74 g, 1.2 mmol) in anhydrous methanol (40 ml). Chromatography ((19:1) CH₂Cl₂—MeOH) afforded 0.22 g (96%) of the title compound as a light brown foam which was converted to the HCl salt to generate N-[2-(5-fluoro-1H-indol-3-yl)ethyl]-N-(8-methoxy-1,2,3,4-tetrahydronaphthalen-2-yl)-N-propylamine hydrochloride salt as a white solid: mp 105° C. decomposed; MS (APCI) m/z 381 ([M+H]⁺); Anal. calculated for C₂₄H₂₉FN₂O.HCl.0.50 H₂O: C, 67.67H, 7.34 N, 6.58; Found: C, 67.87; H, 7.42; N, 6.36.

### Example 157

### N-ethyl-N-[2-(5-fluoro-1H-indol-3-yl)ethyl]-N-(8-methoxy-1,2,3,4-tetrahydronaphthalen-2-yl)amine (“Compound 49”)

The title compound was prepared by generally following the procedure as described above for example 110 using [3-(5-(fluoro-1H-indol-3-yl)-propyl]-(8-methoxy-1,2,3,4-tetrahydronaphthalen-2-yl)-amine (0.2 g, 0.6 mmol), acetaldehyde (0.3 ml, 6 mmol), acetic acid (0.03 ml, 0.6 mmol) and sodium cyanoborohydride (0.07 g, 1.1 mmol) in anhydrous methanol (40 ml). Chromatography ((9:1) CH₂Cl₂—MeOH) afforded 0.18 g (82%) of the title compound as a clear oil which was converted to the HCl salt to generate N-ethyl-N-[2-(5-fluoro-1H-indol-3-yl)ethyl]-N-(8-methoxy-1,2,3,4-tetrahydronaphthalen-2-yl)amine hydrochloride salt as an off-white solid: mp 114° C. decomposed; MS (APCI) m/z 367 ([M+H]⁺); Anal. calculated for C₂₃H₂₇FN₂O.HCl.0.50 H₂O: C, 67.06H, 7.10 N, 6.80; Found: C, 67.02; H, 7.03; N, 6.58.

### Example 158

### N-[3-(1H-indol-3-yl)propyl]-8-methoxy-N-propyl-1,2,3,4-tetrahydronaphthalen-2-amine (“Compound 50”)

The title compound was prepared by generally following the procedure as described above for example 110 using [3-(1H-indol-3-yl)-propyl]-(8-methoxy-1,2,3,4-tetrahydronaphthalen-2-yl)-amine (0.15 g, 0.45 mmol), propionaldehyde (0.32 ml, 4.5 mmol), acetic acid (0.03 ml, 0.45 mmol) and sodium cyanoborohydride (0.06 g, 0.9 mmol) in anhydrous methanol (40 ml). Chromatography ((9:1) CH₂Cl₂—MeOH (1% NH₄OH)) afforded 0.15 g (88%) of the title compound as a clear oil which was converted to the HCl salt to generate N-[3-(1H-indol-3-yl)propyl]-8-methoxy-N-propyl-1,2,3,4-tetrahydronaphthalen-2-amine hydrochloride salt as a light yellow solid: mp 108° C. decomposed; MS (ESI) m/z 377 ([M+H]⁺); Anal. calculated for C₂₅H₃₂N₂O.HCl.0.50 H₂O: C, 71.15; H, 8.12; N, 6.64; Found: C, 71.19; H, 8.14; N, 6.27.

### Example 159

### N-[3-(5-fluoro-1H-indol-3-yl)propyl]-N-(5-fluoro-8-methoxy-1,2,3,4-tetrahydronaphthalen-2-yl)-N-propylamine (“Compound 51”)

[3-(5-fluoro-1H-indol-3-yl)-propyl]-(5-fluoro-8-methoxy-1,2,3,4-tetrahydronaphthalen-2-yl)-amine was prepared by generally following the procedure as described above for example 126 using 5-fluoro-8-methoxy-3,4-dihydro-1H-naphthalen-2-one (0.44 g, 2.3 mmol), 3-(5-fluoro-1H-indol-3-yl)propylamine (0.44 g, 2.3 mmol), acetic acid (0.38 ml, 6.9 mmol) and sodium triacetoxyborohydride (0.72 g, 3.4 mmol) in anhydrous 1,2-dichloroethane (30 ml). Chromatography ((19:1) CH₂Cl₂—MeOH) afforded 0.79 g (93%) of [3-(5-fluoro-1H-indol-3-yl)-prgpyl]-(5-fluoro-8-methoxy-1,2,3,4-tetrahydronaphthalen-2-yl)-amine as an off-white foam. The product was characterized by ¹HNMR.

The title compound was prepared by generally following the procedure as described above for example 110 using [3-(5-fluoro-1H-indol-3-yl)-propyl]-(5-fluoro-8-methoxy-1,2,3,4-tetrahydronaphthalen-2-yl)-amine (0.79 gi 2.1 mmol), propionaldehyde (1.54 ml, 21 mmol), acetic acid (0.12 ml, 2.1 mmol) and sodium cyanoborohydride (0.27 g, 4.2 mmol) in anhydrous methanol (40 ml). Chromatography ((9:1) CH₂Cl₂—MeOH) afforded 0.89 g (100%) of the title compound as a clear oil which was converted to the HCl salt to generate N-[3-(5-fluoro-1H-indol-3-yl)propyl]-N-(5-fluoro-8-methoxy-1,2,3,4-tetrahydronaphthalen-2-yl)-N-propylamine hydrochloride salt as a white solid; mp 107° C. decomposed; MS (ESI)-m/z 413 ([M+H]⁺); Anal. calculated for C₂₅H₃₀F₂N₂O.HCl.0.50 H₂O: C, 65.56; H, 7.04; N, 6.12; Found: C, 65.56; H, 7.01; N, 5.74.

### Examples 159a and 159b

### (+)-5-fluoro-N-[3-(5-fluoro-1H-indol-3-yl)propyl]-8-methoxy-N-propyl-1,2,3,4-tetrahydro-2-naphthalenamine (“Compound Sla”) and (−)-5-fluoro-N-[3-(5-fluoro-1H-indol-3-yl)propyl]-8-methoxy-N-propyl-1,2,3,4-tetrahydro-2-naphthalenamine (“Compound 51b”)

The enantiomers of N-[3-(5-fluoro-1H-indol-3-yl)propyl]-N-(5-fluoro-8-methoxy-1,2,3,4-tetrahydronaphthalen-2-yl)-N-propylamine were separated by chiral HPLC, isolated, and converted to the HCl salt as described above (example 159) for the racemate, generating the following products:

(+)-5-fluoro-N-[3-(5-fluoro-1H-indol-3-yl)propyl]-8-methoxy-N-propyl-1,2,3,4-tetrahydro-2-naphthalenamine hydrochloride salt as a white solid: mp 117° C. decomposed; [α]_{D}²⁵=+62.0° (c=5.7MG/0.570ML, DMSO); MS (ESI) m/z 413 ([M+H]⁺); Anal. calculated for C₂₅H₃₀F₂N₂O.HCl.0.75 H₂O: C, 64.93; H, 7.08; N, 6.06; Found: C, 65.15; H, 6.80; N, 6.00.

(−)-5-fluoro-N-[3-(5-fluoro-1H-indol-3-yl)propyl]-8-methoxy-N-propyl-1,2,3,4-tetrahydro-2-naphthalenamine hydrochloride salt as a white solid: mp 117° C. decomposed; [α]_{D}²⁵=−63.0° (c=5.5MG/0.550ML, DMSO); MS (ESI) m/z 413 ([M+H]⁺); Anal. calculated for C₂₅H₃₀F₂N₂O.HCl.0.50 H₂O: C, 65.56; H, 7.04; N, 6.12; Found: C, 65.49; H, 6.81; N, 6.08.

### Example 160

### 8-fluoro-3-{[3-(6-fluoro-1H-indol-3-yl)propyl]amino}chromane-5-carboxamide (“Compound 52”) and 3-{(cyclopropylmethyl)[3-(6-fluoro-1H-indol-3-yl)propyl]amino}-8-fluorochromane-5-carboxamide (“Compound 52a”)

To 3-amino-8-fluorochromane-5-carboxamide (0.2 g, 0.951 mmol) in anhydrous methanol (16 mL), under nitrogen at room temperature, was added 3-(6-fluoro-1H-indol-3-yl)propanal (0.19 g, 0.998 mmol), acetic acid (0.12 mL, 2.37 mmol) and sodium cyanoborohydride (0.12 g, 1.9 mmol). The reaction mixture was stirred at room temperature overnight. Chromatography ((6:3:1) Hex-EtOAc-MeOH (1% NH₄OH)) afforded 0.24 g (67%) of 8-fluoro-3-{[3-(6-fluoro-1H-indol-3-yl)propyl]amino}chromane-5-carboxamide. The product was characterized by ¹HNMR and LC/MS (MW 385 as expected).

To 8-fluoro-3-{[3-(6-fluoro-1H-indol-3-yl)propyl]amino}chromane-5-carboxamide (0.1 g, 0.26 mmol) in anhydrous methanol (5 mL), under nitrogen at room temperature, was added cyclopropanecarboxaldehyde (0.033 mL, 0.43 mmol), acetic acid (0.055 mL, 0.98 mmol) and sodium cyanoborohydride (0.051 g, 0.8 mmol). The reaction mixture was stirred at room temperature overnight. Chromatography ((5:4:1) EtOAc-Hex-MeOH (1% NH₄OH)) afforded 0.094 g (83%) of 3-{(cyclopropylmethyl)[3-(6-fluoro-1H-indol-3-yl)propyl]amino}-8-fluorochromane-5-carboxamide which was then converted to the HCl salt to generate 3-{(cyclopropylmethyl)[3-(6-fluoro-1H-indol-3-yl)propyl]amino}-8-fluorochromane-5-carboxamide hydrochloride salt as a white solid: mp 123° C./DEC; MS (ES) m/z 440.2; Anal. Calc'd for C₂₅H₂₇F₂N₃O₂.HCl.H₂O: C, 60.79; H, 6.12; N, 8.51. Found: C, 60.96; H, 6.15; N, 8.50.

### Example 161

### 3-{cyclobutyl[3-(6-fluoro-1H-indol-3-yl)propyl]amino}-8-fluorochromane-5-carboxamide (“Compound 53”)

This compound was prepared as described for example 160 (compound 52a) using 8-fluoro-3-{[3-(6-fluoro-1H-indol-3-yl)propyl]amino}chromane-5-carboxamide (0.14 g, 0.34 mmol), cyclobutanone (0.064 mL, 0.85 mmol), acetic acid (0.046 mL, 0.82 mmol), sodium cyanoborohydride (0.043 g, 0.68 mmol) and methanol (6.6 mL). After stirring at room temperature overnight, more cyclobutanone (0.064 mL, 0.85 mmol), acetic acid (0.046 mL, 0.82 mmol) and sodium cyanoborohydride (0.043 g, 0.68 mmol) were added and the reaction mixture stirred for another night. Chromatography ((5:4:1) EtOAc-Hex-MeOH (1% NH₄OH)) afforded 0.16 g (99%) of 3-{cyclobutyl[3-(6-fluoro-1H-indol-3-yl)propyl]amino}-8-fluorochromane-5-carboxamide which was converted to the HCl salt to generate 3-{cyclobutyl[3-(6-fluoro-1H-indol-3-yl)propyl]amino}-8-fluorochromane-5-carboxamide hydrochloride salt as a white solid: mp 126° C./DEC; MS (ES) nl/z 440.2; Anal. Calcd for C₂₅H₂₇F₂N₃O₂.1.10 HCl.H₂O: C, 60.34; H, 6.10; N, 8.44. Found: C, 60.26; H, 5.97; N, 8.43.

### Example 162

### Methyl 8-fluoro-3-{[3-(5-fluoro-1H-indol-3-yl)propyl]amino}chromane-5-carboxylate (“Compound 54”)

To methyl 3-amino-8-fluorochromane-5-carboxylate (1.4 g, 6.21 mmol) in anhydrous methanol (100 mL), under nitrogen at room temperature, was added 3-(5-fluoro-1H-indol-3-yl)propanal (1.25 g, 6.52 mmol), acetic acid (0.8 mL, 14.9 mmol) and sodium cyanoborohydride (0.78 g, 12.4 mmol). The reaction mixture was stirred at room temperature overnight. Chromatography ((6:3:1) Hex-EtOAc-MeOH (1% NH₄OH)) afforded 2.2 g (89%) of desired product which was converted to the HCl salt to generate methyl 8-fluoro-3-{[3-(5-fluoro-1H-indol-3-yl)propyl]amino}chromane-5-carboxylate hydrochloride salt as a slight yellow solid: mp 219° C./dec; MS (ES) m/z 401.2; Anal. Calcd for C₂₂H₂₂F₂N₂O₃.HCl: C, 60.48; H, 5.31; N, 6.41. Found: C, 60.20; H, 4.85; N, 6.16.

### Example 163

### Methyl 3-{cyclobutyl[3-(5-fluoro-1H-indol-3-yl)propyl]amino}-8-fluorochromane-5-carboxylate (“Compound 55”)

To methyl 8-fluoro-3-{[3-(5-fluoro-1H-indol-3-yl)propyl]amino}chromane-5-carboxylate (1.37 g, 3.42 mmol) in anhydrous methanol (40 mL), under nitrogen at room temperature, was added cyclobutanone (0.64 mL, 8.55 mmol), acetic acid (0.42 mL, 8.21 mmol) and sodium cyanoborohydride (0.43 g, 6.84 mmol). The reaction mixture was stirred at room temperature overnight. More cyclobutanone (0.3 mL), acetic acid (0.2 mL) and sodium cyanoborohydride (0.2 g) were added and the reaction mixture stirred for another night. Chromatography ((2:1) Hex-EtOAc) afforded 2.2 g (88%) of desired product which was converted to the HCl salt to generate methyl 3-{cyclobutyl[3-(5-fluoro-1H-indol-3-yl)propyl]amino}-8-fluorochromane-5-carboxylate hydrochloride salt as a foamy gummy solid: mp 110° C./dec; MS (ES) m/z 455.2; Anal. Calcd for C₂₆H₂₈F₂N₂O₃.HCl: C, 63.61; H, 5.95; N, 5.71. Found: C, 63.43; H, 5.82; N, 5.69.

### Example 164

### 3-{cyclobutyl[3-(5-fluoro-1H-indol-3-yl)propyl]amino}-8-fluorochromane-5-carboxylic acid (“Compound 56”)

To methyl 3-{cyclobutyl[3-(5-fluoro-1H-indol-3-yl)propyl]amino}-8-fluorochromane-5-carboxylate (2.2 g, 4.84 mmol) in absolute ethanol (30 mL), was added-2.5 N NaOH in H₂O (2.7 mL, 6.78 mmol). The reaction mixture was brought to reflux and kept under reflux for 1 hr. It was cooled down and concentrated. The residue was taken up in CH₂Cl₂/H₂O and the organic layer separated. The aqueous layer was made neutral with 2N HCl/H₂O and extracted several times with ethyl acetate. The organic extracts were pooled, treated with brine, dried over magnesium sulfate, filtered and concentrated affording 1.35 g (65%) of desired product which was converted to the HCl salt to generate 3-{cyclobutyl[3-(5-fluoro-1H-indol-3-yl)propyl]amino}-8-fluorochromane-5-carboxylic acid hydrochloride salt as a white solid: mp 140° C./dec; MS (ES) m/z 441.3; Anal. Calcd for C₂₅H₂₆F₂N₂O₃.2.00 HCl.1.50 H₂O: C, 55.56; H, 5.78; N, 5.18. Found: C, 55.72; H, 5.78; N, 5.03.

### Example 165

### Methyl 8-fluoro-3-{[4-(5-fluoro-1H-indol-3-yl)butyl]amino}chromane-5-carboxylate (“Compound 57”)

To methyl 3-amino-8-fluorochromane-5-carboxylate (0.8 g, 3.55 mmol) in anhydrous methanol, under nitrogen at room temperature, was added 4-(5-fluoro-1H-indol-3-yl)butanal (0.73 g, 3.55 mmol), acetic acid (0.44 mL, 8.52 mmol) and sodium cyanoborohydride. (0.45 g, 7.1 mmol). The reaction mixture was stirred at room temperature overnight. Chromatography ((6:3:1) Hex-EtOAc-MeOH (1% NH₄OH)) afforded 0.82 g (56%) of desired product which was converted to the HCl salt to generate methyl 8-fluoro-3-{[4-(5-fluoro-1H-indol-3-yl)butyl]amino}chromane-5-carboxylate hydrochloride salt as a white solid: mp 228° C./dec; MS (ES) m/z 415.1; Anal. Calcd for C₂₃H₂₄F₂N₂O₃.HCl: C, 61.27; H, 5.59; N, 6.21. Found: C, 61.11; H, 5.52; N, 6.06.

### Examples 165a and 165b

### Methyl (3S)-8-fluoro-3-{[4-(5-fluoro-1H-indol-3-yl)butyl]amino}chromane-5-carboxylate (“Compound 57a”) and methyl (3R)-8-fluoro-3-{[4-(5-fluoro-1H-indol-3-yl)butyl]amino}chromane-5-carboxylate (“Compound 57b”)

The enantiomers of methyl 8-fluoro-3-{[4-(5-fluoro-1H-indol-3-yl)butyl]amino}chromane-5-carboxylate were separated by chiral HPLC, isolated and converted to the HCl salt generating the following products:

Methyl (3S)-8-fluoro-3-{[4-(5-fluoro-1H-indol-3-yl)butyl]amino}chromane-5-carboxylate hydrochloride salt as a white solid: mp 240° C./dec; [α]_{D}²⁵=−43.6° (c=1% SOLUTION, DMSO); MS (ES) m/z 415.2; Anal. Calcd for C₂₃H₂₄F₂N₂O₃.HCl.0.25 H₂O: C, 60.66; H, 5.64; N, 6.15. Found: C, 60.85; H, 6.05; N, 6.02.

Methyl (3R)-8-fluoro-3-{[4-(5-fluoro-1H-indol-3-yl)butyl]amino}chromane-5-carboxylate hydrochloride salt as a white solid: mp 240° C./dec; [α]_{D}²⁵=+41.44° (c=6.8MG/0.7ML, DMSO); MS (ES) m/z 415.2; Anal. Calcd for C₂₃H₂₄F₂N₂O₃.HCl.0.25 H₂O: C, 60.66; H, 5.64; N, 6.15. Found: C, 60.65; H, 5.80; N, 6.03.

### Example 166

### Methyl 3-{cyclobutyl[4-(5-fluoro-1H-indol-3-yl)butyl]amino}-8-fluorochromane-5-carboxylate (“Compound 58”)

This compound was prepared as described above for example 163 (compound 55). Chromatography ((2:1) Hex-EtOAc) afforded 0.093 g. (69%) of desired product which was converted to the HCl salt to generate methyl 3-{cyclobutyl[4-(5-fluoro-1H-indol-3-yl)butyl]amino}-8-fluorochromane-5-carboxylate hydrochloride salt as a white solid: mp 115° C./dec; MS (ES) m/z 469.2; Anal. Calcd for C₂₇H₃₀F₂N₂O₃.1.20 HCl: C, 63.30; H, 6.14; N, 5.47. Found: C, 63.49; H, 6.09; N, 5.22.

### Examples 166a and 166b

### Methyl (3S)-3-{cyclobutyl[4-(5-fluoro-1H-indol-3-yl)butyl]amino}-8-fluorochromane-5-carboxylate (“Compound 58a”) and Methyl (3R)-3-{cyclobutyl[4-(5-fluoro-1H-indol-3-yl)butyl]amino}-8-fluorochromane-5-carboxylate (“Compound 58b”)

To methyl (3S)-8-fluoro-3-{[4-(5-fluoro-1H-indol-3-yl)butyl]amino}chromane-5-carboxylate (0.43 g, 1.04 mmol) in anhydrous methanol (16 mL), under nitrogen at room temperature, was added cyclobutanone (0.19 mL, 2.6 mmol), acetic acid (0.12 mL, 2.5 mmol) and sodium cyanoborohydride (0.13 g, 2.08 mmol). The reaction mixture was stirred at room temperature overnight. More cyclobutanone (0.19 mL) was added and the reaction mixture stirred at room temperature another night. Chromatography ((6:3:1) Hex-EtOAc-MeOH (1% NH₄OH)) afforded 0.43 g (89%) of desired product which was converted to the HCl salt to generate methyl (3S)-3-{cyclobutyl[4-(5-fluoro-1H-indol-3-yl)butyl]amino}-8-fluorochromane-5-carboxylate hydrochloride salt as a white solid: mp 209° C./dec; [α]_{D}²⁵=+35.82° (c=6.8MG/0.7ML, DMSO); MS (ES) m/z 469.2; Anal. Calcd for C₂₇H₃₀F₂N₂O₃.HCl: C, 64.22; H, 6.19; N, 5.55. Found: C, 63.95; H, 6.18; N, 5.36.

To methyl (3R)-8-fluoro-3-{[4-(5-fluoro-1H-indol-3-yl)butyl]amino}chromane-5-carboxylate (0.38 g, 0.92 mmol) in anhydrous methanol (15 mL), under nitrogen at room temperature, was added cyclobutanone (0.17 mL, 2.3 mmol), acetic acid (0.10 mL, 2.2 mmol) and sodium cyanoborohydride (0.12 g, 1.84 mmol). The reaction mixture was stirred at room temperature overnight. More cyclobutanone (0.17 mL) was added and the reaction mixture stirred at room temperature another night. Chromatography ((6:3:1) Hex-EtOAc-MeOH (1% NH₄OH)) afforded 0.40 g (931%) of desired product which was converted to the HCl salt to generate methyl (3R)-3-{cyclobutyl[4-(5-fluoro-1H-indol-3-yl)butyl]amino}-8-fluorochromane-5-carboxylate hydrochloride salt as a white solid: mp 209° C./dec; [α]_{D}²⁵−33.2° (c=1% SOLUTION, DMSO); MS (ES) nm/z 469.2; Anal. Calcd for C₂₇H₃₀F₂N₂O₃.HCl.0.25 H₂O: C, 63.65; H, 6.23; N, 5.50. Found: C, 63.66; H, 5.86; N, 5.39.

### Example 167

### 3-{cyclobutyl[3-(5-fluoro-1H-indol-3-yl)propyl]amino}-8-fluoro-N-methylchromane-5-carboxamide (“Compound 59”)

To 3-{cyclobutyl[3-(5-fluoro-1H-indol-3-yl)propyl]amino}-8-fluorochromane-5-carboxylic acid (0.1 g, 0.23 mmol) in anhydrous THF (8 mL), under nitrogen at room temperature, was added 1-(3-dimethylaminopropyl)-3-ethylcarbodiimide hydrochloride. (EDC, 0.087 g, 0.45 mmol), 1-hydroxybenzotriazole hydrate (HOBt, 0.66 g, 0.45 mmol) and methylamine (2M/THF, 0.45 mL, 0.91 mmol). The reaction mixture was stirred at room temperature overnight. The reaction mixture was concentrated under vacuum on a rota vap, the residue taken up in dichloromethane/H₂O, the organic layer separated, dried over magnesium sulfate, filtered and concentrated. Chromatography on Biotage Quad using silica gel column and ((6:3:1) Hex-EtOAc-MeOH (1% NH₄OH)) as elution solvent afforded 0.1 g (97%) of desired product which was converted to the HCl salt to generate 3-{cyclobutyl[3-(5-fluoro-1H-indol-3-yl)propyl]amino}-8-fluoro-N-methylchromane-5-carboxamide hydrochloride salt as a white solid: mp 132° C./dec; MS (ES) m/z 454.2.

### Example 168

### 3-{cyclobutyl[3-(5-fluoro-1H-indol-3-yl)propyl]amino}-N-ethyl-8-fluorochromane-5-carboxamide (“Compound 60”)

This compound was prepared as described above for example 167 using ethylamine (2M/THF, 0.45 mL, 0.91 mmol). Chromatography on Biotage Quad afforded 0.092 g (86%) of desired product which was converted to the HCl salt to generate 3-{cyclobutyl[3-(5-fluoro-1H-indol-3-yl)propyl]amino}-N-ethyl-8-fluorochromane-5-carboxamide hydrochloride salt as a white solid: mp 130° C./dec; MS (ES) m/z 468.

### Example 169

### 3-{cyclobutyl[3-(5-fluoro-1H-indol-3-yl)propyl]amino}-8-fluoro-N-propylchromane-5-carboxamide (“Compound 61”)

This compound was prepared as described above for example 167 using propylamine (0.08 mL, 0.91 mmol). Chromatography on Biotage Quad afforded 0.084 g (77%) of desired product which was converted to the HCl salt to generate 3-{cyclobutyl[3-(5-fluoro-1H-indol-3-yl)propyl]amino}-8-fluoro-N-propylchromane-5-carboxamide hydrochloride salt as an off-white solid: mp 67° C./dec; MS (ES) m/z 482.2.

### Example 170

### N-butyl-3-{cyclobutyl[3-(5-fluoro-1H-indol-3-yl)propyl]amino}-8-fluorochromane-5-carboxamide (“Compound 62”)

This compound was prepared as described above for example 167 using butylamine (0.09 mL, 0.91 mmol). Chromatography on Biotage Quad afforded 0.067 g (59%) of desired product which was converted to the HCl salt to generate N-butyl-3-{cyclobutyl[3-(5-fluoro-1H-indol-3-yl)propyl]amino}-8-fluorochromane-5-carboxamide hydrochloride salt as an off-white solid: mp 106° C./dec; MS (ES) m/z 496.

### Example 171

### 3-{cyclobutyl[3-(5-fluoro-1H-indol-3-yl)propyl]amino}-8-fluoro-N-isopropylchromane-5-carboxamide (“Compound 63”)

This compound was prepared as described above for example 167 using isopropylamine (0.08 mL, 0.91 mmol). Chromatography on Biotage Quad afforded 0.1 g (93%) of desired product which was converted to the HCl salt to generate 3-{cyclobutyl[3-(5-fluoro-1H-indol-3-yl)propyl]amino}-8-fluoro-N-isopropylchromane-5-carboxamide hydrochloride salt as a white solid: mp 127° C./dec; MS (ES) m/z 482.2.

### Example 172

### 3-{cyclobutyl[3-(5-fluoro-1H-indol-3-yl)propyl]amino}-N-cyclopropyl-8-fluorochromane-5-carboxamide (“Compound 64”)

This compound was prepared as described above for example 167 using cyclopropylamine (0.07 mL, 0.91 mmol). Chromatography on Biotage Quad afforded 0.092 g (85%) of desired product which was converted to the HCl salt to generate 3-{cyclobutyl[3-(5-fluoro-1H-indol-3-yl)propyl]amino}-N-cyclopropyl-8-fluorochromane-5-carboxamide hydrochloride salt as a white solid: mp 134° C./dec; MS (ES) m/z 480.2.

### Example 173

### N-cyclobutyl-3-{cyclobutyl[3-(5-fluoro-1H-indol-3-yl)propyl]amino}-8-fluorochromane-5-carboxamide (“Compound 65”)

This compound was prepared as described above for example 167 using cyclobutylamine (0.08 mL, 0.91 mmol). Chromatography on Biotage Quad afforded 0.092 g (82%) of desired product which was converted to the HCl salt to generate N-cyclobutyl-3-{cyclobutyl[3-(5-fluoro-1H-indol-3-yl)propyl]amino}-8-fluorochromane-5-carboxamide hydrochloride salt as a white solid: mp 135° C./dec; MS (ES) m/z 494.2.

### Example 174

### 3-{cyclobutyl[3-(5-fluoro-1H-indol-3-yl)propyl]amino}-N-cyclopentyl-8-fluorochromane-5-carboxamide (“Compound 66”)

This compound was prepared as described above for example 167 using cyclopentylamine (0.09 mL, 0.91 mmol). Chromatography on Biotage Quad afforded 0.091 g (79%) of desired product which was converted to the HCl salt to generate 3-{cyclobutyl[3-(5-fluoro-1H-indol-3-yl)propyl]amino}-N-cyclopentyl-8-fluorochromane-5-carboxamide hydrochloride salt as a white solid: mp 133° C./dec; MS (ES) m/z 506.

### Example 175

### 3-{cyclobutyl[3-(5-fluoro-1H-indol-3-yl)propyl]amino}-N-cyclohexyl-8-fluorochromane-5-carboxamide (“Compound 67”)

This compound was prepared as described above for example 167 using cyclohexylamine (0.1 mL, 0.91 mmol). Chromatography on Biotage Quad afforded 0.09 g (76%) of desired product which was converted to the HCl salt to generate 3-{cyclobutyl[3-(5-fluoro-1H-indol-3-yl)propyl]amino}-N-cyclohexyl-8-fluorochromane-5-carboxamide hydrochloride salt as a white solid: mp 134° C./dec; MS (ES) m/z 522.2.

### Example 176

### 3-{cyclobutyl[3-(5-fluoro-1H-indol-3-yl)propyl]amino}-N-(cyclopropylmethyl)-8-fluorochromane-5-carboxamide (“Compound 68”)

This compound was prepared as described above for example 167 using methylcyclopropylamine (0.08 mL, 0.91 mmol). Chromatography on Biotage Quad afforded 0.1 g (92%) of desired product which was converted to the HCl salt to generate 3-{cyclobutyl[3-(5-fluoro-1H-indol-3-yl)propyl]amino}-N-(cyclopropylmethyl)-8-fluorochromane-5-carboxamide hydrochloride salt as a white solid: mp 114° C./dec; MS (ES) m/z 494.2.

### Example 177

### N-benzyl-3-{cyclobutyl[3-(5-fluoro-1H-indol-3-yl)propyl]amino}-8-fluorochromane-5-carboxamide (“Compound 69”)

This compound was prepared as described above for example 167 using benzylamine (0.10 mL, 0.91 mmol). Chromatography on Biotage Quad afforded 0.11 g (93%) of desired product which was converted to the HCl salt to generate N-benzyl-3-{cyclobutyl[3-(5-fluoro-1H-indol-3-yl)propyl]amino}-8-fluorochromane-5-carboxamide hydrochloride salt as a white solid: mp 124° C./dec; MS (ES) m/z 530.2.

### Example 178

### 3-{cyclobutyl[3-(5-fluoro-1H-indol-3-yl)propyl]amino}-8-fluoro-N-phenylchromane-5-carboxamide (“Compound 70”)

This compound was prepared as described above for example 167 using-aniline (0.08 mL, 0.91 mmol). Chromatography on Biotage Quad yielded impure product which was then repurified by flash column chromatography using (3:1) Hex-EtOAc as elution solvent affording 0.087 g (74%) of desired product which was converted to the HCl salt to generate 3-{cyclobutyl[3-(5-fluoro-1H-indol-3-yl)propyl]amino}-8-fluoro-N-phenylchromane-5-carboxamide hydrochloride salt as a white solid: mp 136° C./dec; MS (ES) m/z 516.2.

### Example 179

### 8-fluoro-3-[[3-(5-fluoro-1H-indol-3-yl)propyl](pentyl)amino]chromane-5-carboxamide (“Compound 71”)

This compound was prepared as described above for example 110 (compound 2) using 8-fluoro-3-{[3-(5-fluoro 1H-indol-3-yl)propyl]amino}chromane-5-carboxamide (0.096 g, 0.25 mmol), valeraldehyde (0.032 mL, 0.3 mmol), acetic acid (0.034 mL, 0.6 mmol), sodium cyanoborohydride (0.03 mg, 0.5 mmol) in anhydrous methanol (5 mL). After overnight stirring, more butyraldehyde (0.02 mL), acetic acid (0.018 mL) and sodium cyanoborohydride (0.018 mg) were added, and the reaction mixture stirred at room temperature one more night. Chromatography ((5:4:1) EtOAc-Hex-MeOH (1% NH₄OH)) afforded 0.083 g (73%) of desired product which was converted to the HCl salt to generate 8-fluoro-3-[[3-(5-fluoro-1H-indol-3-yl)propyl](pentyl)amino]chromane-5-carboxamide hydrochloride salt as a white solid: mp 128° C. DEC; MS (ES) m/z 454.2.

### Example 180

### 3-{butyl[3-(5-fluoro-1H-indol-3-yl)propyl]amino}-8-fluorochromane-5-carboxamide (“Compound 72”)

This compound was prepared as described above for example 110 (compound 2) using 8-fluoro-3-{[3-(5-fluoro-1H-indol-3-yl)propyl]amino}chromane-5-carboxamide (0.096 g, 0.25 mmol), butyraldehyde (0.037 mL, 0.3 mmol), acetic acid (0.034 mL, 0.6 mmol), sodium cyanoborohydride (0.03 mg, 0.5 mmol) in anhydrous methanol (5 mL). After overnight stirring, more butyraldehyde (0.02 mL), acetic acid (0.018 mL) and sodium cyanoborohydride (0.018 mg) were added, and the reaction mixture stirred at room temperature one more night. Chromatography ((5:4:1) EtOAc-Hex-MeOH (1% NH₄OH)) afforded 0.093 g (84%) of desired product which was converted to the HCl salt to generate 3-{butyl[3-(5-fluoro-1H-indol-3-yl)propyl]amino}-8-fluorochromane-5-carboxamide hydrochloride salt as a white solid: mp 126° C. DEC; MS (ES). m/z 440.2.

### Example 181

### 3-{cyclobutyl[3-(5-fluoro-1H-indol-3-yl)propyl]amino}-8-fluoro-N,N-dimethylchromane-5-carboxamide (“Compound 73”)

To 3-{cyclobutyl[3-(5-fhuoro-1H-indol-3-yl)propyl]amino}-8-fluorochromane-5-carboxylic acid (0.13 g, 0.29 mmol) in anhydrous THF (10 mL), under nitrogen at room temperature, was added EDC (0.11 g, 0.58 mmol), HOBt (0.078 g, 0.58 mmol) and a 2M solution of dimethylamine in THF (0.58 mL, 1.16 mmol). The reaction mixture was stirred at room temperature overnight. Work up as described above and chromatography ((6:3:1) Hex-EtOAc-MeOH (1% NH₄OH)) afforded 0.12 g (86%) of desired product which was converted to the HCl salt to generate 3-{cyclobutyl[3-(5-fluoro-1H-indol-3-yl)propyl]amino}-8-fluoro-N,N-dimethylchromane-5-carboxamide hydrochloride salt as an off-white solid: mp 126° C./dec; MS (ES) m/z 466.2; Anal. Calcd for C₂₇H₃₁F₂N₃O₂.HCl.H₂O: C, 62.12; H, 6.56; N, 8.05. Found: C, 62.03; H, 6.50; N, 7.90.

### Example 182

### 3-{benzyl[3-(5-fluoro-1H-indol-3-yl)propyllariino}-8-fluorochromane-5-carboxamide (“Compound 74”)

This compound was prepared as described above for example 110 (compound 2) using 8-fluoro-3-{[3-(5-fluoro-1H-indol-3-yl)propyl]amino}chrbmane-5-carboxamide (0.083 g), 0.215 mmol), benzaldehyde (0.08 mL, 0.79 mmol), acetic acid (0.05 mL, 0.87 mmol), sodium cyanoborohydride (0.034 g, 0.50 mmol) in anhydrous methanol (2 mL). After overnight. stirring, more benzaldehyde (0.08 mL, 0.79 mmol), acetic acid (0.05 mL, 0.87 mmol), and sodium cyanoborohydride (0.034 g, 0.50 mmol) were added and the reaction mixture stirred at room temperature for an additional 24 hours. Chromatography ((98:2) CH₂Cl₂—MeOH (5% NH₄OH)) afforded 0.039 g (38%) of desired product which was converted to the HCl salt to generate 3-{benzyl[3-(5-fluoro-1H-indol-3-yl)propyl]amino}-8-fluorochromane-5-carboxamide hydrochloride salt as a light-amber solid: mp 142-150° C. (melts with decomposition); MS (ES) m/z 474.2; Anal. Calcd for C₂₈H₂₇F₂N₃O₂.1.50 HCl.1.30 H₂O: C, 60.74; H, 5.66; N, 7.59. Found: C, 60.45; H, 4.88; N, 7.37.

### Example 183

### 8-fluoro-3-{[2-(5-fluoro-1H-indol-3-yl)ethyl]amino}chromane-5-carboxamide (“Compound 75”)

To 3-amino-8-fluorochromane-5 carboxamide (0.38 g, 1.8 mmol) in anhydrous methanol (29 mL), under nitrogen at room temperature, was added (5-fluoro-1H-indol-3-yl)acetaldehyde (0.33 g, 1.89 mmol), acetic acid (0.23 mL, 4.32 mmol) and sodium cyanoborohydride (0.23 g, 3.6 mmol). The reaction mixture was stirred at room temperature overnight. Chromatography ((5:4:1) EtOAc-Hex-MeOH (1% NIOH)) afforded 0.53 g (79%) of desired product which was converted to the HCl salt to generate 8-fluoro-3-{[2-(5-fluoro-1H-indol-3-yl)ethyl]amino}chromane-5-carboxamide hydrochloride salt as a white solid: mp 134° C./dec; MS (ES) m/z 370.2; Anal. Calcd for C₂₀H₁₉F₂N₃O₂.HCl.0.25 H₂O: C, 58.26; H, 5.01; N, 10.19. Found: C, 58.01; H, 4.94; N, 9.91.

### Example 184

### 3-{ethyl[2-(5-fluoro-1H-indol-3-yl)ethyl]amino}-8-fluorochromane-5-carboxamide (“Compound 76”)

To 8-fluoro-3-{[2-(5-fluoro-1H-indol-3-yl)ethyl]amino}chromane-5-carboxamide (0.1 g, 0.27 mmol) in anhydrous methanol (4.5 mL), under nitrogen at room temperature, was added acetaldehyde (0.018 mL, 0.32 mmol), acetic acid (0.032 mL, 0.65 mmol) and sodium cyanoborohydride (0.034 g, 0.54 mmol). The reaction mixture was stirred at room temperature overnight. Chromatography on Biotage Quad using silica gel column and ((6:3:1) Hex-EtOAc-MeOH (1% NH₄OH)) as elution solvent afforded 0.077 g (72%) of desired product which was converted to the HCl salt to generate 3-{ethyl[2-(5-fluoro-1H-indol-3-yl)ethyl]amino}-8-fluorochromane-5-carboxamide hydrochloride salt as a white solid: mp 127° C./dec; MS (ES) m/z 398.2.

### Example 185

### 8-fluoro-3-[[2-(5-fluoro-1H-indol-3-yl)ethyl](propyl)amino]chromane-5-carboxamide (“Compound 77”)

This compound was prepared as described above for example 184 using propionaldehyde (0.033 mL, 0.46 mmol). Chromatography on Biotage Quad using silica gel column and ((6:3:1) Hex-EtOAc-MeOH (1% NH₄OH)) as elution solvent afforded 0.095 g (85%) of desired product which was converted to the HCl salt to generate 8-fluoro-3-[[2-(5-fluoro-1H-indol-3-yl)ethyl](propyl)amino]chromane-5-carboxamide hydrochloride salt as a white solid: mp 125° C./dec; MS (ES) m/z 412.2.

### Example 186

### 3-{(cyclopropylmethyl)[2-(5-fluoro-1H-indol-3-yl)ethyl]amino}-8-fluorochromane-5-carboxamide (“Compound 78”)

This compound was prepared as described above for example 184 using cyclopropane carboxaldehyde (0.034 mL, 0.46 mmol). Chromatography on Biotage Quad using silica gel column and ((6:3:1) Hex-EtOAc-MeOH (1% NH₄OH)) as elution solvent afforded 0.095 g (83%) of desired product which was converted to the HCl salt to generate 3-{(cyclopropylmethyl)[2-(5-fluoro-1H-indol-3-yl)ethyl]amino}-8-fluorochromane-5-carboxamide hydrochloride salt as a white solid: mp 132° C./dec; MS (ES) m/z 424.2.

### Example 187

### 8-fluoro-3-{[4-(5-fluoro-1H-indol-3-yl)butyl]amino}chromane-5-carboxamide (“Compound 79”)

This compound was prepared as described above for example 183 using 4-(5-fluoro-1H-indol-3-yl)butanal. Chromatography ((5:4:1) EtOAc-Hex-MeOH (1% NH₄OH)) afforded 0.55 g (86%) of desired product which was converted to the HCl salt to generate 8-fluoro-3-{[4-(5-fluoro-1H-indol-3-yl)butyl]amino) chromane-5-carboxamide hydrochloride salt as a white solid: mp 52° C./dec; MS (ES) m/z 398.2; Anal. Calcd for C₂₂H₂₃F₂N₃O₂ 0.50 H₂O: C, 64.69; H, 5.92; N, 10.29. Found: C, 64.89; H, 5.83; N, 10.04.

### Example 188

### 3-{ethyl[4-(5-fluoro-1H-indol-3-yl)butyl]amino}-8-fluorochromane-5-carboxamide (“Compound 80”)

To 8-fluoro-3-{[4-(5-fluoro-1H-indol-3-yl)butyl]amino}chromane-5-carboxamide (0.1 g, 0.25 mmol) in anhydrous methanol (4.0 mL), under nitrogen at room temperature, was-added acetaldehyde (0.017 mL, 0.3 mmol), acetic acid (0.03 mL, 0.6 mmol) and sodium cyanoborohydride (0.032 g, 0.5 mmol). The reaction mixture was stirred at room temperature overnight. Chromatography on Biotage Quad using silica gel column and ((6:3:1) Hex-EtOAc-MeOH (1% NH₄OH)) as elution solvent afforded 0.073 g (69%) of desired product which was converted to the HCl salt to generate 3-{ethyl[4-(5-fluoro-1H-indol-3-yl)butyl]amino}-8-fluorochromane-5-carboxamide hydrochloride salt as a white solid: mp 110° C./dec; MS (ES) m/z 426.3.

### Example 189

### 8-fluoro-3-[[4-(5-fluoro-1H-indol-3-yl)butyl](propyl)amino]chromane-5-carboxamide (“Compound 81”)

This compound was prepared as described above for example 188 using propionaldehyde (0.033 mL, 0.46 mmol). Chromatography on Biotage Quad using silica gel column and ((6:3:1) Hex-EtOAc-MeOH (1% NH₄OH)) as elution solvent afforded 0.093 g (84%) of desired product which was converted to the HCl salt to generate 8-fluoro-3-[[4-(5-fluoro-1H-indol-3-yl)butyl](propyl)amino]chromane-5-carboxamide hydrochloride salt as a white solid: mp 115° C./dec; MS (ES) m/z 440.3.

### Example 190

### 3-{(cyclopropylmethyl)[4-(5-fluoro-1H-indol-3-yl)butyl]amino}-8-fluorochromane-5-carboxamide (“Compound 82”)

This compound was prepared as described above for example 188 using cyclopropane carboxaldehyde (0.032 mL, 0.46 mmol). Chromatography on Biotage Quad using silica gel column and ((6:3:1) Hex-EtOAc-MeOH (1% NH₄OH)) as elution solvent afforded 0.096 g (84%) of desired product which was converted to the HCl salt to generate 3-{(cyclopropylmethyl)[4-(5-fluoro-1H-indol-3-yl)butyl]amino}-8-fluorochromane-5-carboxamide hydrochloride salt as a white solid: mp 115° C./dec; MS (ES) m/z 452.2.

### Example 191

### 3-{cyclobutyl[4-(5-fluoro-1H-indol-3-yl)butyl]amino}-8-fluorochromane-5-carboxamide (“Compound 83”)

This compound was prepared as described above for example 188 using cyclobutanone (0.062 mL, 0.83 mmol). Chromatography on Biotage Quad using silica gel column and ((6:3:1) Hex-EtOAc-MeOH (1% NH₄OH)) as elution solvent afforded 0.10 g (88%) of desired product which was converted to the HCl salt to generate 3-{cyclobutyl[4-(5-fluoro-1H-indol-3-yl)butyl]amino}-8-fluorochromane-5-carboxamide hydrochloride salt as a white solid: mp 122° C./dec; MS (ES) m/z 452.2.

### Examples 191a and 191b

### (3R)-3-{cyclobutyl[4-(5-fluoro-1H-indol-3-yl)butyl]amino}-8-fluorochromane-5-carboxamide (“Compound 83a”) and (3S)-3-{cyclobutyl[4-(5-fluoro-1H-indol-3-yl)butyl]amino}-8-fluorochromane-5-carboxamide (“Compound 83b”)

3-{cyclobutyl[4-(5-fluoro-1H-indol-3-yl)butyl]amino}-8-fluorochromane-5-carboxamide was resynthesized on a larger scale (0.9 g, 2.3 mmol) of starting material as described for example 191 and the enantiomers were separated by chiral HPLC, isolated and converted to the HCl salt generating the following products:

(3R)-3-{cyclobutyl[4-(5-fluoro-1H-indol-3-yl)butyl]amino}-8-fluorochromane-5-carboxamide hydrochloride salt as a white solid: mp 128° C./dec; [α]_{D}²⁵=−29.4° (c=1% SOLUTION, DMSO); MS (ES) m/z 454.2; Anal. Calcd for C₂₆H₂₉F₂N₃O₂.HCl.0.25 H₂O: C, 63.15; H, 6.22; N, 8.50. Found: C, 63.21; H, 5.84; N, 8.39.

(3S)-3-{cyclobutyl[4-(5-fluoro-1H-indol-3-yl)butyl]amino}-8-fluorochromane-5-carboxamide hydrochloride salt as a white solid: mp 128° C./dec; [α]_{D}²⁵=+31.00° (c=1% SOLUTION, DMSO); MS (ES) m/z 454.2; Anal. Calcd for C₂₆H₂₉F₂N₃O₂.HCl.0.25 H₂O: C, 63.15; H, 6.22; N, 8.50. Found: C, 63.20; H, 5.92; N, 8.38.

### Example 192

### 8-fluoro-3-{[2-(5-fluoro-1H-indol-3-yl)ethyl]amino}-N-methylchromane-5-carboxamide (“Compound 84”)

To 3-amino-8-fluoro-N-methylchromane-5-carboxamide (0.41 g, 1.8 mmol) in anhydrous methanol (29 mL), under nitrogen at room temperature, was added (5-fluoro-1H-indol-3-yl)acetaldehyde (0.34 g, 1.92 mmol), acetic acid (0.23 mL, 4.32 mmol) and sodium cyanoborohydride (0.23 g, 3.6 mmol). The reaction mixture was stirred at room temperature overnight. Chromatography ((5:4:1) EtOAc-Hex-MeOH (1% NH₄OH)) afforded 0.58 g (82%) of desired product which was converted to the HCl salt to generate 8-fluoro-3-{[2-(5-fluoro-1H-indol-3-yl)ethyl]amino}-N-methylchromane-5-carboxamide hydrochloride salt as a white solid: mp 148° C./dec; MS (ES) m/z 386.1; Anal. Calcd for C₂₁H₂₁F₂N₃O₂.HCl.0.75 H₂O: C, 57.93; H, 5.44; N, 9.65. Found: C, 57.81; H, 5.47; N, 9.30.

### Example 193

### 3-{ethyl[2-(5-fluoro-1H-indol-3-yl)ethyl]amino}-8-fluoro-N-methylchromane-5-carboxamide (“Compound 85”)

To 8-fluoro-3-{[2-(5-fluoro-1H-indol-3-yl)ethyl]amino}-N-methylchromane-5-carboxamide (0.1 g, 0.26 mmol) in anhydrous methanol (4.2 mL), under nitrogen at room temperature, was added acetaldehyde (0.018 mL, 0.31 mmol), acetic acid (0.03 mL, 0.6 mmol) and sodium cyanoborohydride (0.033 g, 0.52 mmol). The reaction mixture was stirred at room temperature overnight. Chromatography on Biotage Quad using silica gel column and ((6:3:1) Hex-EtOAc-MeOH (1% NH₄OH)) as elution solvent afforded 0.093 g (87%) of desired product which was converted to the HCl salt to generate 3-{ethyl[2-(5-fluoro-1H-indol-3-yl)ethyl]amino}-8-fluoro-N-methylchromane-5-carboxamide hydrochloride salt as a white solid: mp 125° C./dec; MS (ES) m/z 412.2.

### Example 194

### 3-{(cyclopropylmethyl)[2-(5-fluoro-1H-indol-3-yl)ethyl]amino}-8-fluoro-N-methylchromane-5-carboxamide (“Compound 86”)

This compound was prepared as described above for example 193 using cyclopropane carboxaldehyde (0.032 mL, 0.46 mmol). Chromatography on Biotage Quad using silica gel column and ((6:3:1) Hex-EtOAc-MeOH (1% NH₄OH)) as elution solvent afforded 0.056 g (49%) of desired product which was converted to the HCl salt to generate 3-{(cyclopropylmethyl)[2-(5-fluoro-1H-indol-3-yl)ethyl]amino}-8-fluoro-N-methylchromane-5-carboxamide hydrochloride salt as a white solid: mp 138° C./dec; MS (ES) m/z 438.2.

### Example 195

### 3-{cyclobutyl[2-(5-fluoro-1H-indol-3-yl)ethyl]amino}-8-fluoro-N-methylchromane-5-carboxamide (“Compound 87”)

This compound was prepared as described above for example 193 using cyclobutanone (0.062 mL, 0.83 mmol). Chromatography on Biotage Quad using silica gel column and ((6:3:1) Hex-EtOAc-MeOH (1% NH₄OH)) as elution solvent afforded 0.10 g (91%) of desired product which was converted to the HCl salt to generate 3-{cyclobutyl[2-(5-fluoro-1H-indol-3-yl)ethyl]amino}-8-fluoro-N-methylchromane-5-carboxamide hydrochloride salt as a white solid: mp 131° C./dec; MS (ES) m/z 438.2.

### Example 196

### 8-fluoro-3-{[3-(5-fluoro-1H-indol-3-yl)propyl]amino}-N-methylchromane-5-carboxamide (“Compound 88”)

This compound was prepared as described above for example 192 using 3-(5-fluoro-1H-indol-3-yl)propanal. Chromatography ((5:4:1) EtOAc-Hex-MeOH (1% NH₄OH)) afforded 0.52 g (87%) of desired product which was converted to the HCl salt to generate 8-fluoro-3-{[3-(5-fluoro-1H-indol-3-yl)propyl]amino}-N-methylchromane-5-carboxamide hydrochloride salt as a white solid: mp 148° C./dec; MS (ES) m/z 400.2; Anal. Calcd for C₂₂H₂₃F₂N₃O₂.HCl.0.50 H₂O: C, 59.39; H, 5.66; N, 9.44. Found: C, 59.10; H, 5.65; N, 9.11.

### Example 197

### 3-{ethyl[3-(5-fluoro-1H-indol-3-yl)propyl]amino}-8-fluoro-N-methylchromane-5-carboxamide (“Compound 89”)

To 8-fluoro-3-{[3-(5-fluoro-1H-indol-3-yl)propyl]amino}-N-methylchromane-5-carboxamide (0.1 g, 0.25 mmol) in anhydrous methanol (4.0 mL), under nitrogen at room temperature, was added acetaldehyde (0.017 mL, 0.31 mmol), acetic acid (0.03 mL, 0.6 mmol) and sodium cyanoborohydride (0.032 g, 0.5 mmol). The reaction mixture was stirred at room temperature overnight. Chromatography on Biotage Quad using silica gel column and ((6:3:1) Hex-EtOAc-MeOH (1% NH₄OH)) as elution solvent afforded 0.085 g (79%) of desired product which was converted to the HCl salt to generate 3-{ethyl[3-(5-fluoro-1H-indol-3-yl)propyl]amino}-8-fluoro-N-methylchromane-5-carboxamide hydrochloride salt as a white solid: mp 118° C./dec; MS (ES) m/z 426.3.

### Example 198

### 8-fluoro-3-[[3-(5-fluoro-1H-indol-3-yl)propyl](propyl)amino]-N-methylchromane-5-carboxamide (“Compound 90”)

This compound was prepared as described above for example 197 using propionaldehyde (0.033 mL, 0.46 mmol). Chromatography on Biotage Quad using silica gel column and ((6:3:1) Hex-EtOAc-MeOH (1% NH₄OH)) as elution solvent afforded 0.099 g (89%) of desired product which was converted to the HCl salt to generate 8-fluoro-3-[[3-(5-fluoro-1H-indol-3-yl)propyl](propyl)amino]-N-methylchromane-5-carboxamide hydrochloride salt as a white solid: mp 123° C./dec; MS (ES) m/z 440.2.

### Example 199

### 3-{(cyclopropylmethyl)[3-(5-fluoro-1H-indol-3-yl)propyl]amino}-8-fluoro-N-methylchromane-5-carboxamide (“Compound 91”)

This compound was prepared as described above for example 197 using cyclopropane carboxaldehyde (0.032 mL, 0.46 mmol). Chromatography on Biotage Quad using silica gel column and ((6:3:1) Hex-EtOAc-MeOH (1% NH₄OH)) as elution solvent afforded 0.10 g (88%) of desired product which was converted to the HCl salt to generate 3-{(cyclopropylmethyl)[3-(5-fluoro-1H-indol-3-yl)propyl]amino}-8-fluoro-N-methylchromane-5-carboxamide hydrochloride salt as a white solid: mp 120° C./dec; MS (ES) m/z 452.2.

### Example 200

### 8-fluoro-3-{[4-(5-fluoro-1H-indol-3-yl)butyl]amino}-N-methylchromane-5-carboxamide (“Compound 92”)

This compound was prepared as described above for example 196 using 4-(5-fluoro-1H-indol-3-yl)butanal. Chromatography ((5:4:1) EtOAc-Hex-MeOH (1% NH₄OH)), afforded 0.58 g (87%) of desired product which was converted to the HCl salt to generate 8-fluoro-3-{[4-(5-fluoro-1H-indol-3-yl)butyl]amino}-N-methylchromane-5-carboxamide hydrochloride salt as a white solid: mp 130° C./dec; MS (ES) m/z 412.2; Anal. Calcd for C₂₃H₂₅F₂N₃O₂.HCl.0.50 H₂O: C, 60.19; H, 5.93; N, 9.16. Found: C, 60.13; H, 5.71; N, 8.94.

### Example 201

### 8-fluoro-3-[[4-(5-fluoro-1H-indol-3-yl)butyl](propyl)amino]-N-methylchromane-5-carboxamide (“Compound 93”)

To 8-fluoro-3-{[4-(5-fluoro-1H-indol-3-yl)butyl]amino}-N-methylchromane-5-carboxamide (0.1 g, 0.24 mmol) in anhydrous methanol (4.0 mL), under nitrogen at room temperature, was added propionaldehyde (0.031 mL, 0.43 mmol), acetic acid (0.029 mL, 0.58 mmol) and sodium cyanoborohydride (0.03 g, 0.48 mmol). The reaction mixture was stirred at room temperature overnight. Chromatography on Biotage Quad using silica gel column and ((6:3:1) Hex-EtOAc-MeOH (1% NH₄OH)) as elution solvent afforded 0.096 g (88%) of desired product which was converted to the HCl salt to generate 8-fluoro-3-[[4-(5-fluoro-1H-indol-3-yl)butyl](propyl)amino]-N-methylchromane-5-carboxamide hydrochloride salt as a white solid: mp 110° C./dec; MS (ES) m/z 454.2.

### Example 202

### 3-{(cyclopropylmethyl)[4-(5-fluoro-1H-indol-3-yl)butyl]amino}-8-fluoro-N-methylchromane-5-carboxamide (“Compound 94”)

This compound was prepared as described above for example 201 using cyclopropane carboxaldehyde (0.032 mL, 0.46 mmol). Chromatography on Biotage Quad using silica gel column and ((6:3:1) Hex-EtOAc-MeOH (1% NH₄OH)) as elution solvent afforded 0.095 g (85%) of desired product which was converted to the HCl salt to generate 3-{(cyclopropylmethyl)[4-(5-fluoro-1H-indol-3-yl)butyl]amino}-8-fluoro-N-methylchromane-5-carboxamide hydrochloride salt as a white solid: mp 112° C./dec; MS (ES) m/z 466.2.

### Example 203

### 3-{cyclobutyl[4-(5-fluoro-1H-indol-3-yl)butyl]amino}-8-fluoro-N-methylchromane-5-carboxamide (“Compound 95”)

This compound was prepared as described above for example 201 using cyclobutanone (0.062 mL, 0.83 mmol). Chromatography on Biotage Quad using silica gel column and ((6:3:1) Hex-EtOAc-MeOH (1% NH₄OH)) as elution solvent afforded 00.10 g (89%) of desired product which was converted to the HCl salt to generate 3-{cyclobutyl[4-(5-fluoro-1H-indol-3-yl)butyl]amino}-8-fluoro-N-methylchromane-5-carboxamide hydrochloride salt as a white solid: mp 123° C./dec; MS (ES) m/z 466.2.

### Example 204

### 3-{[3-(5-cyano-1H-indol-3-yl)propyl]amino}-8-fluorochromane-5-carboxamide (“Compound 96”)

To a solution of 3-amino-8-fluorochromane-5-carboxamide (0.775 g, 3.69 mmol) and N,N-diisopropylethylamine (1.60 mL, 9.2 mmol) in anhydrous DMSO (20 mL) was added 3(3-bromopropyl)-1H-indole-5-carbonitrile (1.17 g, 4.43 mmol). The reaction mixture was stirred at 80° C. overnight. After cooling to ambient temperature, the reaction mixture was diluted with H₂O (200 mL), and extracted with ethyl acetate (2×200 mL). The combined organic phases were washed with saturated aqueous NaCl, dried over MgSO₄, and evaporated under reduced pressure to a gum. Chromatography ((96:4) CH₂Cl₂—MeOH (5% NHOH)) afforded 0.677 g (47%) of the desired product as an off-white solid. Its identity was confirmed by ¹HNMR.

### Examples 205, 205a and 205b

### 3-[[3-(5-cyano-1H-indol-3-yl)propyl](cyclobutyl)amino]-8-fluorochromane-5-carboxamide (“Compound 97”), (3S)-3-[[3-(5-cyano-1H-indol-3-yl)propyl](cyclobutyl)amino]-8-fluorochromane-5-carboxamide (“Compound 97a”) and (3R)-3-[[3-(5-cyano-1H-indol-3-yl)propyl](cyclobutyl)amino]-8-fluorochromane-5-carboxamide (“Compound 97b”)

Compound 97 was prepared as described above for example 112 (compound 4) using 3-{[3-(5-cyano-1H-indol-3-yl)propyl]amino}-8-fluorochromane-5-carboxamide (0.4 g, 1.02 mmol), cyclobutanone (0.28 mL, 3.71 mmol), acetic acid (0.16 mL, 2.8 mmol), and sodium cyanoborohydride (0.157 g, 2.55 mmol) in anhydrous methanol. (12 mL). After overnight stirring more cyclobutanone (0.28 mL, 3.71 mmol), acetic acid (0.16 mL, 2.8 mmol) and sodium cyanoborohydride (0.157 g, 2.55 mol) were added, and the reaction mixture stirred for another night. Chromatography ((96:4) CH₂Cl₂—MeOH (5% NH₄OH)) afforded 0.381 g (84%) of 3-[[3-(5-cyano-1H-indol-3-yl)propyl](cyclobutyl)amino]-8-fluorochromane-5-carboxamide as a white solid. Its identity was confirmed by ¹HNMR.

The enantiomers of 3-[[3-(5-cyano-1H-indol-3-yl)propyl](cyclobutyl)amino]-8-fluorochromane-5-carboxamide were separated by chiral HPLC, isolated and converted-to the HCl salt generating the following products:

(3S)-3-[[3-(5-cyano-1H-indol-3-yl)propyl](cyclobutyl)amino]-8-fluorochromane-5-carboxamide hydrochloride salt as a white solid: mp 170-177° C. (melts with decomposition); [α]_{D}=+19.0° (c=1% SOLUTION, DMSO); MS (ES) m/z 445.2; Anal. Calcd for C₂₆H₂₇FN₄O₂.HCl.0.80 H₂O: C, 62.78; H, 6.00; N, 11.26. Found: C, 62.69; H, 6.23; N, 10.07.

(3R)-3-[[3-(5-cyano-1H-indol-3-yl)propyl](cyclobutyl)amino]-8-fluorochromane-5-carboxamide hydrochloride salt as a white solid: mp 166-171° C. (melts with decomposition); [α]_{D}²⁵=−32.60 (c=1% SOLUTION, DMSO); MS (ES) m/z 445.2; Anal. Calcd for C₂₆H₂₇FN₄O₂.HCl.0.40 H₂O: C, 63.71; H, 5.92; N, 11.43. Found: C, 64.03; H, 6.28; N, 10.69.

### Examples 206, 206a and 206b

### 3-[[3-(5-cyano-1H-indol-3-yl)propyl](cyclopropylmethyl)amino]-8-fluorochromane-5-carboxamide (“Compound 98”) (3S)-3-[[3-(5-cyano-1H-indol-3-yl)propyl](cyclopropylmethyl)amino]-8-fluorochromane-5-carboxamide (“Compound 98a”) and (3R)-3-[[3-(5-cyano-1H-indol-3-yl)propyl](cyclopropylmethyl)amino]-8-fluorochromane-5-carboxaminde (“Compound 98b”)

Compound 98 was prepared as described above for example 205 using 3-{[3-(5-cyano-1H-indol-3-yl)propyl]amino}-8-fluorochromane-5-carboxamide (0.4 g, 1.02 mmol), cyclopropanecarboxaldehyde (0.28 mL, 3.71 mmol), acetic acid (0.15 mL, 2.7 mmol), and sodium cyanoborohydride (0.14 g, 2.24 mmol) in anhydrous methanol (10 mL). After overnight stirring more cyclopropanecarboxaldehyde (0.28 mL, 3.71 mmol), acetic acid (0.15 mL, 2.7 mmol) and sodium cyanoborohydride (0.14 g, 2.24 mol) were added, and the reaction mixture stirred for another night. Chromatography ((95:5) CH₂Cl₂—MeOH (5% NH₄OH)) afforded 0.41 g (90%) of 3-[[3-(5-cyano-1H-indol-3-yl)propyl](cyclopropylmethyl)amino]-8-fluorochromane-5-carboxamide as an off-white solid. Its identity was confirmed by ¹HNMR.

The enantiomers of 3-[[3-(5-cyano-1H-indol-3-yl)propyl](cyclopropylmethyl) amino]-8-fluorochromane-5-carboxamide were separated by chiral HPLC, isolated and converted to the HCl salt generating the following products:

(3S)-3-[[3-(5-cyano-1H-indol-3-yl)propyl](cyclopropylmetbyl)amino]-8-fluorochromane-5-carboxamide hydrochloride salt as a white solid: mp 162-168° C. (melts with decomposition); [α]_{D}²⁵=+20.00° (c=1% SOLUTION, DMSO); MS (ES) m/z 445.2; Anal. Calcd for C₂₆H₂₇FN₄O₂.HCl.0.50 H₂O: C, 63.47; H, 5.94; N, 11.39. Found: C, 63.68; H, 6.08; N, 10.80.

(3R)-3-[[3-(5-cyano-1H-indol-3-yl)propyl](cyclopropylmethyl)amino]-8-fluorochromane-5-carboxamide hydrochloride salt as a whites solid: mp 160-167° C. (melts with decomposition); [α]_{D}²⁵=−35.8° (c=1% SOLUTION, DMSO); MS (ES) m/z 445.2; Anal. Calcd for C₂₆H₂₇FN₄O₂.HCl.0.20 H₂O: C, 64.18; H, 5.88; N, 11.51. Found: C, 63.26; H, 6.20; N, 10.70.

### Example 207

### 8-fluoro-3-{[3-(7-methoxy-1H-indol-3-yl)propyl]amino}chromane-5-carboxamide (“Compound 99”)

A solution of 3-amino-8-fluorochromane-5-carboxamide (0.22 g, 1.03 mmol) and 3-(7-methoxy-1H-indol-3-yl)propanal (0.23 g, 1.13 mmol) in anhydrous methanol (15 mL) and acetic acid (0.14 mL, 2.5 mmol) was treated with sodium cyanoborohydride (0.13 g, 2.05 mmol). After stirring for 18 hours at ambient temperature the reaction was quenched with 1N aqueous NaOH (2 mL) and extracted with ethyl acetate (4×25 mL). The combined organic phases were washed with saturated aqueous NaCl, dried over MgSO₄, and evaporated under reduced pressure. Chromatography ((95:5) CH₂Cl₂—MeOH (5% NH₄OH)) afforded 0.28 g (68%) of desired product which was converted to the HCl salt to generate 8-fluoro-3-{[3-(7-methoxy-1H-indol-3-yl)propyl]amino}chromane-5-carboxamide hydrochloride salt as a white solid: mp 196-198° C.; MS (ES) m/z 398.1; Anal. Calcd for C₂₂H₂₄FN₃O₃.HCl.0.30 H₂O: C, 60.15; H, 5.87; N, 9.56. Found: C, 60.18; H, 6.08; N, 8.96.

### Example 208

### 8-fluoro-3-[[3-(7-methoxy-1H-indol-3-yl)propyl](propyl)amino]chromane-5-carboxamide (“Compound 100”)

This compound was prepared as described above for example110 (compound 2) using 8-fluoro-3-{[3-(7-methoxy-1H-indol-3-yl)propyl]amino}chromane-5-carboxamide (0.12 g, 0.31 mmol), propionaldehyde (0.082 mL, 1.1 mmol), acetic acid (0.053 mL, 0.93 mmol), and sodium cyanoborohydride (0.049 g, 0.78 mmol) in anhydrous methanol (1.3 mL). The reaction mixture was stirred at room temperature overnight. Chromatography on Biotage Quad using silica gel column and ((92:8) CH₂Cl₂: MeOH(5% NH₄OH) as elution solvent afforded 0.092 g (68%) of desired product which was converted to the HCl salt to generate 8-fluoro-3-[[3-(7-methoxy-1H-indol-3-yl)propyl](propyl)amino]chromane-5-carboxamide hydrochloride salt as a white solid: mp 158-164° C. (melts with decomposition); MS (ES) m/z 439.2.

### Example 209

### 3-{ethyl[3-(7-methoxy-1H-indol-3-yl)propyl]amino}-8-fluorochromane-5-carboxamide (“Compound 101”)

This compound was prepared as described above for example 208 using acetaldehyde (0.064 mL, 1.1 mmol) affording 0.13 g (100%) of desired product which was converted to the HCl salt to generate 3-{ethyl[3-(7-methoxy-1H-indol-3-yl)propyl]amino}-8-fluorochromane-5-carboxamide hydrochloride salt as a white solid: mp 148-153° C. (melts with decomposition); MS (ES) m/z 425.2.

### Example 210

### 3-{cyclobutyl[3-(7-methoxy-1H-indol-3-yl)propyl[amino}-8-fluorochromane-5-carboxamide (“Compound 102”)

This compound was prepared as described above for example 208 using cyclobutanone (0.085 mL, 1.1 mmol) affording 0.11 g (81%) of desired product which was converted to the HCl salt to generate 3-{cyclobutyl[3-(7-methoxy-1H-indol-3-yl)propyl]amino}-8-fluorochromane-5-carboxamide hydrochloride salt as a white solid: mp 156-162° C. (melts with decomposition); MS (ES) m/z 452.2.

### Example 211

### 3-[(cyclopropylmethyl)[3-(7-methoxy-1H-indol-3-yl)propyllaiino)-8-fluorochromane-5-carboxamide (“Compound 103”)

This compound was prepared as described above for example 208 using cyclopropane carboxaldehyde (0.085 mL, 1.1 mmol) affording 0.11 g (80%) of desired product which was converted to the HCl salt to generate 3-{(cyclopropylmethyl)[3-(7-methoxy-1H-indol-3-yl)propyl]amino}-8-fluorochromane-5-carboxamide hydrochloride salt as a white solid: mp 152-158° C. (melts with decomposition); MS (ES) m/z 452.2.

### Example 212

### 8-fluoro-3-{[3-(5-methoxy-1H-indol-3-yl)propyl]amino}chromane-5-carboxamide (“Compound 104”)

A solution of 3-amino-8-fluorochromane-5-carboxamide (0.64 g, 3.02 mmol) and 3-(3-bromopropyl)-5-methoxy-1H-indole (1.05 g, 3.92 mmol) in N,N-diisopropylethylamine (1.3 mL, 7.4 mmol) and anhydrous DMSO (13 mL) was stirred for 20 hours at 85° C. The cooled solution was diluted with 1N aqueous NaOH (70 mL) and extracted with EtOAc (3×75 mL). The combined organic phases were washed with saturated aqueous NaCl, dried over MgSO₄, and evaporated under reduced pressure to a dark oil. Chromatography ((95:5) CH₂Cl₂— methanol(5% NH₄OH) afforded 0.75 g (62%) of desired product which was converted to the HCl salt to generate 8-fluoro-3-{[3-(5-methoxy-1H-indol-3-yl)propyl]amino}chromane-5-carboxamide hydrochloride salt as a reddish-white solid: mp 171-176° C. (melts with decomposition); MS (ES) m/z 398.1; Anal. Calcd for C₂₂H₂₄FN₃O₃.HCl.0.30 H₂O: —C, 60.15; H, 5.87; N, 9.56. Found: C, 59.96; H, 6.24; N, 8.55.

### Example 213

### 3-{ethyl[3-(5-methoxy-1H-indol-3-yl)propyl]aniino}-8-fluorochromane-5-carboxamide (“Compound 105”)

This compound was prepared as described above for example 110 (compound 2) using 8-fluoro-3-{[3-(5-methoxy-1H-indol-3-yl)propyl]amino}chromane-5-carboxamide (0.09 g, 0.23 mmol), acetaldehyde (0.05 mL, 0.89 mmol), acetic acid (0.04 mL, 0.7 mmol), and sodium cyanoborohydride (0.036 g, 0.78 mmol) in anhydrous methanol, (11.0 mL). The reaction mixture was stirred at room temperature overnight. Chromatography on Biotage Quad using silica gel column and ((92:8) CH₂Cl₂: MeOH(5% NH₄OH) as elution solvent afforded 0.082 g (85%) of desired product which was converted to the HCl salt to generate 3-(ethyl[3-(5-methoxy-1H-indol-3-yl)propyl]amino}-8-fluorochromane-5-carboxamide hydrochloride salt as a white solid:

mp 154-162° C. (melts with decomposition); MS (ES) m/z 426.2.

### Example 214

### 8-fluoro-3-[[3-(5-methoxy-1H-indol-3-yl)propyl](propyl)amino]chromane-5-carboxamide (“Compound 106”)

This compound was prepared as described above for example 213 using 8-fluoro-3-{[3-(5-methoxy-1H-indol-3-yl)propyl]amino}chromane-5-carboxamide (0.12 g, 0.31 mmol) and propionaldehyde (0-0.082 mL, 1.1 mmol) affording 0.12 g (86%) of desired product which was converted to the HCl salt to generate 8-fluoro-3-[[3-(5-methoxy-1H-indol-3-yl)propyl](propyl)amino]chromane-5-carboxamide hydrochloride salt as a white solid: mp 145-152° C. (melts with decomposition); MS (ES) m/z 440.2.

### Example 215

### 3-{cyclobutyl[3-(5-methoxy-1H-indol-3-yl)propyl]amino}-8-fluorochromane-5-carboxamide (“Compound 107”)

This compound was prepared as described above for example 213 using cyclobutanone (0.085 mL, 1.1 mmol) affording 0.13 g (93%) of desired product which was converted to the HCl salt to generate 3-{cyclobutyl[3-(5-methoxy-1H-indol-3-yl)propyl]amino}-8-fluorochromane-5-carboxamide hydrochloride salt as a white solid: mp 151-158° C. (melts with decomposition); MS (ES) m/z 452.3.

### Example 216

### 3-{(cyclopropylmethyl)[3-(5-methoxy-1H-indol-3-yl)propyl]amino}-8-fluorochromane-5-carboxamide (“Compound 108”)

This compound was prepared as described above for example 213 using cyclopropanecarboxaldehyde (0.085 mL, 1.1 mmol) affording 0.13 g (96%) of desired product which was converted to the HCl salt to generate 3-{(cyclopropylmethyl)[3-(5-methoxy-1H-indol-3-yl)propyl]amino}-8-fluorochromane-5-carboxamide hydrochloride salt as a white solid: mp 148-154° C. (melts with decomposition); MS m/z 451.2.

### Example 217

### 3-{[3-(7-chloro-1H-indol-3-yl)propyl]amino}-8-fluorochromane-5-carboxamide (“Compound 109”)

A solution of 3-amino-8-fluorochromane-5-carboxamide (0.48 g, 2.30 mmol) and 3-(7-chloro-1H-indol-3-yl)propanal (0.5 g, 2.41 mmol) in anhydrous methanol (32 mL) and acetic acid (0.32 mL, 5.5 mmol) was treated with sodium cyanoborohydride (0.29 g, 4.60 mmol). After stirring for 20 hours at ambient temperature the reaction was quenched with 1N aqueous NaOH (50 mL) and extracted with ethyl acetate (4×50 mL). The combined organic phases are washed with saturated aqueous NaCl, dried over MgSO₄, and evaporated under reduced pressure. Chromatography ((95:5) CH₂Cl₂—MeOH (5% NH₄OH)) afforded 0.73 g (79%) of desired product which was converted to the HCl salt to generate 3-{[3-(7-chloro-1H-indol-3-yl)propyl]amino}-8-fluorochromane-5-carboxamide hydrochloride salt as a white solid: mp 222-224° C.; MS (ES) m/z 402.1; Anal. Calcd for C21H21ClFN302. HCl: C, 57.54; H, 5.06; N, 9.59. Found: C, 57.61; H, 4.87; N, 9.36.

### Example 218

### 3-[[3-(7-chloro-1H-indol-3-yl)propyl](ethyl)amino]-8-fluorochromane-5-carboxamide (“Compound 110”)

This compound was prepared as described for example 208 using 3-{[3-(7-chloro-1H-indol-3-yl)propyl]amino}-8-fluorochromane-5-carboxamide (0.124 g, 0.31 mmol) and acetaldehyde (0.064 mL, 1.1 mmol) affording 0.130 g (98%) of the desired product which was converted to the HCl salt to generate 3-[[3-(7-chloro-1H-indol-3-yl)propyl](ethyl)amino]-8-fluorochromane-5-carboxamide hydrochloride salt as a white solid: mp 146-152° C. (melts with decomposition); MS (ES) m/z 430.2.

### Example 219

### 3-[[3-(7-chloro-1H-indol-3-yl)propyl](cyclobutyl)amino]-8-fluorochromane-5-carboxamide (“Compound 111”)

This compound was prepared as described above for example 218 using cyclobutanone (0.085 mL, 1.1 mmol) affording 0.133 g (97%) of desired product which was converted to the HCl salt to generate 3-[[3-(7-chloro-1H-indol-3-yl)propyl](cyclobutyl)amino]-8-fluorochromane-5-carboxamide hydrochloride salt as a white sold: mp 154-163° C./dec; MS m/z 455.2.

### Example 220

### 3-[[3-(7-chloro-1H-indol-3-yl)propyl](cyiclopropylmethyl)amino]-8-fluorochromane-5-carboxamide (“Compound 112”)

This compound was prepared as described above for example 218 using cyclopropanecarboxaldehyde (0.085 mL, 1.1 mmol) affording 0.136 g (96%) of desired product which was converted to the HCl salt to generate 3-[[3-(7-chloro-1H-indol-3-yl)propyl](cyclopropylmethyl)amino]-8-fluorochromane-5-carboxamide hydrochloride salt as a white solid: mp 147-155° C. (melts with decomposition); MS (ES) m/z 456.2.

### Example 221

### 3-[[3-(7-chloro-1H-indol-3-yl)propyl](propyl)amino]-8-fluorochromane-5-carboxamide (“Compound 113”)

This compound was prepared as described above for example 218 using propionaldehyde (0.082 mL, 1.1 mmol) affording 0.133 g (97%) of desired product which was converted to the HCl salt to generate 3-[[3-(7-chloro-1H-indol-3-yl)propyl](propyl)amino]-8-fluorochromane-5-carboxamide hydrochloride salt as a white solid: mp 145-154° C. (melts with decomposition); MS (ES) m/z 443.2.

### Example 222

### 3-{[3-(5-chloro-1H-indol-3-yl)propyl]amino}-8-fluorochromane-5-carboxamide (“Compound 114”)

A solution of 3-amino-8-fluorochromane-5-carboxamide (0.48 g, 2.30 mmol) and 3-(5-chloro-1H-indol-3-yl)propanal (0.5 g, 2.41 mmol) in anhydrous methanol (32 mL) and acetic acid (0.32 mL, 5.5 mmol) was treated with sodium cyanoborohydride (0.29 g, 4.60 mmol). After stirring for 20 hours at ambient temperature the reaction was quenched with 1N aqueous NaOH (50 mL) and extracted with ethyl acetate (4×50 mL). The combined organic phases were washed with saturated aqueous NaCl, dried over MgSO₄, and evaporated under reduced pressure. Chromatography ((95:5) CH₂Cl₂—MeOH (5% NH₄OH)) afforded 0.60 g (64%) of desired product which was converted to the HCl salt to generate 3-{[3-(5-chloro-1H-indol-3-yl)propyl]amino}-8-fluorochromane-5-carboxamide hydrochloride salt as a white solid: mp 218-221° C.; MS (ES) m/z 402.1; Anal. Calcd for C₂₁H₂₁ClFN₃O₂.HCl: C, 57.54; 14, 5.06; N, 9.59. Found: C, 57.34; H, 5.05; N, 9.24.

### Example 223

### 3-[[3-(5-chloro-1H-indol-3-yl)propyl](ethyl)amino]-8-fluorochromane-5-carboxamide (“Compound 115”)

This compound was prepared as described above for example 218 using 3-{[3-(5-chloro-1H-indol-3-yl)propyl]amino}-8-fluorochromane-5-carboxamide (0.124 g, 0.31 mmol) and acetaldehyde (0.064 mL, 1.1 mmol) to afford 0.132 g (99%) of desired product which was converted to the HCl salt to generate 3-[[3-(5-chloro-1H-indol-3-yl)propyl](ethyl)amino]-8-fluorochromane-5-carboxamide hydrochloride salt as a white solid: mp 149-156° C. (melts with decomposition); MS (ES) m/z 430.2.

### Example 224

### 3-[[3-(5-chloro-1H-indol-3-yl)propyl](propyl)amino]-8-fluorochromane-5-carboxamide (“Compound 116”)

This compound was prepared as described above for example 223 using propionaldehyde (0.082 mL, 1.1 mmol) to afford 0.134 g (98%) of desired product which was converted to the HCl salt to generate 3-[[3-(5-chloro-1H-indol-3-yl)propyl](propyl)amino]-8-fluorochromane-5-carboxamide hydrochloride salt as a white solid: mp 148-154° C. (melts with decomposition); MS (ES) m/z 444.2.

### Example 225

### 3-[[3-(5-chloro-1H-indol-3-yl)propyl](cyclobutyl)amino]-8-fluorochromane-5-carboxamide (“Compound 117”)

This compound was prepared as described above for example 223 using cyclobutanone (0.085 mL, 1.1 mmol) to afford 0.135 g (96%) of desired product which was converted to the HCl salt to generate 3-[[3-(5-chloro-1H-indol-3-yl)propyl](cyclobutyl)amino]-8-fluorochromane-5-carboxamide hydrochloride salt as a white solid: mp 152-160° C. (melts with decomposition); MS (ES) m/z 454.1.

### Example 226

### 3-[[3-(5-chloro-1H-indol-3-yl)propyl](cyclopropylmethyl)amino]-8-fluorochromane-5-carboxamide (“Compound 118”)

This compound was prepared as described above for example 223 using cyclopropanecarboxaldehyde (0.085 mL, 1.1 mmol) to afford 0.136 (96%) of desired product which was converted to the HCl salt to generate 3-[[3-(5-chloro-1H-indol-3-yl)propyl](cyclopropylmethyl)amino]-8-fluorochromane-5-carboxamide hydrochloride salt as a white solid: mp 150-158° C. (melts with decomposition); MS (ES) m/z 454.1.

### Example 227

### 5-fluoro-3-{[3-(5-fluoro-1H-indol-3-yl)propyl]amino}chromane-8-carboxamide (“Compound 119”)

To 3-amino-5-fluorochromane-8-carboxamide (0.27 g, 1.3 mmol) in anhydrous methanol (21 mL), under nitrogen at room temperature, was added 3-(5-fluoro-1H-indol-3-yl)propanal (0.25 g, 1.3 mmol), acetic acid (0.16 mL, 3.1 mmol) and sodium cyanoborohydride (0.16 g, 2.6 mmol). The reaction mixture was stirred at room temperature for 2 hrs. Chromatography (EtOAc followed by (5:4:1) EtOAc-Hex-MeOH (1% NH₄H)) afforded 0.3 g (59%) of desired product which was converted to the HCl salt to generate 5-fluoro-3-{[3-(5-fluoro-1H-indol-3-yl)propyl]amino}chromane-8-carboxamide hydrochloride salt as a white solid: mp 124° C./dec; MS (ES) m/z 386.2; Anal. Calcd for C₂₁H₂₁F₂N₃O₂.1.20 HCl: C, 58.77; H, 5.21; N, 9.79. Found: C, 58.71; H, 4.73; N, 9.55.

### Example 228

### 5-fluoro-3-[[3-(5-fluoro-1H-indol-3-yl)propyl](propyl)amino]chromane-8-carboxamide (“Compound 120”)

To 5-fluoro-3-{[3-(5-fluoro-1H-indol-3-yl)propyl]amino}chromane-8-carboxamide (0.08 g, 0.21 mmol) in anhydrous methanol (3.5 mL), under nitrogen at room temperature, was added propionaldehyde (0.023 mL, 0.31 mmol), acetic acid (0.025 mL, 0.5 mmol) and sodium cyanoborohydride (0.026 g, 0.42 mmol). The reaction mixture was stirred at room temperature overnight. Chromatography on Biotage Quad using silica gel column and ((6:3:1) Hex-EtOAc-MeOH (1% NH₄OH)) as elution solvent afforded 0.076 g (85%) of desired product which was converted to the HCl salt to generate 5-fluoro-3-[[3-(5-fluoro-1H-indol-3-yl)propyl](propyl)amino]chromane-8-carboxamide hydrochloride salt as a white solid: mp 123° C./dec; MS (ES) m/z 428.2.

### Example 229

### 3-{(cyclopropylmethyl)[3-(5-fluoro-1H-indol-3-yl)propyl]amino}-5-fluorochromane-8-carboxamide (“Compound 121”)

This compound was prepared as described above for example 228 using cyclopropanecarboxaldehyde (0.024 mL, 0.31 mmol). Chromatography on Biotage Quad using silica gel column and ((6:3:1) Hex-EtOAc-MeOH (1% NH₄OH)) as elution solvent afforded 0.079 g (86%) of desired product which was converted to the HCl salt to generate 3-{(cyclopropylmethyl)[3-(5-fluoro-1H-indol-3-yl)propyl]amino}-5-fluorochromane-8-carboxamide hydrochloride salt as a white solid: mp 123° C./dec; MS (ES) m/z 440.2.

### Example 230

### 3-{cyclobutyl[3-(5-fluoro-1H-indol-3-yl)propyl]amino}-5-fluorochromane-8-carboxamide (“Compound 122”)

This compound was prepared as described above for example 228 using cyclobutanone (0.078 mL, 1.04 mmol) (2 additions of 0.039 mL). Chromatography on Biotage Quad using silica gel column and ((6:3:1) Hex-EtOAc-MeOH (1% NH₄OH)) as elution solvent afforded 0.075 g (82%) of desired product which was converted to the HCl salt to generate 3-{cyclobutyl[3-(5-fluoro-1H-indol-3-yl)propyl]amino}-5-fluorochromane-8-carboxamide hydrochloride salt as a white solid: mp 132° C./dec; MS (ES) m/z 440.2.

### Example 231

### 5-fluoro-3-{[4-(5-fluoro-1H-indol-3-yl)butyl]amino}chromane-8-carboxamide (“Compound 123”)

This compound was prepared as described above for example 227 using 4-(5-fluoro-1H-indol-3-yl)butanal. Chromatography ((5:4:1) EtOAc-Hex-MeOH (1% NH₄OH)) afforded 0.43 g (83%) of desired product which was converted to the HCl salt to generate 5-fluoro-3-{[4-(5-fluoro-1H-indol-3-yl)butyl]amino}chromane-8-carboxamide hydrochloride salt as a white solid: mp 119° C./dec; MS (ES) nLz 400.2; Anal. Calcd for C₂₂H₂₃F₂N₃O₂.1.20 HCl: C, 59.62; H, 5.50; N, 9.48. Found: C, 59.47; H, 5.39; N, 9.29.

### Example 232

### 5-fluoro-3-[[4-(5-fluoro-1H-indol-3-yl)butyl](propyl)amino]chromane-8-carboxamide (“Compound 124”)

To 5-fluoro-3-{[4-(5-fluoro-1H-indol-3-yl)butyl]amino}chromane-8-carboxamide (0.09 g, 0.23 mmol) in anhydrous methanol (3.8 mL), under nitrogen at room temperature, was added propionaldehyde (0.024 mL, 0.34 mmol), acetic acid (0.027 mL, 0.54 mmol) and sodium cyanoborohydride (0.028 g, 0.45 mmol). The reaction mixture was stirred at room temperature overnight. Chromatography on Biotagee Quad using silica gel column and ((6:3:1) Hex-EtOAc-MeOH (1% NH₄OH)) as elution solvent afforded 0.089 g (89%) of desired product which was converted to the HCl salt to generate 5-fluoro-3-[[4-(5-fluoro-1H-indol-3-yl)butyl](propyl)amino]chromane-8-carboxamide hydrochloride salt as a white solid: mp 116° C./dec; MS (ES) m/z 442.3.

### Example 233

### 3-{(cyclopropylmethyl)[4-(5-fluoro-1H-indol-3-yl)butyl]amino}-5-fluorochromane-8-carboxamide (“Compound 125”)

This compound was prepared as described above for example 232 using cyclopropane carboxaldehyde (0.025 mL, 0.34 mmol). Chromatography on Biotage Quad using silica gel column and ((6:3:1) Hex-EtOAc-MeOH (1% NH₄OH)) as elution solvent afforded 0.088 g (86%) of desired product which was converted to the HCl salt to generate 3-{(cyclopropylmethyl)[4-(5-fluoro-1H-indol-3-yl)butyl]amino}-5-fluorochromane-8-carboxamide hydrochloride salt as a white solid: mp 116° C./dec; MS (ES) m/z 454.3.

### Example 234

### 3-{cyclobutyl[4-(5-fluoro-1H-indol-3-yl)butyl]amino{-5-fluorochromane-8-carboxamide (“Compound 126”)

This compound was prepared as described above for example 232 using cyclobutanone (0.084 mL, 1.12 mmol) (2 additions of 0.042 mL). Chromatography on Biotage Quad using silica gel column and ((6:3:1) Hex-EtOAc-MeOH (1% NH₄OH)) as elution solvent afforded 0.083 g (82%) of desired product which was converted to the HCl salt to generate 3-{cyclobutyl[4-(5-fluoro-1H-indol-3-yl)butyl]amino}-5-fluorochromane-8-carboxamide hydrochloride salt as a white solid: mp 130° C./dec; MS (ES) m/z 454.3.

### Example 235

### 3-{ethyl[4-(5-fluoro-1H-indol-3-yl)butyl]naino}-5-fluorochromane-8-carboxamide (“Compound 127”)

This compound was prepared as described above for example 232 using acetaldehyde (0.018 mL, 0.34 mmol). Chromatography on Biotage Quad using silica gel column and ((6:3:1) Hex-EtOAc-MeOH (1% NH₄OH)) as elution solvent afforded 0.087 g (90%) of desired product which was converted to the HCl salt to generate 3-{ethyl[4-(5-fluoro-1H-indol-3-yl)butyl]amino}-5-fluorochromane-8-carboxamide hydrochloride salt as a white solid: mp 112° C./dec; MS (ES) m/z 428.2.

### Example 236

### 3-{[3-(5,7-difluoro-1H-indol-3-yl)propyl]amino}-5-fluorochromane-8-carboxamide (“Compound 128”)

This compound was prepared as described above for example 227 using 3-(5,7-difluoro-1H-indol-3-yl)propanal. Chromatography (EtOAc followed by (5:4:1) EtOAc-Hex-MeOH (1% NH₄OH)) afforded 0.47 g (89%) of desired product which was converted to the HCl salt to generate 3-{[3-(5,7-difluoro-1H-indol-3-yl)propyl]amino}-5-fluorochromane-8-carboxamide hydrochloride salt as a beige solid: mp 149° C./dec; MS (ES) m/z 404.2; Anal. Calcd for C₂₁H₂₀F₃N₃O₂.1.20 HCl: C, 56.41; H, 4.78; N, 9.40. Found: C, 56.05; H, 4.72; N, 9.11.

### Example 237

### 3-[[3-(5,7-difluoro-1H-indol-3-yl)propyl](propyl)amino]-5-fluorochromane-8-carboxamide (“Compound 129”)

To 3-{[3-(5,7-difluoro-1H-indol-3-yl)propyl]amino}-5-fluorochromane-8-carboxamide (0.095 g, 0.24 mmol) in anhydrous methanol (4.0 mL), under nitrogen at room temperature, was added propionaldehyde (0.025 mL, 0.35 mmol), acetic acid (0.028 mL, 0.56 mmol) and sodium cyanoborohydride (0.03 g, 0.47 mmol). The reaction mixture was stirred at room temperature overnight. Chromatography on Biotage Quad using silica gel column and ((6:3:1) Hex-EtOAc-MeOH (1% NH₄OH)) as elution solvent afforded 0.072 g (69%) of desired product which was converted to the HCl salt to generate 3-[[3-(5,7-difluoro-1H-indol-3-yl)propyl](propyl)amino]-5-fluorochromane-8-carboxamide hydrochloride salt as a white solid: mp 130° C./dec; MS (ES) m/z 446.2.

### Example 238

### 3-{(cyclopropylmethyl)[3-(5,7-difluoro-1H-indol-3-yl)propyl]amino}-5-fluorochromane-8-carboxamide (“Compound 130”)

This compound was prepared as described above for example 237 using cyclopropane carboxaldehyde (0.026 mL, 0.35 mmol). Chromatography on Biotage Quad using silica gel column and ((6:3:1) Hex-EtOAc-MeOH (1% NH₄OH)) as elution solvent afforded 0.083 g (77%) of desired product which was converted to the HCl salt to generate 3-{(cyclopropylmethyl)[3-(5,7-difluoro-1H-indol-3-yl)propyl]amino}-5-fluorochromane-8-carboxamide hydrochloride salt as a white solid: mp 124° C./dec; MS (ES) m/z 458.2.

### Example 239

### 3-{cyclobutyl[3-(5,7-difluoro-1H-indol-3-yl)propyl]amino}-5-fluorochromane-8-carboxamide (“Compound 131”)

This compound was prepared as described above for example 237 using cyclobutanone (0.088 mL, 1.17 mmol) (2 additions of 0.044 mL). Chromatography on Biotage Quad using silica gel column and ((6:3:1) Hex-EtOAc-MeOH (1% NH₄OH)) as elution solvent afforded 0.077 g (72%) of desired product which was converted to the HCl salt to generate 3-{cyclobutyl[3-(5,7-difluoro-1H-indol-3-yl)propyl]amino}-5-fluorochromane-8-carboxamide hydrochloride salt as a white solid: mp 136° C./dec; MS (ES) m/z 458.2.

### Example 240

### 3-[[3-(5,7-difluoro-1H-indol-3-yl)propyl](ethyl)amino]-5-fluorochromane-8-carboxamide (“Compound 132”)

This compound was prepared as described above for example 237 using acetaldehyde (0.02 mL, 0.35 mmol). Chromatography on Biotage Quad using silica gel column and ((6:3:1) Hex-EtOAc-MeOH (1% NH₄OH)) as elution solvent afforded 0.087 g (85%) of desired product which was converted to the HCl salt to generate 3-[[3-(5,7-difluoro-1H-indol-3-yl)propyl](ethyl)amino]-5-fluorochromane-8-carboxamide hydrochloride salt as a white solid: mp 125° C./dec; MS (ES) m/z 432.2.

### Example 241

### 3-{[3-(5-fluoro-1H-indol-3-yl)propyl]amino}chromane-5-carboxamide (“Compound 133”)

A solution of the starting 3-amino-chroman-5-carboxylic acid amide (9 mmole, 1.730 g) in methanol (155 mL) was treated under dry nitrogen with 3-(5-fluoro-1H-indol-3-yl)-propionaldehyde (9.46 mmole, 1.8 g), acetic acid (0.56 mL) and sodium cyanoborohydride (17.9 mmole, 1.125 g) at ambient temperature under stirring. The reaction mixture was stirred at ambient temperature overnight, quenched with 1N sodium hydroxide to pH 10, concentrated in vacuo and the residue partitioned between water and ethyl acetate. The organic layer was separated, washed with brine, dried over magnesium sulfate, filtered and evaporated in vacuo. to yield ˜4.0 g of the crude product. Flash chromatography of the crude material on silica gel (70 g) using a solvent mixture of ethyl acetate, hexane and 2% ammonia in methanol (5:4:1) gave 0.85 g of the bis-adduct and 1.37 g (42%) of the desired title compound as a white amorphous powder: mp 153-5° C.; MS (ES) m/z 368.2.

### Examples 241a and 241b

### (3S)-3-{[3-(5-fluoro-1H-indol-3-yl)propyl]amino}chromane-5-carboxamide (“Compound 133a”) and (3R)-3-{[3-(5-fluoro-1H-indol-3-yl)propyl]amino}chromane-5-carboxamide (“Compound 133b”)

The racemic 3-{[3-(5-fluoro-1H-indol-3-yl)propyl]amino}chromane-5-carboxamide was subjected to chiral separation on a Chiralcel column. Elution with a mobile phase of 75% ethanol in hexane and detection with a 289 nm detector gave both enantiomers as a white hardened foam in >99.9% purity:
(3S)-3-{[3-(5-fluoro-1H-indol-3-yl)propyl]amino}chromane-5-carboxamide (more polar enantiomer): MS (ES) m/z 368.2
(3R)-3-{[3-(5-fluoro-1H-indol-3-yl)propyl]amino}chromane-5-carboxamide (less polar enantiomer): MS (ES) m/z 368.2.

### Example 242

### 3-{cyclobutyl[3-(5-fluoro-1H-indol-3-yl)propyl]amino}chromane-5-carboxamide (“Compound 134”)

A solution of the starting 3-{[3-(5-fluoro-1H-indol-3-yl)propyl]amino}chromane-5-carboxamide (0.45 mmole, 168 mg) in methanol (8 mL) was treated with cyclobutanone (1.23 mmole, 86 mg), acetic acid (00.1 mL) and sodium cyanoborohydride (1 mmole, 63 mg) at ambient temperature under stirring. The reaction mixture was stirred for 14 h at ambient temperature, after which it was treated again with cyclobutanone (1.23 mmole, 86 mg), acetic acid (00.1 mL) and sodium cyanoborohydride (1 mmole, 63 mg). After stirring for another 20 h the mixture was quenched with 1N sodium hydroxide to pH 10, concentrated in vacuo and the residue partitioned between water and ethyl, acetate. The organic layer was separated, washed with brine, dried over magnesium sulfate, filtered and evaporated in vacuo. The residue was dissolved in ether and triturated with ethereal hydrochloric acid. The precipitated product was filtered, washed with ether and dried to yield 180 mg (95%) of the title compound: mp 102-5° C.; MS (ES) m/z 422.3.

### Examples 242a and 242b

### (−)-3-{cyclobutyl[3-(5-fluoro-1H-indol-3-yl)propyl]amino}chromane-5-carboxamide (“Compound 134a”) and (+)-3-{cyclobutyl[3-(5-fluoro-1H-indol-3-yl)propyl]amino}chromane-5-carboxamide (“Compound 134b”)

A solution of one enantiomer of 3-{[3-(5-fluoro-1H-indol-3-yl)propyl]amino}chromane-5-carboxamide (0.38 mmole, 140 mg) in methanol (6 mL) was treated under dry nitrogen and under stirring at ambient temperature with cyclobutanone (1 mmole, 71 mg), followed by acetic acid (0.07 mL) and sodium cyanoborohydride (0.8 mmole, 51 mg). The reaction mixture was stirred for 16 h at ambient temperature, after which it was treated again with cyclobutanone (1 mmole, 71 mg), followed by acetic acid (0.07 mL) and sodium cyanoborohydride (0.8 mmole, 51 mg). After stirring for another 24 h a third addition of cyclobutanone (1 mmole, 71 mg), followed by acetic acid (0.07 mL) and sodium cyanoborohydride (0.8 mmole, 51 mg) was carried out and stirring was continued for 24 h after which the mixture was quenched with 1N sodium hydroxide to pH 10, concentrated in vacuo and the residue partitioned between water and ethyl acetate. The organic layer was separated, washed with brine, dried over magnesium sulfate, filtered and evaporated in vacuo. The residue was flash chromatographed on silica gel (5 g). Elution with 2% methanol in ethyl acetate gave 120 mg (75%) of (−)-3-{cyclobutyl[3-(5-fluoro-1H-indol-3-yl)propyl]amino}chromane-5-carboxamide as a dense colorless gum; MS (ES) m/z 422.2; [α]_{D}²⁵=−34.10 (c=1% SOLUTION, MeOH).

A solution of the other enantiomer of 3-{[3-(5-fluoro-1H-indol-3-yl)propyl]amino}chromane-5-carboxamide (0.41 mmole, 150 mg) in methanol (7 mL) was treated under dry nitrogen and under stirring at ambient temperature with cyclobutanone (1.1 mmole, 77 mg), followed by acetic acid (0.08 mL) and sodium cyanoborohydride (0.86 mmole, 54 mg). The reaction mixture was stirred for 16 h at ambient temperature, after which, it was treated again with cyclobutanone (1.1 mmole, 77 mg), followed by acetic acid (0.08 mL) and sodium cyanoborohydride (0.86 mmole, 54 mg). After stirring for another 24 h a third addition of cyclobutanone (1.1 mmole, 77 mg), followed by acetic acid (0.08 mL) and sodium cyanoborohydride (0.86 mmole, 54 mg) was carried out and stirring was continued for 24 h after which the mixture was quenched with 1N sodium hydroxide to pH 10, concentrated in vacuo and the residue partitioned between water and ethyl acetate. The organic layers was separated, washed with brine, dried over magnesium sulfate, filtered and evaporated in vacuo. The residue was flash chromatographed on silica gel (5 g). Elution with 2% methanol in ethyl acetate gave 140 mg (80%) of (+)-3-{cyclobutyl[3-(5-fluoro-1H-indol-3-yl)propyl]amino}chromane-5-carboxamide as a dense colorless foam: MS (ES) m/z 422.2; [α]_{D}²⁵=+33.2° (c=1% SOLUTION, MeOH);

### Example 243

### Methyl 3-{[3-(5,7-difluoro-1H-indol-3-yl)propyl]amino}chromane-5-carboxylate (“Compound 135”)

The starting methyl 3-aminochromane-5-carboxylate (6.03 mmole, 1.25 g) was dissolved in methanol (30 mL) and treated under stirring at 0° C., consecutively with 3′-(5,7-difluoro-1H-indol-3-yl)propanal (6.39 mmole, 1.34 g), acetic acid (0.45 mL) and sodium cyanoborohydride. (12.06 mmole, 760 mg). The reaction mixture was stirred at ambient temperature for 6 h and evaporated in vacuo. The residue was partitioned between aqueous 5% sodium bicarbonate and ethyl acetate. The aqueous phase was back-washed with ethyl acetate., The combined organic layers were washed with brine, dried over magnesium sulfate, filtered and evaporated to dryness in vacuo. The residue was flash chromatographed on silica gel. Elution with 5% methanol in chloroform containing a few drops aqueous ammonia afforded 1.3 g (54%) of the title compound: MS (ES) m/z 401.1.

### Example 244

### Methyl 3-{cyclobutyl[3-(5,7-difluoro-1H-indol-3-yl)propyl]amino}chromane-5-carboxylate (“Compound 136”)

The starting methyl 3-{[3-(5,7-difluoro-1H-indol-3-yl)propyl]amino}chromane-5-carboxylate (2.65 mmole, 1.06 g) was dissolved in methanol (30 mL) and treated consecutively. with cyclobutanone (6.6 mmole, 0.5 mL), acetic acid (0.5 mL) and sodium cyanoborohydride (5.29 mmole, 330 mg). The reaction mixture was stirred for 8 h. Cyclobutanone (6.6 mmole, 0.5 mL), acetic acid (0.5 mL) and sodium cyanoborohydride (5.29 mmole, 330 mg) were added again and stirring continued for 10 h. A third addition of cyclobutanone (6.6 mmole, 0.5 mL), acetic acid (0.5 mL) and sodium cyanoborohydride (5.29 mmole, 330 mg) was carried out and stirring continued for 10 h. After evaporation in vacuo the residue was flash chromatographed on silica gel. Elution with 5% methanol in chloroform containing a few drops aqueous ammonia afforded 1.3 g (93%) of the title compound as a yellow oil: MS (ES) m/z 453.1.

### Example 245

### 3-{cyclobutyl[3-(5,7-difluoro-1H-indol-3-yl)propyl]amino}chromane-5-carboxylic acid (“Compound 137”)

A solution of methyl 3-{cyclobutyl[3-(5,7-difluoro-1H-indol-3-yl)propyl]amino}chromane-5-carboxylate (2.2 mmole, 1 g) in tetrahydrofuran (15 mL) was treated at once with 2N sodium hydroxide (8 mL) under stirring. The reaction mixture was stirred at 46° C. for 140 h, cooled to ambient temperature and then diluted with ethyl acetate and washed with water. The separated organic layer was washed with brine, dried over magnesium sulfate, filtered and evaporated in vacuo to dryness to afford 960 mg (98%) of the title compound: mp 130-131° C.; MS (ES) m/z 441.2.

### Example 246

### 3-{cyclobutyl[3-(5,7-difluoro-1H-indol-3-yl)propyl]amino}-N-methylchromane-5-carboxamide (“Compound 138”)

A solution of 3-{cyclobutyl[3-(5,7-difluoro-1H-indol-3-yl)propyl]amino}chromane-5-carboxylic acid (0.227 mmole, 100 mg) in tetrahydrofuran (8 mL) was treated at ambient temperature with 2M methylamine in THF (0.908 mmole, 0.454 mL) followed by 1-hydroxybenzotriazole (0.454 mmole, 61.4 mg) and 1-[3-(dimethylamino)propyl]-3-ethylcarbodiimide hydrochloride (0.454 mmole, 87.1 mg). The reaction mixture was stirred at ambient temperature for 6 h after which additional methylamine (2M in THF, 0.227 mmole, 114 mg) was added. Stirring was continued for 14 h. The reaction mixture was then diluted with ethyl acetate, washed twice with aqueous 5% sodium bicarbonate solution. The aqueous phase was back-washed with ethyl acetate and the combined organic layers washed with brine, dried over magnesium sulfate, filtered and evaporated to dryness. The residue was purified using the Biotage in conjunction with LC-MS to yield 64 mg (62%) of the desired title compound: MS (ES) m/z 454.2.

### Example 247

### 3-{cyclobutyl[3-(5,7-difluoro-1H-indol-3-yl)propyl]amino}-N-ethylchromane-5-carboxamide (“Compound 139”)

This compound was prepared as described above for example 246 using ethylamine. Yield: 80 mg (76%): MS (ES) m/z 466.2.

### Example 248

### 3-{cyclobutyl[3-(5,7-difluoro-1H-indol-3-yl)propyl]amino}-N-propylchromane-5-carboxamide (“Compound 140”)

This compound was prepared as described above for example 246 using propylamine. Yield: 45 mg (41%): MS (ES) m/z 482.3.

### Example 249

### 3-{cyclobutyl[3-(5,7-difluoro-1H-indol-3-yl)propyl]amino}-N-isopropylchromane-5-carboxamide (“Compound 141”)

This compound was prepared as described above for example 246 using isopropyl amine. Yield: 43 mg (40%): MS (ES) m/z 482.3.

### Example 250

### 3-{cyclobutyl[3-(5,7-difluoro-1H-indol-3-yl)propyl]amino}-N-cyclopropylchromane-5-carboxamide (“Compound 142”)

This compound was prepared as described above for example 246 using cyclopropyl amine. Yield: 58 mg (53%): MS (ES) m/z 480.3.

### Example 251

### N-cyclobutyl-3-{cyclobutyl[3-(5,7-difluoro-1H-indol-3-yl)propyl]amino}chromane-5-carboxamide (“Compound 143”)

This compound was prepared as described above for example 246 using cyclobutyl amine. Yield: 63 mg (56%): MS (ES) m/z 494.3.

### Example 252

### 3-{cyclobutyl[3-(5,7-difluoro-1H-indol-3-yl)propyl]amino}-N-(cyclopropylmethyl)chromane-5-carboxamide (“Compound 144”)

This compound was prepared as described above for example 246 using cyclo propanemethylamine. Yield: 78 mg (70%): MS (ES) m/z 494.3.

### Examples 253a and 253b

### (3S)-3-{cyclobutyl[4-(5-fluoro-1H-indol-3-yl)butyl]amino}-8-fluoro-N-methylchromane-5-carboxamide (“Compound 145a”) and (3R)-3-{cyclobutyl[4-(5-fluoro-1H-indol-3-yl)butyl]amino}-8-fluoro-N-methylchromane-5-carboxamide (“Compound 145b”)

Methyl (3S)-3-{cyclobutyl[4-(5-fluoro-1H-indol-3-yl)butyl]amino}-8-fluorochromane-5-carboxylate was converted to (3S)-3-{cyclobutyl[4-(5-fluoro-1H-indol-3-yl)butyl]amino}-8-fluorochromane-5-carboxylic acid as described above for example 164 generating 0.32 g (85%) of desired product. The product was characterized by ¹HNMR.

To (3S)-3-{cyclobutyl[4-(5-fluoro-1H-indol-3-yl)butyl]amino}-8-fluorochromane-5-carboxylic acid (0.32 g, 0.7 mmol) in anhydrous THF (25 mL), under nitrogen at room temperature, was added EDC (0.27 g, 1.4 mmol), HOBt (0.19 g, 1.4 mmol) and a 2M solution of methylamine in THF (1.4 mL, 2.8 mmol). The cloudy solution was stirred at room temperature overnight. Chromatography ((6:3:1) Hex-EtOAc-MeOH (1% NH₄OH) afforded 0.3 g (93%) of desired product which was converted to the HCl salt to generate (3S)-3-{cyclobutyl[4-(5-fluoro-1H-indol-3-yl)butyl]amino}-8-fluoro-N-methylchromane-5-carboxamide hydrochloride salt as a white solid: mp 125° C./dec; [α]_{D}²⁵=+24.2° (c=1% SOLUTION, DMSO); MS (ES) m/z 468.3; Anal. Calcd for C₂₇H₃₁F₂N₃O₂.HCl.0.25 H₂O: C, 63.77; H, 6.44; N, 8.26. Found: C, 63.51; H, 6.46; N, 8.09.

Methyl (3R)-3-{cyclobutyl[4-(5-fluoro-1H-indol-3-yl)butyl]amino}-8-fluorochromane-5-carboxylate was converted to (3R)-3-{cyclobutyl[4-(5-fluoro-1H-indol-3-yl)butyl]amino}-8-fluorochromane-5-carboxylic acid as described above for example 164 generating 0.29 g (80%) of desired product. The product was characterized by ¹HNMR.

The title compound 145b was prepared as described above for compound 145a using (3R)-3-{cyclobutyl[4-(5-fluoro-1H-indol-3-yl)butyl]amino}-8-fluorochromane-5-carboxylic acid as starting material. Chromatography ((6:3:1) Hex-EtOAc-MeOH (1%. NH₄OH) afforded 0.31 g (100%) of desired product which was converted to the HCl salt to generate (3R)-3-{cyclobutyl[4-(5-fluoro-1H-indol-3-yl)butyl]amino}-8-fluoro-N-methylchromane-5-carboxamide hydrochloride salt as a white solid: mp 125° C./dec; [α]_{D}²⁵=−22.2° (c-1% SOLUTION, DMSO); MS (ES) m/z 468.3; Anal. Calcd for C₂₇H₃₁F₂N₃O₂.HCl.0.25 H₂O: C, 63.77; H, 6.44; N, 8.26. Found: C, 63.55; H, 6.61; N, 8.22.

### Example 254

### (3R)-3-{[3-(5,7-difluoro-1H-indol-3-yl)propyl]amino}-8-fluorochromane-5-carboxamide (“Compound 146”)

A solution of the starting (3R)-3-amino-8-fluorochromane-5-carboxamide L-(+)-tartrate (3.5 mmole, 1.26 g) in methanol (60 mL) was treated under dry nitrogen and under stirring at ambient temperature with 3-(5,7-difluoro-1H-indol-3-yl)propanal (3.6 mmole, 754 mg), followed by acetic acid (0.41 mL) and sodium cyanoborohydride (7.1 mmole, 447 mg). The reaction mixture was stirred for 16 h at ambient temperature, after which it was quenched with 1N sodium hydroxide to pH 10, concentrated in vacuo and the residue partitioned between water and ethyl acetate. The organic layer was separated, washed with brine, dried over magnesium sulfate, filtered and evaporated in vacuo to afford 1.41 g (−100%) of the title compound as an off-white gum. MS (ES) m/z 402.3.

### Example 255

### (3R)-3-{(cyclopropylmethyl)[3-(5,7-difluoro-1H-indol-3-yl)propyl]amino}-8-fluorochromane-5-carboxamide (“Compound 147”)

A solution of the starting (3R)-3-{[3-(5,7-difluoro-1H-indol-3-yl)propyl]amino}-8-fluorochromane-5-carboxamide (1.5 mmole. 605 mg) in methanol (40 mL) was treated under dry nitrogen and under stirring at ambient temperature with cyclopropane carboxaldehyde (4.5 mmole, 330 mg), followed by acetic acid (0.5 mL) and sodium cyanoborohydride (3.8 mmole, 240 mg). The reaction mixture was stirred for 18 h at ambient temperature, after which it was quenched with 1N sodium hydroxide to pH 10, concentrated in vacuo and the residue partitioned between water and ethyl, acetate. The organic layer was separated, washed with brine, dried over magnesium sulfate, filtered and evaporated in vacuo. The residue was flash chromatographed on silica gel (40 g). Elution with 2% methanol in ethyl acetate gave 620 mg (90%) of the desired title compound. The base was converted to the hydrochloride salt in ethyl acetate using ethereal hydrochloric acid: mp 147-51° C.; MS (ES) m/z 458.2.

### Example 256

### 3-{(cyclopropylmethyl)[3-(5-fluoro-1H-indol-3-yl)propyl]amino}chromane-5-carboxamide (“Compound 148”)

A solution of the starting 3-{[3-(5-fluoro-1H-indol-3-yl)propyl]amino}chromane-5-carboxamide (0.45 mmole, 168 mg) in methanol (8 mL) was treated with cyclopropane carboxaldehyde (1.23 mmole, 86 mg), acetic acid (00.1 mL) and sodium cyanoborohydride (1 mmole, 63 mg) at ambient temperature under stirring. The reaction mixture was stirred for 14 h at ambient temperature, after which it was treated again with cyclopropane carboxaldehyde (1.23 mmole, 86 mg), acetic acid (00.1 mL) and sodium cyanoborohydride (1 mmole, 63 mg). After stirring for another 20 h the mixture was quenched with 1N sodium hydroxide to pH 10, concentrated in vacuo and the residue partitioned between water and ethyl acetate. The organic layer was separated, washed with brine, dried over magnesium sulfate, filtered and evaporated in vacuo. The residue was dissolved in ether, triturated with ethereal hydrochloric acid and evaporated in vacuo to yield 130 mg (31%) of the title compound as a white foam: MS (ES) m/z 422.3.

### Example 257

### 3-{ethyl[3-(5-fluoro-1H-indol-3-yl)propyl]amino}chromane-5-carboxamide (“Compound 149”)

A solution of the starting 3-{[3-(5-fluoro-1H-indol-3-yl)propyl]amino}chromane-5-carboxamide (0.45 mmole, 168 mg) in methanol (8 mL) was treated with acetaldehyde (1.23 mmole, 54 mg), acetic acid (0.1 mL) and sodium cyanoborohydride (1 mmole, 63 mg) at ambient temperature under stirring. The reaction mixture was stirred for 14 h at ambient temperature, after which it was treated again with acetaldehyde (1.23 mmole, 54 mg), acetic acid (0.1 mL) and sodium cyanoborohydride (1 mmole, 63 mg). After stirring for another 20 h the mixture was quenched with 1N sodium hydroxide to pH 10, concentrated in vacuo and the residue partitioned between water and ethyl acetate. The organic layer was separated, washed with brine, dried over magnesium sulfate, filtered and evaporated in vacuo. The residue was dissolved in ether, triturated with ethereal hydrochloric acid and evaporated in vacuo to yield 140 mg (35%) of the title compound as white foam: MS (ES) m/z 396.2.

### Example 258

### 3-[[3-(5-fluoro-1H-indol-3-yl)propyl](propyl)amino]chromane-5-carboxamide (“Compound 150”)

A solution of the starting 3-{[3-(5-fluoro-1H-indol-3-yl)propyl]amino}chromane-5-carboxamide (0.45 mmole, 168 mg) in methanol (8 mL) was treated with propionaldehyde (1.23 mmole, 72 mg), acetic acid (0.1 mL) and sodium cyanoborohydride (1 mmole, 63 mg) at ambient temperature under stirring. The reaction mixture was stirred for 14 h at ambient temperature, after which it was treated again with propionaldehyde (1.23 mmole, 72 mg), acetic acid (00.1 mL) and sodium cyanoborohydride (1 mmole, 63 mg). After stirring for another 20 h the mixture was quenched with 1N sodium hydroxide to pH 10, concentrated in vacuo and the residue partitioned between water and ethyl acetate. The organic layer was separated, washed with brine, dried over magnesium sulfate, filtered and evaporated in vacuo. The residue was dissolved in ether, triturated with ethereal hydrochloric acid and evaporated in vacuo to yield 210 mg (51%) of the title compound as a colorless oil: MS (ES) m/z 410.2.

### Example 259

### 3-[[3-(5-fluoro-1H-indol-3-yl)propyl](isobutyl)amino]chromane-5-carboxamide (“Compound 151”)

A solution of the starting 3-{[3-(5-fluoro-1H-indol-3-yl)propyl]amino}chromane-5-carboxamide (0.45 mmole, 168 mg) in methanol (8 mL) was treated with 2-methyl-propionaldehyde (1.23 mmole, 89 mg), acetic acid (0.1 mL) and sodium cyanoborohydride (1 mmole, 63 mg) at ambient temperature under stirring. The reaction mixture was stirred for 14 h at ambient temperature, after which it was treated again with 2-methyl-propionaldehyde (1.23 mmole, 89 mg), acetic acid (00.1 mL) and sodium cyanoborohydride (1 mmole, 63 mg). After stirring for another 20 h the mixture was quenched with 1N sodium hydroxide to pH 10, concentrated in vacuo and the residue partitioned between water and ethyl acetate. The organic layer was separated, washed with brine, dried over magnesium sulfate, filtered and evaporated in vacuo. The residue was dissolved in ether, triturated with ethereal hydrochloric acid and evaporated in vacuo to yield 320 mg (76%) of the title compound as a colorless oil: MS (ES) m/z 424.3.

### Example 260

### 8-fluoro-3-{[(3R)-3-(5-fluoro-1H-indol-3-yl)butyl]amino}chromane-5-carboxamide (“Compound 152”)

A solution of 3-amino-8-fluorochromane-5-carboxamide (0.5 g, 2.39 mmol) and (3R)-3-(5-fluoro-1H-indol-3-yl)butanal (0.515 g, 2.51 mmol) in anhydrous methanol (38 mL) and acetic acid (0.33 mL, 5.8 mmol) was treated with sodium cyanoborohydride (300 mg, 4.78 mmol). After stirring for 17 hours at ambient temperature the reaction was quenched with 1N aqueous NaOH (50 mL) and extracted with ethyl acetate (4×50 mL). The combined organic phases were washed with saturated aqueous NaCl, dried over MgSO₄, and evaporated under reduced pressure. Chromatography ((9:1) CH₂Cl₂—MeOH (5% NH₄OH)) afforded 0.723 g (76%) of desired product. The diastereomers were separated by HPLC and isolated to generate the following products:

(3R)-8-fluoro-3-{[(3R)-3-(5-fluoro-1H-indol-37yl)butyl]amino}chromane-5-carboxamide (P6995-180-1) as a white solid.

(3S)-8-fluoro-3-{[(3R)-3-(5-fluoro-1H-indol-3-yl)butyl]amino}chromane-5-carboxamide (P6995-180-2) as a white solid.

### Examples 260a and 260b

### (3R)-3-{cyclobutyl[(3R)-3-(5-fluoro-1H-indol-3-yl)butyl]amino}-8-fluorochromane-5-carboxamide (“Compound 152a”) and (3S)-3-{cyclobutyl[(3R)-3-(5-fluoro-1H-indol-3-yl)butyl]amino}-8-fluorochromane-5-carboxamide (“Compound 152b”)

A solution of (3R)-8-fluoro-3-{[(3R)-3-(5-fluoro-1H-indol-3-yl)butyl]amino}chromane-5-carboxamide (85 mg, 0.21 mmol) in anhydrous methanol (3 mL) was treated with acetic acid (0.034 mL, 0.60 mmol), cyclobutanone (0.057 mL, 0.76 mmol), and sodium cyanoborohydride (33 mg, 0.53 mmol) and stirred for 17 hours at ambient temperature. The reaction was quenched with 1N aqueous NaOH (10 mL) and extracted with ethyl acetate (4×10 mL). The combined organic phases are washed with saturated aqueous NaCl, dried over MgSO₄ and evaporated under reduced pressure. Chromatography ((98:2) CH₂Cl₂—MeOH (5% NH₄OH)) afforded 0.093 g (96%) of desired product which was converted to the HCl salt to generate (3R)-3-{cyclobutyl[(3R)-3-(5-fluoro-1H-indol-3-yl)butyl]amino}-8-fluorochromane-5-carboxamide hydrochloride salt as an off-white solid: mp 168-174° C. (melts with decomposition); [α]_{D}²⁵=24.8° (c=1% SOLUTION, DMSO); MS (ES) m/z 454.0; Anal. Calcd for C₂₆H₂₉F₂N₃O₂.HCl.H₂O: C, 61.47; H, 6.35; N, 8.27. Found: C, 61.11; H, 6.06; N, 8.00.

Similarly, a solution of (3S)-8-fluoro-3-{[(3R)-3-(5-fluoro-1H-indol-3-yl)butyl]amino}chromane-5-carboxamide (90 mg, 0.23 mmol) in anhydrous methanol (3 mL) was treated with acetic acid (0.036 mL, 0.63 mmol), cyclobutanone (0.060 mL, 0.80 mmol), and sodium cyanoborohydride (35 mg, 0.56 mmol) and stirred for 17 hours at ambient temperature. Chromatography ((98:2) CH₂Cl₂—MeOH (5% NH₄OH)) afforded. 0.095 g (93%) of desired product which was converted to the HCl salt to generate (3S)-3-{cyclobutyl[(3R)-3-(5-fluoro-1H-indol-3-yl)butyl]amino}-8-fluorochromane-5-carboxamide hydrochloride salt as an off-white solid: mp 164-172° C. (melts with decomposition); [α]_{D}²⁵=+21.4° (c=1% SOLUTION, DMSO); MS (ES) m/z 452.0; Anal. Calcd for C₂₆H₂₉F₂N₃O₂.HCl.H₂O: C, 61.47; H, 6.35; N, 8.27. Found: C, 61.22; H, 5.92; N, 8.11.

### Example 261

### 8-fluoro-3-{[(3S)-3-(5-fluoro-1H-indol-3-yl)butyl]amino}chromane-5-carboxamide (“Compound 153”)

A solution of 3-amino-8-fluorochromane-5-carboxamide (0.5 g, 2.39 mmol) and (3S)-3-(5-fluoro-1H-indol-3-yl)butanal (0.515 g, 2.51 mmol) in anhydrous methanol (38 mL) and acetic acid (0.33 mL, 5.8 mmol) was treated with sodium cyanoborohydride (300 mg, 4.78 mmol). Chromatography ((92:8) CH₂Cl₂—MeOH (5% NH₄OH)) afforded 0.845 g (89%) of desired product. The diastereomers were separated by HPLC and isolated to generate the following products:
(3R)-8-fluoro-3-{[(3S)-3-(5-fluoro-1H-indol-3-yl)butyl]amino}chromane-5-carboxamide (P6995-181-1) as a white solid.
(3S)-8-fluoro-3-{[(3S)-3-(5-fluoro-1H-indol-3-yl)butyl]amino}chromane-5-carboxamide (P6995-181-2) as a white solid.

### Examples 261a and 261b

### (3R)-3-{cyclobutyl[(3S)-3-(5-fluoro-1H-indol-3-yl)butyl]amino}-8-fluorochromane-5-carboxamide (“Compound 153a”) and (3S)-3-{cyclobutyl[(3S)-3-(5-fluoro-1H-indol-3-yl)butyl]amino}-8-fluorochromane-5-carboxamide (“Compound 153b”)

Compound 153a was prepared as described above for example 260a using (3R)-8-fluoro-3-{[(3S)-3-(5-fluoro-1H-indol-3-yl)butyl]amino}chromane-5-carboxamide as starting material. Chromatography ((98:2) CH₂Cl₂—MeOH (5% NH₄OH)) afforded 0.095 g (93%) of desired product which was converted to the HCl salt to generate (3R)-3-{cyclobutyl[(3S)-3-(5-fluoro-1H-indol-3-yl)butyl]amino}-8-fluorochromane-5-carboxamide hydrochloride salt as an off-white solid: mp 160-168° C. (melts with decomposition; [α]_{D}²⁵=−24.40 (c=1%, DMSO); MS (ES) m/z 454.3; Anal. Calcd for C₂₆H₂₉F₂N₃O₂, HCl H₂O: C, 61.47; H, 6.35; N, 8.27. Found: C, 61.09; H, 6.07; N, 8.18.

Similarly, compound 153b was prepared as described above for example 260b using (3S)-8-fluoro-3-{[(3S)-3-(5-fluoro-1H-indol-3-yl)butyl]amino}chromane-5-carboxamide as starting material. Chromatography ((98:2) CH₂Cl₂—MeOH (5% NH₄OH)) afforded 0.094 g (87%) of desired product which was converted to the HCl salt to generate (3S)-3-{cyclobutyl[(3S)-3-(5-fluoro-1H-indol-3-yl)butyl]amino}-8-fluorochromane-5-carboxamide hydrochloride salt as a white solid: mp 162-168° C.; [α]_{D}²⁵=+26.60 (c=1%, DMSO); MS (ES) m/z 454.3; Anal. Calcd for C₂₆H₂₉F₂N₃O₂.HCl.H₂O: C, 61.47; H, 6.35; N, 8.27. Found: C, 61.05; H, 6.33; N, 8.13.

### Example 262

### 3-[3-(5,7-Difluoro-1H-indol-3-yl)-1-methyl-propylamino]-8-fluoro-chroman-5-carboxylic acid amide (“Compound 154”)

To a solution of 4-(5,7-difluoro-1H-indol-3-yl)-butan-2-one (430 mg, 1.93 mmol) in THF (8 mL), was added 3-amino-8-fluorochromane-5-carboxamide (405 mg, 1.93 mmol), sodium triacetoxyborohydride (612 mg, 2.89 mmol), and acetic acid (0.1 mL, 1.9 mmol). The reaction mixture was stirred at room temperature for 1 day, then was quenched with saturated aqueous NaHCO₃ solution (10 mL). The aqueous mixture was extracted with EtOAc (3×10 mL). The combined organic layers were washed with H₂O (3×10 mL) and brine (3×10 mL). Purification by flash chromatography on silica gel (18:1:1 EtOAc:Et₃N:hexanes) afforded a clean mixture of stereoisomeric products. Preparative HPLC (Primesphere CN, 5×25 cm, 20% MeOH/CH₃Cl in hexane/diethylamine) afforded 388 mg of diastereomer 1 (as a racemate) and 325 mg of diastereomer 2 (as a racemate).

The enantiomers of diasteromer 1 were separated by chiral HPLC on Chiralcel AS (2×25 cm, 35% EtOH/diethylamine in hexane/diethylamine to afford 130 mg of enantiomer 1 (labeled as D1E1) and 170 mg of enantiomer 2 (labeled as D1E2).

The enantiomers of diastereomer 2 were also separated by chiral HPLC on Chiralcel AS (2×25 cm, 25% isopropanol/diethylamine in hexane/diethylamine) to a afford 107 mg of enantiomer 1 (D2E1) and 90 mg of enantiomer 2 (D2E2).

### Examples 262a, 262b, 262c and 262d

### Isomers 1, 2, 3 and 4 of 3-{(cyclopropylmethyl)[3-(5,7-difluoro-1H-indol-3-yl)-1-methylpropyl]amino}-8-fluorochromane-5-carboxamide (“Compounds 154a, 154b, 154c and 154d”)

Isomer 1 of compound 154 was prepared as follows: a mixture of 3-[3-(5,7-difluoro-1H-indol-3-yl)-1-methyl-propyl]amino}-8-fluoro-chroman-5-carboxylic acid amide (isomer D1E1, 130 mg, 0.32 mmol), cyclopropanecarboxaldehyde (0.14 mL, 1.87 mmol), sodium triacetoxyborohydride (132 mg, 0.62 mmol), and acetic acid (0.043 mL, 0.75 mmol) in THF (2 mL) was stirred at room temperature for 1 day. The reaction was quenched by the addition of saturated aqueous NaHCO₃ solution (5 mL) and extracted with EtOAc (3×5 mL). The combined organic layers were washed with H₂O (3×5 mL) and brine: (3×5 mL), then were dried over Na₂SO₄, filtered, and concentrated in vacuo. Chromatography (48:1:1 CH₂Cl₂:MeOH:NH₄OH) afforded 130 mg (88%) of Isomer 1 (compound 154a) of the title compound, which was converted to its HCl salt, an off-white solid: mp 160-165° C.; MS (ES) m/z 472.1 [M+H]⁺; [α]_{D}=−9.87° (c=0.79, DMSO).

Isomer 2 of compound 154 was converted to the title compound as described above for isomer 1 using isomer D1E2 (170 mg, 0.41 mmol), and was isolated in 78% yield after chromatography. The hydrochloride salt was isolated as an off-white solid: mp 180-185° C.; MS. ES m/z 472.1. [M+H]⁺; [α]_{D}=+8.33° (c=0.96, DMSO).

Isomer 3 of compound 154 was converted to the title compound as described above for isomer 1 using isomer D2E1 (107 mg, 0.26 mmol), and was isolated in 91% yield after chromatography. The hydrochloride salt was isolated as an off-white solid: mp 175-180° C.; MS ES m/z 472.1 [M+H]⁺; [α]_{D}=−49.88° (c=0.83, DMSO).

Isomer 4 of compound 154 was converted to the title compound as described above for isomer 1 using isomer D2E2 (90 mg, 0.22 mmol), and was isolated in 69% yield after chromatography. The hydrochloride salt was isolated as an off-white solid: mp 170-175° C.; MS ES m/z 472.1 [M+H]⁺; [a]_{D}=+44.290 (c=6.98, DMSO).

### Example 263

### (3R)-8-fluoro-3-{[3-(5-fluoro-1H-indol-3-yl)-2-methylpropyl]amino}chromane-5-carboxamide (“Compound 155”)

A solution of (3R)-3-amino-8-fluorochromane-5-carboxamide L-(+)-tartrate (1.20 g, 3.33 mmol) in anhydrous methanol (40 mL) and. acetic acid (0.27 mL, 4.7 mmol) was treated with 3-(5-fluoro-1H-indol-3-yl)-2-methylpropanal (720 mg, 3.5 mmol) and sodium cyanoborohydride (420 mg, 6.7 mmol). After stirring at ambient temperature for 16 hours the reaction was quenched with 1N aqueous NaOH (40 mL) and the methanol was removed under reduced pressure. The residue was extracted with ethyl acetate (3×40 ml). The combined ethyl acetate fractions were washed with saturated aqueous NaCl, dried over MgSO₄, and concentrated under reduced pressure. Chromatography ((97:3) CH₂Cl₂—MeOH (5% NH₄OH)) afforded 1.07 g (80%) of desired product as a white solid. The product was characterized by ¹HNMR.

### Examples 263a and 263b

### Isomers 1 and 2 of (3R)-3-{(cyclopropylmethyl)[3-(5-fluoro-1H-indol-3-yl)-2-methylpropyl]amino}-8-fluorochromane-5-carboxamide (“Compounds 155a and 155b”)

A solution of (3R)-8-fluoro-3-{[3-(5-fluoro-1H-indol-3-yl)-2-methylpropyl]amino}chromane-5-carboxamide (200 mg, 0.50 mmol) in anhydrous methanol (6.5 mL) was treated with glacial acetic acid (0.094 mL, 1.3 mmol), cyclopropanecarboxaldehyde (0.14 mL, 1.9 mmol), and sodium cyanoborohydride (63 mg, 1.0 mmol) and stirred for 16 hours at ambient temperature. Chromatography ((97:3) CH₂Cl₂—MeOH (5% NH₄OH)) afforded 0.214 g (94%) of desired product as a white solid. The diastereomers were separated by HPLC, isolated and converted to the HCl salt generating the following products:

Isomer 1 (Compound 155a) as a white solid: mp 155-159° C. (melts with decomposition); [α]_{D}²⁵=−22.2° (c=1% SOLUTION, DMSO); MS (ES) m/z 454.2.

Isomer 2 (Compound 155b) as a white solid: mp 155-160° C. (melts with decomposition); [α]_{D}²⁵=−59.60 (c=1% SOLUTION, DMSO); MS (ES) m/z 454.2.

### Example 264

### 8-fluoro-3-{[2-(7-methoxy-1-benzofuran-3-yl)ethyl]amino}chromane-5-carboxamide (“Compound 156”)

A solution of 3-amino-8-fluorochromane-5-carboxamide (0.5 g, 2.38 mmol) and N,N-diisopropylethylamine (0.83 mL, 4.8 mmol) in anhydrous DMSO (11.5 mL) was treated with 3-(2-bromoethyl)-7-methoxy-1-benzofuran (0.76 g, 2.98 mmol). The solution was heated to 80° C. and stirred for 17 hours at this temperature. After cooling to ambient temperature the reaction was treated with 1N aq. NaOH (˜60 mL) and extracted with ethyl acetate (3×50 mL). The combined organic phases were washed with saturated aqueous NaCl, dried over MgSO₄, and evaporated under reduced pressure to a gum. Chromatography ((97:3) CH₂Cl₂—MeOH (5% NH₄OH)) afforded 0.608 g (66%) of desired product as an amber solid: mp 172-174° C.; MS (ES) m/z 383.2; Anal. Calcd for C₂₁H₂₁FN₂O₄: C, 65.62; H, 5.51; N, 7.29. Found: C, 65.43; H, 5.50; N, 7.14.

### Example 265

### 3-{ethyl[2-(7-methoxy-1-benzofuran-3-yl)ethyl]amino}-8-fluorochromane-5-carboxamide (“Compound 157”)

A solution of 8-fluoro-3-{[2-(7-methoxy-1-benzofuran-3-yl)ethyl]amino}chromane-5-carboxamide (110 mg, 0.286 mmol) in anhydrous methanol (1.2 mL) was treated with acetic acid (0.05 mL, 0.80 mmol), acetaldehyde (0.06 mL, 1.07 mmol), and sodium cyanoborohydride (45 mg, 0.72 mmol), and stirred at ambient temperature for 16 hours. The reaction was quenched with 25 mL 1N aqueous NaOH and extracted with ethyl acetate (3×25 mL). The combined ethyl acetate phases were washed with saturated aqueous NaCl solution, dried over MgSO₄, and evaporated under reduced pressure to ˜5 mL solution. This residue was filtered through a Varian Bond Elut cartridge containing 5 g SCX resin. Elution with ((1:49) triethylamine-EtOAc) afforded 103 mg (87%) of desired product which was converted to the HCl salt to generate 3-{ethyl[2-(7-methoxy-1-benzofuran-3-yl)ethyl]amino}-8-fluorochromane-5-carboxamide hydrochloride salt as a white solid: mp 124-132° C.; MS (ES) m/z 413.2.

### Example 266

### 8-fluoro-3-[[2-(7-methoxy-1-benzofuran-3-yl)ethyl](propyl)amino]chromane-5-carboxamide (“Compound 158”)

This compound was prepared as described above for example 265 using propionaldehyde (0.08 mL, 1.10 mmol) affording 108 mg (88%) of desired product which was converted to the HCl salt to generate 8-fluoro-3-[[2-(7-methoxy-1-benzofuran-3-yl)ethyl](propyl)amino]chromane-5-carboxamide hydrochloride salt as a light-amber solid: mp 125-132° C.; MS (ES) m/z 427.2.

### Example 267

### 3-{cyclobutyl[2-(7-methoxy-1-benzofuran-3-yl)ethyl]amino}-8-fluorochromane-5-carboxamide (“Compound 159”)

This compound was prepared. as described above for example 265 using cyclobutanone (0.08 mL, 1.1 mmol) affording 116 mg (93%) of desired product which was converted to the HCl salt to generate 3-{cyclobutyl[2-(7-methoxy-1-benzofuran-3-yl)ethyl]amino}-8-fluorochromane-5-carboxamide hydrochloride salt as a light-amber solid: mp 135-145° C. (melts with decomposition); MS (ES) m/z 437.2.

### Example 268

### 3-{(cyclopropylmethyl)[2-(7-methoxy-1-benzofuran-3-yl)ethyl]amino}-8-fluorochromane-5-carboxamide (“Compound 160”)

This compound was prepared as described above for example 265 using cyclopropanecarboxaldehyde (0.080 mL, 1.1 mmol) affording 0.114 g (91%) of desired product which was converted to the HCl salt to generate 3-{(cyclopropylmethyl)[2-(7-methoxy-1-benzofuran-3-yl)ethyl]amino}-8-fluorochromane-5-carboxamide hydrochloride salt as a light-amber solid: mp 130-138° C. (melts with decomposition); MS (ES) m/z 437.2.

### Example 269

### 8-fluoro-3-{[3-(7-methoxy-1-benzofuran-3-yl)propyl]amino}chromane-5-carboxamide (“Compound 161”)

This compound was prepared as described above for example 264 using 3-(3-bromopropyl)-7-methoxy-1-benzofuran. Chromatography ((97:3) CH₂Cl₂—MeOH (5% NH₄OH)) afforded 0.945 g (62%) of desired product as a tan solid: mp 163-166° C.; MS (ES) m/z 397.2.

### Example 270

### 3-{ethyl[3-(7-methoxy-1-benzofuran-3-yl)propyl]amino}-8-fluorochromane-5-carboxamide (“Compound 162”)

This compound was prepared as described above for example 265 using 8-fluoro-3-{[3-(7-methoxy-1-benzofuran-3-yl)propyl]amino}chromane-5-carboxamide (114 mg, 0.286 mmol) and acetaldehyde (0.060 mL, 1.1 mmol) affording 0.064 g (52%) of desired product which was converted to the HCl salt to generate 3-{ethyl[3-(7-methoxy-1-benzofuran-3-yl)propyl]amino}-8-fluorochromane-5-carboxamide hydrochloride salt as a light-amber solid: mp 125-128° C.; MS (ES) m/z 425.2.

### Example 271

### 8-fluoro-3-[[3-(7-methoxy-1-benzofuran-3-yl)propyl](propyl)amino]chromane-5-carboxamide (“Compound 163”)

This compound was prepared as described above for example 265 using propionaldehyde (0.08 mL, 1.1 mmol) affording 114 mg (91%) of desired product which was converted to the HCl salt to generate 8-fluoro-3-[[3-(7-methoxy-1-benzofuran-3-yl)propyl](propyl)amino]chromane-5-carboxamide hydrochloride salt as a light-amber solid: mp 123-129° C.; MS (ES) m/z 441.2.

### Example 272

### 3-{cyclobutyl[3-(7-methoxy-1-benzofuran-3-yl)propyl]amino}-8-fluorochromane-5-carboxamide (“Compound 164”)

This compound was prepared as described above for example 265 using cyclobutanone (0.080 mL, 1.1 mmol) affording 0.128 g (99%) of desired product which was converted to the HCl salt to generate 3-{cyclobutyl[3-(7-methoxy-1-benzofuran-3-yl)propyl]amino}-8-fluorochromane-5-carboxamide hydrochloride salt as a light-amber solid: mp 131-140° C. (melts with decomposition); MS (ES) m/z 453.2.

### Example 273

### 3-{(cyclopropylmethyl)[3-(7-methoxy-1-benzofuran-3-yl)propyl]amino}-8-fluorochromane-5-carboxamide (“Compound 165”)

This compound was prepared as described above for example 265 using cyclopropanecarboxaldehyde (0.080 mL, 1.1 mmol) affording 0.115 g (89%) of desired product which was converted to the HCl salt to generate 3-{(cyclopropylmethyl)[3-(7-methoxy-1-benzofuran-3-yl)propyl]amino}-8-fluorochromane-5-carboxamide hydrochloride salt as a light-amber solid: mp 125-132° C.; MS (ES) m/z 453.2.

### Example 274

### 3-{butyl[3-(7-methoxy-1-benzofuran-3-yl)propyl]amino}-8-fluorochromane-5-carboxamide (“Compound 166”)

This compound was prepared as described above for example 265 using butyraldehyde (0.10 mL, 1.10 mmol). After work-up the residue was filtered through a Varian Bond Elut cartridge containing 5 g SCX resin. Elution with ((1:49) triethylamine-EtOAc) afforded 95 mg (92%) of desired product which was converted to the HCl salt to generate 3-{butyl[3-(7-methoxy-1-benzofuran-3-yl)propyl]amino}-8-fluorochromane-5-carboxamide hydrochloride salt as a light-amber solid: mp 110-117° C. (melts with decomposition); MS (ES) m/z 453.2.

### Example 275

### 8-fluoro-3-{[4-(7-methoxy-1-benzofuran-3-yl)butyl]amino}chromane-5-carboxamide (“Compound 167”)

This compound was prepared as described above for example 264 using 3-(4-bromobutyl)-7-methoxy-1-benzofuran. Chromatography ((97:3) CH₂Cl₂—MeOH (5% NH₄OH)) afforded 0.82 g (56%) of desired product as a white solid: mp 138-141° C.; MS (ES) m/z 413.2.

### Example 276

### 3-{ethyl[4-(7-methoxy-1-benzofuran-3-yl)butyl]amino}-8-fluorochromane-5-carboxamide (“Compound 168”)

This compound was prepared as described above for example 265 using 8-fluoro-3-{[4-(7-methoxy-1-benzofuran-3-yl)butyl]amino}chromane-5-carboxamide (118 mg, 0.286 mmol) and acetaldehyde (0.060 ml, 1.1 mmol) affording 0.087 g (69%) of desired product which was converted to the HCl salt to generate 3-{ethyl[4-(7-methoxy-1-benzofuran-3-yl)butyl]amino}-8-fluorochromane-5-carboxamide hydrochloride salt as a light-amber solid: mp 112-118° C.; MS (ES) m/z 441.2.

### Example 277

### 8-fluoro-3-[[4-(7-methoxy-1-benzofuran-3-yl)butyl](propyl)amino] chromane-5-carboxamide (“Compound 169”)

This compound was prepared as described above for example 265 using propionaldehyde (0.080 ml, 1.1 mmol) affording 0.120 g (92%) of desired product which was converted to the HCl salt to generate 8-fluoro-3-[[4-(7-methoxy-1-benzofuran-3-yl)butyl](propyl)amino]chromane-5-carboxamide hydrochloride salt as a light-amber solid: mp 99-110° C. (melts with decomposition) MS (ES) m/z 455.2.

### Example 278

### 3-{(cyclopropylmethyl)[4-(7-methoxy-1-benzofuran-3-yl)butyl]amino}-8-fluorochromane-5-carboxamide (“Compound 170”)

This compound was prepared as described above for example 265 using cyclopropanecarboxaldehyde (0.080 ml, 1.1 mmol) affording 0.124 g (93%) of desired product which was converted to the HCl salt to generate 3-{(cyclopropylmethyl)[4-(7-methoxy-1-benzofuran-3-yl)butyl]amino}-8-fluorochromane-5-carboxamide hydrochloride salt as a light-amber solid: mp 114-120° C.; MS (ES) m/z 465.2.

### Example 279

### 3-{cyclobutyl[4-(7-methoxy-1-benzofuran-3-yl)butyl]amino}-8-fluorochromane-5-carboxamide (“Compound 171”)

This compound was prepared as described above for example 265 using cyclobutanone (0.080 ml, 1.11 mmol) affording 0.12 g (97%) of desired product which was converted to the HCl salt to generate 3-{cyclobutyl[4-(7-methoxy-1-benzofuran-3-yl)butyl]amino}-8-fluorochromane-5-carboxamide hydrochloride salt as a light-arriber solid: mp 122-127° C. (melts with decomposition); MS (ES) m/z 465.2.

### Example 280

### (3R)-8-fluoro-3-[[4-(7-methoxy-1-benzofuran-3-yl)butyl](propyl)aminochromane-5-carboxamide (“Compound 172”)

This compound was prepared as described above for example 277 using (3R)-8-fluoro-3-1{[4-(7-methoxy-11-benzofuran-3-yl)butyl]amino}chromane-5-carboxamide (665 mg, 1.61 mmol) in anhydrous methanol (16 mL), and treated with acetic acid (0.28 mL, 4.8 mmol), propionaldehyde (0.29 mL, 4.0 mmol), and sodium cyanoborohydride (0.25 g, 4.0 mmol) at ambient temperature for 2 hours. Chromatography ((98:2) CH₂Cl₂—MeOH (5% NH₄OH)) afforded 0.76 g (100%) of desired product which was converted to the HCl salt to generate (3R)-8-fluoro-3-[[4-(7-methoxy-1-benzofuran-3-yl)butyl](propyl)amino]chromane-5-carboxamide hydrochloride salt as a white solid: mp 103-111° C.; [α]_{D}²⁵=−31.2° (c=1% SOLUTION, DMSO); MS (ESI) m/z 453.

### Example 281

### 8-fluoro-3-1[(6-fluoro-2,3,4,9-tetrahydro-1H-carbazol-3-yl)methyl]amino}chromane-5-carboxamide (“Compound 173”)

Glacial acetic acid (0.45 mL) and sodium cyanoborohydride (0.45 g) were added to a solution of 3-amino-8-fluorochromane-5-carboxamide (0.50 g, 2.4 mmol) in dry methanol (30 mL). To this solution was added 6-fluoro-2,3,4,9-tetrahydro-1H-carbazole-3-carbaldehyde (1.1 g, 5.0 mmol)) in dry methanol (10 mL). The reaction mixture was stirred at ambient temperature under nitrogen overnight. The reaction was quenched with 1N NaOH (20 mL). The solvent was removed under reduced pressure and the residue was partitioned between water and ethyl acetate. The organic portion was dried (MgSO₄) and evaporated and the residue was chromatographed on silica gel eluting with 1-10% methanol in dichloromethane to give 725.5 mg (74%) of the title compound: MS (ES) m/z 412.2.

### Examples 282a, 282b, 282c and 282d

### Isomers 1, 2, 3 and 4 of 3-{cyclobutyl[(6-fluoro-2,3,4,9-tetrahydro-1H-carbazol-3-yl)methyl]amino}-8-fluorochromane-5-carboxamide (“Compounds 174a, 174b, 174c and 174d”)

8-fluoro-3-{[(6-fluoro-2,3,4,9-tetrahydro-1H-carbazol-3-yl)methyl]amino}chromane-5-carboxamide (675 mg, 1.6 mmol) was dissolved in dry methanol (30 mL). To the solution was added cyclobutanone (630 mg, 0.7 mL), glacial acetic acid (586 mg, 0.5 mL), and sodium cyanoborohydride (566 mg, 9 mmol). The clear solution was allowed to stir at ambient temperature for 48 h. Additional cyclobutanone (0.7 mL) was added and stirring was continued until no remaining starting material was observed by TLC. The reaction mixture was quenched, with 1 N NaOH. The volatiles were removed under reduced pressure and the residue was partitioned between ethyl acetate and water. The organic phase was separated, dried (MgSO₄) and evaporated to give a residue which was purified by chiral HPLC [column: Chiralcel AD, 0.46×25 cm; mobile phase: 1:1 hexane:ethanol] to give the four diastereoisomers below as the free bases, which were then converted to the HCl salt to generate:

(−)-3-{cyclobutyl[(6-fluoro-2,3,4,9-tetrahydro-1H-carbazol-3-yl)methyl]amino}-8-fluorochromane-5-carboxamide hydrochloride salt (isomer 1, compound 174a, WAY-255317-A-1, P6864-221-1): mp 192° C.; [a]D=26.20 (c=1% SOLUTION, MeOH); MS (ES) m/z 466.2.

(+)-3-{cyclobutyl[(6-fluoro-2,3,4,9-tetrahydro-1H-carbazol-3-yl)methyl]amino}-8-fluorochromane-5-carboxamide hydrochloride salt (isomer 2, compound 174b, WAY-255318-A-1, P6864-221-2): mp 218° C.; [α]_{D}²⁵=+27.40 (c=1% SOLUTION, MeOH); MS (ES) m/z 464.2.

(+)-3-{cyclobutyl[(6-fluoro-2,3,4,9-tetrahydro-1H-carbazol-3-yl)methyl]amino}-8-fluorochromane-5-carboxamide hydrochloride salt (isomer 3, compound 174c, WAY-255319-A-1, P6864-221-3): mp 227° C.; [α]_{D}²⁵=+50.20 (c=1% SOLUTION, MeOH); MS (ES) m/z 464.2.

(−)-3-{cyclobutyl[(6-fluoro-2,3,4,9-tetrahydro-1H-carbazol-3-yl)methyl]amino}-8-fluorochromane-5-carboxamide hydrochloride salt (isomer 4, compound 174d, WAY-255320-A-1, P6864-221-4): mp 210° C.; [α]_{D}²⁵=50.2° (c=1% SOLUTION, MeOH); MS (ES) m/z 464.2.

### Examples 283a and 283b

### (−)-(3R)-8-fluoro-3-({[6-fluoro-2,3,4,9-tetrahydro-1H-carbazol-3-yl]methyl]amino)chromane-5-carboxamide (“Compound 175a”) and (+)-(3R)-8-fluoro-3-({[6-fluoro-2,3,4,9-tetrahydro-1H-carbazol-3-yl]methyl]amino)chromane-5-carboxamide (“Compound 175b”)

A solution of (3R)-3-amino-8-fluorochromane-5-carboxamide L-(+)-tartrate (0.203 g, 0.563 mmol) and 6-fluoro-2,3,4,9-tetrahydro-1H-carbazole-3-carbaldehyde (128 mg, 0.591 mmol) in anhydrous methanol (7.5 mL) and acetic acid (0.045 mL, 0.78 mmol) was treated with sodium cyanoborohydride (71 mg, 1.1 mmol). After stirring for 18 hours at ambient temperature the reaction was quenched with 1N aqueous NaOH (15 mL) and extracted with ethyl acetate (4×15 mL). The combined organic phases were washed with saturated aqueous NaCl, dried over MgSO₄ and evaporated under reduced pressure. Chromatography ((94:6) CH₂Cl₂—MeOH (5% NH₄OH)) afforded 0.18 g (78%) of desired product as a white solid. The diastereomers were separated by HPLC, isolated as free base, and converted to the HCl salt to generate:
(−)-(3R)-8-fluoro-3-({[6-fluoro-2,3,4,9-tetrahydro-1H-carbazol-3-yl]methyl}amino)chromane-5-carboxamide hydrochloride salt as a white solid: mp 197-202° C.; [α]_{D}²⁵=−52.8° (c=1% SOLUTION, DMSO); MS (ES) m/z 412.1.
(+)-(3R)-8-fluoro-3-({[6-fluoro-2,3,4,9-tetrahydro-1H-carbazol-3-yl]methyl}amino)chromane-5-carboxamide hydrochloride salt-as a white solid: mp 198-202° C.; [α]_{D}²⁵=+59.80 (c=1% SOLUTION, DMSO); MS (ES) m/z 412.1.

### Examples 284a and 284b

### (−)-(3R)-3-(ethyl{[6-fluoro-2,3,4,9-tetrahydro-1H-carbazol-3-yl]methyl}amino)-8-fluorochromane-5-carboxamide (“Compound 176a”) and (+)-(3R)-3-(ethyl{[6-fluoro-2,3,4,9-tetrahydro-1H-carbazol-3-yl]methyl]amino}-8-fluorochromane-5-carboxamide (“Compound 176b”)

These compounds were prepared as described above for example 282-using the desired starting material (−)-(3R)-8-fluoro-3-({[6-fluoro-2,3,4,9-tetrahydro-1H-carbazol-3-yl]methyl}amino)chromane-5-carboxamide (for Compound 176a) or (+)-(3R)-8-fluoro-3-({[6-fluoro-2,3,4,9-tetrahydro-1H-carbazol-3-yl]methyl}amino)chromane-5-carboxamide (for compound 176b), and acetaldehyde. Chromatography on Biotage Quad ((93:7) CH₂Cl₂—MeOH (5% NH₄OH)) afforded the following desired products:

0.066 g (77%) of (−)-(3R)-3-(ethyl{[6-fluoro-2,3,4,9-tetrahydro-1H-carbazol-3-yl]methyl}amino)-8-fluorochromane-5-carboxamide which was converted to the HCl salt to generate a white solid: mp 175-180° C.; [α]_{D}²⁵=−80.6° (c=1% SOLUTION, DMSO); MS (ES) m/z 440.2.

0.083 g (97%) of (+)-(3R)-3-(ethyl{[6-fluoro-2,3,4,9-tetrahydro-1H-carbazol-3-yl]methyl}amino)-8-fluorochromane-5-carboxamide which was converted to the HCl salt to generate a white solid: mp 174-178° C.; [α]_{D}²⁵=+14.8° (c=−1% SOLUTION, DMSO); MS (ES) m/z 440.2.

### Examples 285a and 285b

### (−)-(3R)-8-fluoro-3-[{[6-fluoro-2,3,4,9-tetrahydro-1H-carbazol-3-yl]methyl}(propyl)amino]chromane-5-carboxamide (“Comopund 177a”) and (+)-(3R)-8-fluoro-3-[{[6-fluoro-2,3,4,9-tetrahydro-1H-carbazol-3-yl]methyl}(propyl)amino]chromane-5-carboxamide (“Compound 177b”)

These compounds were prepared as described above for example 284 using propionaldehyde. Chromatography on Biotage Quad ((93:7) CH₂Cl₂—MeOH (5% NH₄OH)) afforded the following desired products:

0.085 g (96%) of (−)-(3R)-8-fluoro-3-[{[6-fluoro-2,3,4,9-tetrahydro-1H-carbazol-3-yl]methyl}(propyl)amino]chromane-5-carboxamide which was converted to the HCl salt to generate a white solid: mp 174-180° C.; [α]_{D}²⁵=−90.8° (c=1% SOLUTION, DMSO); MS (ES) m/z 454.2.

0.072 g (82%) of (+)-(3R)-8-fluoro-3-[{[6-fluoro-2,3,4,9-tetrahydro-1H-carbazol-3-yl]methyl}(propyl)amino]chromane-5-carboxamide which was converted to the HCl salt to generate a white solid: mp 173-178° C.; [α]_{D}²⁵=+12.4° (c=1% SOLUTION, DMSO); MS (ES) m/z 454.2.

### Examples 286a and 286b

### (−)-(3R)-3-((cyclopropylmethyl){[6-fluoro-2,3,4,9-tetrahydro-1H-carbazol-3-yl]methyl}amino)-8-fluorochromane-5-carboxamide (“Compound 178a”) and (+)-(3R)-3-((cyclopropylmethyl){[6-fluoro-2,3,4,9-tetrahydro-1H-carbazol-3-yl]methyl}amino)-8-fluorochromane-5-carboxamide (“Compound 178b”)

These compounds were prepared as described above for example 284 using cyclopropanecarboxaldehyde. Chromatography on Biotage Quad ((93:7) CH₂Cl₂—MeOH (5% NH₄OH)) afforded the following desired products:

0.071 g (79%) of (−)-(3R)-3-((cyclopropylmethyl){[6-fluoro-2,3,4,9-tetrahydro-1H-carbazol-3-yl]methyl}amino)-8-fluorochromane-5-carboxamide which was converted to the HCl salt to generate a white solid: mp 175-179° C.; [α]_{D}²⁵=−89.0° (c=1% SOLUTION, DMSO); MS (ES) m/z 466.2.

0.083 g (92%) of (+)-(3R)-3-((cyclopropylmethyl){[6-fluoro-2,3,4,9-tetrahydro-1H-carbazol-3-yl]methyl}amino)-8-fluorochromane-5-carboxamide which was converted to the HCl salt to generate a white solid: mp 174-178° C.; [α]_{D}₂₅=+10.00° (c=1% SOLUTION, DMSO); MS (ES) m/z 466.2.

### Examples 287a and 287b

### (+)-(3R)-8-fluoro-3-({[6-fluoro-2,3,4,9-tetrahydro-1H-carbazol-2-yl]methyl}amino)chromane-5-carboxamide (“Compound 179a”) and (−)-(3R)-8-fluoro-3-({[6-fluoro-2,3,4,9-tetrahydro-1H-carbazol-2-yl]methyl}amino)chromane-5-carboxamide (“Compound 179b”)

These compounds were prepared as described above for examples 283a and 283b using 6-fluoro-2,3,4,9-tetrahydro-1H-carbazole-2-carbaldehyde. Chromatography ((95:5) CH₂Cl₂—MeOH (5% NH₄OH)) afforded 0.169 g (73%) of desired product as a white solid. The diastereomers were separated by HPLC, isolated as free base, and converted to the HCl salt to generate:
(+)-(3R)-8-fluoro-3-({[6-fluoro-2,3,4,9-tetrahydro-1H-carbazol-2-yl]methyl}amino)chromane-5-carboxamide hydrochloride salt as a light tan solid: mp 184-188° C. (melts with decomposition); [α]_{D}²⁵=+49.00 (c=1% SOLUTION, DMSO); MS (ES) m/z 410.1.
(−)-(3R)-8-fluoro-3-({[6-fluoro-2,3,4,9-tetrahydro-1H-carbazol-2-yl]methyl}amino)chromane-5-carboxamide hydrochloride salt as an off-white solid: mp 186-191° C. (melts with decomposition); [α]_{D}²⁵=−25.2° (c=1% SOLUTION, DMSO); MS (ES) m/z 410.1.

### Examples 288a and 288b

### 3-[(1,4-cis)-4-(5-Methoxy-chroman-3-ylamino)-cyclohexyl]-1H-indole-5-carbonitrile (“Compound 180a”) and 3-[(1,4-trans)-4-(5-Methoxy-chroman-3-ylamino)-cyclohexyl]-1H-indole-5-carbonitrile (“Compound 180b”)

To 3-amino-5-methoxychroman (0.5 g, 2.36 mmol) in anhydrous 1,2-dichloroethane (20 mL), under nitrogen at room temperature, was added 3-(4-oxocyclohexyl)-1H-indole-5-carbonitrile (0.66 g, 2.6 mmol), acetic acid (0.24 mL, 4.72 mmol), and sodium triacetoxyborohydride (0.75 g, 3.54 mmol). The reaction mixture was stirred at room temperature overnight. The reaction mixture was quenched with 1N NaOH/H₂O and extracted with methylene chloride, dried over sodium sulfate, filtered and concentrated under vacuum. Chromatography ((19:1) EtOAc-MeOH) afforded 0.1.56 g (16%) of a yellow oil which was converted to the oxalate salt to generate 3-[(1,4-cis)-4-(5-Methoxy-chroman-3-ylamino)-cyclohexyl]-1H-indole-5-carbonitrile oxalate salt as a yellow solid: mp>140° C.; MS (ESI) m/z 402; Anal. Calcd for 1.00 C₂₅H₂₇N₃O₂+1.00 C₂H₂O₄+0.25 H₂O: C, 65.93; H, 5.94; N, 8.54. Found: C, 65.14; H, 5.98; N, 8.20.

Chromatography also afforded 0.12 g (13%) of a light yellow oil which was converted to the oxalate salt to generate 3-[(1,4-trans)-4-(5-Methoxy-chroman-3-ylamino)-cyclohexyl]-1H-indole-5-carbonitrile oxalate salt as an off-white solid: mp>140° C.; MS (ESI) m/z 402; Anal. Calcd for 1.00 C₂₅H₂₇N₃O₂+1.00 C₂H₂O₄+0.75 H₂O: C, 65.93; H, 5.94; N, 8.54. Found: C, 64.21; H, 5.73; N, 8.23.

### Examples 289a and 289b

### cis-N-[4-(5-fluoro-1H-indol-3-yl)cyclohexyl]-5-methoxychroman-3-amine (“Compound 181a”) and trans-N-[4-(5-fluoro-1H-indol-3-yl)cyclohexyl]-5-methoxychroman-3-amine (“Compound 181b”)

To 3-amino-5-methoxychroman (0.5 g, 2.79 mmol) in anhydrous 1,2-dichloroethane (25 mL), under nitrogen at room temperature, was added 4-(5-fluoro-1H-indol-3-yl)cyclohexanone (0.65 g, 2.79 mmol), acetic acid (0.29 mL, 5.58 mmol), and sodium triacetoxyborohydride (0.83 g, 3.9 mmol). The reaction mixture was stirred at room temperature overnight. The reaction mixture was quenched with 1N NaOH/H₂O and extracted with methylene chloride, dried over magnesium sulfate, filtered and concentrated under vacuum. Chromatography ((6:3:1) Hex-EtOAc-MeOH (1% NH₄OH)) afforded 0.71 g (65%) of a sticky gum which was converted to the HCl salt to generate cis-N-[4-(5-fluoro-1H-indol-3-yl)cyclohexyl]-5-methoxychroman-3-amine hydrochloride salt as an off-white solid: mp 156.5° C./dec; MS (ESI) m/z 393; Anal. Calcd for C₂₄H₂₇FN₂O₂.1.10 HCl: C, 66.33; H, 6.52; N, 6.45. Found: C, 66.42; H, 6.48; N, 6.36.

Chromatography also afforded 0.28 g (26%) of a gummy solid which was converted to the HCl salt to generate trans-N-[4-(5-fluoro-1H-indol-3-yl)cyclohexyl]-5-methoxychroman-3-amine hydrochloride salt as a white solid: mp 265° C./dec; MS (ESI) m/z 393; Anal. Calcd for C₂₄H₂₇FN₂O₂.HCl: C, 66.89; H, 6.55; N, 6.50. Found: C, 66.67; H, 6.65; N, 6.38.

### Example 290

### cis-N-[4-(5-fluoro-1H-indol-3-yl)cyclohexyl]-5-methoxy-N-propylchroman-3-amine (“Compound 182”)

To cis-N-[4-(5-fluoro-1H-indol-3-yl)cyclohexyl]-5-methoxychroman-3-amine (0.15 g, 0.38 mmol) in anhydrous methanol (6.4 mL), under nitrogen at room temperature, was added propionaldehyde (0.03 mL, 0.42 mmol), acetic acid (0.05 mL, 0.91 mmol) and sodium cyanoborohydride (0.048 g, 0.76 mmol). The reaction mixture was stirred at room temperature overnight. More propionaldehyde (0.055 mL, 0.76 mmol) was added and the reaction kept at 45° C. overnight. Chromatography ((6:3:1) Hex-EtOAc-MeOH (1% NH₄OH)) afforded 0.11 g (67%) of cis-N-[4-(5-fluoro-1H-indol-3-yl)cyclohexyl]-5-methoxy-N-propylchroman-3-amine which was converted to the HCl salt to generate cis-N-[4-(5-fluoro-1H-indol-3-yl)cyclohexyl]-5-methoxy-N-propylchroman-3-amine hydrochloride salt as an off-white solid: mp 148° C./dec; MS (ESI) m/z 437; Anal. Calcd for C₂₇H₃₃FN₂O₂.HCl: C, 68.56; H, 7.24; N, 5.92. Found: C, 68.55; H, 7.21; N, 5.80.

### Example 291

### trans-N-[4-(5-fluoro-1H-indol-3-yl)cyclohexyl]-5-methoxy-N-propylchroman-3-amine (“Compound 183”)

To trans-N-[4-(5-fluoro-1H-indol-3-yl)cyclohexyl]-5-methoxychroman-3-amine (0.15 g, 0.38 mmol) in anhydrous methanol (6.4 mL), under nitrogen at room temperature, was added propionaldehyde (0.03 mL, 0.42 mmol), acetic acid (0.05 mL, 0.91 mmol) and sodium cyanoborohydride (0.048 g, 0.76 mmol). The reaction mixture was stirred at room temperature overnight. More propionaldehyde (0.14 mL, 1.9 mmol) was added and the reaction mixture stirred at room temperature overnight. Chromatography ((6.5:2.5:1) Hex-EtOAc-MeOH (1% NH₄OH)) afforded 0.11 g (67%) of trans-N-[4-(5-fluoro-1H-indol-3-yl)cyclohexyl]-5-methoxy-N-propylchroman-3-amine which was converted to the HCl salt to generate trans-N-[4-(5-fluoro-1H-indol-3-yl)cyclohexyl]-5-methoxy-N-propylchroman-3-amine hydrochloride salt as a white solid: mp 159° C./dec; MS (ESI) m/z 437; Anal. Calcd for C₂₇H₃₃FN₂O₂.HCl: C, 68.56; H, 7.24; N, 5.92. Found: C, 68.80; H, 7.16; N, 5.84.

### Example 292

### 8-Fluoro-3-{[3-(1H-indol-1-yl)propyl]amino}chromane-5-carboxamide hydrochloride salt (“Compound 184”)

To a solution of indole (1 mmole) in anhydrous N,N-dimethylformamide (5 mL) kept under nitrogen was added sodium hydride (60% dispersion in oil, 0.044 g). After stirring at room temperature for 30 minutes the solution was cooled in an ice bath and 1,3-propanediol ditosylate (1.93 g) was added. After two additional hours at room temperature the mixture was diluted with ethyl acetate and water. The pH was quickly adjusted to neutral with 1.5 N aqueous potassium bisulfate. The organic phase was washed with brine, dried over anhydrous magnesium sulfate and evaporated to provide a yellowish solid. Flash chromatography of the residue on silica gel Merck-60 eluting with a gradient from 100% hexane to 15% ethyl acetate in hexane provided 1-[3-(toluene-4-sulfonyl)-propyl]-1H-indole (0.233 g) as an oil that solidified upon standing in the cold. It was used as such in the next step. MS [(+)ESI, m/z]: 330.07 [M+H]⁺.

To a solution of 1-[3-(toluene-4-sulfonyl)-propyl]-1H-indole (0.582 mmole) in anhydrous acetonitrile (10 mL) was added 3-amino-8-fluoro-chroman-5-carboxylic acid amide (1 equivalent) followed by solid potassium bicarbonate (0.097 g). The mixture was stirred under nitrogen and heated first to 55° C. for 1.5 hours and then to 80° C. overnight. Additional 3-amino-8-fluoro-chroman-5-carboxylic acid amide (0.1 equivalents) was added and the heating resumed for 2.5 hours. The reaction mixture was diluted with ethyl acetate and washed with water and brine. The organic phase was dried over anhydrous magnesium sulfate and evaporated to yield a light brown solid. Flash chromatography of the residue on silica gel Merck-60 eluting with a gradient from 100% dichloromethane to 10% ethyl acetate in dichloromethane followed by a gradient from 1% to 4% methanolic ammonia in dichloromethane provided the title compound (0.079 g) as a white solid, m.p. 155-156° C. MS [(+)ESI, m/z]: 368.2 [M+H]⁺. MS [(−)ESI, m/z]: 366.2 [M−H]⁻. HPLC (Chromolith Monolith, 0.46×10 cm column, sample dissolved in dimethylsulfoxide, acetonitrile/water (0.1% trifluoroacetic acid) gradient, 254 nm detection): R_{f}=1.53 min.

The hydrochloride salt was prepared by adding 1 equivalent of 1N hydrochloric acid in diethylether to a solution of the base in ethyl acetate, m.p. 132-133° C. (dec). MS [(+)ESI, m/z]: 368.2 [M+H]⁺. MS [(−)ESI, m/z]: 366.2 [M−H]⁻. HPLC (Chromolith Monolith, 0.46×10 cm column, sample dissolved in dimethylsulfoxide, acetonitrile/water (0.1% trifluoroacetic acid) gradient, 254 nm detection): Rₜ=1.53 min.

### Example 293

### 8-Fluoro-3-[4-(indol-1-yl)-butylamino]-chroman-5-carboxylic acid amide (“Compound 185”)

To a solution of the appropriate N-(4-bromobutyl)indoles of examples 96-99 (4 mmole) in dimethylsulfoxide (20 mL) was added 3-amino-8-fluoro-chroman-5-carboxylic acid amide (1 eq.) followed by N,N′-diisopropylethyl amine (Hünig's base, 1.2 eq.). The reaction mixture was stirred under nitrogen at 85° C. for 5 hours and then overnight at room temperature, diluted with ethyl acetate and washed with aqueous sodium bicarbonate. The aqueous phase was extracted with ethyl acetate (1×) and the pooled organic extracts were dried with anhydrous magnesium sulfate and evaporated to dryness. Purification was carried out by flash chromatography using a Biotage Quad 12/25 (Dyax Corp) with KP Sil 32-63 mM, 60 Å cartridges and the crude product was preabsorbed. Elution with a gradient from 100% dichloromethane to 4% methanolic ammonia in dichloromethane provided the title product as a pale yellow foam (63.5% yield). MS [(+)ESI, m/z]: 400.17 [M+H]⁺. MS [(−)ESI, m/z]: 398.2 [M−H]⁻. HPLC (Chromolith Monolith, 0.46×10 cm column, sample dissolved in ethanol, acetonitrile/water (0.1% trifluoroacetic acid) gradient, 254 nm detection): Rₜ=1.7 min.

### Example 294

### 8-Fluoro-3-[4-(5-fluoro-indol-1-yl)-butylamino]-chroman-5-carboxylic acid, amide (“Compound 186”)

This compound was obtained generally following the procedure for example 293. Obtained as a pale yellow foam (58% yield). MS [(+) ESI, m/z]: 400.2 [M+H]⁺. MS [(−)ESI, m/z]: 398.2 [M−H]⁻. HPLC (Chromolith Monolith, 0.46×10 cm column, sample dissolved in dimethylsulfoxide; acetonitrile/water (0.1% trifluoroacetic acid) gradient, 254 nm detection): Rₜ 1.7 min.

### Example 295

### 8-Fluoro-3-[4-(6-fluoro-indol-1-yl)-butylamino]-chroman-5-carboxylic acid amide (“Compound 187”)

This compound was obtained generally following the procedure for example 293. Obtained as a white solid (60% yield), m.p. 146-1480C. MS [(+)ESI, m/z]: 400.2 [M+H]⁺. MS [(−)ESI, m/z]: 398.2 [M−H]⁻. HPLC (Chromolith Monolith, 0.46×10 cm column, sample dissolved in acetonitrile, acetonitrile/water (0.1% trifluoroacetic acid) gradient, 254 nm detection): 1.69 min.

### Example 296

### 8-Fluoro-3-{[4-(7-fluoro-1H-indol-1-yl)butyl]amino}chromane-5-carboxamide hydrochloride salt (“Compound 188”)

This compound was obtained generally following the procedure for example 293. The free base was obtained as a white solid, m.p. 166-168° C. MS [(+)ESI, m/z]: 400.2 [M+H]⁺. MS [(−)ESI, m/z]: 398.2 [M−H]⁻. HPLC (Chromolith Monolith, 0.46×10 cm column, sample dissolved in ethanol, acetonitrile/water (0.1% trifluoroacetic acid) gradient, 254 nm detection): Rₜ=1.69 min.

The hydrochloride salt was obtained as an off-white amorphous solid by the addition of 1 equivalent of 1N hydrochloric acid in diethyl ether to an ethyl acetate/methanol solution of the free base. MS [(+)ESI, m/z]: 400.1 [M+H]⁺. MS [(−)ESI, m/z]: 398.1 [M−H]⁻. HPLC (Chromolith Monolith, 0.46×10 cm column, sample dissolved in dimethylsulfoxide, acetonitrile/water (0.1% trifluoroacetic acid) gradient, 254 nm detection): Rₜ=1.70 min.

### Example 297

### 3-{Ethyl[4-(7-fluoro-1H-indol-1-yl)butyl]amino}-8-fluorochromane-5-carboxamide hydrochloride salt (“Compound 189”)

To a solution of the appropriately substituted chromane-5-carboxamides of examples 293-296 (0.25-0.497 mmole) in methanol (18 mL/mmole) was added sequentially 1.2 equivalents of the appropriate aldehyde (ketone), 2.4 equivalents of glacial acetic acid and 2 equivalents of sodium cyanoborohydride. The mixture was stirred at room temperature under nitrogen for 3.5 hours and assayed by TLC. Whenever starting material was present additional aldehyde (ketone), glacial acetic acid and sodium cyanoborohydride was added and the stirring was continued until TLC showed completion of the reaction. The reaction mixture was then quenched with 1N sodium hydroxide, diluted with water (3-5 mL) and extracted with ethyl acetate (3×20 mL). The extracts were dried over anhydrous magnesium sulfate and evaporated to dryness. Purification of compounds of examples 297-300 below was carried out by flash chromatography using a Biotage Quad 12/25 (Dyax Corp) with KP Sil 32-63 mM, 60 Å cartridges and preabsorbing the crude product. Eluting with, hexane/ethyl acetate/methanolic ammonia (65:30:5) provided the pure products. Alternatively, purification of compounds of examples 301-312 was carried out by preparative reverse phase HPLC (Primesphere C18, 5×25 cm column, sample dissolved in acetonitrile, mobile phase: 30% acetonitrile in water (0.1% trifluoroacetic acid, flow rate 100 mL/min). The combined pure fractions were evaporated to small volume, basified with 1N sodium hydroxide and extracted with ethyl acetate. The extracts were dried over anhydrous magnesium sulfate, evaporated to dryness and the residue triturated with diethyl ether/hexane. The free base was obtained as a colorless glass (89.6% yield). MS [(+)ESI, m/z]: 428.2 [M+H]⁺. MS [(−)ESI, m/z]: 426.2 [M−H]⁻. HPLC (Chromolith Monolith, 0.46×10 cm column, sample dissolved in dimethylsulfoxide, acetonitrile/water (0.1% trifluoroacetic acid) gradient, 254 nm detection); Rₜ=1.8 min. The hydrochloride salt was prepared as an amorphous white solid by the addition of 1 equivalent of 1N hydrochloric acid in diethylether to a solution of the free base in ethyl acetate. MS [(+)ESI, m/z]: 428.2 [M+H]⁺. MS [(−)ESI, m/z]: 426.2 [M−H]⁻. HPLC (Chromolith Monolith, 0.46×10 cm column, sample dissolved in dimethylsulfoxide, acetonitrile/water (0.1% trifluoroacetic acid) gradient, 254 nm detection); Rₜ=1.78 min.

### Example 298

### 8-Fluoro-3-[[4-(7-fluoro-1H-indol-1-yl)butyl](propyl)amino]chromane-5-carboxamide hydrochloride salt (“Compound 190”)

This compound was prepared by generally following the procedure of example 297. The free base was obtained as a colorless glass (quantitative yield). MS [(+)ESI, m/z]: 442.2 [M+H]⁺. MS [(−)ESI, m/z]: 440.2 [M−H]⁻. HPLC (Chromolith Monolith, 0.46×10 cm column, sample dissolved in dimethylsulfoxide, acetonitrile/water (0.1% trifluoroacetic acid) gradient, 254 nm detection): Rₜ=1.89 min.

The hydrochloride salt was prepared as an amorphous white solid by the addition of 1 equivalent of 1N hydrochloric acid in diethylether to a solution of the free base in ethyl acetate. MS [(+)ESI, m/z]: 442.2 [M+H]⁺. MS [(−)ESI, m/z]: 440.2 [M−H]⁻. HPLC (Chromolith Monolith, 0.46×10 cm column, sample dissolved in dimethylsulfoxide, acetonitrile/water (0.1% trifluoroacetic acid) gradient, 254 nm detection); Rₜ=1.88 min.

### Example 299

### 3-{(Cyclopropylmethyl)[4-(7-fluoro-1H-indol-1-yl)butyl]amino}-8-fluorochromane-5-carboxamide hydrochloride salt (“Compound 191”)

This compound was prepared by generally following the procedure of example 297. The free base was obtained as a colorless glass (97% yield). MS [(+)ESI, m/z]: 454.2 [M+H]⁺. MS [(−)ESI, m/z]: 452.2 [M−H]⁻. HPLC (Chromolith Monolith, 0.46×10 cm column, sample dissolved in dimethylsulfoxide, acetonitrile/water (0.1% trifluoroacetic acid) gradient, 254 nm detection): Rₜ=1.89 min.

The hydrochloride salt was prepared as an amorphous white solid by the addition of 1 equivalent of 1N hydrochloric acid in diethylether to a solution of the free base in ethyl acetate. MS [(+)ESI, m/z]: 454.2 [M+H]⁺. MS [(−)ESI, m/z]: 452.2 [M−H]⁻. HPLC (Chromolith Monolith, 0.46×10 cm column, sample dissolved in dimethylsulfoxide, acetonitrile/water (0.1% trifluoroacetic acid) gradient, 254 nm detection); Rₜ=1.88 min.

### Example 300

### 3-{Cyclobutyl[4-(7-fluoro-1H-indol-1-yl)butyl]amino}-8-fluorochromane-5-carboxamide hydrochloride salt (“Compound 192”)

This compound was prepared by generally following the procedure of example 297. The free base was obtained as a colorless glass (93% yield). MS [(+)ESI, m/z]: 454.2 [M+H]⁺. MS [(−)ESI, m/z]: 452.2 [M−H]⁻. HPLC (Chromolith Monolith, 0.46×10 cm column, sample dissolved in dimethylsulfoxide, acetonitrile/water (0.1% trifluoroacetic acid) gradient, 254 nm detection): Rₜ=1.87 min.

The hydrochloride salt was prepared as an amorphous white solid by the addition of 1 equivalent of 1N hydrochloric acid in diethyl ether to a solution of the free base in ethyl acetate. MS [(+)ESI, m/z]: 454.2 [M+H]⁺. MS [(−)ESI, m/z]: 452.2 [M−H]⁻. HPLC (Chromolith Monolith, 0.46×10 cm column, sample dissolved in dimethylsulfoxide, acetonitrile/water (0.1% trifluoroacetic acid) gradient, 254 nm detection); Rₜ=1.88 min.

### Example 301

### 3-{Ethyl[4-(6-fluoro-1H-indol-1-yl)butyl]amino}-8-fluorochromane-5-carboxamide hydrochloride salt (“Compound 193”)

This compound was prepared by generally following the procedure of example 297. The free base was obtained as a glassy white foam (71% yield). MS [(+)ESI, m/z]: 428.2 [M+H]⁺. MS [(−)ESI, m/z]: 426.2 [M−H]⁻. HPLC (Chromolith Monolith, 0.46×10 cm column, sample dissolved in dimethylsulfoxide, methanol/water (0.1% trifluoroacetic acid) gradient, 254 nm detection); Rₜ=2.9 min.

The hydrochloride salt was prepared as an amorphous off-white solid by the addition of 1 equivalent of 1N hydrochloric acid in diethyl ether to a solution of the free base in ethyl acetate. MS [(+)ESI, m/z]: 428.2 [M+H]⁺. MS [(−)ESI, m/z]: 426.2 [M−H]⁻. HPLC (Chromolith Monolith, 0.46×10 cm column, sample dissolved in dimethylsulfoxide, methanol/water (0.1% trifluoroacetic acid) gradient, 254 nm detecti on); Rₜ=2.9 min.

### Example 302

### 8-Fluoro-3-[[4-(6-fluoro-1H-indol-1-yl)butyl](propyl)amino]chromane-5-carboxamide hydrochloride salt (“Compound 194”)

This compound was prepared by generally following the procedure of example 297. The free base was obtained as a glassy foam (73% yield). MS [(+)ESI, m/z]: 442.2 [M+H]⁺. MS [(−)ESI, m/z]: 440.2 [M−H]⁻. HPLC (Chromolith Monolith, 0.46×10 cm column, sample dissolved in dimethylsulfoxide, methanol/water (0.1% trifluoroacetic acid) gradient, 254 nm detection): Rₜ=3.0 min.

The hydrochloride salt was prepared as an amorphous off-white solid by the addition of 1 equivalent of 1N hydrochloric acid in diethyl ether to a solution of the free base in ethyl acetate. MS [(+)ESI, m/z]: 442.2 [M+H]⁺. MS [(−)ESI, m/z]: 440.2 [M−H]⁻. HPLC (Chromolith Monolith, 0.46×10 cm column, sample dissolved in dimethylsulfoxide, methanol/water (0.1% trifluoroacetic acid) gradient, 254 nm detection); Rₜ=2.9 min.

### Example 303

### 3-{(Cyclopropylmethyl)[4-(6-fluoro-1H-indol-1-yl)butyl]amino}-8-fluorochromane-5-carboxamide hydrochloride salt (“Compound 195”)

This compound was prepared by generally following the procedure of example 297. The free base was obtained as a glassy foam (81.6% yield). MS [(+)ESI, m/z]: 454.2 [M+H]⁺. MS [(−)ESI, m/z]: 452.2 [M−H]⁻. HPLC (Chromolith Monolith, 0.46×10 cm column, sample dissolved in dimethylsulfoxide, acetonitrile/water (0.1% trifluoroacetic acid) gradient, 254 nm detection): Rₜ=3.0 min.

The hydrochloride salt was prepared as an amorphous off-white solid by the addition of 1 equivalent of 1N hydrochloric acid in diethyl ether to a solution of the free base in ethyl acetate. MS [(+)ESI, m/z]: 454.2 [M+H]⁺. MS [(−)ESI, m/z): 452.2 [M−H]⁻. HPLC (Chromolith Monolith, 0.46×10 cm column, sample dissolved in dimethylsulfoxide, methanol/water (0.1% trifluoroacetic acid) gradient, 254 nm detection); Rₜ=2.9 min.

### Example 304

### 3-{Cyclobutyl[4-(6-fluoro-1H-indol-1-yl)butyl]amino}-8-fluorochromane-5-carboxamide hydrochloride salt (“Compound 196”)

This compound was prepared by generally following the procedure of example 297. The free base was obtained as a glassy foam (75% yield). MS [(+)ESI, m/z]: 454.2 [M+H]⁺. MS [(−)ESI, m/z]: 452.2 [M−H]⁻. HPLC (Chromolith Monolith, 0.46×10 cm column, sample dissolved in acetonitrile, acetonitrile/water (0.1% trifluoroacetic acid) gradient, 254 nm detection); Rₜ=1.9 min.

The hydrochloride salt was prepared as an amorphous off-white solid by the addition of 1 equivalent of 1N hydrochloric acid in diethyl ether to a solution of the free base in ethyl acetate. MS [(+)ESI, m/z]: 454.2 [M+H]⁺. MS [(−)ESI, m/z]: 452.2 [M−H]⁻. HPLC (Chromolith Monolith, 0.46×10 cm column, sample dissolved in dimethylsulfoxide, methanol/water (0.1% trifluoroacetic acid) gradient, 254 nm detection); Rₜ=2.9 min.

### Example 305

### 3-{Ethyl[4-(5-fluoro-1H-indol-1-yl)butyl]amino}-8-fluorochromane-5-carboxamide (“Compound 197”)

This compound was prepared by generally following the procedure of example 297. The free base was obtained as a glassy foam (63% yield). MS [(+)ESI, m/z]: 428.2 [M+H]⁺. MS [(−)ESI, m/z]: 426.2 [M−H]⁻.

### Example 306

### 8-Fluoro-3-[[4-(5-fluoro-1H-indol-1-yl)butyl](propyl)amino]chromane-5-carboxamide hydrochloride salt (“Compound 198”)

This compound was prepared by generally following the procedure of example 297. The free base was obtained as a glassy foam (63% yield). MS [(+)ESI, m/z]: 442.2.[M+H]⁺. MS [(−)ESI, m/z]: 440.2 [M−H]⁻. HPLC (Chromolith Monolith, 0.46×10 cm column, sample dissolved in dimethylsulfoxide, methanol/water (0.1% trifluoroacetic acid) gradient, 254 nm detection): Rₜ=3.0 min.

The hydrochloride salt was prepared as an amorphous off-white solid by the addition of 1 equivalent of 1N hydrochloric acid in diethyl ether to a solution of the free base in ethyl acetate. MS [(+)ESI, m/z]: 442.2 [M+H]⁺. MS [(−)ESI, m/z]: 440.2 [M−H]⁻. HPLC (Chromolith Monolith, 0.46×10 cm column, sample dissolved in acetonitrile, acetonitrile/water (0.1% trifluoroacetic acid) gradient, 254 nm detection); R_{f}=1.83 min.

### Example 307

### 3-{(Cyclopropylmethyl)[4-(5-fluoro-1H-indol-1-yl)butyl]amino}-8-fluorochromane-5-carboxamide hydrochloride salt (“Compound 199”)

This compound was prepared by generally following the procedure of example 297. The free base was obtained as a glassy foam (71% yield). MS [(+)ESI, m/z]: 454.2 [M+H]⁺. MS [(−)ESI, m/z]: 452.2 [M−H]⁻. HPLC (Chromolith Monolith, 0.46×10 cm column, sample dissolved in dimethylsulfoxide, methanol/water (0.1% trifluoroacetic acid) gradient, 254 nm detection): Rₜ=3.0 min.

The hydrochloride salt was prepared as an amorphous off-white solid by the addition of 1 equivalent of 1N hydrochloric acid in diethyl ether to a solution of the free base in ethyl acetate. MS [(+)ESI, m/z]: 454.2 [M+H]⁺. MS [(−)ESI, m/z]: 452.2 [M−H]⁻. HPLC (Chromolith Monolith, 0.46×10 cm column, sample dissolved in acetonitrile, methanol/water (0.1% trifluoroacetic acid) gradient, 254 nm detection); Rₜ=1.87 min.

### Example 308

### 3-{Cyclobutyl[4-(5-fluoro-1H-indol-1-yl)butyl]amino}-8-fluorochromane-5-carboxamide hydrochloride salt (“Compound 200”)

This compound was prepared by generally following the procedure of example 297. The free base was obtained as a glassy foam (71% yield). MS [(+)ESI, m/z]: 454.2 [M+H]⁺. MS [(−)ESI, m/z]: 452.2 [M−H]⁻. HPLC (Chromolith Monolith, 0.46×10 cm column, sample dissolved in dimethylsulfoxide, methanol/water (0.1% trifluoroacetic acid) gradient, 254 nm detection): Rₜ=3.0 min.

The hydrochloride salt was prepared as an amorphous off-white solid by the addition of 1 equivalent of 1N hydrochloric acid in diethyl ether to a solution of the free base in ethyl acetate. MS [(+)ESI, m/z]: 454.2 [M+H]⁺. MS [(−)ESI, m/z]: 452.2 [M−H]⁻. HPLC (Chromolith Monolith, 0.46×10 cm column, sample dissolved in dimethylsulfoxide, methanol/water (0.1% trifluoroacetic acid) gradient, 254 nm detection); Rₜ=2.94 min.

### Example 309

### 3-{Ethyl[4-(4-fluoro-1H-indol-1-yl)butyl]amino}-8-fluorochromane-5-carboxamide hydrochloride salt (“Compound 201”)

This compound was prepared by generally following the procedure of example 297. The free base was obtained as a glassy foam (53% yield). The hydrochloride salt was prepared as an amorphous off-white solid by the addition of 1 equivalent of 1N hydrochloric acid in diethyl ether to a solution of the free base in ethyl acetate. MS [(+)ESI, m/z]: 428.2 [M+H]⁺. MS [(−)ESI, m/z]: 426.2 [M−H]⁻. HPLC (Chromolith Monolith, 0.46×10 cm column, sample dissolved in dimethylsulfoxide, methanol/water (0.1% trifluoroacetic acid) gradient, 254 nm detection); Rₜ=2.9 min.

### Example 310

### 8-Fluoro-3-[[4-(4-fluoro-1H-indol-1-yl)butyl](propyl)amino]chromane-5-carboxamide hydrochloride salt (“Compound 202”)

This compound was prepared by generally following the procedure of example 297. The free base was obtained as a glassy foam (69% yield). MS [(+)ESI, m/z]: 442.2 [M+H]⁺. HPLC (Chromolith Monolith, 0.46×10 cm column, sample dissolved in dimethylsulfoxide, methanol/water (0.1% trifluoroacetic acid) gradient, 254 nm detection): Rₜ=3.0 min.

The hydrochloride salt was prepared as an amorphous off-white solid by the addition of 1 equivalent of 1N hydrochloric acid in diethyl ether to a solution of the free base in ethyl acetate. MS [(+)ESI, m/z]: 442.2 [M+H]⁺. MS [(−)ESI, m/z]: 440.3 [M−H]⁻. HPLC (Chromolith Monolith, 0.46×10 cm column, sample dissolved in acetonitrile, methanol/water (0.1% trifluoroacetic acid) gradient, 254 nm detection); R. =1.85 min.

### Example 311

### 3-{(Cyclopropylmethyl)[4-(4-fluoro-1H-indol-1-yl)butyl]amino}-8-fluorochromane-5-carboxamide hydrochloride salt (“Compound 203”)

This compound was prepared by generally following the procedure of example 297. The free base was obtained as a glassy foam (73% yield). MS [(+)ESI, m/z]: 454.0 [M+H]⁺. HPLC (Chromolith Monolith, 0.46×10 cm column, sample dissolved in dimethylsulfoxide, methanol/water (0.1% trifluoroacetic acid) gradient, 254 nm detection): R. =3.0 min:

The hydrochloride salt was prepared as an amorphous off-white solid by the addition of 1 equivalent of 1N hydrochloric acid in diethyl ether to a solution of the free base in ethyl acetate. MS [(+)ESI, m/z]: 454.3 [M+H]⁺. MS [(−)ESI, m/z]: 452.3 [M−H]⁻. HPLC (Chromolith Monolith, 0.46×10 cm column, sample dissolved in dimethylsulfoxide, methanol/water (0.1% trifluoroacetic acid) gradient, 254 nm detection); Rₜ=3.0 min.

### Example 312

### 3-{Cyclobutyl[4-(4-fluoro-1H-indol-1-yl)butyl]amino}-8-fluorochromane-5-carboxamide hydrochloride salt (“Compound 204”)

This compound was prepared by generally following the procedure of example 297. The free base was obtained as a glassy foam (68% yield). MS [(+)ESI, m/z]: 454.2 [M+H]⁺. MS [(−)ESI, m/z]: 452.2 [M−H]⁻. HPLC (Chromolith Monolith, 0.46×10 cm column, sample in dimethylsulfoxide, methanol/water (0.1% trifluoroacetic acid) gradient, 254 nm detection): Rₜ=3.0 min.

The hydrochloride salt was prepared as an amorphous off-white solid by the addition of 1 equivalent of 1N hydrochloric acid in diethyl ether to a solution of the free base in ethyl acetate. MS [(+)ESI, m/z]: 454.2 [M+H]⁺. MS {(−)ESI, m/z]: 452.3 [M−H]⁻. HPLC (Chromolith Monolith, 0.46×10 cm column, sample in acetonitrile, acetonitrilel/water (0.1% trifluoroacetic acid) gradient, 254 nm detection); Rₜ=1.86 min.

### Example 313

### Testing Affinity of Compounds for the 5-HT Transporter

A protocol similar to that used by Cheetham et al. (Neuropharmacol., 1993, 32: 737) was used to determine the affinity of the compounds of the invention for the serotonin transporter. The compound's ability to displace ³H-paroxetine from male rat cortical membranes was determined using a Tom Tech filtration device to separate bound from free ³H-paroxetine and Wallac 1205 Beta Plate® counter to quantitate bound radioactivity. Kᵢs thus determined for standard clinical antidepressants are 1.96 nM for fluoxetine, 14.2 nM for imipramime and 67.6 nM for zimelidine. A strong correlation has been found between ³H-paroxetine binding in rat frontal cortex and ³H-serotonin uptake inhibition.

High affinity for the serotonin 5-HT_{1A} receptor was established by testing the claimed compound's ability to displace [³H] 8-OH-DPAT (dipropylarinotetralin) from the 5-HT_{1A} serotonin receptor following a modification of the procedure of Hall et al., (J. Neurochem., 1985, 44: 1685) which utilizes CHO cells stably transfected with human 5-HT_{1A} receptors. The 5-HT_{1A} affinities for the compounds of the invention are reported below as Kᵢs.

The agonist or antagonist activity at 5-HT_{1A} receptors was established by using two different assays. The ³⁵S-GTPγS binding assay similar to that used by Lazareno and Birdsall (Br. J. Pharmacol., 1993, 109: 1120) was used to determine the test compound's ability to affect the binding of ³⁵S-GTPγS to membranes containing cloned human 5-HT_{1A} receptors. Agonists produce an increase in binding whereas antagonists produce no increase but rather reverse the effects of the standard agonist 8-OH-DPAT. The test compound's maximum stimulatory effect is represented as the Eₘₐₓ, while its potency is defined by the EC₅₀. The test compound's maximum inhibitory effect is represented as the Imax, while its potency is defined by the IC₅₀. The second assay measured cAMP accumulation upon binding of the ligand to the 5-HT_{1A} receptor. Antagonists block the effect of the standard agonist 8-OH-DPAT resulting in an increase in cAMP accumulation while agonists have the reverse effect. The test compound's maximum stimulatory or inhibitory effect is represented as the Eₘₐₓ while its potency is defined by either IC₅₀ for an antagonist or EC₅₀ for an agonist. [³H]-8-OH-DPAT was used to determine maximum agonist or antagonist response in both functional assays.

The results of the three standard experimental test procedures described above were as follows:

| | | | | | 5-HT_{1A} |
|---|---|---|---|---|---|
| | | | 5-HT_{1A} | 5-HT_{1A} | Function |
| | 5-HT_{1A} | 5-HT | Function | Function | cAMP |
| | Receptor | Transporter | GTP_{γ}S | GTP_{γ}S | EC₅₀ or IC₅₀ |
| | Affinity | Affinity | EC₅₀ (nM) | IC₅₀ (nM) | (nM) |
| Compound | Kᵢ (nM) | Kᵢ (nM) | (Emax) | (Imax) | (Emax) |
| 1 | 212.20 | 7.00 | 5000 (100) | Not Tested | EC₅₀ 228.50 (79.5) |
| 1a | 46.30 | 26.20 | 993 (87) | Not Tested | EC₅₀ 95.85 (84.5) |
| 1b | 47% @ 1 μM | 76.00 | Not Tested | Not Tested | Not Tested |
| 2 | 2.94 | 6.00 | 168.20 (77) | Not Tested | EC₅₀ 10.40 (83.5) |
| 2a | 1.48 | 8.50 | Not Tested | 404.9 (77.4) | EC₅₀ 18.53 (77.5) |
| 2b | 2.21 | 3.86 | 36.28 (80) | Not Tested | EC₅₀ 19.86 (97) |
| 3a | 35.07 | 5.00 | Not Tested | 1295 (79) | IC₅₀ 134.65 (0) |
| 3b | 15.53 | 4.50 | Not Tested | 665 (35.8) | EC₅₀ 31.37 (90.5) |
| 4 | 10.16 | 2.60 | Not Tested | 185 (100) | IC₅₀ 61.03 (0) |
| 4a | 265.50 | 4.93 | Not Tested | 3208 (100) | IC₅₀ 926 (0) |
| 4b | 1.23 | 1.53 | Not Tested | 43.95 (100) | IC₅₀ 27.38 (0) |
| 5 | 1.48 | 3.60 | 24.5 (28.4) | 290.7 (88.2) | IC₅₀ 65.55 (0) |
| 5a | 2.04 | 4.10 | Not Tested | 44.1 (100) | IC₅₀ 70.10 (0) |
| 5b | 0.10 | 3.70 | 35.6 (18) | 31.85 (82) | EC₅₀ 16.30 (69) |
| 6 | 30% @ 1 μM | 18.90 | Not Tested | 4841 (87) | Not Tested |
| 7 | 6.21 | 3.20 | Not Tested | 1649 (100) | IC₅₀ 59.70 (0) |
| 8 | 103.00 | 259.00 | Not Tested | 1377 (100) | IC₅₀ 478.50 (0) |
| 9a | 1.73 | 3.34 | Not Tested | 206.77 (100) | IC₅₀ 61.15 (0) |
| 9b | 68.90 | 12.15 | Not Tested | 6000 (74) | IC₅₀ 197 (0) |
| 10 | 6.25 | 10.90 | 30.5 (85) | Not Tested | EC₅₀ 16.30 (70.5) |
| 11 | 73.75 | 161.00 | Not Tested | 344.5 (62) | IC₅₀ 403.00 (0) |
| 12a | 5.19 | 22.00 | Not Tested | 323 (96) | IC₅₀ 47.45 (0) |
| 12b | 19.39 | 50.00 | 366 (73.5) | Not Tested | EC₅₀ 62.62 (78) |
| 12c | 48.77 | 121.00 | Not Tested | 3000 (100) | IC₅₀ 73.69 (0) |
| 12d | | 152.00 | Not Tested | 6000 (13) | Not Tested |
| 13a | 24.30 | 23.50 | Not Tested | 534 (100) | IC₅₀ 76.56 (0) |
| 13b | 151.70 | 37.00 | 1470 (56.5) | Not Tested | EC₅₀ 138.8 (68) |
| 13c | 119.30 | 118.00 | Not Tested | 2517 (92) | IC₅₀ 316.45 (0) |
| 13d | Not Tested | 112.00 | Not Tested | Not Tested | Not Tested |
| 14a | 41.65 | 13.60 | Not Tested | 2211 (59) | IC₅₀ 146.50 (0) |
| 14b | 1.88 | 9.50 | Not Tested | 477 (100) | IC₅₀ 109.50 (0) |
| 15a | 1.88 | 10.20 | Not Tested | 332.7 (100) | IC₅₀ 72.70 (0) |
| 15b | 31.65 | 46.70 | 107.9 (32) | Not Tested | EC₅₀ 32.40 (73) |
| 16a | 37.25 | 0.90 | Not Tested | 3000 (100) | IC₅₀ 187.00 (0) |
| 16b | 128.00 | 0.95 | 807.5 (20) | Not Tested | EC₅₀ 164.00 (64.5) |
| 17 | 0.88 | 159.00 | 93.3 (42) | Not Tested | EC₅₀ 76.20 (74.5) |
| 18 | 3.72 | 127.00 | 232.1 (100) | Not Tested | EC₅₀ 11.02 (93.5) |
| 19 | 16.05 | 405.00 | 317.3 (100) | Not Tested | EC₅₀ 81.03 (86.5) |
| 20 | 0.37 | 575.00 | 35.7 (84) | Not Tested | EC₅₀ 62.15 (88) |
| 21 | 0.38 | 310.50 | 28.1 (98.5) | Not Tested | EC₅₀ 9.51 (90.5) |
| 22 | 4.77 | 35.70 | 224.3 (82) | 1180 (31) | EC₅₀ 31.03 (100) |
| 23 | 15.85 | 9.00 | 99.50 (100) | Not Tested | EC₅₀ 39.33 (80.5) |
| 24 | 8.87 | 9.75 | 48.33 (91.7) | Not Tested | EC₅₀ 7.02 (88.5) |
| 25 | 24.28 | 16.50 | 155.0 (88) | Not Tested | EC₅₀ 1.06 (93.5) |
| 26 | 19.40 | 12.50 | 210.5 (79.5) | Not Tested | EC₅₀ 23.60 (83.5) |
| 27 | 12.61 | 13.50 | 116.5 (97) | Not Tested | EC₅₀ 16.37 (88.5) |
| 28 | 10.97 | 17.50 | 166.5 (88) | Not Tested | EC₅₀ 36.5 (86) |
| 29 | 7.32 | 27.00 | 205.3 (70) | Not Tested | EC₅₀ 77.06 (99) |
| 30 | 3.62 | 12.60 | 94.6 (83) | Not Tested | EC₅₀ 27.70 (94) |
| 31 | 2.33 | 2.20 | 53.5 (99) | Not Tested | EC₅₀ 26.30 (96) |
| 32 | 2.78 | 1.10 | 122.9 (100) | Not Tested | EC₅₀ 85.39 (95) |
| 33 | 40.01 | 6.80 | 3.62 (69) | Not Tested | EC₅₀ 95.17 (91) |
| 34 | 15.56 | 13.00 | Not Tested | 2132 (76) | EC₅₀ 61.89 (37) |
| 35 | 2.14 | 41.00 | Not Tested | 489 (80) | IC₅₀ 23.36 (0) |
| 36 | 11.66 | Not Tested | Not Tested | 668 (56) | EC₅₀ 12.25 (61) |
| 37 | 1.68 | 4.30 | 146.2 (88) | Not Tested | EC₅₀ 14.05 (91) |
| 38 | 0.15 | 29.00 | 25.8 (90) | Not Tested | EC₅₀ 8.84 (97.5) |
| 39 | 36.20 | 69.00 | 5839 (100) | Not Tested | EC₅₀ 108.00 (70.5) |
| 40 | 80.80 | 3.80 | Not Tested | 3773 (46) | IC₅₀ (0) |
| 41 | 57.25 | 148.00 | 3455 (84) | Not Tested | EC₅₀ 162.00 (78.5) |
| 42 | 50.45 | 54.00 | 763 (84) | Not Tested | EC₅₀ 154.50 (72.5) |
| 43 | 1.02 | 40.90 | 51.3 (100) | Not Tested | EC₅₀ 10.80 (91.5) |
| 44 | 21.35 | 47.20 | Not Tested | 242.4 (59) | EC₅₀ 70.00 (80.5) |
| 45 | 2.16 | 5.25 | 56 (72) | Not Tested | EC₅₀ 77.25 (89.5) |
| 46 | 14.60 | 7.60 | 107.2 (36) | 218 (81) | IC₅₀ 898.50 (0) |
| 47 | 4.12 | 2.16 | 10.4 (100) | Not Tested | EC₅₀ 3.50 (91.5) |
| 47a | 1.55 | 0.83 | 5.60 (80.5) | Not Tested | EC₅₀ 7.61 (99) |
| 47b | 0.34 | 1.10 | 19.4 (87) | Not Tested | EC₅₀ 1.55 (100) |
| 48 | 5.98 | 6.50 | 24.0 (93) | Not Tested | EC₅₀ 35.00 (89) |
| 49 | 7.62 | 6.50 | 15.0 (88) | Not Tested | EC₅₀ 35.50 (93) |
| 50 | 2.13 | 14.00 | 4.00 (90) | Not Tested | EC₅₀ 2.52 (98) |
| 51 | 11.40 | 2.42 | 15.3 (95) | Not Tested | EC₅₀ 13.00 (83) |
| 51a | 1.71 | 1.15 | 13.0 (69) | Not Tested | EC₅₀ 16.50 (100) |
| 51b | 14.20 | 1.12 | Not Tested | 48.0 (46) | IC₅₀ 50.00 (0) |
| 52a | 2.37 | 163.00 | | 983.40 (90) | IC₅₀ 118.0 (0) |
| 53 | 5.02 | 237.00 | | 512.50 (100) | IC₅₀ 486 (0) |
| 54 | 6.25 | 10.90 | 30.50 (85) | | EC₅₀ 16.3 (70.5) |
| 55 | 73.75 | 161.00 | | 344.50 (62) | IC₅₀ 403.0 (0) |
| 56 | 4% @ 1 μM | 498.50 | | 5000 (50) | Not tested |
| 57 | 14.15 | 4.69 | 247.00 (89) | | EC₅₀ 87.1 (91.5) |
| 57a | 113.00 | 4.21 | 1749 (96) | | EC₅₀ 337.0 (81) |
| 57b | 19.45 | 12.20 | 641.0 (100) | | EC₅₀ 180.0 (93) |
| 58 | 202.00 | 35.60 | 8020 (49) | | Not tested |
| 58a | 0% @ 1 μM | 46.65 | | 1962 (25) | Not tested |
| 58b | 138.00 | 29.25 | | 626.0 (94) | Not tested |
| 59 | 5.35 | 5.05 | | 812.00 (100) | IC₅₀ 166.5 (0) |
| 60 | 17.75 | 4.86 | | 1403 (100) | IC₅₀ 116.0 (0) |
| 61 | 195.5 | 2.40 | | 800.00 | IC₅₀ 593.0 (0) |
| 62 | 40% @ 1 μM | 4.55 | | 1902 (50) | Not tested |
| 63 | 46.10 | 3.56 | | 2167 (76) | IC₅₀ 449.0 (0) |
| 64 | 19.15 | 4.89 | | 395.00 (90) | IC₅₀ 288.50 (0) |
| 65 | 213.50 | 3.98 | | 91.50 (48) | IC₅₀ 358.0 (0) |
| 66 | 41% @ 1 μM | 5.65 | 7496 (38) | | Not tested |
| 67 | 44% @ 1 μM | 19.80 | | 1966 (76) | Not tested |
| 68 | 160.50 | 4.40 | | 1300 (50) | Not tested |
| 69 | 3% @ 1 μM | 15.05 | Not tested | Not tested | Not tested |
| 70 | 36% @ 1 μM | 37.20 | 1800 (22) | | Not tested |
| 71 | 44% @ 1 μM | 212.00 | | 8000 (13) | Not tested |
| 72 | 38.75 | 52.50 | | 3250 (43) | IC₅₀ 10000 (0) |
| 73 | 372.00 | 28.80 | | 5730 (40) | IC₅₀ 155.5 (0) |
| 74 | 20% @ 1 μM | 641.50 | Not tested | Not tested | Not tested |
| 75 | 0% @ 1 μM | 1028.50 | | 2152 (40) | Not tested |
| 76 | 31.30 | 52.00 | | 470.00 (68) | IC₅₀ 68.20 (0) |
| 77 | 18.05 | 60.50 | | 2572 (100) | IC₅₀ 68.35 (0) |
| 78 | 6.60 | 83.00 | | 384.00 (100) | IC₅₀ 97.10 (0) |
| 79 | 43% @ 1 μM | 4.46 | | 2656 (63) | Not tested |
| 80 | 311.50 | 0.15 | | 1000 (100) | IC₅₀ 1370 (0) |
| 81 | 4.64 | 6.15 | | 775 (100) | IC₅₀ 125.0 (0) |
| 82 | 1.64 | 3.51 | | 162.35 (100) | IC₅₀ 51.10 (0) |
| 83 | 4.80 | 14.60 | | 351.00 (100) | IC₅₀ 23.60 (0) |
| 83a | 3.43 | 13.15 | | 504.50 (100) | IC₅₀ 173.5 (0) |
| 83b | 111.50 | 16.90 | | 2503 (100) | IC₅₀ 5800 (0) |
| 84 | 0% @ 1 μM | 83.00 | Not tested | Not tested | Not tested |
| 85 | 46% @ 1 μM | 35.40 | | 3582 (72) | Not tested |
| 86 | 8.82 | 70.00 | | 773.00 (100) | IC₅₀ 228.5 (0) |
| 87 | 7.01 | 26.50 | | 513.00 (100) | IC₅₀ 172.0 (0) |
| 88 | 54.60 | 12.20 | 1781 (94) | | Not tested |
| 89 | 21.25 | 1.08 | 110.0 (62) | | Not tested |
| 90 | 0.81 | 3.37 | 34.50 (47) | 617.00 (33) | IC₅₀ 73.75 (0) |
| 91 | 2.28 | 3.33 | | 191.00 (77) | IC₅₀ 88.35 (0) |
| 92 | 47% @ 1 μM | 7.95 | 1175 (50) | | Not tested |
| 93 | 7.67 | 2.49 | | 304.0 (100) | IC₅₀ 228.0 (0) |
| 94 | 1.66 | 1.27 | | 141.20 (100) | IC₅₀ 58.40 (0) |
| 95 | 10.03 | 6.40 | | 422.00 (100) | IC₅₀ 121.10 (0) |
| 97a | 258.00 | 9.00 | Not tested | Not tested | IC₅₀ 580.5 (0) |
| 97b | 1.98 | 2.44 | | 317.00 (100) | IC₅₀ 71.05 (0) |
| 98a | 4.72 | 3.20 | | 586.00 (51) | IC₅₀ 112.95 (0) |
| 98b | 6.80 | 5.50 | | 456.00 (99) | IC₅₀ 162.0 (0) |
| 99 | 48% @ 1 μM | 524.50 | | 4018 (100) | Not tested |
| 100 | 3.85 | 141.50 | 67.60 (100) | | EC₅₀ 61.5 (87) |
| 101 | 0.50 | 642.50 | 102.70 (99) | | EC₅₀ 45.05 (68.5) |
| 102 | 3.22 | 1111.50 | | 557.00 (100) | IC₅₀ 269.50 (0) |
| 103 | 3.54 | 976.00 | | 1000 (95) | IC₅₀ 172.5 (0) |
| 104 | 16.70 | 4046.00 | Not tested | Not tested | Not tested |
| 105 | 24.30 | 236.00 | Not tested | Not tested | EC₅₀ 96.70 (81) |
| 106 | 0.61 | 53% @ 1 μM | 53.10 (47) | 111.40 (42) | EC₅₀ 2.53 (99) |
| 107 | 1.19 | 834.00 | | 253.00 (100) | IC₅₀ 45.10 (0) |
| 108 | 1.17 | 882.00 | | 94.20 (100) | IC₅₀ 45.45 (0) |
| 109 | 27.70 | 69.00 | Not tested | Not tested | Not tested |
| 110 | 6.45 | 4.59 | 244.00 (61) | 196.00 (59) | EC₅₀ 36.40 (75) |
| 111 | 26.05 | 66.00 | | 1600 (100) | IC₅₀ 1060 (0) |
| 112 | 0.74 | 61.00 | | 241.00 (100) | IC₅₀ 56.85 (0) |
| 113 | 2.02 | 51.00 | 87.20 (56) | | EC₅₀ 16.00 (91) |
| 114 | 97.05 | 69.50 | 1317.00 (64) | | EC₅₀ 244.0 (67.5) |
| 115 | 6.04 | 21.40 | 52.20 (38) | 1986 (72) | EC₅₀ 46.00 (77) |
| 116 | 1.45 | 151.00 | 61.20 (50) | 843.00 (45) | EC₅₀ 7.82 (80) |
| 117 | 3.98 | 75.50 | | 735.00 (100) | IC₅₀ 271.50 (0) |
| 118 | 1.49 | 41.45 | | 133.90 (69) | IC₅₀ 168.50 (0) |
| 119 | 36.95 | 14.20 | 3162.0 (100) | | Not tested |
| 120 | 138.85 | 22.50 | 1100.00 (67) | | Not tested |
| 121 | 67.70 | 41.85 | 3820 (70) | | Not tested |
| 122 | 0% @ 1 μM | 71.00 | Not tested | Not tested | Not tested |
| 123 | 280.00 | 3.81 | 2262 (100) | | Not tested |
| 124 | 48% @ 1 μM | 9.90 | 698.00 (41) | | Not tested |
| 125 | 86.50 | 6.80 | 2797.00 (63) | | EC₅₀ 366.50 (64) |
| 126 | 0% @ 1 μM | 17.10 | Not tested | Not tested | Not tested |
| 127 | 326.00 | 9.90 | 1294.00 (50) | | EC₅₀ 781.00 (81.5) |
| 128 | 47% @ 1 μM | 54.50 | 608.00 (59) | | Not tested |
| 129 | 170.00 | 6.80 | 9332.00 (100) | | EC₅₀ 706.5 (75.5) |
| 130 | 74.30 | 166.50 | 788.00 (39) | | EC₅₀ 14.80 (72) |
| 131 | 49% @ 1 μM | 161.50 | Not tested | Not tested | Not tested |
| 132 | 95.60 | 50.25 | 2215.00 (98) | | EC₅₀ 111.55 (63) |
| 133 | 65.15 | 23.65 | 3001.00 (100) | | EC₅₀ 128.00 (84) |
| 133a | 51.80 | 28.50 | 5000 (60) | | EC₅₀ 127.75 (53) |
| 133b | 38.60 | 43.20 | 1000 (83) | | Not tested |
| 134 | 1.75 | 8.67 | 21.80 (28) | 184.50 (69) | EC₅₀ 19.20 (81) |
| 134a | 0.66 | 2.75 | 66.00 (30) | 191.00 (60) | EC₅₀ 3.87 (89) |
| 134b | 29.60 | 7.20 | 581.00 (71) | | EC₅₀ 44.5 (66.5) |
| 135 | 19.85 | 58.00 | 441.00 (100) | | EC₅₀ 274.0 (98) |
| 136 | 27.65 | 202.50 | 1330.00 (95) | | EC₅₀ 635.0 (89) |
| 137 | 0% @ 1 μM | 421.00 | Not tested | Not tested | Not tested |
| 138 | 1.27 | 3.34 | 43.00 (100) | | Not tested |
| 139 | 4.83 | 3.90 | 180.00 (81) | | Not tested |
| 140 | 39.20 | 3.78 | 1025.00 (65) | | Not tested |
| 141 | 7.69 | 3.52 | 287.00 (99) | | Not tested |
| 142 | 4.03 | 3.88 | 122.00 (54) | | Not tested |
| 143 | 31.90 | 4.40 | 1390.00 (100) | | EC₅₀ 523.00 (91) |
| 144 | 169.50 | 5.50 | 7599 (73) | | EC₅₀ 4990 (100) |
| 145a | 288.50 | 16.10 | | 5469 (100) | IC₅₀ 668.0 (0) |
| 145b | 6.29 | 8.63 | | 1271.5 (100) | IC₅₀ 848.0 (0) |
| 147 | 3.04 | 10.65 | Not tested | Not tested | IC₅₀ 505.0 (0) |
| 148 | 0.33 | 3.25 | 8.04 (81) | | EC₅₀ 0.54 (100) |
| 149 | 2.72 | 9.25 | 44.90 (79) | | EC₅₀ 3.65 (70) |
| 150 | 0.93 | 18.65 | 13.00 (74) | | EC₅₀ 1.23 (100) |
| 151 | 101.95 | 1190.5 | Not tested | Not tested | EC₅₀ 168.0 (94) |
| 152a | 20.00 | 181.00 | | 2199 (100) | IC₅₀ 544.0 (0) |
| 152b | 322.00 | 182.00 | | 5000 (50) | IC₅₀ 727.5 (0) |
| 153a | 20.15 | 61.50 | | 3614 (100) | IC₅₀ 727.5 (0) |
| 153b | 124.00 | 122.00 | | 1446 (64) | IC₅₀ 165.5 (0) |
| 154a | 6.35 | 65.00 | | 235.00 (100) | Not tested |
| 154b | 88.15 | 217.00 | Not tested | Not tested | Not tested |
| 154c | 24.85 | 67.50 | | 1988 (100) | IC₅₀ 3090 (0) |
| 154d | 2% @ 1 μM | 4.81 | | 7071 (71) | Not tested |
| 155a | 4.16 | 48.80 | | 151.00 (100) | IC₅₀ 106.0 (0) |
| 155b | 127.50 | 144.50 | Not tested | Not tested | IC₅₀ 846.0 (0) |
| 156 | 28% @ 1 μM | 381.00 | Not tested | Not tested | Not tested |
| 157 | 83.85 | 768.00 | 1200 (70) | | IC₅₀ 10000 (0) |
| 158 | 231.50 | 120.50 | 10590 (59) | | IC₅₀ 10000 (0) |
| 159 | 30% @ 1 μM | 149.50 | | 5000 (25) | Not tested |
| 160 | 33.80 | 127.00 | 1017.00 (51) | | IC₅₀ 948.0 (0) |
| 161 | 43.50 | 233.50 | | 3000 (100) | IC₅₀ 607.00 (0) |
| 162 | 2.09 | 61.50 | 45.80 (87) | | IC₅₀ 10000 (0) |
| 163 | 0.74 | 539.50 | 4.56 (52) | | IC₅₀ 10000 (0) |
| 164 | 11.95 | 428.00 | | 445.60 (100) | IC₅₀ 116.0 (0) |
| 165 | 1.06 | 913.00 | 211.20 (22) | 115.50 (91) | IC₅₀ 52.30 (0) |
| 166 | 14.20 | 497.75 | Not tested | Not tested | IC₅₀ 300.5 (0) |
| 167 | 31.25 | 44.90 | | 1824.0 (100) | IC₅₀ 136.5 (0) |
| 168 | 3.49 | 187.50 | 242.70 (76) | | IC₅₀ 50.75 (0) |
| 169 | 1.48 | 528.50 | | 265.30 (100) | IC₅₀ 11.35 (0) |
| 170 | 0.72 | 456.50 | | 157.70 (100) | IC₅₀ 102.55 (0) |
| 171 | 4.27 | 11174.00 | | 403.90 (100) | IC₅₀ 108.5 (0) |
| 172 | 497.00 | 515.75 | Not tested | Not tested | IC₅₀ 379.0 (0) |
| 174a | 49% @ 1 μM | 855.00 | | 8000 (50) | Not tested |
| 174b | 0.85 | 192.00 | | 1445 (67) | IC₅₀ 64.1 (0) |
| 174c | 185.00 | 1098.50 | 978.00 (55) | | IC₅₀ 607.0 (0) |
| 174d | 73.35 | 322.00 | | 4750 (100) | IC₅₀ 304.0 (0) |
| 175a | 38.05 | 20.90 | | 429.00 (100) | IC₅₀ 1480 (0) |
| 175b | 31.40 | 47.60 | | 174.00 (55) | IC₅₀ 4130 (0) |
| 176a | 392.00 | 619.00 | | 4259 (92) | IC₅₀ 878.5 (0) |
| 176b | 24.60 | 165.00 | | 827.00 (88) | IC₅₀ 669.5 (0) |
| 177a | 60.30 | 5241.00 | | 2950 (100) | IC₅₀ 832.0 (0) |
| 177b | 7.34 | 180.50 | | 181.00 (74) | IC₅₀ 36.2 (0) |
| 178a | 117.50 | 347.00 | | 8398 (100) | IC₅₀ 1260 (0) |
| 178b | 9.48 | 116.00 | | 611.0 (99) | IC₅₀ 224.5 (0) |
| 179a | 48% @ 1 μM | 33.20 | | 9031 (100) | Not tested |
| 179b | 24.20 | 85.50 | | 6037 (100) | IC₅₀ 956.5 (0) |
| 180a | 19% @ 1 μM | 13.00 | 4234 (100) | | Not tested |
| 180b | 20% @ 1 μM | 2.46 | 925.00 (50.5) | | Not tested |
| 181a | 37% @ 1 μM | 26.00 | 40003 (79.4) | | Not tested |
| 181b | 46% @ 1 μM | 3.07 | | 114.70 (44.6) | Not tested |
| 182 | 236.70 | 128.00 | 892.70 (64.5) | | EC₅₀ 184.35 (65) |
| 183 | 183.45 | 53% @ 1 μM | 10000 (35) | | EC₅₀ 302.0 (65.5) |
| 184 | 46% @ 1 μM | 611.00 | Not tested | Not tested | Not tested |
| 189 | 37.40 | 27.15 | | 233.00 (100) | EC₅₀ 286.0 (77.5) |
| 190 | 16.60 | 32.90 | | 3598 (84) | EC₅₀ 321.0 (59) |
| 191 | 19.25 | 38.70 | | 204.00 (74) | Not tested |
| 192 | 39.95 | 39.30 | | 1754.00 (86) | Not tested |
| 193 | 21.55 | 15.60 | Not tested | Not tested | EC₅₀ 117.0 (89) |
| 194 | 10.65 | 70.00 | Not tested | Not tested | EC₅₀ 74.1 (81) |
| 195 | 3.25 | 12.50 | | 371.00 (100) | EC₅₀ 21.9 (58) |
| 196 | 12.83 | 123.50 | | 428.00 (100) | Not tested |
| 198 | 6.54 | 114.00 | Not tested | Not tested | EC₅₀ 67.7 (82) |
| 199 | 5.44 | 77.00 | Not tested | Not tested | EC₅₀ 74.7 (72) |
| 200 | 10.78 | 241.50 | | 1114.00 (100) | Not tested |
| 201 | 42.75 | 95.00 | Not tested | Not tested | EC₅₀ 210.0 (92) |
| 202 | 14.00 | 210.50 | Not tested | Not tested | EC₅₀ 137.0 (78) |
| 203 | 9.64 | 161.00 | | 6061 (80) | EC₅₀ 216.0 (72) |
| 204 | 17.35 | 288.50 | Not tested | Not tested | Not tested |

Like the antidepressants fluoxetine, paroxetine and sertraline, the compounds of this invention have the ability to block the reuptake of the brain neurotransmitter serotonin. They are thus useful for the treatment of diseases commonly treated by the administration of serotonin selective reuptake inhibitor (SSRI) antidepressants, such as depression, (including but not limited to major depressive disorder, childhood depression and dysthymia), anxiety, panic disorder, post-traumatic stress disorder, premenstrual dysphoric disorder (also known as premenstrual syndrome), attention deficit disorder (with and without hyperactivity), obsessive compulsive disorder, social anxiety disorder, generalized anxiety disorder, obesity, eating disorders such as anorexia nervosa, bulimia nervosa, vasomotor flushing, cocaine and alcohol addiction, sexual dysfunction, cognitive deficits resulting from neurodegenerative disorders like Alzheimer's disease, and related illnesses. Moreover, some of the compounds of this invention have potent affinity for and antagonist activity at brain 5-HT_{1A} serotonin receptors. Fairly recent clinical trials employing drug mixtures (e.g. fluoxetine and pindolol) have demonstrated a more rapid onset of antidepressant efficacy for a treatment combining SSRI activity and 5-HT_{1A} antagonism (Blier and Bergeron, J. Clin. Psychopharmacol., 1995, 15(3): 217-22; F. Artigas et al., Trends Neurosci., 1996, 19(9): 378-83; Tome et al., J. Affect Disord., 1997, 44(2-3): 101-9).

The compounds of this invention are thus interesting and useful for treating depressive illnesses.

When ranges are used herein for physical properties, such as molecular weight, or chemical properties, such as chemical formulae, all combination and subcombinations of ranges of specific embodiments therein are intended to be included.

The disclosure of each patent, patent application, and publication cited or described in this document are hereby incorporated herein by reference, in their entirety.

Those skilled in the art will appreciate that numerous changes and modifications can be made to the preferred embodiments of the invention and that such changes and modifications can be made without departing from the spirit of the invention. It is, therefore, intended that the appended claims cover all such equivalent variations as fall within the true spirit and scope of the invention.

1. A compound of formula I: 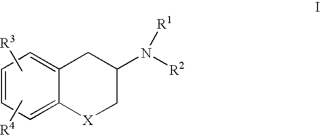
or a prodrug, stereoisomer or pharmaceutically-acceptable salt thereof;
wherein:
X is O or CH₂;
R¹ is hydrogen, alkyl, cycloalkyl or oxetane;
R² is —(CH₂)ₐ—R⁵, M, 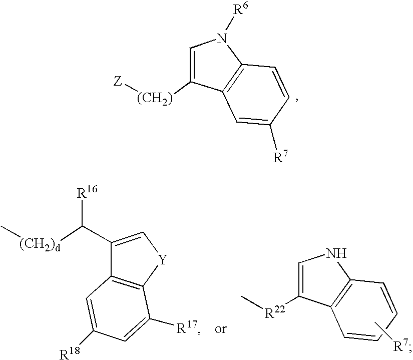
where a is an integer of 2 to 4 and R⁵ is A, B, C, D, K L, or U;
a is an integer of 2 and R⁵ is E, G or J;
a is an integer of 3 or 4 and R⁵ is P; 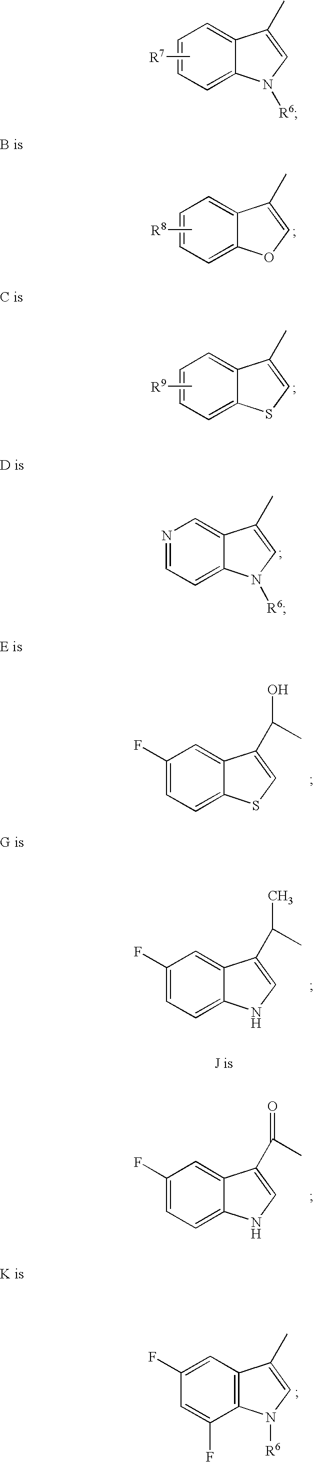 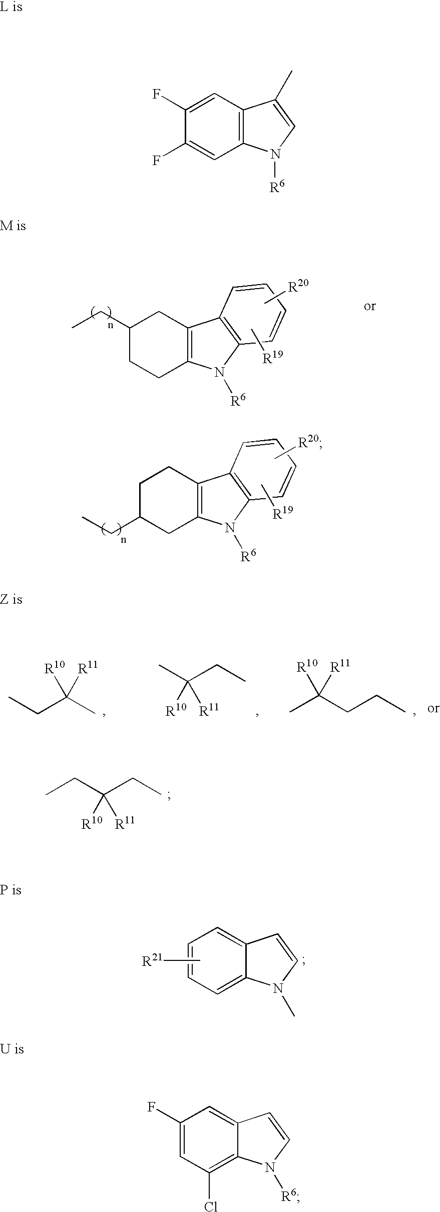
R³ is —OCH₃, —COR¹², —SO₂NR¹³R¹⁴ or heterocycle;
R⁴ is hydrogen or halo;
R⁶ is hydrogen or alkyl;
R⁷ is hydrogen, fluoro, chloro, cyano or alkoxy at either the 4-, 5-, 6-, or 7-position;
R⁸ is hydrogen, halo, C₁-C₃ alkoxy or C₁-C₃ alkyl;
R⁹ is hydrogen, halo, C₁-C₃ alkoxy or C₁-C₃ alkyl;
R¹⁰ is hydrogen and R¹¹ is methyl; or R¹⁰ and R¹¹ are methyl;
R¹² is C₁-C₄ alkyl, alkoxy or NR¹³R¹⁴;
R¹³ and R¹⁴ are independently hydrogen, alkyl, cycloalkyl, methylcyclopropyl, phenyl, or benzyl;
R¹⁹ and R²⁰ are independently hydrogen, fluoro, chloro, cyano, or C₁-C₆ alkyl at either the 5-, 6-, 7-, or 8-position;
R²¹ is hydrogen or fluoro at either the 4-, 5-, 6-, or 7-position;
R²² is a 3- to 7-membered ring;
n is an integer of 1 or 2;
Y is O, S, or NH;
wherein, when Y is O, then
R¹⁶ is hydrogen;
R¹⁷ is hydrogen or OCH₃;
R¹⁸ is hydrogen; and
d is an integer of 2 or 3;
when Y is S, then
R¹⁶ is hydrogen or hydroxyl;
R¹⁷ is hydrogen;
R¹⁸ is hydrogen or fluoro; and
d is an integer of 2;
when Y is NH, then
R¹⁶ is keto or methyl;
R¹⁷ is hydrogen;
R¹⁸ is fluoro; and
d is an integer of 2.

2. A compound according to claim 1 of formula Ia: 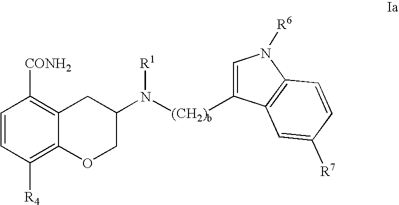
wherein:
R¹ is ethyl, propyl, cyclobutyl, or methylcyclopropyl;
R⁴ is chloro or fluoro;
R⁶ is hydrogen or methyl;
R⁷ is hydrogen, fluoro or cyano; and
b is an integer of 3 or 4.

3. A compound according to claim 1 of formula Ib: 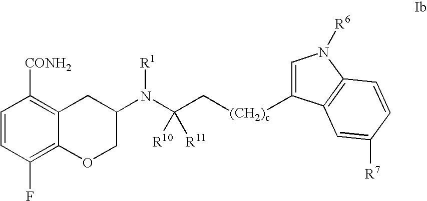
wherein:
R¹ is ethyl, propyl, cyclobutyl, or methylcyclopropyl;
R⁶ is hydrogen or methyl;
R⁷ is hydrogen, fluoro or cyano;
R¹⁰ is hydrogen;
R¹¹ is methyl; and
c is an integer of 1 or 2.

4. A compound according to claim 1 of formula Ic: 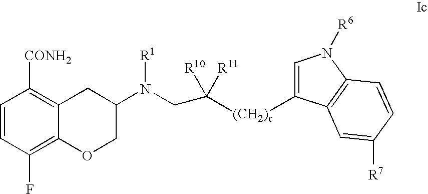
wherein:
R¹ is ethyl, propyl, cyclobutyl, or methylcyclopropyl;
R⁶ is hydrogen or methyl;
R⁷ is hydrogen, fluoro or cyano;
R¹⁰ is hydrogen;
R¹¹ is methyl; and
c is an integer of 1 or 2.

5. A compound according to claim 1 of formula Id: 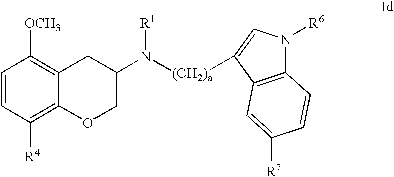
wherein:
R¹ is methyl, ethyl, propyl, isopropyl, 2-methylpropyl, cyclopropyl, cyclobutyl, cyclopentyl, methylcyclopropyl, or methylcyclobutyl;
R⁴ is hydrogen or fluoro;
R⁶ is hydrogen or methyl;
R⁷ is hydrogen, fluoro or cyano; and
a is an integer of 2 to 4.

6. A compound according to claim 1 of formula Ie: 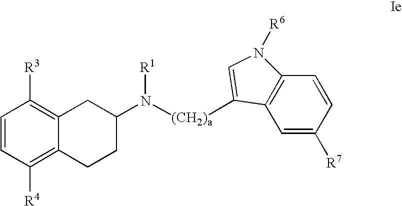
wherein:
R¹ is ethyl, propyl, cyclobutyl, or methylcyclopropyl;
R⁶ is hydrogen or methyl;
R⁷ is hydrogen or fluoro;
R³ is —OCH₃ or —COR¹²;
R¹² is C₁-C₄ alkyl, alkoxy, or NR¹³R¹⁴;
R¹³ and R¹⁴ are independently hydrogen or alkyl;
R⁴ is hydrogen or fluoro; and
a is an integer of 2 to 4.

7. A compound according to claim 1 of formula If: 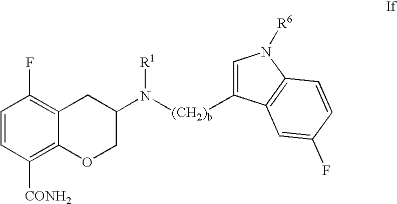
wherein:
R¹ is propyl, cyclobutyl or methylcyclopropyl;
R⁶ is hydrogen or methyl; and
b is an integer of 3 or 4.

8. A compound according to claim 1 of formula Ig: 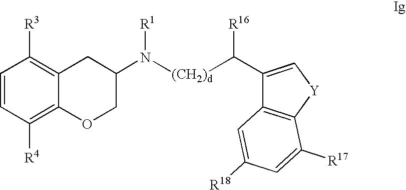
wherein:
R¹ is hydrogen, ethyl, propyl, cyclobutyl or methylcyclopropyl;
R³ is —OCH₃ or —CONH₂;
R⁴ is hydrogen or fluoro;
Y is O, S or NH;
wherein, when Y is O, then
R¹⁶ is hydrogen;
R¹⁷ is hydrogen or OCH₃;
R¹⁸ is hydrogen; and
d is an integer of 1, 2 or 3;
when Y is S, then
R¹⁶ is hydrogen or hydroxyl;
R¹⁷ is hydrogen,
R¹⁸ is hydrogen or fluoro; and
d is an integer of 2;
when Y is NH, then
R¹⁶ is keto or methyl;
R¹⁷ is hydrogen;
R¹⁸ is fluoro; and
d is an integer of 2.

9. A compound according to claim 1 of formula Ih: 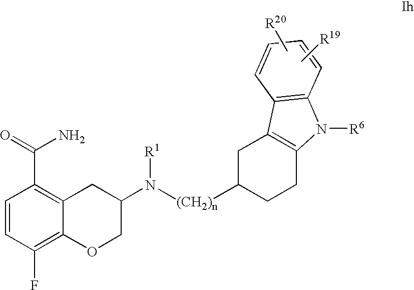
wherein:
R¹ is hydrogen, propyl, methylcyclopropyl and cyclobutyl;
R⁶ is hydrogen or methyl;
R¹⁹ and R²⁰ are independently hydrogen, fluoro or cyano at either the 5-, 6-, 7- or 8-position; and
n is an integer of 1 or 2.

10. A compound according to claim 1 of formula Ij: 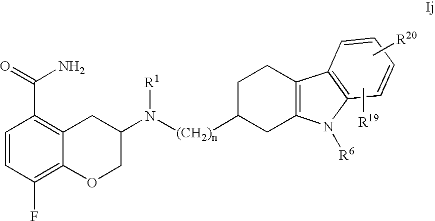
wherein:
R¹ is hydrogen, propyl, methylcyclopropyl and cyclobutyl;
R⁶ is hydrogen or methyl;
R¹⁹ and R²⁰ are independently hydrogen, fluoro or cyano at either the 5-, 6-, 7- or 8-position; and
n is an integer of 1 or 2.

11. A compound according to claim 1 of formula Ik: 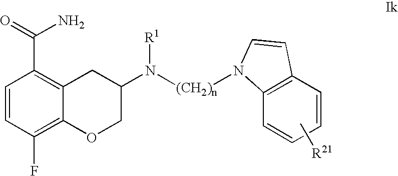
wherein:
R¹ is hydrogen, ethyl, propyl, cyclobutyl, or methylcyclopropyl;
R²¹ is hydrogen or fluoro at either the 4-, 5-, 6- or 7-position; and
b is an integer of 3 or 4.

12. A compound according to claim 1 of formula Im: 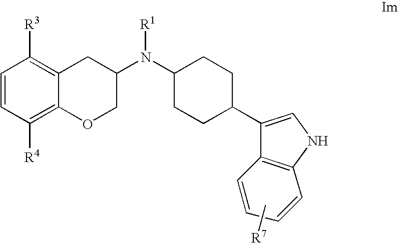
wherein:
R¹ is hydrogen, ethyl, propyl, methylcyclopropyl or cyclobutyl;
R³ is —OCH₃, or CONH₂;
R⁴ is hydrogen or fluoro; and
R⁷ is hydrogen or fluoro at either the 4-, 5-, 6-, or 7-position.

13. A compound according to claim 1, wherein said R¹ is hydrogen, alkyl, cycloalkyl and methylcyclopropyl.

14. A compound according to claim 1, wherein said R² is —(CH)ₐ—R⁵ or.

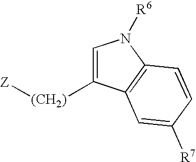

15. A compound according to claim 14, wherein said R⁵ is A or K.

16. A compound according to claim 1, wherein said R³ is —COR¹².

17. A compound according to claim 1, wherein said R⁴ is fluoro or chloro.

18. A compound according to claim 1, wherein said R⁶ is hydrogen or methyl.

19. A compound according to claim 1, wherein said R⁷ is fluoro, cyano, or hydrogen.

20. A compound according to claim 1, wherein said R⁸ is hydrogen or —OCH₃.

21. A compound according to claim 1, wherein said R⁹ is hydrogen or fluoro.

22. A compound according to claim 1, wherein said R¹² is —OCH₃, NH₂ or NHMe.

23. A compound according to claim 1, wherein said R¹³ is hydrogen.

24. A compound according to claim 1, wherein said R¹⁴ is hydrogen or methyl.

25. A compound according to claim 1, wherein said Z is

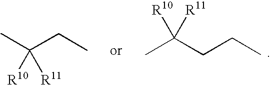

26. A compound according to claim 1, wherein said X is oxygen or methylene.

27. A compound according to claim 1, wherein said R¹⁶ is hydrogen when Y is O or S.

28. A compound according to claim 1, wherein said R¹⁶ is methyl when Y is NH.

29. A compound according to claim 1, wherein said R¹⁷ is hydrogen when Y is O, S, or NH.

30. A compound according to claim 1, wherein said R¹⁷ is methoxy when Y is O.

31. A compound according to claim 1, wherein said R¹⁹ is fluoro.

32. A compound according to claim 1, wherein said R²⁰ is fluoro.

33. A compound according to claim 1, wherein said R²¹ is fluoro.

34. A compound according to claim 1, wherein said R²² is a 4-, 5-, or 6-membered ring.

35. A compound according to claim 1, wherein said compound is

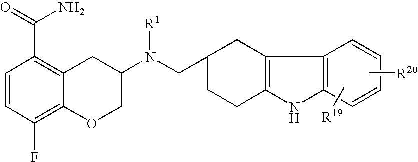

36. A compound according to claim 1, wherein said compound is:
8-fluoro-3-{[3-(5-fluoro-1H-indol-3-yl)propyl]amino}chromane-5-carboxamide;
(+)-8-fluoro-3-{[3-(5-fluoro-1H-indol-3-yl)propyl]amino}chromane-5-carboxamide;
(−)-8-fluoro-3-{[3-(5-fluoro-1H-indol-3-yl)propyl]amino}chromane-5-carboxamide;
8-fluoro-3-{[3-(5-fluoro-1H-indol-3-yl)propyl](propyl)amino}chromane-5-carboxamide;
(−)-8-fluoro-3-{[3-(5-fluoro-1H-indol-3-yl)propyl](propyl)amino}-3,4-dihydro-2H-chromene-5-carboxamide;
(+)-8-fluoro-3-{[3-(5-fluoro-1H-indol-3-yl)propyl](propyl)amino}-3,4-dihydro-2H-chromene-5-carboxamide;
(−)-3-{ethyl[3-(5-fluoro-1H-indol-3-yl)propyl]amino-8-fluoro-3,4-dihydro-2H-chromene-5-carboxamide;
(+)-3-{ethyl[3-(5-fluoro-1H-indol-3-yl)propyl]amino}-8-fluoro-3,4-dihydro-2H-chromene-5-carboxamide;
3-{cyclobutyl[3-(5-fluoro-1H-indol-3-yl)propyl]amino}-8-fluorochromane-5-carboxamide;
(+)-3-{cyclobutyl[3-(5-fluoro-1H-indol-3-yl)propyl)amino)-8-fluorochromane-5-carboxamide;
(−)₃-{cyclobutyl[3-(5-fluoro-1H-indol-3-yl)propyl]amino}-8-fluorochromane-5-carboxamide;
3-{cyclopropylmethyl[3-(5-fluoro-1H-indol-3-yl)propyl]amino}-8-fluorochromane-5-carboxamide;
(−)-3-{cyclopropylmethyl[3-(5-fluoro-1H-indol-3-yl)propyl]amino}-8-fluoro chromane-5-carboxamide;
(+)-3-{cyclopropylmethyl[3-(5-fluoro-1H-indol-3-yl)propyl]amino}-8-fluoro chromane-5-carboxamide;
3-{(1-cyclopropylethyl)[3-(5-fluoro-1H-indol-3-yl)propyl]amino}-8-fluorochromane-5-carboxamide;
8-chloro-3-{cyclobutyl[3-(5-fluoro-1H-indol-3-yl)propyl]amino}chromane-5-carboxamide;
3-{cyclobutyl[3-(5-fluoro-1H-indol-3-yl)-3-oxopropyl]amino}-8-fluorochromane-5-carboxamide;
(−)-3-{cyclobutyl[3-(5,7-difluoro-1H-indol-3-yl)propyl]amino}-8-fluorochromane-5-carboxamide;
(+)-3-{cyclobutyl[3-(5,7-difluoro-1H-indol-3-yl)propyl]amino}-8-fluorochromane-5-carboxamide;
methyl-8-fluoro-3-{[3-(5-fluoro-1H-indol-3-yl)propyl]amino}chromane-5-carboxylate;
methyl-3-{cyclobutyl[3-(5-fluoro-1H-indol-3-yl)propyl]amino}-8-fluorochromane-5-carboxylate;
8-fluoro-3-{[3-(5-fluoro-1H-indol-3-yl)-1-methylpropyl](propyl)amino}-3,4-dihydro-2H-chromene-5-carboxamide;
(3R)-8-fluoro-3-[[(1S)-3-(5-fluoro-1H-indol-3-yl)-1-methylpropyl](propyl)amino]chromane-5-carboxamide;
(3R)-8-fluoro-3-[[(1R)-3-(5-fluoro-1H-indol-3-yl)-1-methylpropyl] (propyl)amino]chromane-5-carboxamide;
(3S)-8-fluoro-3-[[(1R)-3-(5-fluoro-1H-indol-3-yl)-1-methylpropyl](propyl)amino]chromane-5-carboxamide;
(3S)-8-fluoro-3-[[(1S)-3-(5-fluoro-1H-indol-3-yl)-1-methylpropyl](propyl)amino]chromane-5-carboxamide;
3-{[3-(5-cyano-1H-indol-3-yl)-1-methylpropyl](propyl)amino}-8-fluoro-3,4-dihydro-2H-chromene-5-carboxamide;
(3R)-3-[[(1R)-3-(5-cyano-1H-indol-3-yl)-1-methylpropyl](propyl)amino]-8-fluorochromane-5-carboxamide;
(3S)-3-[[(1S)-3-(5-cyano-1H-indol-3-yl)-1-methylpropyl](propyl)amino]-8-fluorochromane-5-carboxamide;
(3R)-3-[[(1S)-3-(5-cyano-1H-indol-3-yl)-1-methylpropyl](propyl)amino]-8-fluorochromane-5-carboxamide;
(3S)-3-[[(1R)-3-(5-cyano-1H-indol-3-yl)-1-methylpropyl](propyl)amino]-8-fluorochromane-5-carboxamide;
8-fluoro-3-{[4-(5-fluoro-1H-indol-3-yl)-1-methylbutyl](propyl)amino}-chromane-5-carboxamide;
(+)-8-fluoro-3-{[4-(5-fluoro-1H-indol-3-yl)-1-methylbutyl](propyl)amino}-chromane-5-carboxamide;
(−)-8-fluoro-3-{[4-(5-fluoro-1H-indol-3-yl)-1-methylbutyl](propyl)amino}-chromane-5-carboxamide;
(−)-3-{(cyclopropylmethyl)[3-(5-fluoro-1H-indol-3-yl]-1-methylpropyl]amino}-8-fluorochromane-5-carboxamide;
(+)-3-{(cyclopropylmethyl)[3-(5-fluoro-1H-indol-3-yl)-1-methylpropyl]amino}-8-fluorochromane-5-carboxamide;
(−)-3-{ethyl[3-(5-fluoro-1H-indol-3-yl)-1-methylpropyl]amino}-8-fluorochromane-5-carboxamide;
(+)-3-{ethyl[3-(5-fluoro-1H-indol-3-yl)-1-methylpropyl]amino}-8-fluorochromane-5-carboxamide;
8-fluoro-3-{[3-(5-fluoro-1-benzothien-3-yl)-3-hydroxypropyl](propyl)amino}chromane-5-carboxamide;
N-[3-(1-benzothien-3-yl)propyl]-N-ethyl-5-methoxychroman-3-amine;
N-[3-(5-fluoro-1-benzothien-3-yl)propyl]-5-methoxy-N-propylchroman-3-amine;
3-{[3-(1-benzofuran-3-yl)propyl](propyl)amino}-8-fluorochromane-5-carboxamide;
N-(3-(1-benzofuran-3-yl)propyl]-N-ethyl-5-methoxychroman-3-amine;
N-[4-(1-benzofuran-3-yl)butyl]-N-ethyl-N-(5-methoxy-3,4-dihydro-2H-chromene-3-yl) amine;
[3-(5-fluoro-1H-indol-3-yl)propyl]-N-(5-methoxy-chroman-3-yl)propylamine;
[3-(5-fluoro-1H-indol-3-yl)propyl]((3R)-5-methoxychroman-3-yl)propylamine;
[3-(5-fluoro-1H-indol-3-yl)propyl]((3S)-5-methoxychroman-3-yl)propylamine;
[3-(5-fluoro-1H-indol-3-yl)propyl]-(8-fluoro-5-methoxychroman-3-yl)propylamine;
(3S)-8-fluoro-N-[3-(5-fluoro-1H-indol-3-yl)propyl]-5-methoxy-N-propylchroman-3-amine;
(3R)-8-fluoro-N-[3-(5-fluoro-1H-indol-3-yl)propyl]-5-methoxy-N-propylchroman-3-amine;
N-[2-(5-fluoro-1H-indol-3-yl)ethyl]-5-methoxy-N-propylchroman-3-amine;
N-[4-(5-fluoro-1H-indol-3-yl)butyl]-5-methoxy-N-propylchroman-3-amine;
N-ethyl-N-[3-(5-fluoro-1H-indol-3-yl)propyl]-5-methoxychroman-3-amine;
N-ethyl-N-[4-(5-fluoro-1H-indol-3-yl)butyl]-5-methoxychroman-3-amine;
N-[3-(5-fluoro-1H-indol-3-yl)propyl]-5-methoxy-N-methylchroman-3-amine;
N-cyclobutyl-N-[3-(5-,fluoro-1H-indol-3-yl)propyl]-5-methoxychroman-3-amine;
(3R)-N-cyclobutyl-N-[3-(5-fluoro-1H-indol-3-yl)propyl]-5-methoxy-3,4-dihydro-2H-chromen-3-amine;
N-cyclobutyl-N-[4-(5-fluoro-1H-indol-3-yl)butyl]-N-(5-methoxy-3,4-dihydro-2H-chromen-3-yl)amine;
N-(cyclopropylmethyl)-N-[3-(5-fluoro-1H-indol-3-yl)propyl]-N-(5-methoxy-3,4-dihydro-2H-chromen-3-yl)amine;
N-(cyclopropylmethyl)-N-[3-(5-fluoro-1-methyl-1H-indol-3-yl)propyl]-N-(5-methoxy-3,4-dihydro-2H-chromen-3-yl)amine;
N-(cyclopentyl)-N-[3-(5-fluoro-1H-indol-3yl)propyl]-N-(5-methoxy-3,4-dihydro-2H-chromen-3-yl)amine;
N-[3-(5-fluoro-1H-indol-3-yl)propyl]-N-isopropyl-N-(5-methoxy-3,4-dihydro-2H-chromen-3-yl) amine;
N-cyclopropyl-N-[3-(5-fluoro-1H-indol-3-yl)propyl]-N-(5-methoxy-3,4-dihydro-2H-chromen-3-yl)amine;
N-(cyclobutylmethyl)-N-[3-(5-fluoro-1H-indol-3-yl)propyl]-N-(5-methoxy-3,4-dihydro-2H-chromen-3-yl)amine;
N-(cyclopropylmethyl)-N-[3-(1H-indol-3-yl)propyl]-N-(5-methoxy-3,4-dihydro-2H-chromen-3-yl)amine;
N-cyclobutyl-N-[3-(1H-indol-3-yl)propyl]-N-(5-methoxy-3,4-dihydro-2H-chromen-3-yl)amine;
3-{3-[(cyclopropylmethyl)(5-methoxy-3,4-dihydro-2H-chromen-3-yl)amino]propyl}-1H-indole-5-carbonitrile;
3-{3-[cyclobutyl(5-methoxy-3,4-dihydro-2H-chromen-3-yl)amino]propyl}-1H-indole-5-carbonitrile;
N-[3-(5-fluoro-1H-indol-3yl)propyl]-N-(8-methoxy-1,2,3,4-tetrahydronaphthalen-2-yl)-N-propylamine;
(−)-N-[3-(5-fluoro-1H-indol-3yl)propyl]-8-methoxy-N-propyl-1,2,3,4-tetrahydronaphthalen-2-amine;
(+)-N-[3-(5-fluoro-1H-indol-3yl)propyl]-8-methoxy-N-propyl-1,2,3,4-tetrahydronaphthalen-2-amine;
N-[2-(5-fluoro-1H-indol-3-yl)ethyl]-N-(8-methoxy-1,2,3,4-tetrahydronaphthalen-2-yl)-N-propylamine;
N-ethyl-N-[2-(5-fluoro-1H-indol-3-yl)ethyl]-N-(8-methoxy-1,2,3,4-tetrahydronaphthalen-2-yl)amine;
N-[3-(1H-indol-3-yl)propyl]-8-methoxy-N-propyl-1,2,3,4-tetrahydronaphthalen-2-amine;
N-[3-(5-fluoro-1H-indol-3-yl)propyl]-N-(5-fluoro-8-methoxy-1,2,3,4-tetrahydronaphthalen-2-yl-N-propylamine;
(+)-5-fluoro-N-[3-(5-fluoro-1H-indol-3-yl)propyl]-8-methoxy-N-propyl-1,2,3,4-tetrahydro-2-naphthalenlamine;
(−)-5-fluoro-N-[3-(5-fluoro-1H-indol-3-yl)propyl]-8-methoxy-N-propyl-1,2,3,4-tetrahydro-2-naphthalenamine;
8-fluoro-3-{[3-(6-fluoro-1H-indol-3-yl)propyl]amino}chromane-5-carboxamide;
3-{(cyclopropylmethyl)[3-(6-fluoro-1H-indol-3-yl)propyl]amino}-8-fluorochromane-5-carboxamide;
3-{cyclobutyl[3-(6-fluoro-1H-indol-3-yl)propyl]amino}-8-fluorochromane-5-carboxamide;
Methyl 8-fluoro-3-{[3-(5-fluoro-1H-indol-3-yl)propyl]amino}chromane-5-carboxylate;
Methyl 3-{cyclobutyl[3-(5-fluoro-1H-indol-3-yl)propyl]amino}-8-fluorochromane-5-carboxylate;
3-{cyclobutyl[3-(5-fluoro-1H-indol-3-yl)propyl]amino}-8-fluorochromane-5-carboxylic acid;
Methyl 8-fluoro-3-{[4-(5-fluoro-1H-indol-3-yl)butyl]amino}chromane-5-carboxylate;
Methyl (3S)-8-fluoro-3-{[4-(5-fluoro-1H-indol-3-yl)butyl]amino}chromane-5-carboxylate;
Methyl (3R)-8-fluoro-3-{[4-(5-fluoro-1H-indol-3-yl)butyl]amino}chromane-5-carboxylate;
Methyl 3-{cyclobutyl[4-(5-fluoro-1H-indol-3-yl)butyl]amino}-8-fluorochromane-5-carboxylate;
Methyl (3S)-3-{cyclobutyl[4-(5-fluoro-1H-indol-3-yl)butyl]amino}-8-fluorochromane-5-carboxylate;
Methyl (3R)-3-{cyclobutyl[4-(5-fluoro-1H-indol-3-yl)butyl]amino}-8-fluorochromane-5-carboxylate;
3-{cyclobutyl[3-(5-fluoro-1H-indol-3-yl)propyl]amino}-8-fluoro-N-methylchromane-5-carboxamide;
3-{cyclobutyl[3-(5-fluoro-1H-indol-3-yl)propyl]amino}-N-ethyl-8-fluorochromane-5-carboxamide;
3-{cyclobutyl[3-(5-fluoro-1H-indol-3-yl)propyl]amino}-8-fluoro-N-propylchromane-5-carboxamide;
N-butyl-3-{cyclobutyl[3-(5-fluoro-1H-indol-3-yl)propyl]amino}-8-fluorochromane-5-carboxamide;
3-{cyclobutyl[3-(5-fluoro-1H-indol-3-yl)propyl]amino}-8-fluoro-N-isopropylchromane-5-carboxamide;
3-{cyclobutyl[3-(5-fluoro-1H-indol-3-yl)propyl]amino}-N-cyclopropyl-8-fluorochromane-5-carboxamide;
N-cyclobutyl-3-{cyclobutyl[3-(5-fluoro-1H-indol-3-yl)propyl]amino}-8-fluorochromane-5-carboxamide;
3-{cyclobutyl[3-(5-fluoro-1H-indol-3-yl)propyl]amino}-N-cyclopentyl-8-fluorochromane-5-carboxamide;
3-{cyclobutyl[3-(5-fluoro-1H-indol-3-yl)propyl]amino}-N-cyclohexyl-8-fluorochromane-5-carboxamide;
3-{cyclobutyl[3-(5-fluoro-1H-indol-3-yl)propyl]amino}-N-(cyclopropylmethyl)-8-fluorochromane-5-carboxamide;
N-benzyl-3-{cyclobutyl[3-(5-fluoro-1H-indol-3-yl)propyl]amino}-8-fluorochromane-5-carboxamide;
3-{cyclobutyl[3-(5-fluoro-1H-indol-3-yl)propyl]amino}-8-fluoro-N-phenylchromane-5-carboxamide
8-fluoro-3-[[3-(5-fluoro-1H-indol-3-yl)propyl](pentyl)amino]chromane-5-carboxamide;
3-{butyl[3-(5-fluoro-1H-indol-3-yl)propyl]amino}-8-fluorochromane-5-carboxamide;
3-{cyclobutyl[3-(5-fluoro-1H-indol-3-yl)propyl]amino}-8-fluoro-N,N-dimethylchromane-5-carboxamide;
3-{benzyl[3-(5-fluoro-1H-indol-3-yl)propyl]amino}-8-fluorochromane-5-carboxamide;
8-fluoro-3-{[2-(5-fluoro-1H-indol-3-yl)ethyl]amino)chromane-5-carboxamide;
3-{ethyl[3-(5-fluoro-1H-indol-3-yl)propyl]amino}-8-fluorochromane-5-carboxamide;
8-fluoro-3-[[2-(5-fluoro-1H-indol-3-yl)ethyl](propyl)amimo]chromane-5-carboxamide;
3-{(cyclopropylmethyl)[2-(5-fluoro-1H-indol-3-yl)ethyl]amino}-8-fluorochromane-5-carboxamide;
8-fluoro-3-{[4-(5-fluoro-1H-indol-3-yl)butyl]amino}chromane-5-carboxamide;
3-{ethyl[4-(5-fluoro-1H-indol-3-yl)butyl]amino}-8-fluorochromane-5-carboxamide;
8-fluoro-3-[[4-(5-fluoro-1H-indol-3-yl)butyl](propyl)amino]chromane-5-carboxamide;
3-{(cyclopropylmethyl)[4-(5-fluoro-1H-indol-3-yl)butyl]amino}-8-fluorochromane-5-carboxamide;
3-{cyclobutyl[4-(5-fluoro-1H-indol-3-yl)butyl]amino}-8-fluorochromane-5-carboxamide;
(3R)-3-{cyclobutyl[4-(5-fluoro-1H-indol-3-yl)butyl]amino}-8-fluorochromane-5-carboxamide;
(3S)-3-{cyclobutyl[4-(5-fluoro-1H-indol-3-yl)butyl]amino}-8-fluorochromane-5-carboxamide;
8-fluoro-3-{[2-(5-fluoro-1H-indol-3-yl)ethyl]amino}-N-methylchromane-5-carboxamide;
3-{ethyl[2-(5-fluoro-1H-indol-3-yl)ethyl]amino}-8-fluoro-N-methylchromane-5-carboxamide;
3-{(cyclopropylmethyl)[2-(5-fluoro-1H-indol-3-yl)ethyl]amino}-8-fluoro-N-methylchromane-5-carboxamide;
3-{cyclobutyl[2-(5-fluoro-1H-indol-3-yl)ethyl]amino}-8-fluoro-N-methylchromane-5-carboxamide;
8-fluoro-3-{[3-(5-fluoro-1H-indol-3-yl)propyl]amino}-N-methylchromane-5-carboxamide;
3-{ethyl[3-(5-fluoro-1H-indol-3-yl)propyl]amino}-8-fluoro-N-methylchromane-5-carboxamide;
8-fluoro-3-[[3-(5-fluoro-1H-indol-3-yl)propyl](propyl)amino]-N-methylchromane-5-carboxamide;
3-{(cyclopropylmethyl)[3-(5-fluoro-1H-indol-3-yl)propyl]amino}-8-fluoro-N-methylchromane-5-carboxamide;
8-fluoro-3-{[4-(5-fluoro-1H-indol-3-yl)butyl]amino}-N-methylchromane-5-carboxamide;
8-fluoro-3-[[4-(5-fluoro-1H-indol-3-yl)butyl](propyl)amino]-N-methylchromane-5-carboxamide;
3-{(cyclopropylmethyl)[4-(5-fluoro-1H-indol-3-yl)butyl]amino}-8-fluoro-N-methylchromane-5-carboxamide;
3-{cyclobutyl[4-(5-fluoro-1H-indol-3-yl)butyl]amino}-8-fluoro-N-methylchromane-5-carboxamide;
3-{[3-(5-cyano-1H-indol-3-yl)propyl]amino}-8-fluorochromane-5-carboxamide;
3-[[3-(5-cyano-1H-indol-3-yl)propyl](cyclobutyl)amino]-8-fluorochromane-5-carboxamide;
(3S)-3-[[3-(5-cyano-1H-indol-3-yl)propyl](cyclobutyl)amino]-8-fluorochromane-5-carboxamide;
(3R)-3-[[3-(5-cyano-1H-indol-3-yl)propyl](cyclobutyl)amino]-8-fluorochromane-5-carboxamide;
3-[[3-(5-cyano-1H-indol-3-yl)propyl](cyclopropylmethyl)amino]-8-fluorochromane-5-carboxamide;
(3S)-3-[[3-(5-cyano-1H-indol-3-yl)propyl](cyclopropylmethyl)amino]-8-fluorochromane-5-carboxamide
(3R)-3-[[3-(5-cyano-1H-indol-3-yl)propyl](cyclopropylmethyl)amino]-8-fluorochromane-5-carboxamide;
8-fluoro-3-{[3-(7-methoxy-1H-indol-3-yl)propyl]amino}chromane-5-carboxamide;
8-fluoro-3-[[3-(7-methoxy-1H-indol-3-yl)propyl](propyl)amino]chromane-5-carboxamide;
3-{ethyl[3-(7-methoxy-1H-indol-3-yl)propyl]amino}-8-fluorochromane-5-carboxamide;
3-{cyclobutyl[3-(7-methoxy-1H-indol-3-yl)propyl]amino}-8-fluorochromane-5-carboxamide;
3-{(cyclopropylmethyl)[3-(7-methoxy-1H-indol-3-yl)propyl]amino}-8-fluorochromane-5-carboxamide;
8-fluoro-3-{[3-(5-methoxy-1H-indol-3-yl)propyl]amino}chromane-5-carboxamide;
3-{ethyl[3-(5-methoxy-1H-indol-3-yl)propyl]amino}-8-fluorochromane-5-carboxamide;
8-fluoro-3-[[3-(5-methoxy-1H-indol-3-yl)propyl](propyl)amino]chromane-5-carboxamide;
3-{cyclobutyl[3-(5-methoxy-1H-indol-3-yl)propyl]amino}-8-fluorochromane-5-carboxamide;
3-{(cyclopropylmethyl)[3-(5-methoxy-1H-indol-3-yl)propyl]amino}-8-fluorochromane-5-carboxamide;
3-{[3-(7-chloro-1H-indol-3-yl)propyl]amino}-8-fluorochromane-5-carboxamide;
3-[[3-(7-chloro-1H-indol-3-yl)propyl](ethyl)amino]-8-fluorochromane-5-carboxamide;
3-[[3-(7-chloro-1H-indol-3-yl)propyl](cyclobutyl)amino]-8-fluorochromane-5-carboxamide
3-[[3-(7-chloro-1H-indol-3-yl)propyl](cyclopropylmethyl)amino]-8-fluorochromane-5-carboxamide
3-[[3-(7-chloro-1H-indol-3-yl)propyl](propyl)amino]-8-fluorochromane-5-carboxamide;
3-{[3-(5-chloro-1H-indol-3-yl)propyl]amino}-8-fluorochromane-5-carboxamide;
3-[[3-(5-chloro-1H-indol-3-yl)propyl](ethyl)amino]-8-fluorochromane-5-carboxamide;
3-[[3-(5-chloro-1H-indol-3-yl)propyl](propyl)amino]-8-fluorochromane-5-carboxamide;
3-[[3-(5-chloro-1H-indol-3-yl)propyl](cyclobutyl)amino]-8-fluorochromane-5-carboxamide;
3-[[3-(5-chloro-1H-indol-3-yl)propyl](cyclopropylmethyl)amino]-8-fluorochromane-5-carboxamide;
5-fluoro-3-{[3-(5-fluoro-1H-indol-3-yl)propyl]amino}chromane-8-carboxamide;
5-fluoro-3-[[3-(5-fluoro-1H-indol-3-yl)propyl](propyl)amino]chromane-8-carboxamide;
3-{(cyclopropylmethyl)[3-(5-fluoro-1H-indol-3-yl)propyl]amino}-5-fluorochromane-8-carboxamide;
3-{cyclobutyl[3-(5-fluoro-1H-indol-3-yl)propyl]amino}-5-fluorochromane-8-carboxamide;
5-fluoro-3-{[4-(5-fluoro-H-indol-3-yl)butyl]amino}chromane-8-carboxamide;
5-fluoro-3-[[4-(5-fluoro-1H-indol-3-yl)butyl](propyl)amino]chromane-8-carboxamide;
3-{(cyclopropylmethyl)[4-(5-fluoro-1H-indol-3-yl)butyl]amino}-5-fluorochromane-8-carboxamide;
3-{cyclobutyl[4-(5-fluoro-1H-indol-3-yl)butyl]amino}-5-fluorochromane-8-carboxamide;
3-{ethyl[4-(5-fluoro-1H-indol-3-yl)butyl]amino}-5-fluorochromane-8-carboxamide;
3-({[3-(5,7-difluoro-1H-indol-3-yl)propyl]amino}-5-fluorochromane-8-carboxamide;
3-[[3-(5,7-difluoro-1H-indol-3-yl)propyl](propyl)amino]-5-fluorochromane-8-carboxamide;
3-{(cyclopropylmethyl)[3-(5,7-difluoro-1H-indol-3-yl)propyl]amino}-5-fluorochromane-8-carboxamide;
3-{cyclobutyl[3-(5,7-difluoro-1H-indol-3-yl)propyl]amino}-5-fluorochromane-78-carboxamide;
3-[[3-(5,7-difluoro-1H-indol-3-yl)propyl](ethyl)amino]-5-fluorochromane-8-carboxamide;
3-{[3-(5-fluoro-1H-indol-3-yl)propyl]amino}chromane-5-carboxamide;
(3S)-3-{[3-(5-fluoro-1H-indol-3-yl)propyl]amino}chromane-5-carboxamide;
(3R)-3-{[3-(5-fluoro-1H-indol-3-yl)propyl]amino}chromane-5-carboxamide;
3-{cyclobutyl[3-(5-fluoro-1H-indol-3-yl)propyl]amino}chromane-5-carboxamide;
(−)-3-{cyclobutyl[3-(5-fluoro-1H-indol-3-yl)propyl]amino}chromane-5-carboxamide;
(+)-3-{cyclobutyl[3-(5-fluoro-1H-indol-3-yl)propyl]amino}chromane-5-carboxamide;
Methyl 3-{[3-(5,7-difluoro-1H-indol-3-yl)propyl]amino}chromane-5-carboxylate;
Methyl 3-{cyclobutyl[3-(5,7-difluoro-1H-indol-3-yl)propyl]amino}chromane-5-carboxylate;
3-{cyclobutyl[3-(5,7-difluoro-1H-indol-3-yl)propyl]amino}chromane-5-carboxylic acid;
3-{cyclobutyl[3-(5,7-difluoro-1H-indol-3-yl)propyl]amino}-N-methylchromane-5-carboxamide;
3-{cyclobutyl[3-(5,7-difluoro-1H-indol-3-yl)propyl]amino}-N-ethylchromane-5-carboxamide;
3-{cyclobutyl[3-(5,7-difluoro-1H-indol-3-yl)propyl]amino}-N-propylchromane-5-carboxamide;
3-{cyclobutyl[3-(5,7-difluoro-1H-indol-3-yl)propyl]amino}-N-isopropylchromane-5-carboxamide;
3-{cyclobutyl[3-(5,7-difluoro-1H-indol-3-yl)propyl]amino}-N-cyclopropylchromane-5-carboxamide;
N-cyclobutyl-3-{cyclobutyl[3-(5,7-difluoro-1H-indol-3-yl)propyl]amino}chromane-5-carboxamide;
3-{cyclobutyl[3-(5,7-difluoro-1H-indol-3-yl)propyl]amino}-N-(cyclopropylmethyl)chromane-5-carboxamide;
(3S)-3-{cyclobutyl[4-(5-fluoro-1H-indol-3-yl)butyl]amino}-8-fluoro-N-methylchromane-5-carboxamide;
(3R)-3-{cyclobutyl[4-(5-fluoro-1H-indol-3-yl)butyl]amino}-8-fluoro-N-methylchromane-5-carboxamide;
(3R)-3-{[3-(5,7-difluoro-1H-indol-3-yl)propyl]amino}-8-fluorochromane-5-carboxamide;
(3R)-3-{(cyclopropylmethyl)[3-(5,7-difluoro-1H-indol-3-yl)propyl]amino}-8-fluorochromane-5-carboxamide;
3-{(cyclopropylmethyl)[3-(5-fluoro-1H-indol-3-yl)propyl]amino}chromane-5-carboxamide;
3-{ethyl[3-(5-fluoro-1H-indol-3-yl)propyl]amino}chromane-5-carboxamide;
3-[[3-(5-fluoro-1H-indol-3-yl)propyl](propyl)amino]chromane-5-carboxamide;
3-[[3-(5-fluoro-1H-indol-3-yl)propyl](isobutyl)amino]chromane-5-carboxamide;
8-fluoro-3-{[(3R)-3-(5-fluoro-1H-indol-3-yl)butyl]amino}chromane-5-carboxamide;
(3R)-3-{cyclobutyl[(3R)-3-(5-fluoro-1H-indol-3-yl)butyl]amino}-8-fluorochromane-5-carboxamide;
(3S)-3-{cyclobutyl[(3R)-3-(5-fluoro-1H-indol-3-yl)butyl]amino}-8-fluorochromane-5-carboxamide;
8-fluoro-3-{[(3S)-3-(5-fluoro-1H-indol-3-yl)butyl]amino}chromane-5-carboxamide;
(3R)-3-{cyclobutyl[(3S)-3-(5-fluoro-1H-indol-3-yl)butyl]amino}-8-fluorochromane-5-carboxamide;
(3S)-3-{cyclobutyl[(3S)-3-(5-fluoro-1H-indol-3-yl)butyl]amino}-8-fluorochromane-5-carboxamide;
3-[3-(5,7-Difluoro-1H-indol-3-yl)-1-methyl-propylamino]-8-fluoro-chroman-5-carboxylic acid amide;
(3R)-3-{(cyclopropylmethyl)[(1R)-3-(5,7-difluoro-1H-indol-3-yl)-1-methylpropyl]amino}-8-fluorochromane-5-carboxamide;
(3R)-3-{(cyclopropylmethyl)[(1S)-3-(5,7-difluoro-1H-indol-3-yl)-1-methylpropyl]amino}-8-fluorochromane-5-carboxamide;
(3S)-3-{(cyclopropylmethyl)[(1S)-3-(5,7-difluoro-1H-indol-3-yl)-1-methylpropyl]amino}-8-fluorochromane-5-carboxamide;
(3S)-3-{(cyclopropylmethyl)[(1R)-3-(5,7-difluoro-1H-indol-3-yl)-1-methylpropyl]amino}-8-fluorochromane-5-carboxamide;
(3R)-8-fluoro-3-{[3-(5-fluoro-1H-indol-3-yl)-2-methylpropyl]amino}chromane-5-carboxamide;
(3R)-3-{(cyclopropylmethyl)[(2S)-3-(5-fluoro-1H-indol-3-yl)-2-methylpropyl]amino}-8-fluorochromane-5-carboxamide;
(3R)-3-{(cyclopropylmethyl)[(2R)-3-(5-fluoro-1H-indol-3-yl)-2-methylpropyl]amino}-8-fluorochromane-5-carboxamide;
5-fluoro-1H-indol-3-yl)-2-methylpropyl]amino}-8-fluorochromane-5-carboxamide;
8-fluoro-3-{[2-(7-methoxy-1-benzofuran-3-yl)ethyl]amino}chromane-5-carboxamide;
3-{ethyl[2-(7-methoxy-1-benzofuran-3-yl)ethyl]amino}-8-fluorochromane-5-carboxamide;
8-fluoro-3-[[2-(7-methoxy-1-benzofuran-3-yl)ethyl](propyl)amino]chromane-5-carboxamide;
3-{cyclobutyl[2-(7-methoxy-1-benzofuran-3-yl)ethyl]amino}-8-fluorochromane-5-carboxamide;
3-{(cyclopropylmethyl)[2-(7-methoxy-1-benzofuran-3-yl)ethyl]amino}-8-fluorochromane-5-carboxamide;
8-fluoro-3-{[3-(7-methoxy-1-benzofuran-3-yl)propyl]amino}chromane-5-carboxamide;
3-{ethyl[3-(7-methoxy-1-benzofuran-3-yl)propyl]amino}-8-fluorochromane-5-carboxamide;
8-fluoro-3-[[3-(7-methoxy-1-benzofuran-3-yl)propyl](propyl)amino]-chromane-5-carboxamide;
3-{cyclobutyl[3-(7-methoxy-1-benzofuran-3-yl)propyl]amino}-8-fluorochromane-5-carboxamide;
3-{(cyclopropylmethyl)[3-(7-methoxy-1-benzofuran-3-yl)propyl]amino}-8-fluorochromane-5-carboxamide;
3-{butyl[3-(7-methoxy-1-benzofuran-3-yl)propyl]amino}-8-fluorochromane-5-carboxamide;
8-fluoro-3-{[4-(7-methoxy-1-benzofuran-3-yl)butyl]amino}chromane-5-carboxamide;
3-{ethyl[4-(7-methoxy-1-benzofuran-3-yl)butyl]amino}-8-fluorochromane-5-carboxamide;
8-fluoro-3-[[4-(7-methoxy-1-benzofuran-3-yl)butyl](propyl)amino]chromane-5-carboxamide;
3-{(cyclopropylmethyl)[4-(7-methoxy-1-benzofuran-3-yl)butyl]amino}-8-fluorochromane-5-carboxamide;
3-{cyclobutyl[4-(7-methoxy-1-benzofuran-3-yl)butyl]amino}-8-fluorochromane-5-carboxamide;
(3R)-8-fluoro-3-[[4-(7-methoxy-1-benzofuran-3-yl)butyl](propyl)amino]chromane-5-carboxamide;
8-fluoro-3-{[(6-fluoro-2,3,4,9-tetrahydro-1H-carbazol-3-yl)methyl]amino}chromane-5-carboxamide;
(3R)-3-(cyclobutyl{[(3S)-6-fluoro-2,3,4,9-tetrahydro-1H-carbazol-3-yl]methyl}amino)-8-fluorochromane-5-carboxamide;
(3R)-3-(cyclobutyl{[(3R)-6-fluoro-2,3,4,9-tetrahydro-1H-carbazol-3-yl]methyl}amino)-8-fluorochromane-5-carboxamide;
(3S)-3-(cyclobutyl{[(3S)-6-fluoro-2,3,4,9-tetrahydro-1H-carbazol-3-yl]methyl}amino)-8-fluorochromane-5-carboxamide;
(3S)-3-(cyclobutyl{[(3R)-6-fluoro-2,3,4,9-tetrahydro-1H-carbazol-3-yl]methyl}amino)-8-fluorochromane-5-carboxamide;
(−)-(3R)-8-fluoro-3-({[6-fluoro-2,3,4,9-tetrahydro-1H-carbazol-3-yl]methyl}amino)chromane-5-carboxamide;
(+)-(3R)-8-fluoro-3-({[6-fluoro-2,3,4,9-tetrahydro-1H-carbazol-3-yl]methyl}amino)chromane-5-carboxamide;
(−)-(3R)-3-(ethyl{[6-fluoro-2,3,4,9-tetrahydro-1H-carbazol-3-yl]methyl}amino)-8-fluorochromane-5-carboxamide;
(+)-(3R)-3-(ethyl{[6-fluoro-2,3,4,9-tetrahydro-1H-carbazol-3-yl]methyl}amino)-8-fluorochromane-5-carboxamide;
(−)-(3R)-8-fluoro-3-[{[6-fluoro-2,3,4,9-tetrahydro-1H-carbazol-3-yl]methyl}(propyl)amino]chromane-5-carboxamide;
(+)-(3R)-8-fluoro-3-[{[6-fluoro-2,3,4,9-tetrahydro-1H-carbazol-3-yl]methyl}(propyl)amino]chromane-5-carboxamide;
(−)-(3R)-3-((cyclopropylmethyl){[6-fluoro-2,3,4,9-tetrahydro-1H-carbazol-3-yl]methyl}amino)-8-fluorochromane-5-carboxamide;
(+)-(3R)-3-((cyclopropylmethyl){[6-fluoro-2,3,4,9-tetrahydro-1H-carbazol-3-yl]methyl}amino)-8-fluorochromane-5-carboxamide;
(+)-(3R)-8-fluoro-3-({[6-fluoro-2,3,4,9-tetrahydro-1H-carbazol-2-yl]methyl}amino)chromane-5-carboxamide;
(−)-(3R)-8-fluoro-3-({[6-fluoro-2,3,4,9-tetrahydro-1H-carbazol-2-yl]methyl}amino)chromane-5-carboxamide;
3-[(1,4-cis)-4-(5-Methoxy-chroman-3-ylamino)-cyclohexyl]-1H-indole-5-carbonitrile;
3-[(1,4-trans)-4-(5-Methoxy-chroman-3-ylamino)-cyclohexyl]-1H-indole-5-carbonitrile;
cis-N-[4-(5-fluoro-1H-indol-3-yl)cyclohexyl]-5-methoxychroman-3-amine;
trans-N-[4-(5-fluoro-1H-indol-3-yl)cyclohexyl]-5-methoxychroman-3-amine;
cis-N-[4-(5-fluoro-1H-indol-3-yl)cyclohexyl]-5-methoxy-N-propylchroman-3-amine;
trans-N-[4-(5-fluoro-1H-indol-3-yl)cyclohexyl]-5-methoxy-N-propylchroman-3-amine;
8-Fluoro-3-{[3-(1H-indol-1-yl)propyl]amino}chromane-5-carboxamide;
8-Fluoro-3-[4-(indol-1-yl)-butylamino]-chroman-5-carboxylic acid amide;
8-Fluoro-3-[4-(5-fluoro-indol-1-yl)-butylamino]-chroman-5-carboxylic acid amide;
8-Fluoro-3-[4-(6-fluoro-indol-1-yl)-butylamino]-chroman-5-carboxylic acid amide;
8-Fluoro-3-{[4-(7-fluoro-1H-indol-1-yl)butyl]amino}chromane-5-carboxamide,
3-{Ethyl[4-(7-fluoro-1H-indol-1-yl)butyl]amino}-8-fluorochromane-5-carboxamide;
8-Fluoro-3-[[4-(7-fluoro-1H-indol-1-yl)butyl](propyl)amino}chromane-5-carboxamide;
3-{(Cyclopropylmethyl)[4-(7-fluoro-1H-indol-1-yl)butyl]amino}-8-fluorochromane-5-carboxamide;
3-{Cyclobutyl[4-(7-fluoro-1H-indol-1-yl)butyl]amino}-8-fluorochromane-5-carboxamide;
3-{Ethyl[4-(6-fluoro-1H-indol-1-yl)butyl]amino}-8-fluorochromane-5-carboxamide;
8-Fluoro-3-[[4-(6-fluoro-1H-indol-1-yl)butyl](propyl)amino]chromane-5-carboxamide;
3-{(Cyclopropylmethyl)[4-(6-fluoro-1H-indol-1-yl)butyl]amino}-8-fluorochromane-5-carboxamide;
3-{Cyclobutyl[4-(6-fluoro-1H-indol-1-yl)butyl]amino}-8-fluorochromane-5-carboxamide;
3-{Ethyl[4-(5-fluoro-1H-indol-1-yl)butyl]amino}-8-fluorochromane-5-carboxamide;
8-Fluoro-3-[[4-(5-fluoro-1H-indol-1-yl)butyl](propyl)amino]chromane-5-carboxamide;
3-{(Cyclopropylmethyl)[4-(5-fluoro-1H-indol-1-yl)butyl]amino}-8-fluorochromane-5-carboxamide;
3-{Cyclobutyl[4-(5-fluoro-1H-indol-1-yl)butyl]amino}-8-fluorochromane-5-carboxamide;
3-{Ethyl[4-(4-fluoro-1H-indol-1-yl)butyl]amino}-8-fluorochromane-5-carboxamide,
8-Fluoro-3-t [4-(4-fluoro-1H-indol-1-yl)butyl]propyl)amino]chromane-5-carboxamide;
3-{(Cyclopropylmethyl)[4-(4-fluoro-1H-indol-1-yl)butyl]amino}-8-fluorochromane-5-carboxamide; or
3-{Cyclobutyl[4-(4-fluoro-1H-indol-1-yl)butyl]amino}-8-fluorochromane-5-carboxamide.

37. A compound comprising:

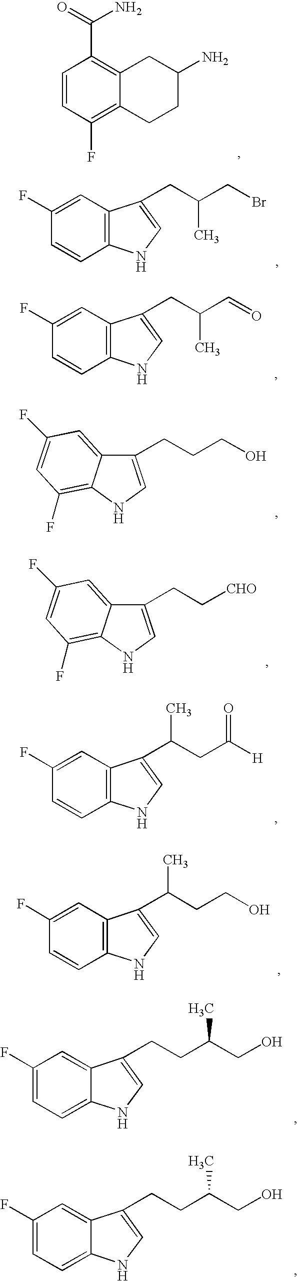

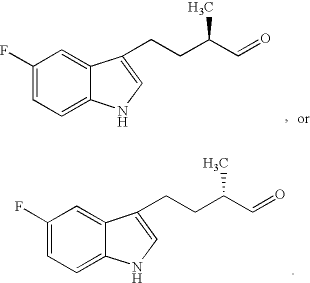

38. A composition comprising:
the compound of claim 1; and
one or more pharmaceutically-acceptable carriers.

39. A method of treating a patient suspected of suffering from a serotonin disorder, comprising the step of administering to the patient a therapeutically effective amount of the compound of claim 1.

40. The method according to claim 39, wherein said serotonin disorder is depression, anxiety, panic disorder, post-traumatic stress disorder, premenstrual dysphoric disorder, attention deficit disorder, obsessive compulsive disorder, social anxiety disorder, generalized anxiety disorder, obesity, anorexia nervosa, bulimia nervosa, vasomotor flushing, cocaine addiction, alcohol addiction, sexual dysfunction, or a cognitive deficit resulting from a neurodegenerative disorder.

41. The method according to claim 39, wherein said serotonin disorder is depression.

42. The method according to claim 39, wherein said serotonin disorder is anxiety.

43. A method of agonizing 5-HT_{1A} receptors in a patient in need thereof, comprising the step of administering to the patient a therapeutically effective amount of a compound of claim 1.

44. A method of antagonizing 5-HT_{1A} receptors in a patient in need thereof, comprising the step of administering to the patient a therapeutically effective amount of a compound of claim 1.

45. A method of inhibiting the reuptake of serotonin in a patient in need thereof, comprising administering to the patient a therapeutically effective amount of a compound of claim 1.
